(19) United States
(12) Patent Application Publication
(10) Pub. No.: US 2024/0269305 A1
(43) Pub. Date: Aug. 15, 2024

(54) STING AGONIST SUITABLE AS PAYLOAD OF ANTIBODY DRUG CONJUGATE

(71) Applicant: HITGEN INC., Chengdu (CN)

(72) Inventors: Jin LI, Chengdu (CN); Fei PAN, Chengdu (CN); Rong MA, Chengdu (CN); Sen GAO, Chengdu (CN); Rongfeng WU, Chengdu (CN); Xiaoguang BAI, Chengdu (CN)

(21) Appl. No.: 18/586,429

(22) Filed: Feb. 24, 2024

Related U.S. Application Data
(63) Continuation of application No. PCT/CN2022/114884, filed on Aug. 25, 2022.

(30) Foreign Application Priority Data
Aug. 26, 2021 (CN) 202110986776.5

(51) Int. Cl. A61K 47/68

(52) U.S. Cl. CPC A61K 47/6803; A61K 47/6889

(57) ABSTRACT

Disclosed are a compound-linker conjugate and a class of compounds having the STING activation activity, as well as their applications in the preparation of antibody drug conjugates. The compound-linker conjugate is represented by

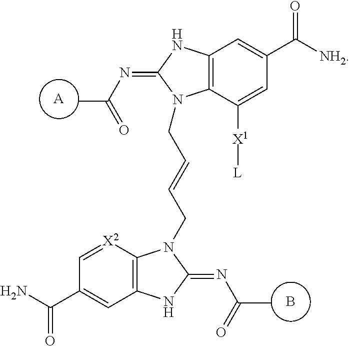

The compounds are represented by

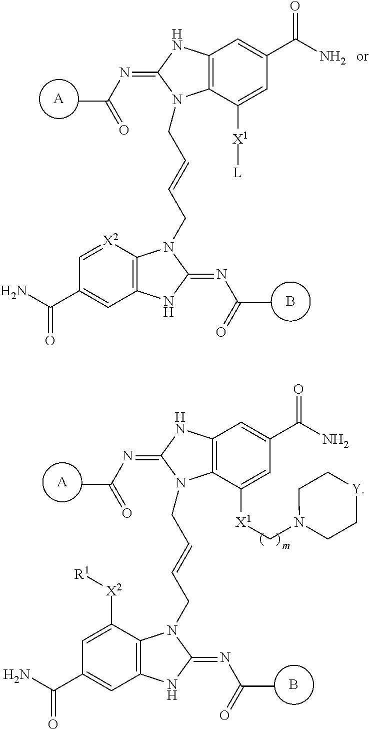

The compounds are suitable as payloads of the antibody drug conjugates.

### CROSS-REFERENCE TO RELATED APPLICATIONS

This application is a continuation of International Patent Application No. PCT/CN2022/114884, filed on Aug. 25, 2022, which claims the benefit of priority from Chinese Patent Application No. 202110986776.5, filed on Aug. 26, 2021. The content of the aforementioned application, including any intervening amendments made thereto, is incorporated herein by reference in its entirety.

### TECHNICAL FIELD

The present disclosure relates to antibody-drug conjugates, and more particularly to a class of small-molecule STING agonists suitable as payloads of the antibody drug conjugates and an application thereof in the preparation of antibody drug conjugates.

### BACKGROUND

STING (stimulator of interferon genes, e.g., TMEM173 and MITA) is a key intracellular molecule in response to DNA invasion, which can recognize the signal from cytoplasmic DNA receptors under the stimulation of cytoplasmic DNA, and further plays a key role in triggering the production of interferon. The DNA recognition receptor in host cells transmits the signal to the node molecule STING after recognizing the exogenous or endogenous “non-self” DNA, and then the SING will be rapidly dimerized and transferred from the endoplasmic reticulum to the nucleosome. IRF3 and NKκB pathways are upregulated by the activation of STING, thereby triggering the production of interferon-β and other cytokines.

Antibody drug conjugate combines the unique targeting ability of antibody with the cytotoxic effect of drug via a chemical linker with unstable bond. Therefore, the antibody drug conjugate represents a class of important biological drugs which are designed for targeted therapy of subjects in various disease symptoms. By means of the target specificity of the antibody, the payload is selectively delivered to the target site and released, thereby enabling the high-dose targeted drug therapies.

### SUMMARY

The present disclosure provides a compound-linker conjugate of formula (I):

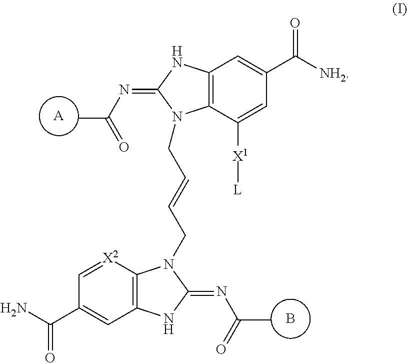

wherein
ring A and ring B are each independently

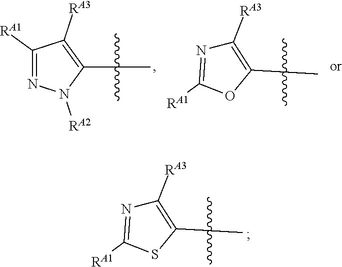

R^{A1} and R^{A3} are independently selected from the group consisting of hydrogen, halogen, —CN, unsubstituted —C₁₋₆ alkyl and halogenated —C₁₋₆ alkyl;
R^{A2} is selected from the group consisting of hydrogen, unsubstituted —C₁₋₆ alkyl and halogenated —C₁₋₆ alkyl;
X¹ is O or S;
X² is N or CR^{X};
R^{X} is selected from the group consisting of hydrogen, halogen, —CN, unsubstituted —C₁₋₆ alkyl, halogenated —C₁₋₆ alkyl, —R¹, —OR¹, —SR¹ and —NR¹R^{1′};
R¹ and R^{1′} are each independently selected from the group consisting of hydrogen, —C₁₋₈ alkyl, —C₁₋₈ alkylene-NR²R^{2′}, —C₁₋₈ alkylene-OR², —C₀₋₈ alkylene-(3-10 membered cycloalkyl), —C₀₋₈ alkylene-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in an alkylene group are adapted to be replaced with an oxygen atom;
R² and R^{2′} are each independently selected from the group consisting of hydrogen, —C₁₋₆ alkyl, —C₀₋₈ alkylene-(3-10 membered cycloalkyl), —C₀₋₈ alkyl-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in an alkylene group are adapted to be replaced with an oxygen atom;
L is selected from -(L¹)_{q}-W;
q is an integer selected from 1-100;
L¹ is each independently selected from the group consisting of CRR, C(O), O, S, S(O), S(O)₂, NR, —CR═CR—, —C≡C—, P(O)R, P(O)OR, 3-10 membered cycloalkane, 3-10 membered heterocycloalkane, 5-10 membered aromatic ring, 5-10 membered aromatic heterocyclic ring, 5-12 membered spiro ring, 5-12 membered spiro heterocyclic ring, 5-12 membered bridged ring and 5-12 membered bridged heterocyclic ring; wherein cycloalkane, heterocycloalkane, aromatic ring, aromatic heterocyclic ring, spiro ring, spiro heterocyclic ring, bridged ring, and bridged heterocyclic ring are unsubstituted or substituted by one, two or three R^{L1};
R^{L1} is each independently selected from the group consisting of hydrogen, halogen, ═O, cyano, nitro, unsubstituted —C₁₋₆ alkyl, halogenated —C₁₋₆ alkyl, —OR, —NRR, —C₀₋₄ alkylene-(3-10 membered cycloalkyl) and —C₀₋₄ alkylene-(3-10 membered heterocycloalkyl);
R is each independently selected from the group consisting of hydrogen, halogen, cyano, unsubstituted —C₁₋₆ alkyl, unsubstituted —C₂₋₆ alkenyl, unsubstituted —C₂₋₆ alkynyl, halogenated —C₁₋₆ alkyl, halogenated —C₂₋₆ alkenyl, halogenated —C₂₋₆ alkynyl, —C₀₋₄ alkylene-OR^{R1}, —C₀₋₄ alkylene-OC(O)R^{R1}, —C₀₋₄ alkylene-SR^{R1}, —C₀₋₄ alkylene-S(O)₂R^{R1}, —C₀₋₄ alkylene-S(O)R^{R1}, —C₀₋₄ alkylene-S(O)₂NR^{R1}R^{R2}, —C₀₋₄ alkylene-S(O)NR^{R1}R^{R2}, —C₀₋₄ alkylene-S(O)(NH)R^{R1}, —C₀₋₄ alkylene-S(O)(NH)NR^{R1}R^{R2}, —C₀₋₄ alkylene-C(O)R^{R1}, —C₀₋₄ alkylene-C(O)OR^{R1}, —C₀₋₄ alkylene-C(O)NR^{R1}R^{R2}, —C₀₋₄ alkylene-NR^{R1}R^{R2}, —C₀₋₄ alkylene-NR^{R1}C(O)R^{R2}, —C₀₋₄ alkylene-NR^{R1}S(O)₂R^{R2}, C₀₋₄ alkylene-NR^{R1}S(O)R^{R2}, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring);
R^{R1} and R^{R2} are independently selected from the group consisting of hydrogen, unsubstituted —C₁₋₆ alkyl, unsubstituted —C₂₋₆ alkenyl, unsubstituted —C₂₋₆ alkynyl, —OH, —NH₂, halogenated —C₁₋₆ alkyl, halogenated —C₂₋₆ alkenyl, halogenated —C₂₋₆ alkynyl, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring); and
W is

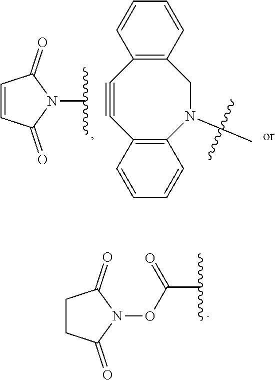

In some embodiments, L is -M-L^{A}-L^{B}-W;
M is selected from the group consisting of —C₁₋₈ alkylene-, —C₀₋₈ alkylene-(3-10 membered cycloalkyl)-, —C₀₋₈ alkylene-(3-10 membered heterocycloalkyl)-, —C₀₋₈ alkylene-(5-12 membered spirocycloalkyl)-, —C₀₋₈ alkylene-(5-12 membered spiroheterocycloalkyl)-, —C₀₋₈ alkylene-(5-12 membered bridged cycloalkyl)- and —C₀₋₈ alkylene-(5-12 membered bridged heterocycloalkyl)-; wherein one or two carbon atoms in an alkylene group are adapted to be replaced with an oxygen atom;
L^{A} is selected from the group consisting of —C(O)—C₁₋₈ alkylene-NH—, —C(O)—C₁₋₈ alkylene-N(C₁₋₆ alkyl)-, —C(O)O—C₁₋₈ alkylene-NH—, —C(O)O—C₁₋₈ alkylene-N(C₁₋₆ alkyl)-, —C(O)NH—C₁₋₈ alkylene-NH—, —C(O)NH—C₁₋₈ alkylene-N(C₁₋₆ alkyl)-, —C₁₋₈ alkylene-NH—, —C₁₋₈ alkylene-N(C₁₋₆ alkyl)-, —C(O)—C₁₋₈ alkylene-(3-10 membered cycloalkyl)-, —C(O)O—C₁₋₈ alkylene-(3-10 membered cycloalkyl)-, —C(O)NH—C₁₋₈ alkylene-(3-10 membered cycloalkyl)-, —C₁₋₈ alkylene-(3-10 membered cycloalkyl)-, —C(O)—C₁₋₈ alkylene-(3-10 membered heterocycloalkyl)-, —C(O)O—C₁₋₈ alkylene-(3-10 membered heterocycloalkyl)-, —C(O)NH—C₁₋₈ alkylene-(3-10 membered heterocycloalkyl)-, —C₁₋₈ alkylene-(3-10 membered heterocycloalkyl)-, —C(O)—C₁₋₈ alkylene-(5-12 membered spirocycloalkyl)-, —C(O)O—C₁₋₈ alkylene-(5-12 membered spirocycloalkyl)-, —C(O)NH—C₁₋₈ alkylene-(5-12 membered spirocycloalkyl)-, —C₁₋₈ alkylene-(5-12 membered spirocycloalkyl)-, —C(O)—C₁₋₈ alkylene-(5-12 membered spiroheterocycloalkyl)-, —C(O)O—C₁₋₈ alkylene-(5-12 membered spiroheterocycloalkyl)-, —C(O)NH—C₁₋₈ alkylene-(5-12 membered spiroheterocycloalkyl)-, —C₁₋₈ alkylene-(5-12 membered spiroheterocycloalkyl), —C(O)—C₁₋₈ alkylene-(5-12 membered bridged cycloalkyl)-, —C(O)O—C₁₋₈ alkylene-(5-12 membered bridged cycloalkyl)-, —C(O)NH—C₁₋₈ alkylene-(5-12 membered bridged cycloalkyl)-, —C₁₋₈ alkylene-(5-12 membered bridged cycloalkyl)-, —C(O)—C₁₋₈ alkylene-(5-12 membered bridged heterocycloalkyl)-, —C(O)O—C₁₋₈ alkylene-(5-12 membered bridged heterocycloalkyl)-, —C(O)NH—C₁₋₈ alkylene-(5-12 membered bridged heterocycloalkyl)-, —C₁₋₈ alkylene-(5-12 membered bridged heterocycloalkyl)-, —C(O)—C₁₋₈ alkylene-O—, —C(O)O—C₁₋₈ alkylene-O—, —C(O)NH—C₁₋₈ alkylene-O—, —C₁₋₈ alkylene-O—, —S(O)₂-C₁₋₈ alkylene-NH—, —S(O)₂-C₁₋₈ alkylene-N(C₁₋₆ alkyl)-, —S(O)₂O—C₁₋₈ alkylene-NH—, —S(O)₂O—C₁₋₈ alkylene-N(C₁₋₆ alkyl)-, —S(O)₂NH—C₁₋₈ alkylene-NH—, —S(O)₂NH—C₁₋₈ alkylene-N(C₁₋₆ alkyl)-, —S(O)₂—C₁₋₈ alkylene-(3-10 membered cycloalkyl)-, —S(O)₂O—C₁₋₈ alkylene-(3-10 membered cycloalkyl)-, —S(O)₂NH—C₁₋₈ alkylene-(3-10 membered cycloalkyl)-, —S(O)₂—C₁₋₈ alkylene-(3-10 membered heterocycloalkyl)-, —S(O)₂O—C₁₋₈ alkylene-(3-10 membered heterocycloalkyl)-, —S(O)₂NH—C₁₋₈ alkylene-(3-10 membered heterocycloalkyl)-, —S(O)₂—C₁₋₈ alkylene-(5-12 membered spirocycloalkyl)-, —S(O)₂O—C₁₋₈ alkylene-(5-12 membered spirocycloalkyl)-, —S(O)₂NH—C₁₋₈ alkylene-(5-12 membered spirocycloalkyl)-, —S(O)₂—C₁₋₈ alkylene-(5-12 membered spiroheterocycloalkyl)-, —S(O)₂O—C₁₋₈ alkylene-(5-12 membered spiroheterocycloalkyl)-, —S(O)₂NH—C₁₋₈ alkylene-(5-12 membered spiroheterocycloalkyl)-, —S(O)₂—C₁₋₈ alkylene-(5-12 membered bridged cycloalkyl)-, —S(O)₂O—C₁₋₈ alkylene-(5-12 membered bridged cycloalkyl)-, —S(O)₂NH—C₁₋₈ alkylene-(5-12 membered bridged cycloalkyl)-, —S(O)₂—C₁₋₈ alkylene-(5-12 membered bridged heterocycloalkyl)-, —S(O)₂O—C₁₋₈ alkylene-(5-12 membered bridged heterocycloalkyl)-, —S(O)₂NH—C₁₋₈ alkylene-(5-12 membered bridged heterocycloalkyl)-, —S(O)₂—C₁₋₈ alkylene-O—, —S(O)₂O—C₁₋₈ alkylene-O—, —S(O)₂NH—C₁₋₈ alkylene-O— and a chemical bond; wherein one or two carbon atoms in an alkylene group are adapted to be replaced with an oxygen atom;
L^{B} is -(L¹)ₚ-;
p is an integer selected from 1-50;
L¹ is each independently selected from the group consisting of CRR, C(O), O, S, S(O), S(O)₂, NR, —CR═CR—, —C≡C—, P(O)R, P(O)OR, 3-10 membered cycloalkane, 3-10 membered heterocycloalkane, 5-10 membered aromatic ring, 5-10 membered aromatic heterocyclic ring, 5-12 membered spiro ring, 5-12 membered spiro heterocyclic ring, 5-12 membered bridged ring and 5-12 membered bridged heterocyclic ring; wherein cycloalkane, heterocycloalkane, aromatic ring, aromatic heterocyclic ring, spiro ring, spiro heterocyclic ring, bridged ring and bridged heterocyclic ring are unsubstituted or substituted by one, two or three R^{L1};
R^{L1} is each independently selected from the group consisting of hydrogen, halogen, ═O, cyano, nitro, unsubstituted —C₁₋₆ alkyl, halogenated —C₁₋₆ alkyl, —OR, —NRR, —C₀₋₄ alkylene-(3-10 membered cycloalkyl) and —C₀₋₄ alkylene-(3-10 membered heterocycloalkyl);
R is each independently selected from the group consisting of hydrogen, halogen, cyano, unsubstituted —C₁₋₆ alkyl, unsubstituted —C₂₋₆ alkenyl, unsubstituted —C₂₋₆ alkynyl, halogenated —C₁₋₆ alkyl, halogenated —C₂₋₆ alkenyl, halogenated —C₂₋₆ alkynyl, —C₀₋₄ alkylene-OR^{R1}, —C₀₋₄ alkylene-OC(O)R^{R1}, —C₀₋₄ alkylene-SR^{R1}, —C₀₋₄ alkylene-S(O)₂R^{R1}, —C₀₋₄ alkylene-S(O)R^{R1}, —C₀₋₄ alkylene-S(O)₂NR^{R1}R^{R2}, —C₀₋₄ alkylene-S(O)NR^{R1}R^{R2}, —C₀₋₄ alkylene-S(O)(NH)R^{R1}, —C₀₋₄ alkylene-S(O)(NH)NR^{R1}R^{R2}, —C₀₋₄ alkylene-C(O)R^{R1}, —C₀₋₄ alkylene-C(O)OR^{R1}, —C₀₋₄ alkylene-C(O)NR^{R1}R^{R2}, —C₀₋₄ alkylene-NR^{R1}R^{R2}, —C₀₋₄, alkylene-NR^{R1}C(O)R^{R2}, C₀₋₄ alkylene-NR^{R1}S(O)₂R^{R2}, C₀₋₄ alkylene-NR^{R1}S(O)R^{R2}, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring);
R^{R1} and R^{R2} are independently selected from the group consisting of hydrogen, unsubstituted —C₁₋₆ alkyl, unsubstituted —C₂₋₆ alkenyl, unsubstituted —C₂₋₆ alkynyl, —OH, —NH₂, halogenated —C₁₋₆ alkyl, halogenated —C₂₋₆ alkenyl, halogenated —C₂₋₆ alkynyl —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring); and
W is

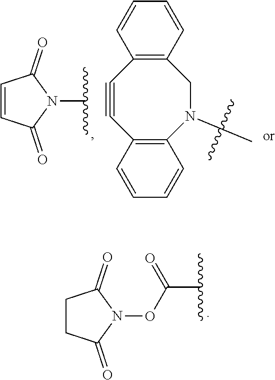

In some embodiments, M is selected from the group consisting of

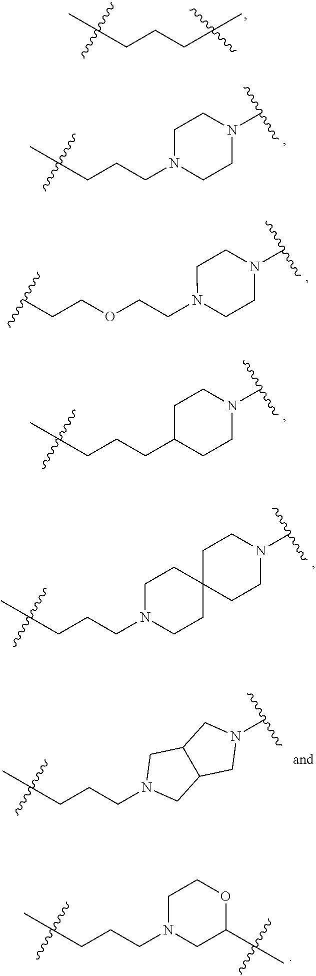

In some embodiments, L^{A} is selected from the group consisting of

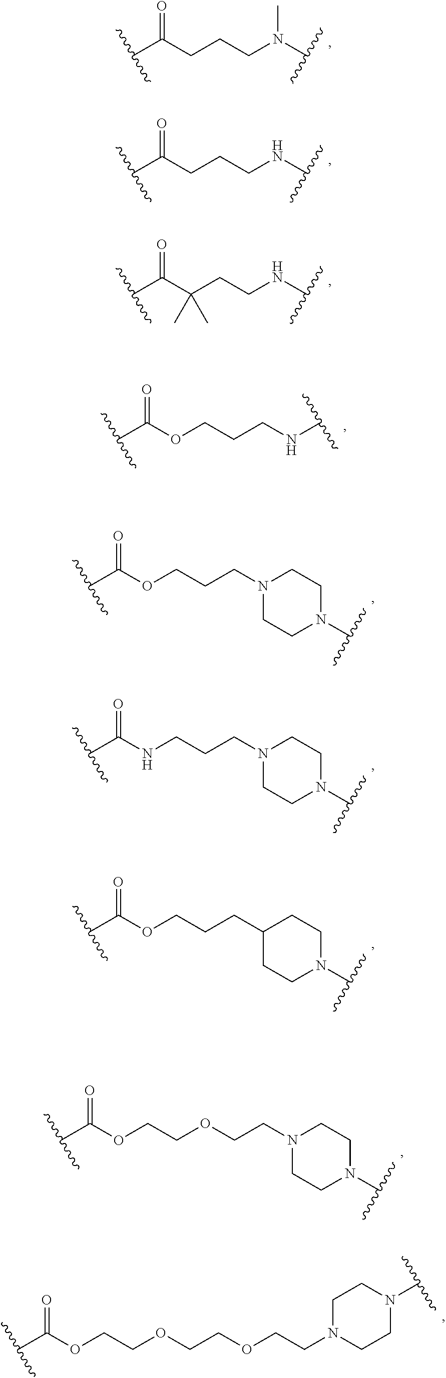

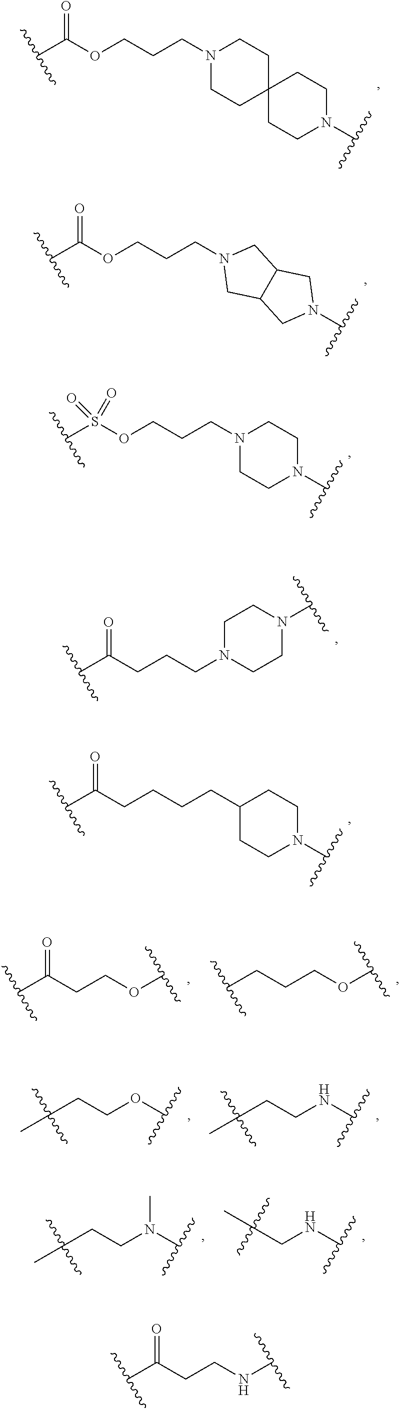

and a chemical bond.

In some embodiments, -L^{B}-W is selected from the group consisting of

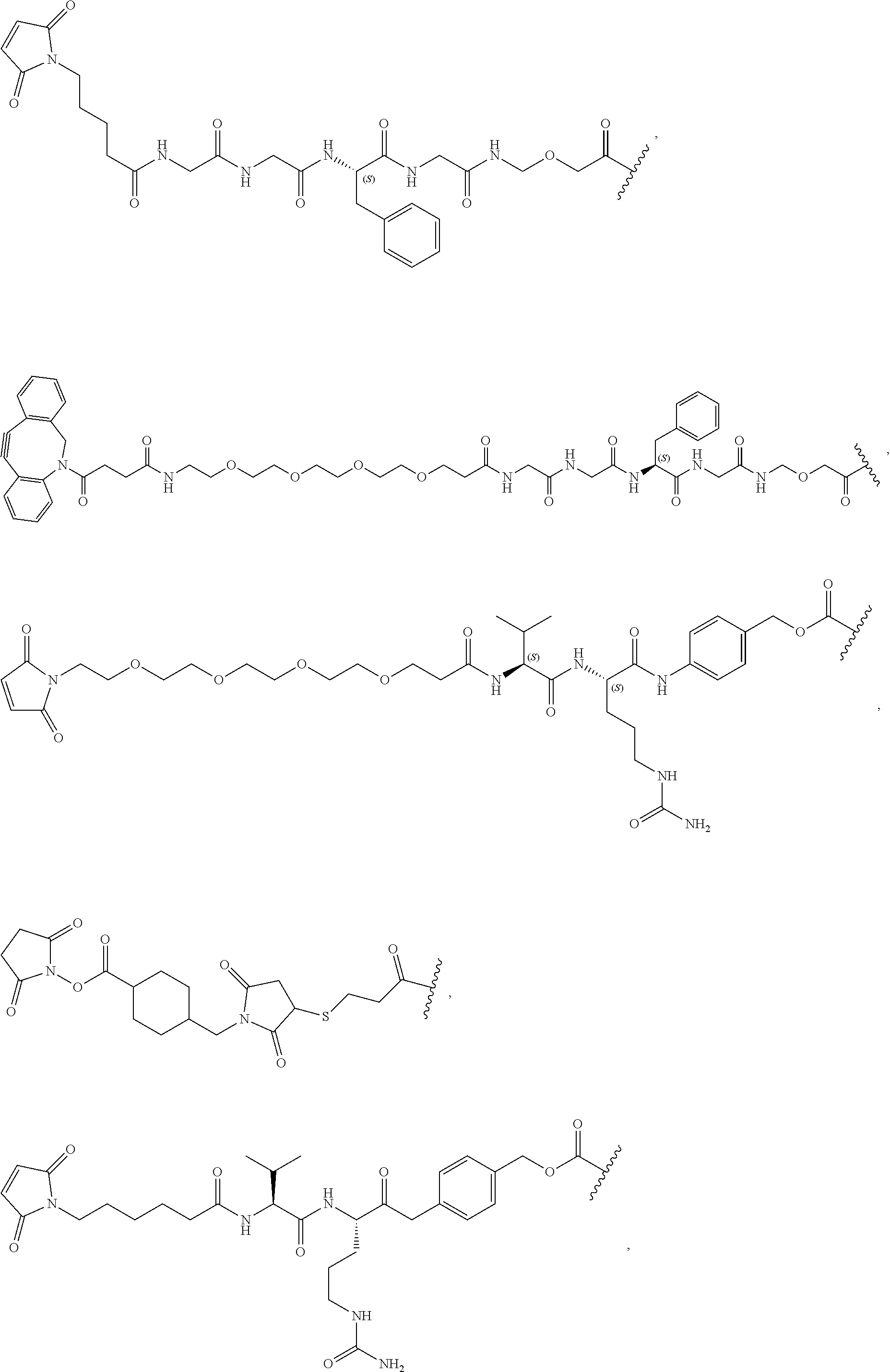

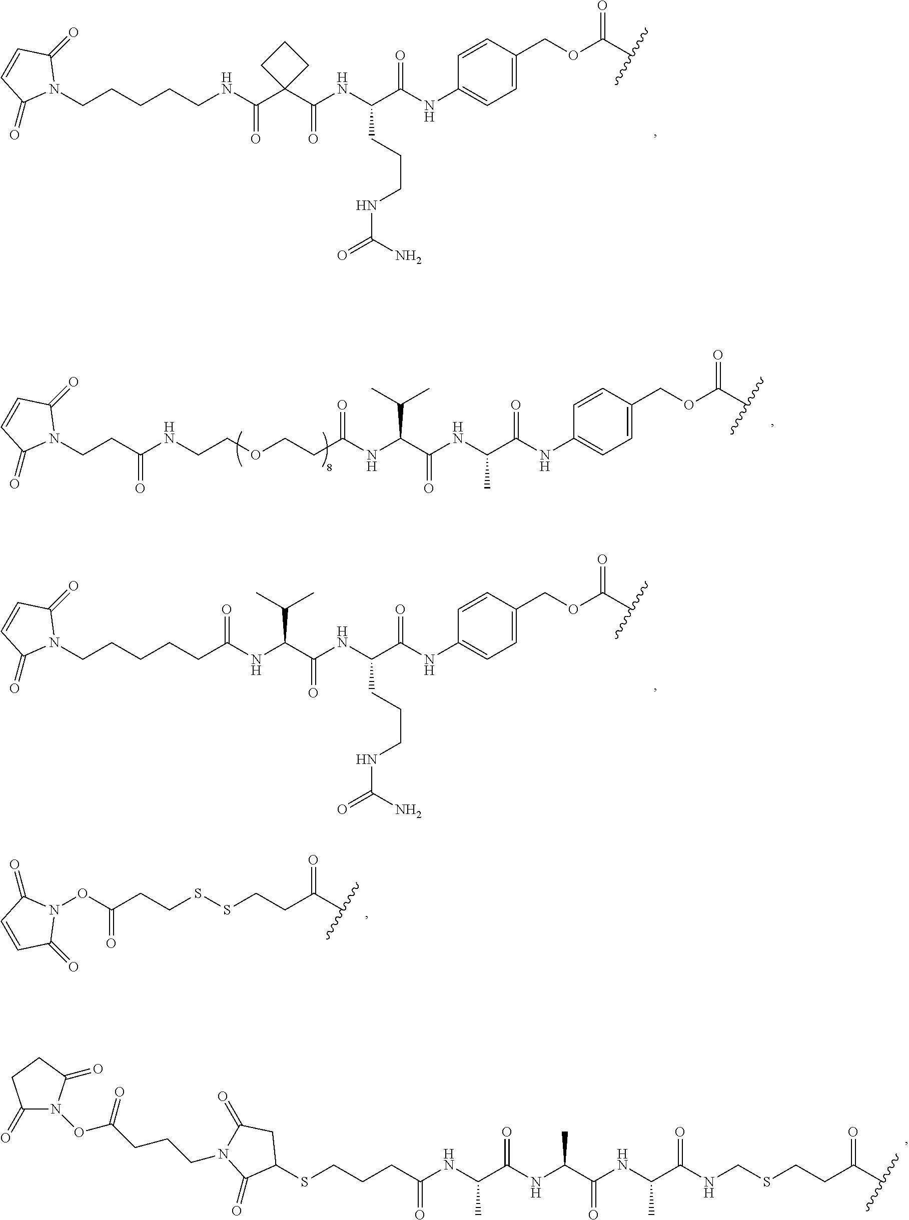

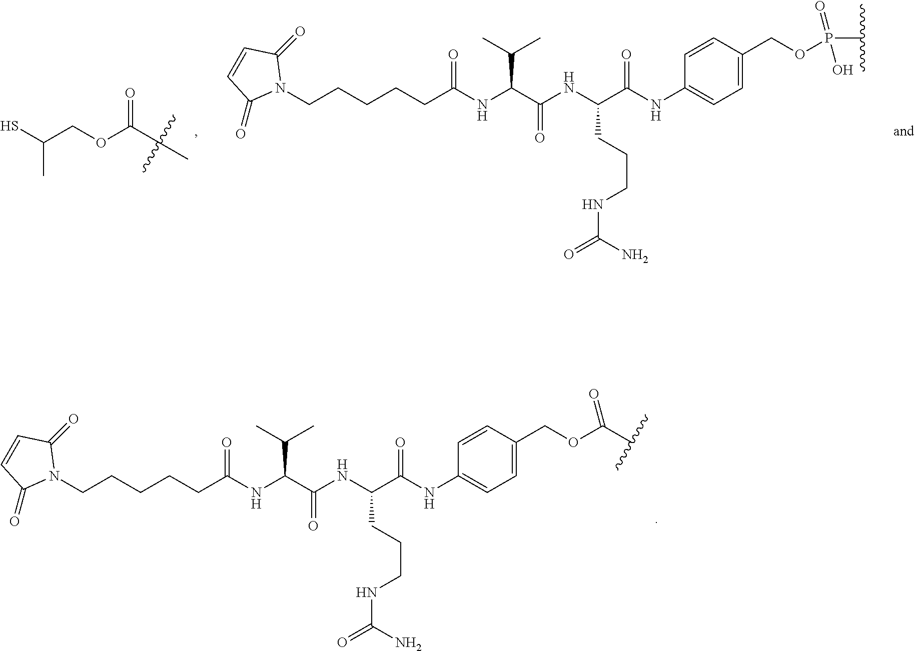

In some embodiments, the ring A and the ring B are independently selected from the group consisting of

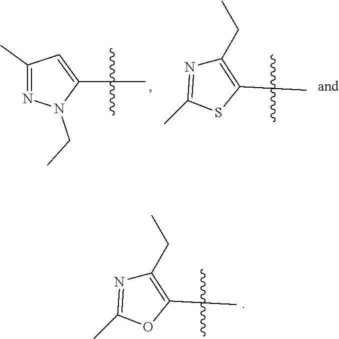

In some embodiments, X² is N or CR^{X};
R^{X} is —OR¹ or —SR¹; and
R¹ is methyl or -propyl-OH.

In some embodiments, the compound-linker conjugate is represented by the formula (Ia).

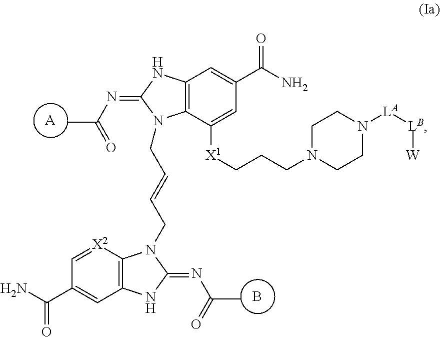

wherein the ring A and ring B are independently selected from the group consisting of

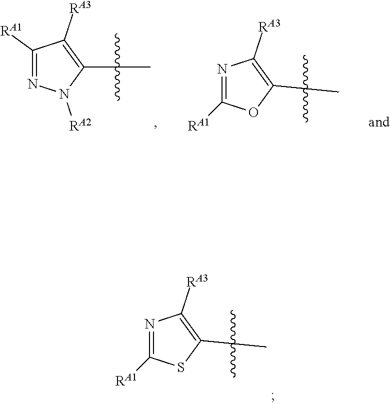

R^{A1} and R^{A3} are independently selected from the group consisting of hydrogen, halogen, —CN, unsubstituted —C₁₋₆ alkyl and halogenated —C₁₋₆ alkyl;
R^{A2} is selected from the group consisting of hydrogen, unsubstituted —C₁₋₆ alkyl and halogenated —C₁₋₆ alkyl;
X¹ is O or S;
X² is N or CR^{X};
R^{X} is selected from the group consisting of hydrogen, halogen, —CN, unsubstituted —C₁₋₆ alkyl, halogenated —C₁₋₆ alkyl, —R¹, —OR¹, —SR¹ and —NR¹R^{1′};
R¹ and R^{1′} are independently selected from the group consisting of hydrogen, —C₁₋₈ alkyl, —C₁₋₈ alkylene-NR²R^{2′}, —C₁₋₈ alkylene-OR², —C₀₋₈ alkylene-(3-10 membered cycloalkyl), —C₀₋₈ alkylene-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein, one or two carbon atoms in the alkylene group are adapted to be replaced with an oxygen atom;
R² and R^{2′} are independently selected from the group consisting of hydrogen, —C₁₋₆ alkyl, —C₀₋₈ alkylene-(3-10 membered cycloalkyl), —C₀₋₈ alkylene-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in the alkylene group are adapted to be replaced with an oxygen atom;
L^{A} is selected from the group consisting of —C(O)—C₁₋₈ alkylene-NH—, —C(O)—C₁₋₈ alkylene-N(C₁₋₆ alkyl)-, —C(O)O—C₁₋₈ alkylene-NH—, —C(O)O—C₁₋₈ alkylene-N(C₁₋₆ alkyl)-, —C(O)NH—C₁₋₈ alkylene-NH—, —C(O)NH—C₁₋₈ alkylene-N(C₁₋₆ alkyl)-, —C₁₋₈ alkylene-NH—, —C₁₋₈ alkylene-N(C₁₋₆ alkyl)-, —C(O)—C₁₋₈ alkylene-(3-10 membered cycloalkyl)-, —C(O)O—C₁₋₈ alkylene-(3-10 membered cycloalkyl)-, —C(O)NH—C₁₋₈ alkylene-(3-10 membered cycloalkyl)-, —C₁₋₈ alkylene-(3-10 membered cycloalkyl)-, —C(O)—C₁₋₈ alkylene-(3-10 membered heterocycloalkyl)-, —C(O)O—C₁₋₈ alkylene-(3-10 membered heterocycloalkyl)-, —C(O)NH—C₁₋₈ alkylene-(3-10 membered heterocycloalkyl)-, —C₁₋₈ alkylene-(3-10 membered heterocycloalkyl)-, —C(O)—C₁₋₈ alkylene-(5-12 membered spirocycloalkyl)-, —C(O)O—C₁₋₈ alkylene-(5-12 membered spirocycloalkyl)-, —C(O)NH—C₁₋₈ alkylene-(5-12 membered spirocycloalkyl)-, —C₁₋₈ alkylene-(5-12 membered spirocycloalkyl)-, —C(O)—C₁₋₈ alkylene-(5-12 membered spiroheterocycloalkyl)-, —C(O)O—C₁₋₈ alkylene-(5-12 membered spiroheterocycloalkyl)-, —C(O)NH—C₁₋₈ alkylene-(5-12 membered spiroheterocycloalkyl)-, —C₁₋₈ alkylene-(5-12 membered spiroheterocycloalkyl), —C(O)—C₁₋₈ alkylene-(5-12 membered bridged cycloalkyl)-, —C(O)O—C₁₋₈ alkylene-(5-12 membered bridged cycloalkyl)-, —C(O)NH—C₁₋₈ alkylene-(5-12 membered bridged cycloalkyl)-, —C₁₋₈ alkylene-(5-12 membered bridged cycloalkyl)-, —C(O)—C₁₋₈ alkylene-(5-12 membered bridged heterocycloalkyl)-, —C(O)O—C₁₋₈ alkylene-(5-12 membered bridged heterocycloalkyl)-, —C(O)NH—C₁₋₈ alkylene-(5-12 membered bridged heterocycloalkyl)-, —C₁₋₈ alkylene-(5-12 membered bridged heterocycloalkyl)-, —C(O)—C₁₋₈ alkylene-O—, —C(O)O—C₁₋₈ alkylene-O—, —C(O)NH—C₁₋₈ alkylene-O—, —C₁₋₈ alkylene-O—, —S(O)₂—C₁₋₈ alkylene-NH—, —S(O)₂—C₁₋₈ alkylene-N(C₁₋₆ alkyl)-, —S(O)₂O—C₁₋₈ alkylene-NH—, —S(O)₂O—C₁₋₈ alkylene-N(C₁₋₆ alkyl)-, —S(O)₂NH—C₁₋₈ alkylene-NH—, —S(O)₂NH—C₁₋₈ alkylene-N(C₁₋₆ alkyl)-, —S(O)₂—C₁₋₈ alkylene-(3-10 membered cycloalkyl)-, —S(O)₂O—C₁₋₈ alkylene-(3-10 membered cycloalkyl)-, —S(O)₂NH—C₁₋₈ alkylene-(3-10 membered cycloalkyl)-, —S(O)₂—C₁₋₈ alkylene-(3-10 membered heterocycloalkyl)-, —S(O)₂O—C₁₋₈ alkylene-(3-10 membered heterocycloalkyl)-, —S(O)₂NH—C₁₋₈ alkylene-(3-10 membered heterocycloalkyl)-, —S(O)₂—C₁₋₈ alkylene-(5-12 membered spirocycloalkyl)-, —S(O)₂O—C₁₋₈ alkylene-(5-12 membered spirocycloalkyl)-, —S(O)₂NH—C₁₋₈ alkylene-(5-12 membered spirocycloalkyl)-, —S(O)₂—C₁₋₈ alkylene-(5-12 membered spiroheterocycloalkyl)-, —S(O)₂O—C₁₋₈ alkylene-(5-12 membered spiroheterocycloalkyl)-, —S(O)₂NH—C₁₋₈ alkylene-(5-12 membered spiroheterocycloalkyl)-, —S(O)₂—C₁₋₈ alkylene-(5-12 membered bridged cycloalkyl)-, —S(O)₂O—C₁₋₈ alkylene-(5-12 membered bridged cycloalkyl)-, —S(O)₂NH—C₁₋₈ alkylene-(5-12 membered bridged cycloalkyl)-, —S(O)₂—C₁₋₈ alkylene-(5-12 membered bridged heterocycloalkyl)-, —S(O)₂O—C₁₋₈ alkylene-(5-12 membered bridged heterocycloalkyl)-, —S(O)₂NH—C₁₋₈ alkylene-(5-12 membered bridged heterocycloalkyl)-, —S(O)₂—C₁₋₈ alkylene-O—, —S(O)₂O—C₁₋₈ alkylene-O—, —S(O)₂NH—C₁₋₈ alkylene-O— and a chemical bond; wherein one or two carbon atoms in the alkylene group are adapted to be replaced with an oxygen atom;
L^{B} is -(L¹)ₚ-;
p is an integer selected from 1-50;
L¹ is each independently selected from the group consisting of CRR, C(O), O, S, S(O), S(O)₂, NR, —CR═CR—, —C≡C—, P(O)R, P(O)OR, 3-10 membered cycloalkane, 3-10 membered heterocycloalkane, 5-10 membered aromatic ring, 5-10 membered aromatic heterocyclic ring, 5-12 membered spiro ring, 5-12 membered spiro heterocyclic ring, 5-12 membered bridged ring and 5-12 membered bridged heterocyclic ring; wherein cycloalkane, heterocycloalkane, aromatic ring, aromatic heterocyclic ring, spiro ring, spiro heterocyclic ring, bridged ring, and bridged heterocyclic ring are unsubstituted or substituted by one, two or three R^{L1};
R^{L1} is each independently selected from the group consisting of hydrogen, halogen, ═O, cyano, nitro, unsubstituted —C₁₋₆ alkyl, halogenated —C₁₋₆ alkyl, —OR, —NRR, —C₀₋₄ alkylene-(3-10 membered cycloalkyl) and —C₀₋₄ alkylene-(3-10 membered heterocycloalkyl);
R is each independently selected from the group consisting of hydrogen, halogen, cyano, unsubstituted —C₁₋₆ alkyl, unsubstituted —C₂₋₆ alkenyl, unsubstituted —C₂₋₆ alkynyl, halogenated —C₁₋₆ alkyl, halogenated —C₂₋₆ alkenyl, halogenated —C₂₋₆ alkynyl, —C₀₋₄ alkylene-OR^{R1}, —C₀₋₄ alkylene-OC(O)R^{R1}, —C₀₋₄ alkylene-SR^{R1}, —C₀₋₄ alkylene-S(O)₂R^{R1}, —C₀₋₄ alkylene-S(O)RR, —C₀₋₄ alkylene-S(O)₂NR^{R1}R^{R2}, —C₀₋₄ alkylene-S(O)NR^{R1}R^{R2}, —C₀₋₄ alkylene-S(O)(NH)R^{R1}, —C₀₋₄ alkylene-S(O)(NH)NR^{R1}R^{R2}, —C₀₋₄ alkylene-C(O)R^{R1}, —C₀₋₄ alkylene-C(O)OR^{R1}, —C₀₋₄ alkylene-C(O)NR^{R1}R^{R2}, —C₀₋₄ alkylene-NR^{R1}R^{R2}, —C₀₋₄ alkylene-NR^{R1}C(O)R^{R2}, —C₀₋₄ alkylene-NR^{R1}S(O)₂R^{R2}, C₀₋₄ alkylene-NR^{R1}S(O)R^{R2}, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring);
R^{R1} and R^{R2} are independently selected from the group consisting of hydrogen, unsubstituted —C₁₋₆ alkyl, unsubstituted —C₂₋₆ alkenyl, unsubstituted —C₂₋₆ alkynyl, —OH, —NH₂, halogenated —C₁₋₆ alkyl, halogenated —C₂₋₆ alkenyl, halogenated —C₂₋₆ alkynyl, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring); and
W is selected from the group consisting of

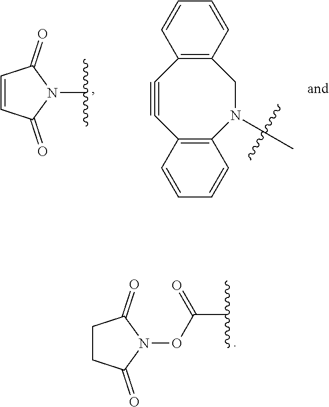 [SMILES: *N1C(=O)C=CC1=O ; *N1Cc2ccccc2C#Cc2ccccc21 ; *C(=O)ON1C(=O)CCC1=O]

In some embodiments, in the formula (Ia), the ring A and ring B are independently selected from the group consisting of

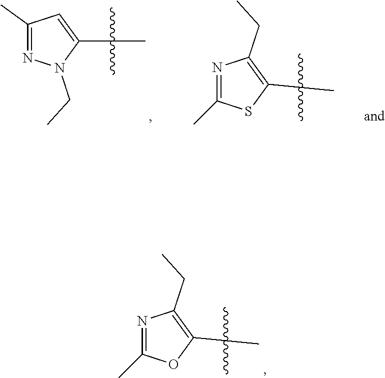

and preferably, at least one of the ring A and the ring B is

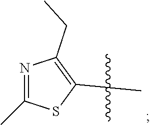 [SMILES: *c1sc(C)nc1CC]

X² is N or CR^{X};
R^{X} is —OR¹, or —SR¹;
R¹ is methyl, or -propylidene-OH;
L^{A} is selected from the group consisting of

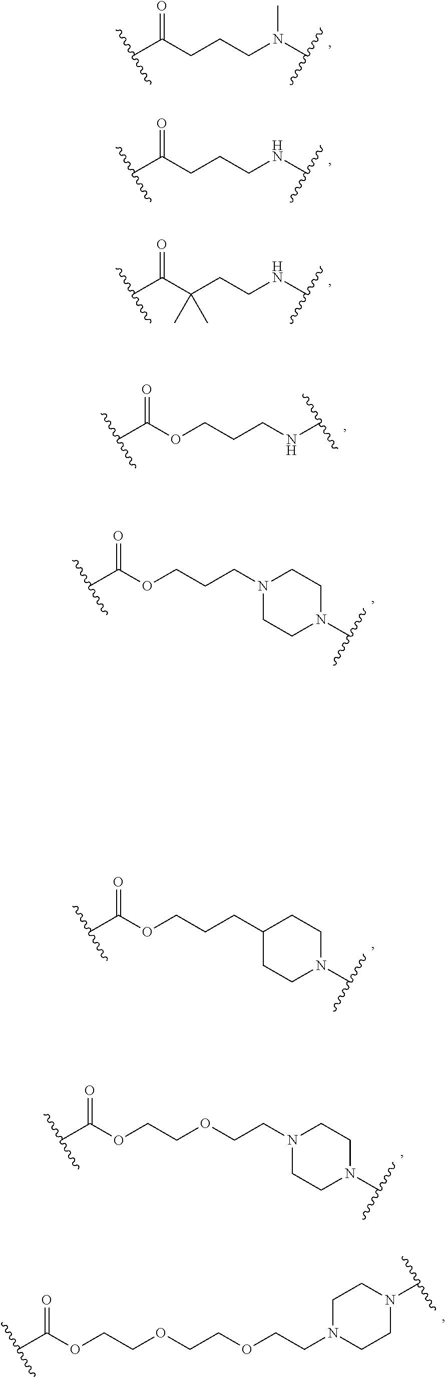

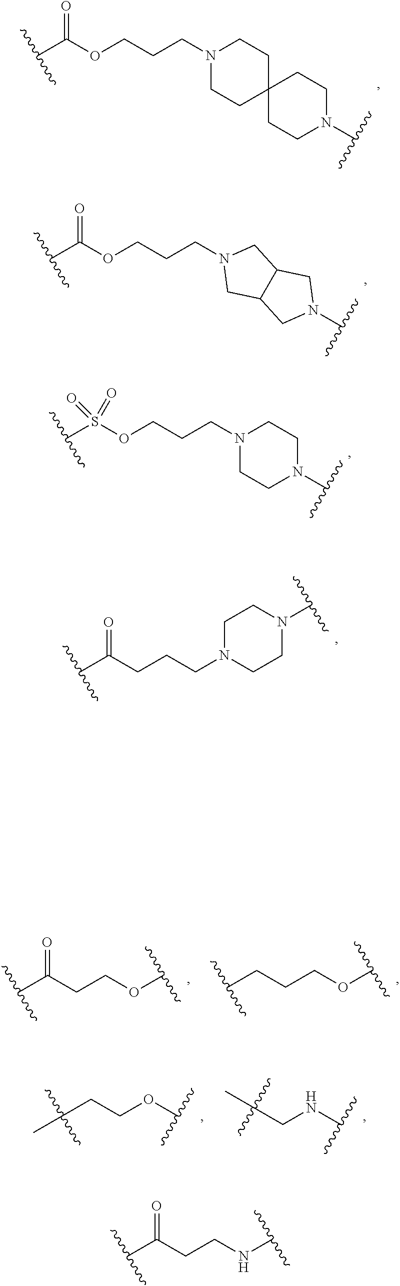

and a chemical bond;
-L^{B}-W is selected from the group consisting of:

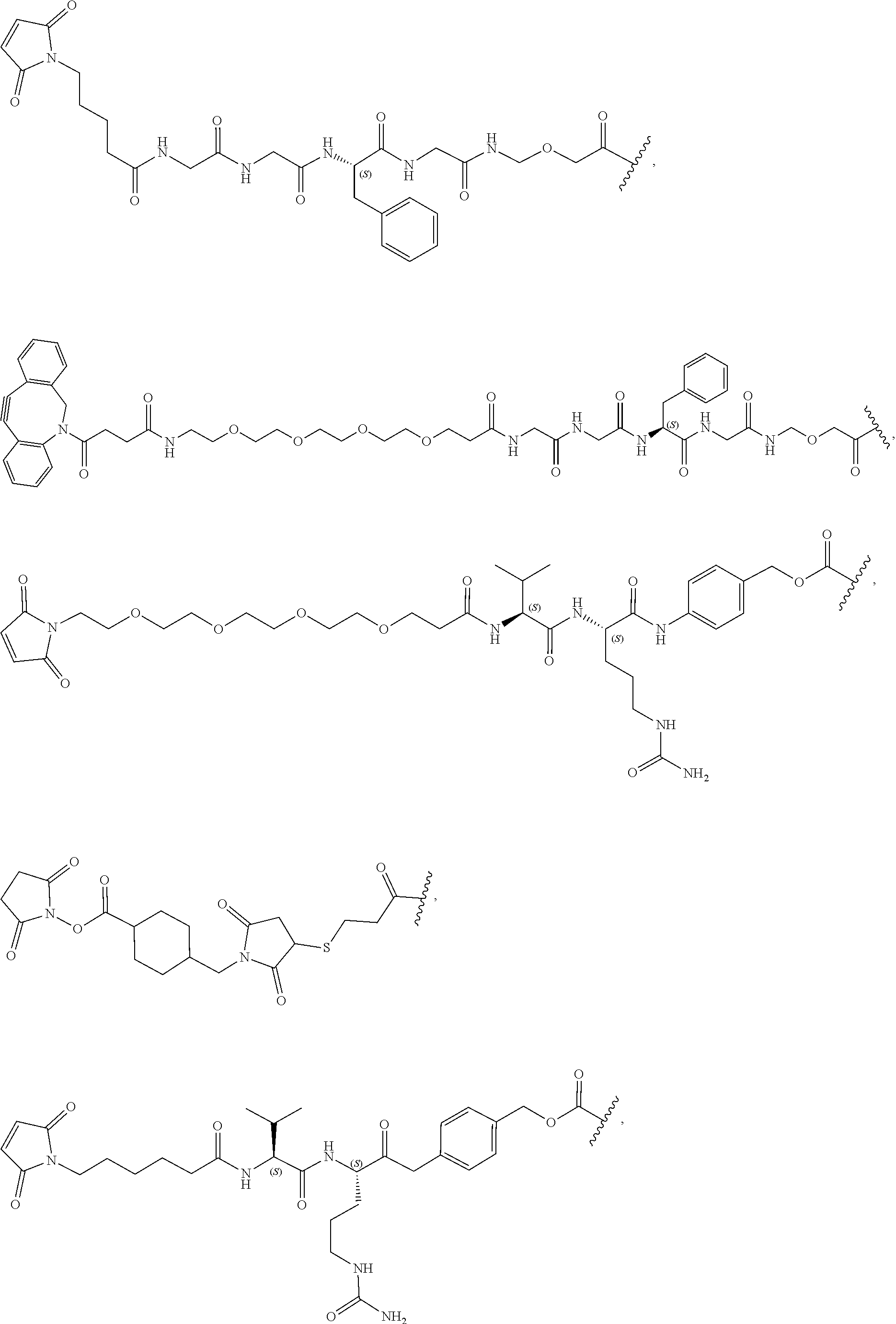

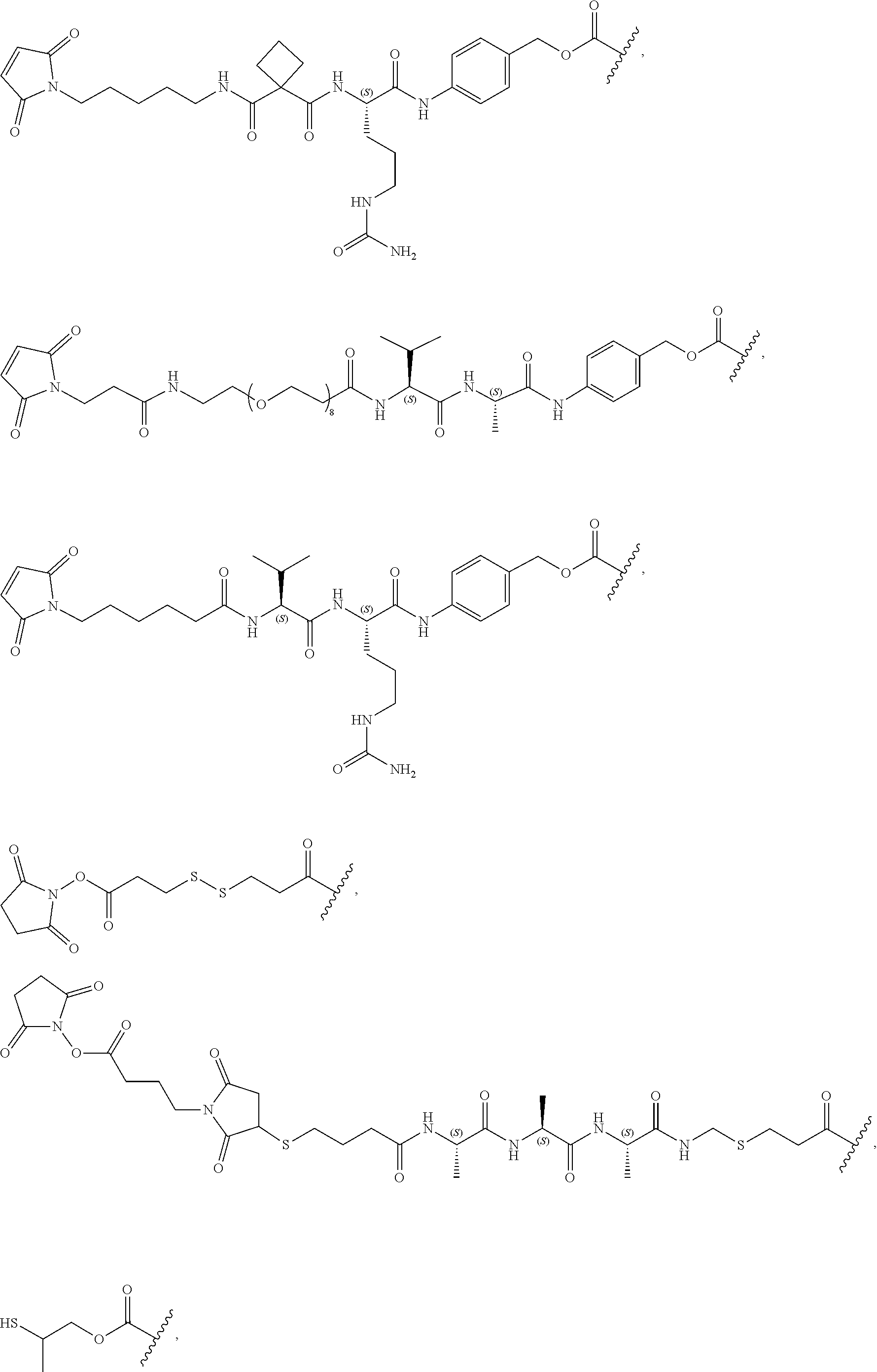

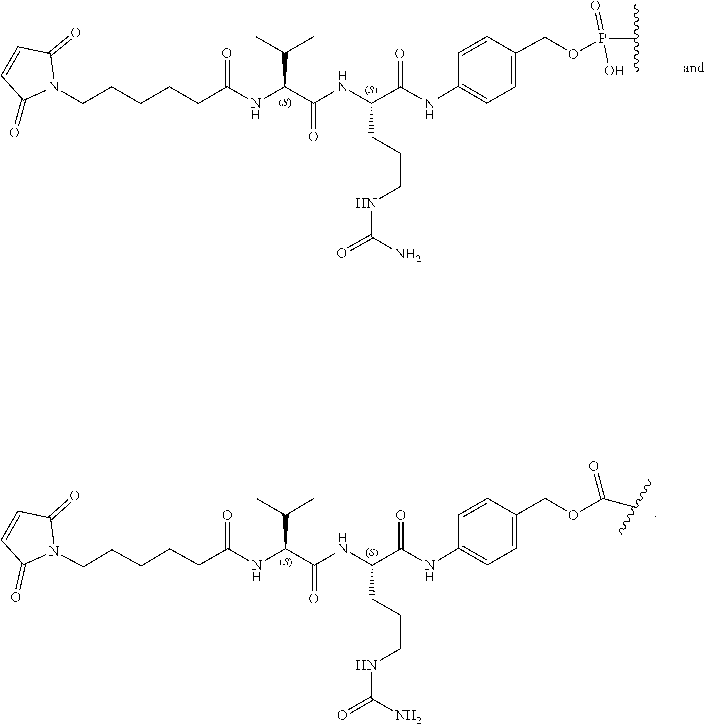

In some embodiments, the compound of formula (Ia) is represented by the formula (Ib):

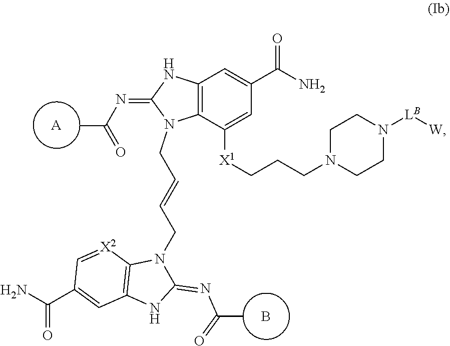

wherein the ring A, ring B, X¹, X², L^{B}, and W are defined as above.

In some embodiments, the compound-linker conjugate is selected from the group consisting of:

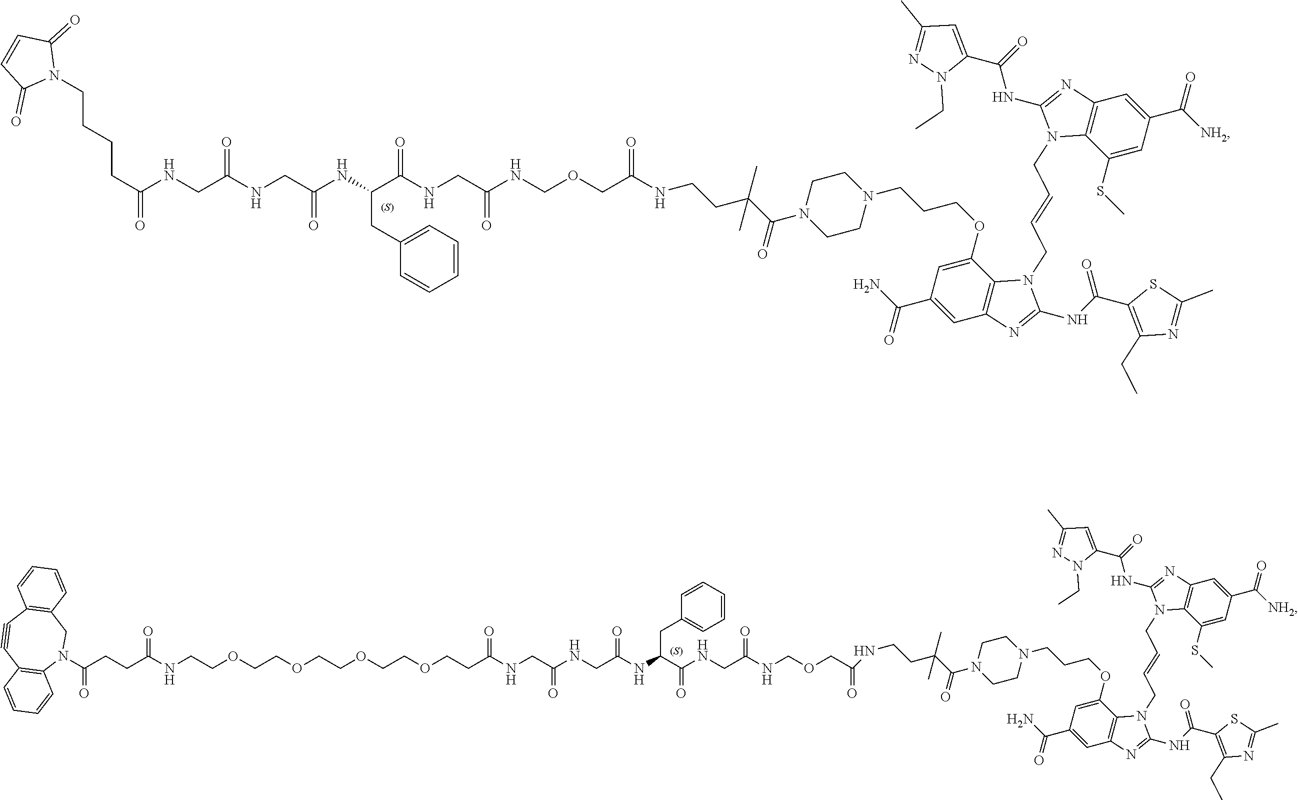

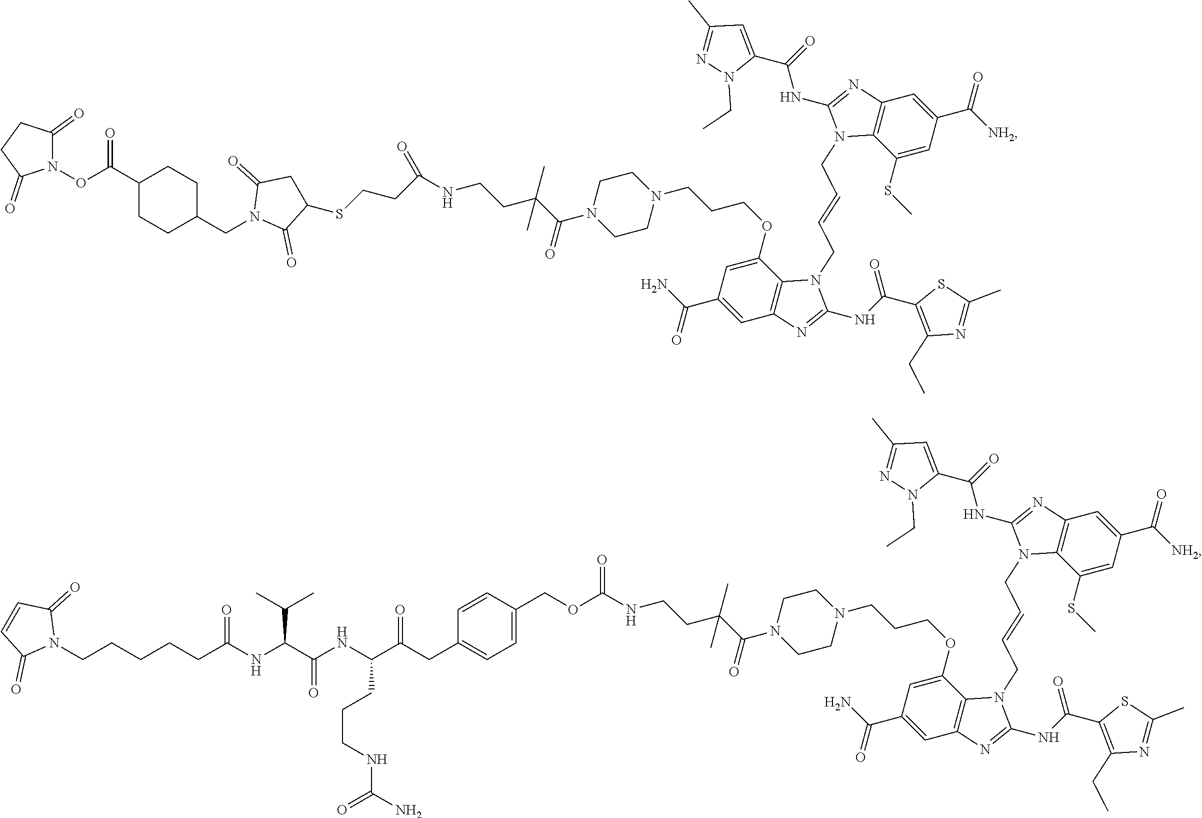 [SMILES: CCc1nc(C)sc1C(=O)Nc1nc2cc(C(N)=O)cc(OCCCN3CCN(C(=O)C(C)(C)CCNC(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)C(C)(C)CCNC(=O)OCc4ccc(CC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21]

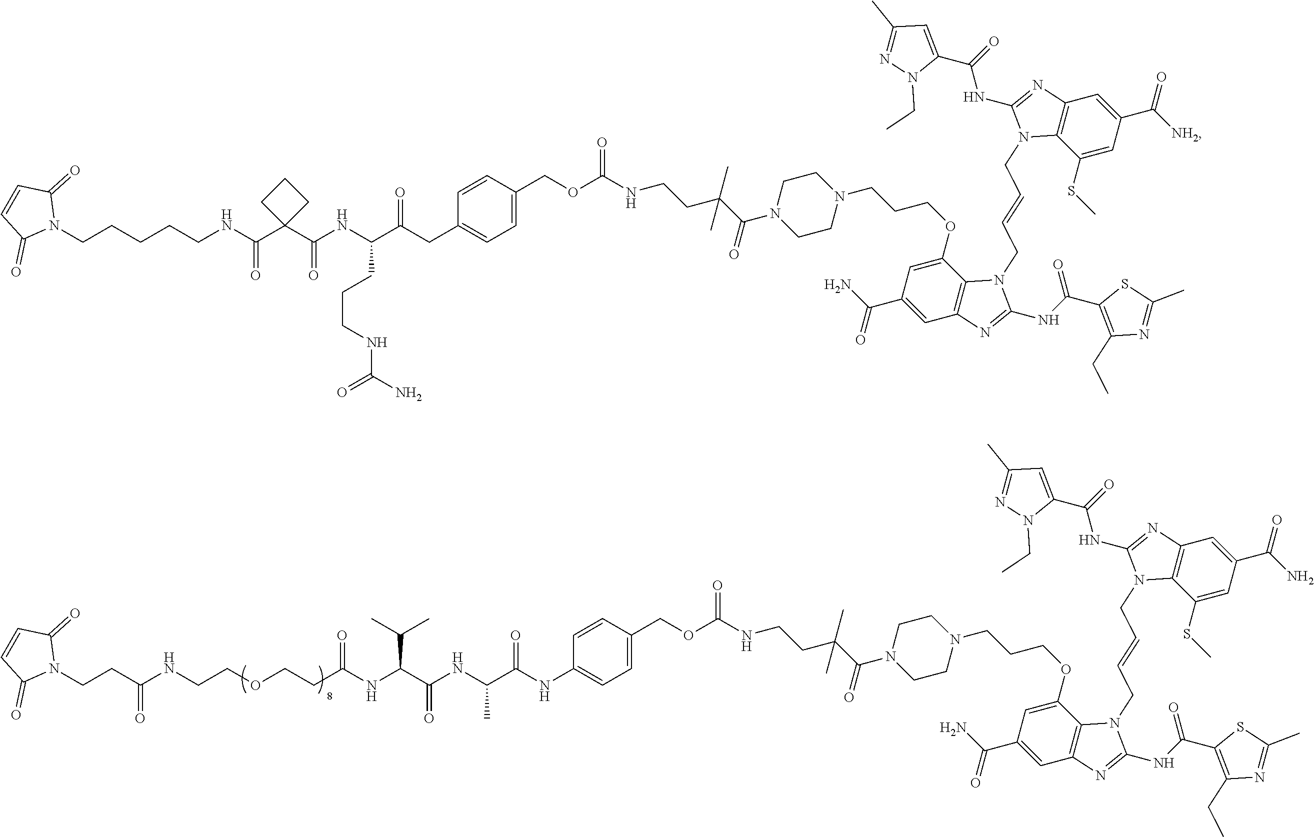

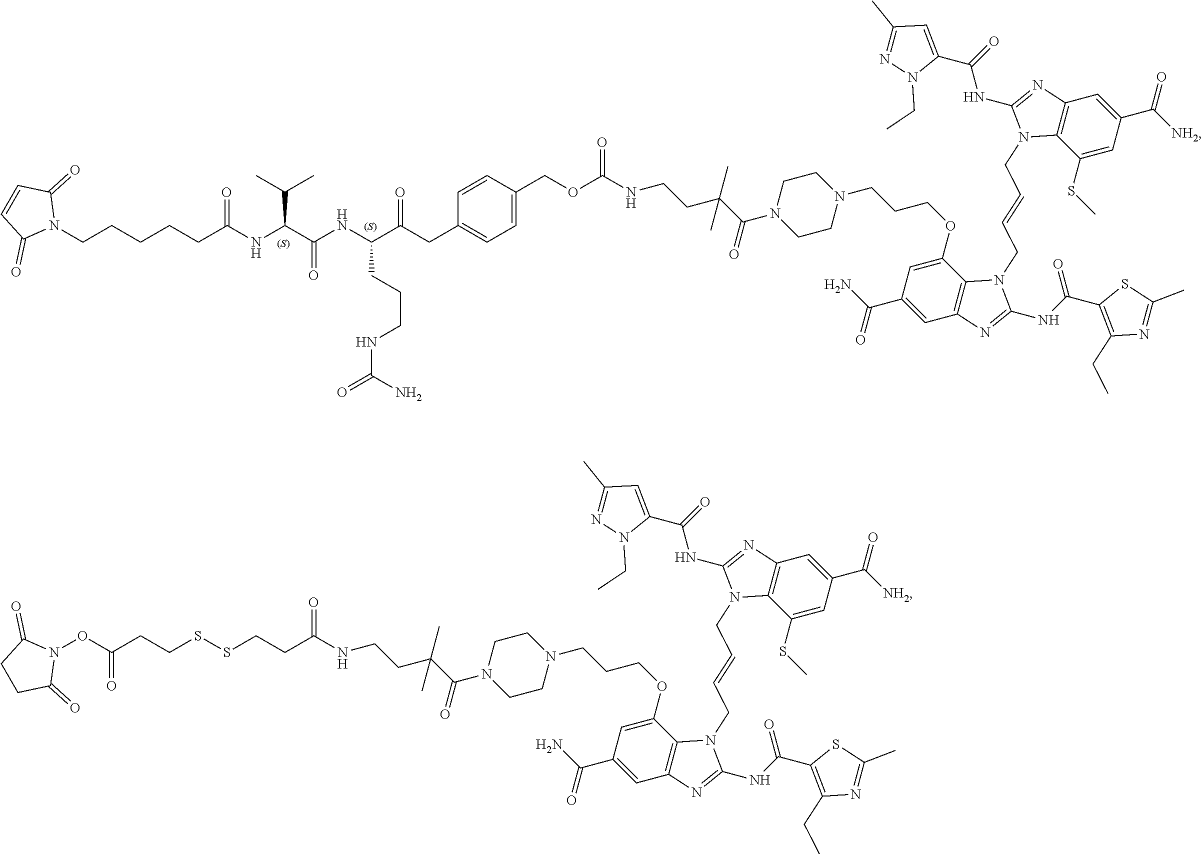

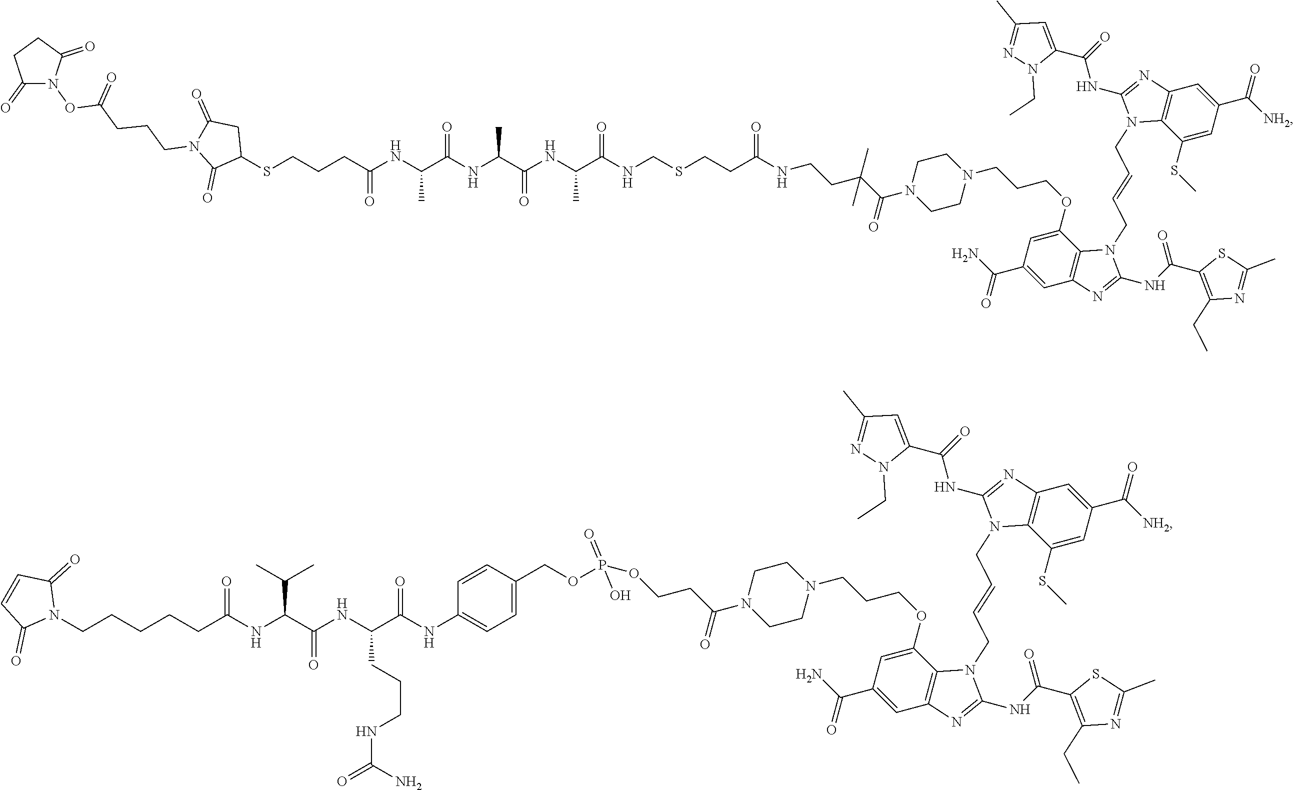 [SMILES: CCc1nc(C)sc1C(=O)Nc1nc2cc(C(N)=O)cc(OCCCN3CCN(C(=O)C(C)(C)CCNC(=O)CCSCNC(=O)[C@H](C)NC(=O)[C@H](C)NC(=O)[C@H](C)NC(=O)CCCSC4CC(=O)N(CCCC(=O)ON5C(=O)CCC5=O)C4=O)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)CCOP(=O)(O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21]

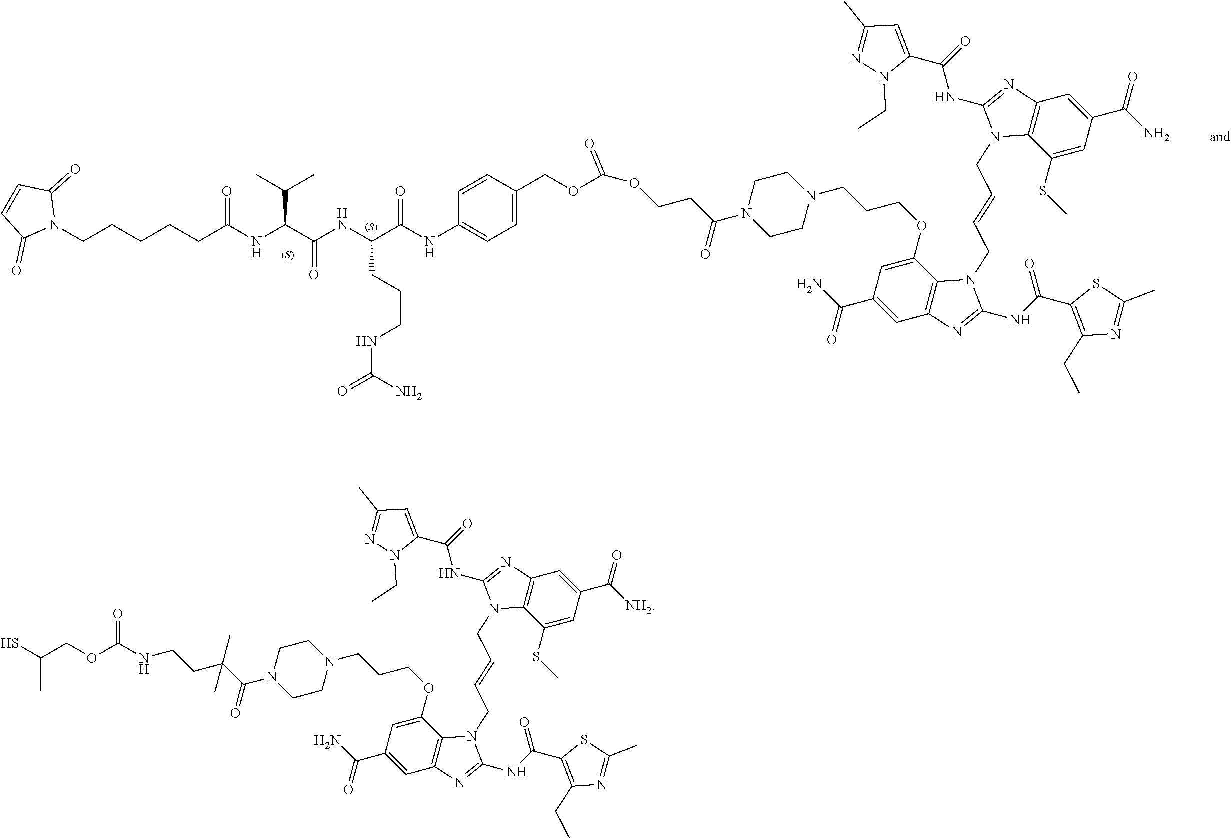

In some embodiments, the compound-linker conjugate is selected from the group consisting of:

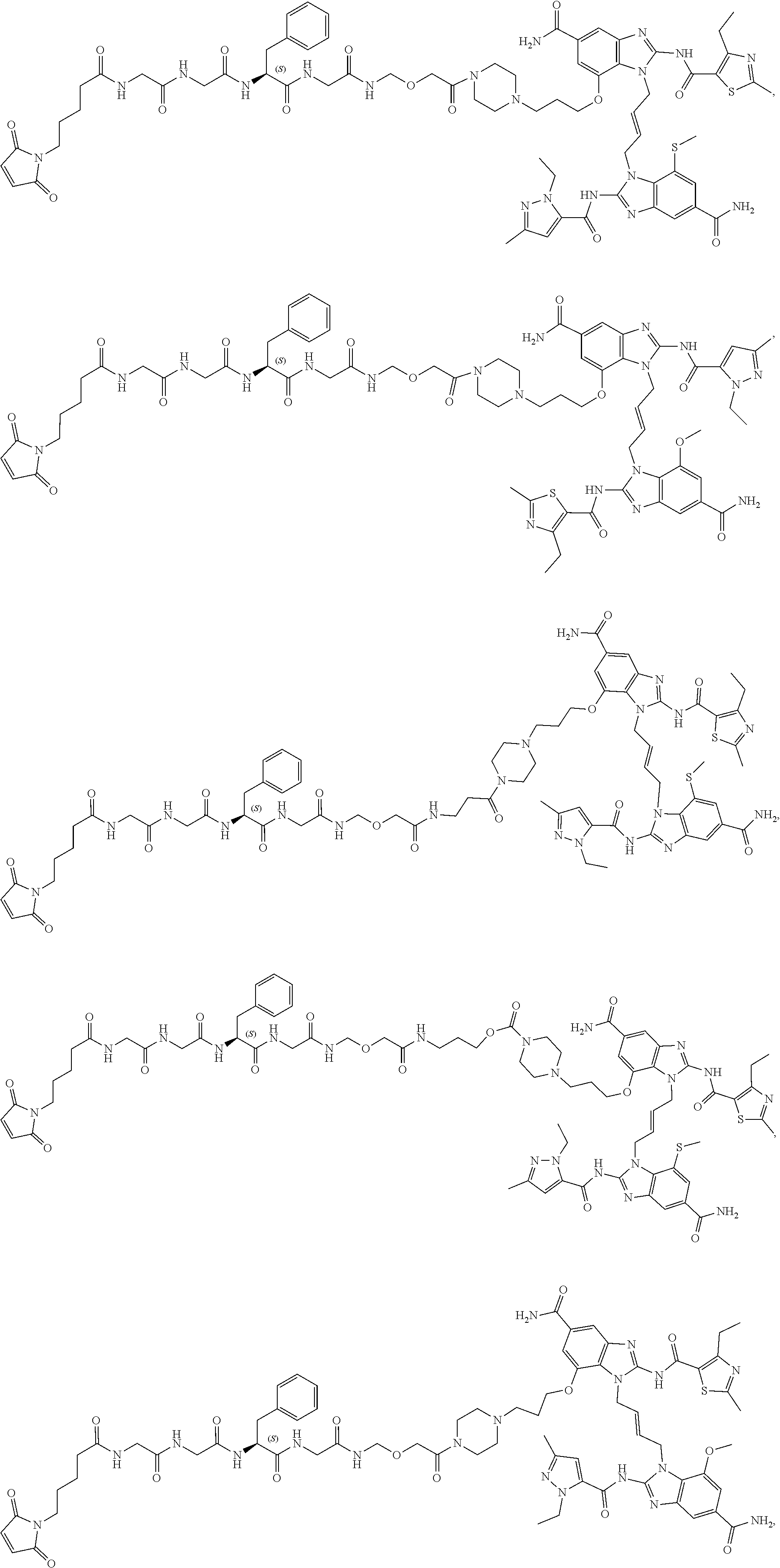

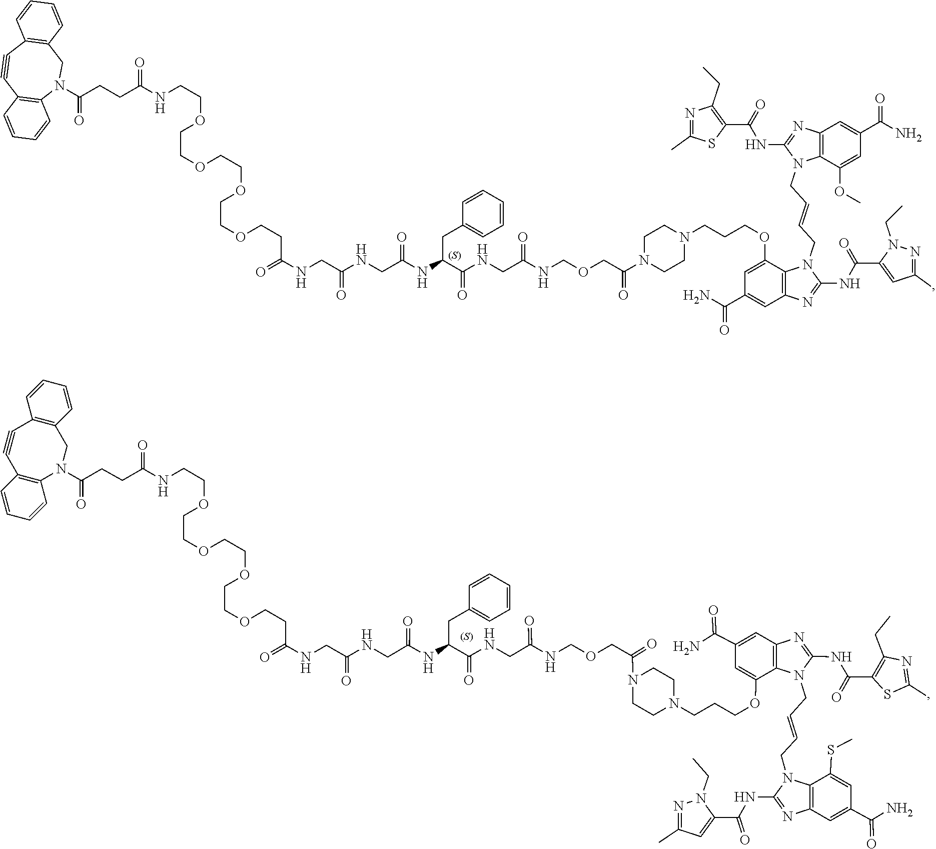

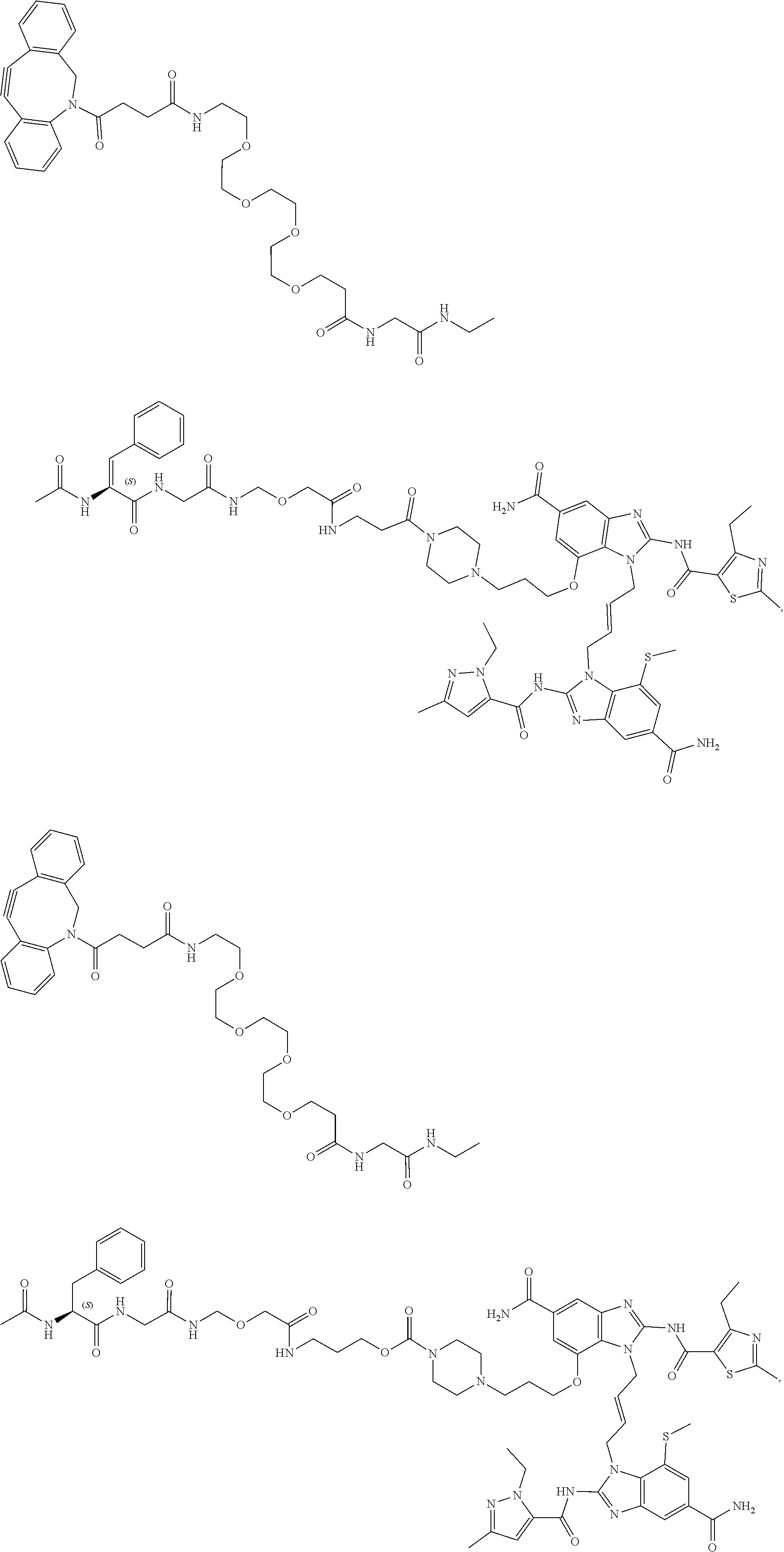

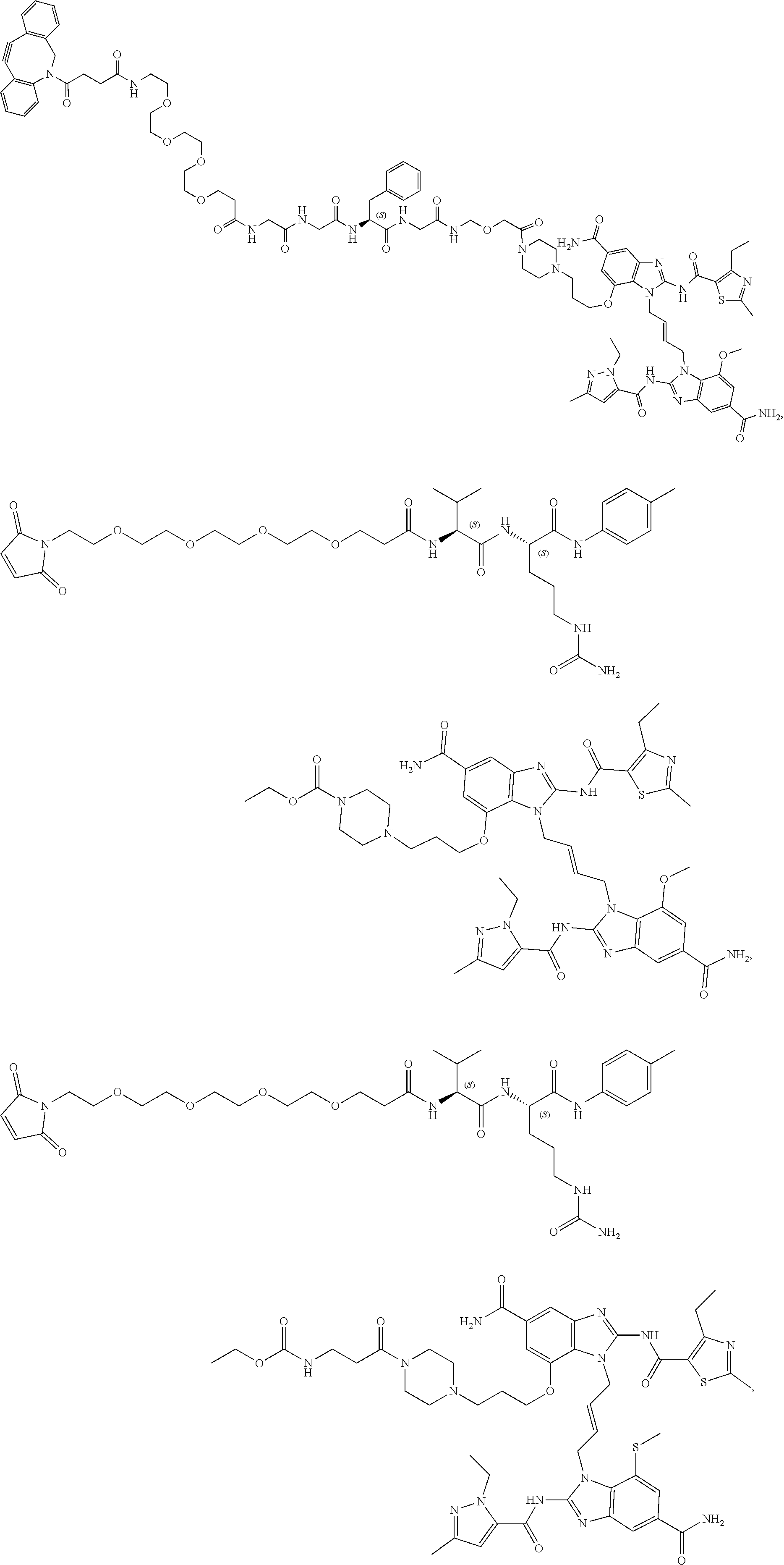

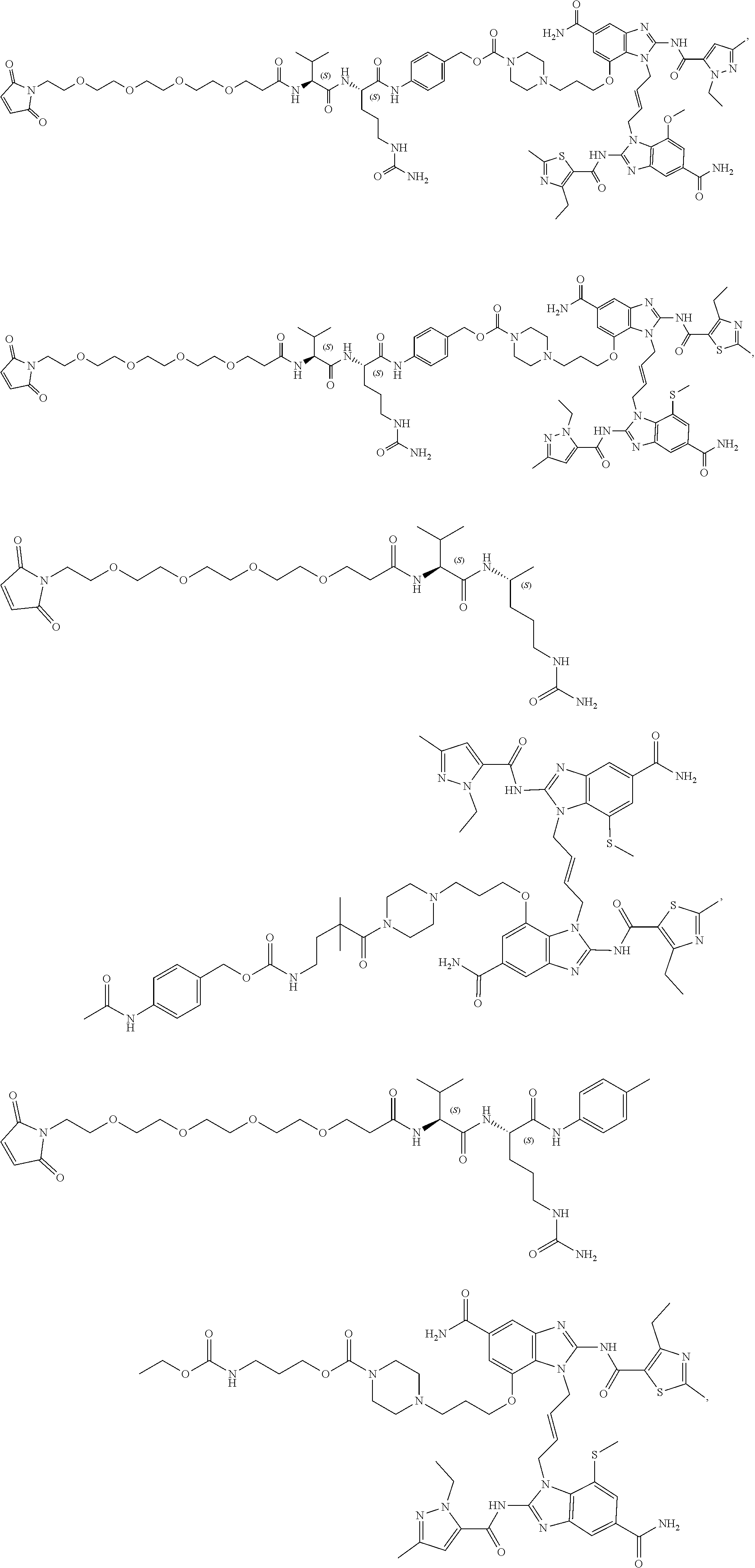

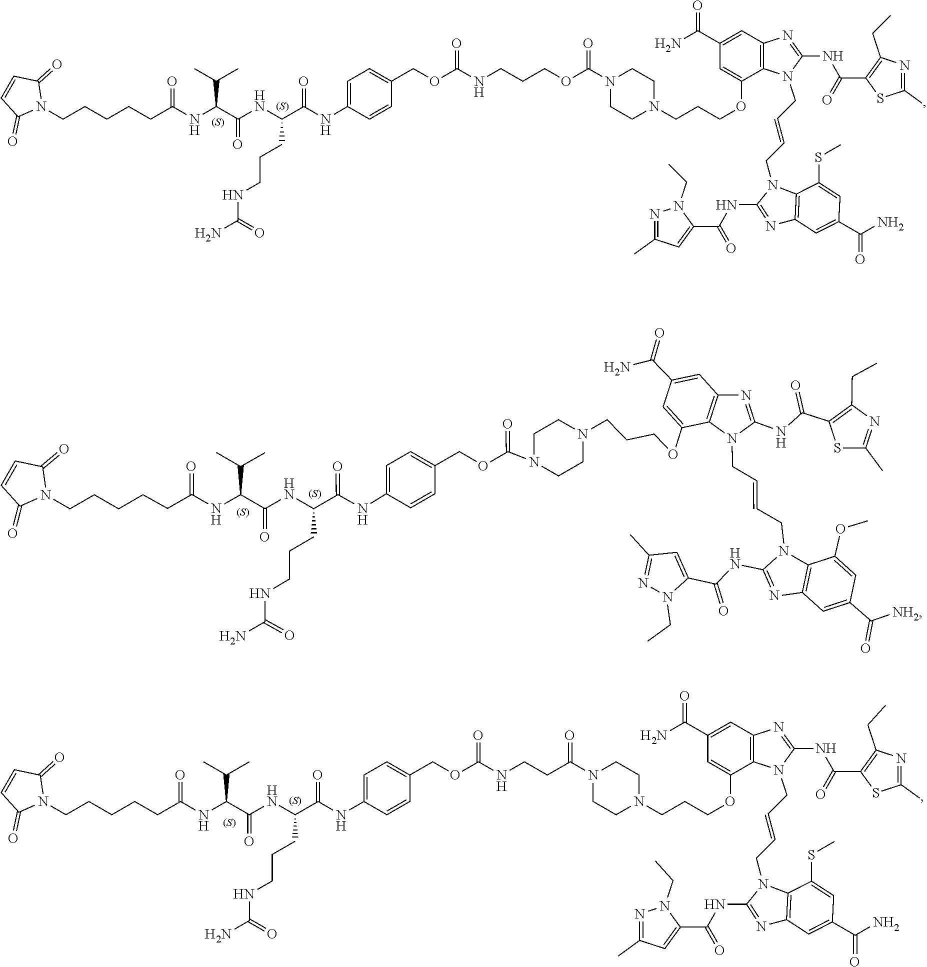

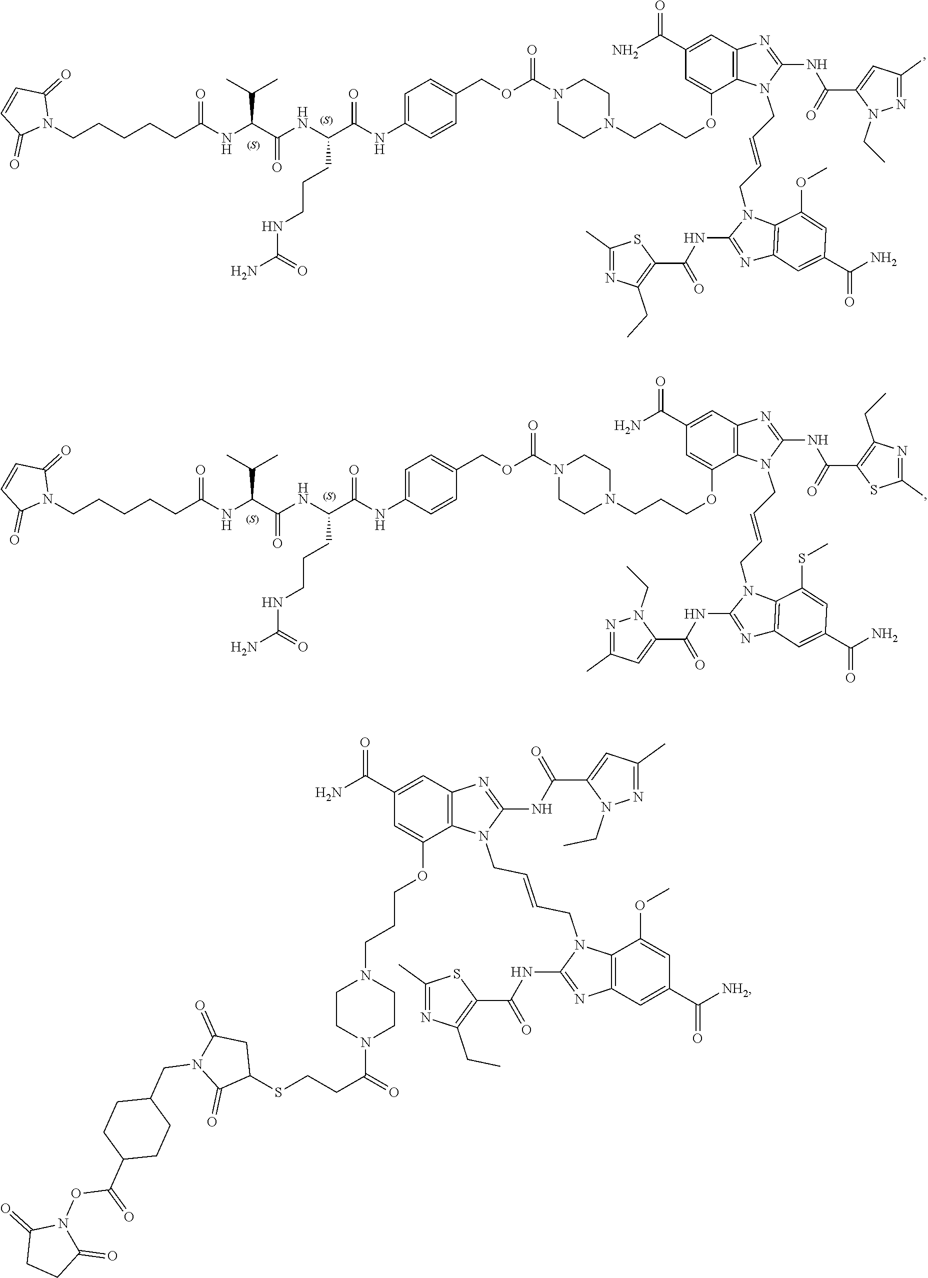

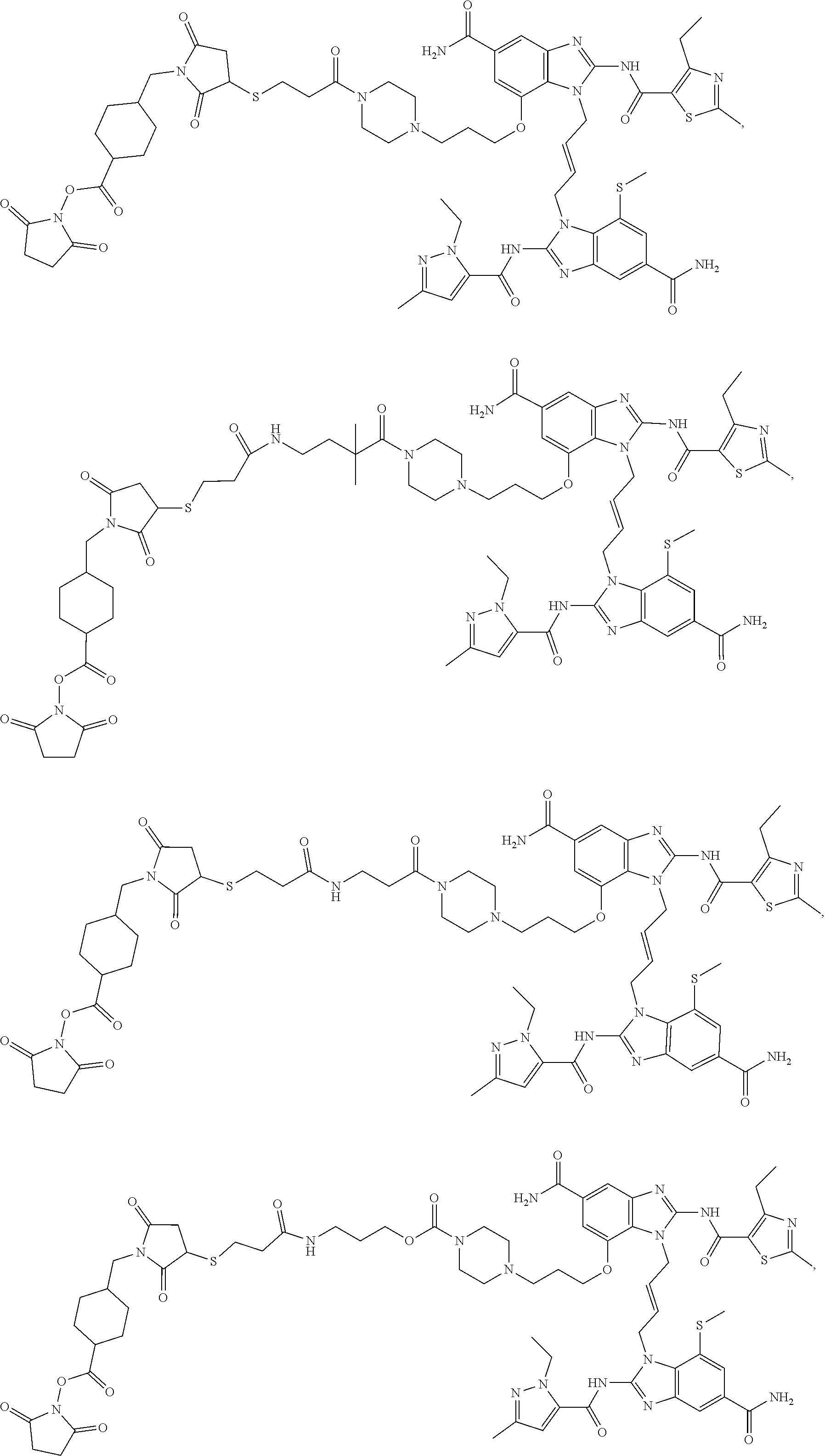

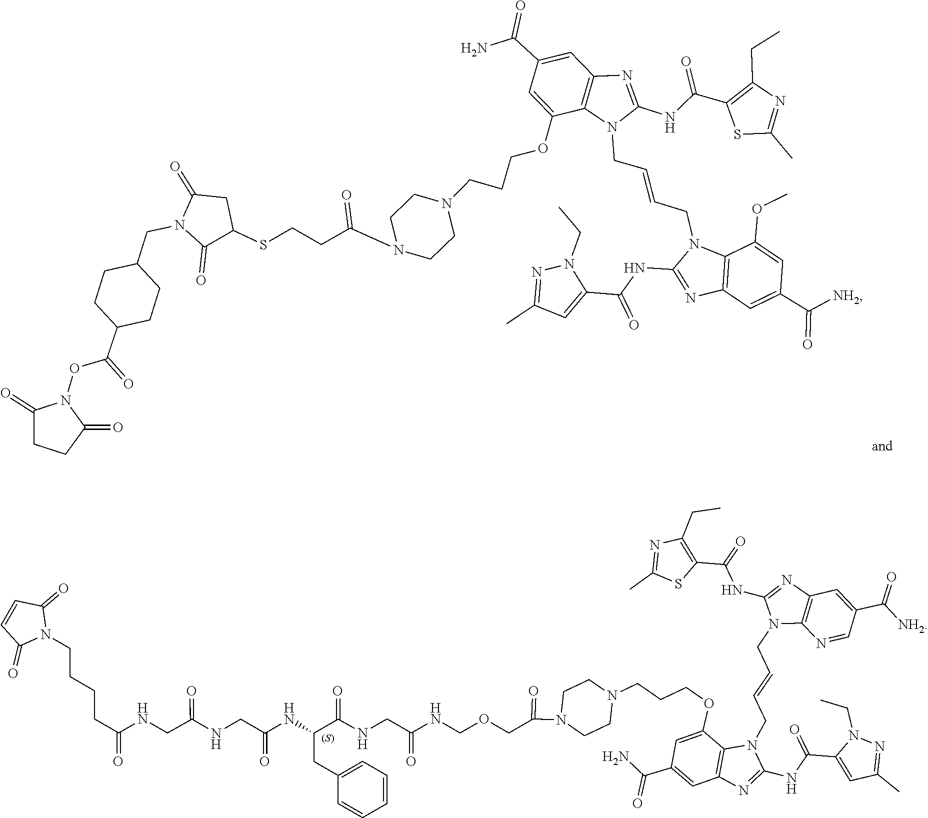

The present disclosure further provides a compound of formula (II), or a deuterated compound, a stereoisomer, or a pharmaceutically acceptable salt thereof:

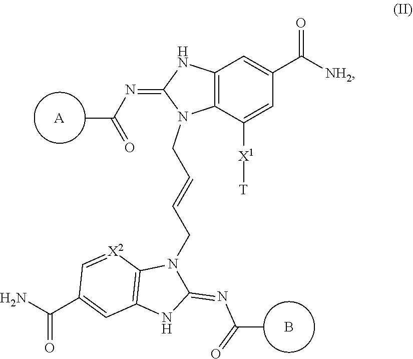

wherein ring A and ring B are independently selected from the group consisting of

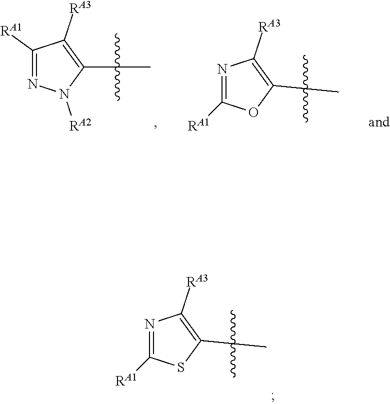

R^{A1} and R^{A3} are independently selected from the group consisting of hydrogen, halogen, —CN, unsubstituted —C₁₋₆ alkyl and halogenated —C₁₋₆ alkyl;
R^{A2} is selected from the group consisting of hydrogen, unsubstituted —C₁₋₆ alkyl and halogenated —C₁₋₆ alkyl;
X¹ is O or S;
X² is N or CR^{X};
R^{X} is selected from the group consisting of hydrogen, halogen, —CN, unsubstituted —C₁₋₆ alkyl, halogenated —C₁₋₆ alkyl, —R¹, —OR¹, —SR¹ and —NR¹R^{1′};
R¹ and R^{1′} are independently selected from the group consisting of hydrogen, —C₁₋₈ alkyl, —C₁₋₈ alkylene-NR²R^{2′}, —C₁₋₈ alkylene-OR², —C₀₋₈ alkylene-(3-10 membered cycloalkyl), —C₀₋₈ alkylene-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in the alkylene group are adapted to be replaced with an oxygen atom;
R² and R^{2′} are independently selected from the group consisting of hydrogen, —C₁₋₆ alkyl, —C₀₋₈ alkylene-(3-10 membered cycloalkyl), —C₀₋₈ alkyl-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in the alkylene group are adapted to be replaced with an oxygen atom;
T is -M-L^{T};
M is selected from the group consisting of —C₁₋₈ alkylene-, —C₀₋₈ alkylene-(3-10 membered cycloalkyl)-, —C₀₋₈ alkylene-(3-10 membered heterocycloalkyl)-, —C₀₋₈ alkylene-(5-12 membered spirocycloalkyl)-, —C₀₋₈ alkylene-(5-12 membered spiroheterocycloalkyl)-, —C₀₋₈ alkylene-(5-12 membered bridged cycloalkyl)- and —C₀₋₈ alkylene-(5-12 membered bridged heterocycloalkyl)-; wherein one or two carbon atoms in the alkylene group are adapted to be replaced with an oxygen atom;
L^{T} is selected from the group consisting of —C(O)—C₁₋₈ alkylene-NH₂, —C(O)—C₁₋₈ alkylene-NH(C₁₋₆ alkyl), —C(O)O—C₁₋₈ alkylene-NH₂, —C(O)O—C₁₋₈ alkylene-NH(C₁₋₆ alkyl), —C(O)NH—C₁₋₈ alkylene-NH₂, —C(O)NH—C₁₋₈ alkylene-NH(C₁₋₆ alkyl), —C₁₋₈ alkylene-NH₂, —C₁₋₈ alkylene-NH(C₁₋₆ alkyl), —C(O)—C₁₋₈ alkylene-(3-10 membered cycloalkyl), —C(O)O—C₁₋₈ alkylene-(3-10 membered cycloalkyl), —C(O)NH—C₁₋₈ alkylene-(3-10 membered cycloalkyl), —C₁₋₈ alkylene-(3-10 membered cycloalkyl), —C(O)—C₁₋₈ alkylene-(3-10 membered heterocycloalkyl), —C(O)O—C₁₋₈ alkylene-(3-10 membered heterocycloalkyl), —C(O)NH—C₁₋₈ alkylene-(3-10 membered heterocycloalkyl), —C₁₋₈ alkylene-(3-10 membered heterocycloalkyl), —C(O)—C₁₋₈ alkylene-(5-12 membered spirocycloalkyl), —C(O)O—C₁₋₈ alkylene-(5-12 membered spirocycloalkyl), —C(O)NH—C₁₋₈ alkylene-(5-12 membered spirocycloalkyl), —C₁₋₈ alkylene-(5-12 membered spirocycloalkyl), —C(O)—C₁₋₈ alkylene-(5-12 membered spiroheterocycloalkyl), —C(O)O—C₁₋₈ alkylene-(5-12 membered spiroheterocycloalkyl), —C(O)NH—C₁₋₈ alkylene-(5-12 membered spiroheterocycloalkyl), —C₁₋₈ alkylene-(5-12 membered spiroheterocycloalkyl), —C(O)—C₁₋₈ alkylene-(5-12 membered bridged cycloalkyl), —C(O)O—C₁₋₈ alkylene-(5-12 membered bridged cycloalkyl), —C(O)NH—C₁₋₈ alkylene-(5-12 membered bridged cycloalkyl), —C₁₋₈ alkylene-(5-12 membered bridged cycloalkyl), —C(O)—C₁₋₈ alkylene-(5-12 membered bridged heterocycloalkyl), —C(O)O—C₁₋₈ alkylene-(5-12 membered bridged heterocycloalkyl), —C(O)NH—C₁₋₈ alkylene-(5-12 membered bridged heterocycloalkyl), —C₁₋₈ alkylene-(5-12 membered bridged heterocycloalkyl), —C(O)—C₁₋₈ alkylene-OH, —C(O)O—C₁₋₈ alkylene-OH, —C(O)NH—C₁₋₈ alkylene-OH, —C₁₋₈ alkylene-OH, —S(O)₂—C₁₋₈ alkylene-NH₂, —S(O)₂—C₁₋₈ alkylene-NH(C₁₋₆ alkyl), —S(O)₂O—C₁₋₈ alkylene-NH₂, —S(O)₂O—C₁₋₈ alkylene-NH(C₁₋₆ alkyl), —S(O)₂NH—C₁₋₈ alkylene-NH₂, —S(O)₂NH—C₁₋₈ alkylene-NH(C₁₋₆ alkyl), —S(O)₂—C₁₋₈ alkylene-(3-10 membered cycloalkyl), —S(O)₂O—C₁₋₈ alkylene-(3-10 membered cycloalkyl), —S(O)₂NH—C₁₋₈ alkylene-(3-10 membered cycloalkyl), —S(O)₂—C₁₋₈ alkylene-(3-10 membered heterocycloalkyl), —S(O)₂O—C₁₋₈ alkylene-(3-10 membered heterocycloalkyl), —S(O)₂NH—C₁₋₈ alkylene-(3-10 membered heterocycloalkyl), —S(O)₂—C₁₋₈ alkylene-(5-12 membered spirocycloalkyl), —S(O)₂O—C₁₋₈ alkylene-(5-12 membered spirocycloalkyl), —S(O)₂NH—C₁₋₈ alkylene-(5-12 membered spirocycloalkyl), —S(O)₂—C₁₋₈ alkylene-(5-12 membered spiroheterocycloalkyl), —S(O)₂O—C₁₋₈ alkylene-(5-12 membered spiroheterocycloalkyl), —S(O)₂NH—C₁₋₈ alkylene-(5-12 membered spiroheterocycloalkyl), —S(O)₂—C₁₋₈ alkylene-(5-12 membered bridged cycloalkyl), —S(O)₂O—C₁₋₈ alkylene-(5-12 membered bridged cycloalkyl), —S(O)₂NH—C₁₋₈ alkylene-(5-12 membered bridged cycloalkyl), —S(O)₂—C₁₋₈ alkylene-(5-12 membered bridged heterocycloalkyl), —S(O)₂O—C₁₋₈ alkylene-(5-12 membered bridged heterocycloalkyl), —S(O)₂NH—C₁₋₈ alkylene-(5-12 membered bridged heterocycloalkyl), —S(O)₂—C₁₋₈ alkylene-OH, —S(O)₂O—C₁₋₈ alkylene-OH and —S(O)₂NH—C₁₋₈ alkylene-OH; wherein one or two carbon atoms in the alkylene group are adapted to be replaced with an oxygen atom.

In some embodiments, M is selected from the group consisting of

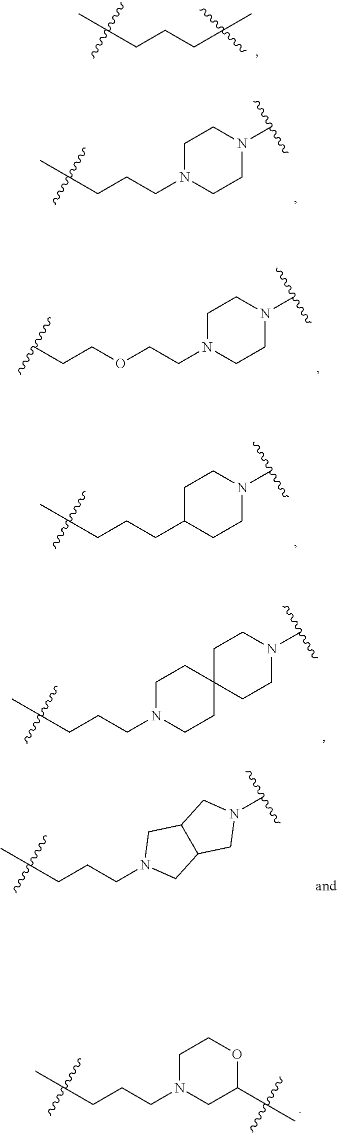

In some embodiments, L^{T} is selected from the group consisting of

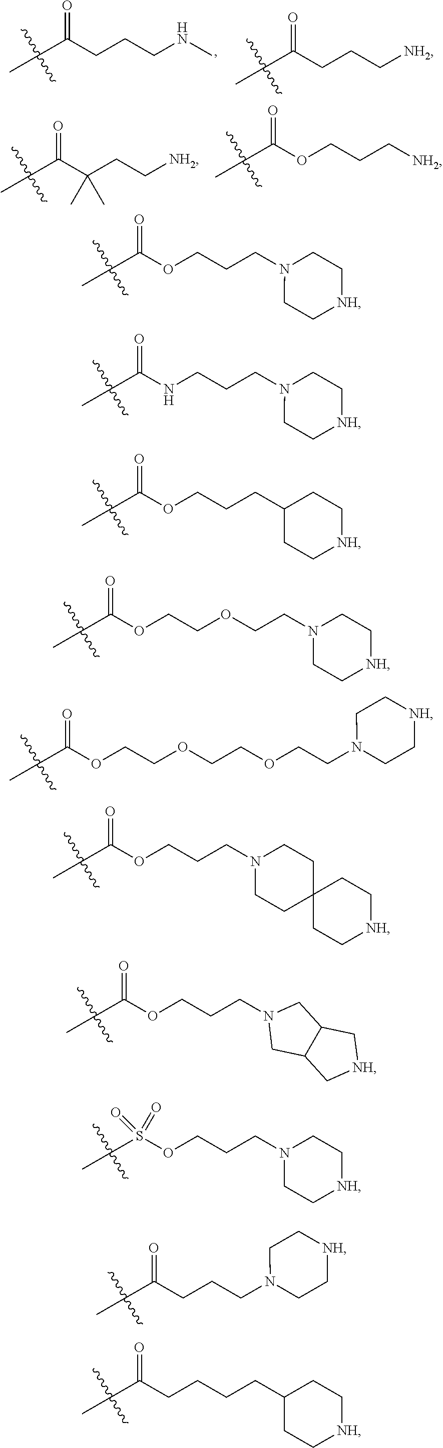

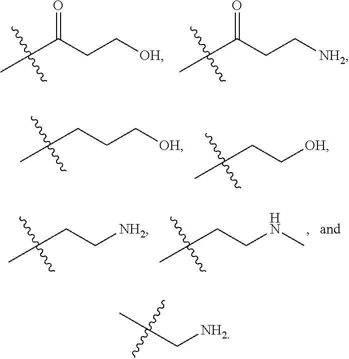

In some embodiments, the ring A and the ring B are independently selected from the group consisting of

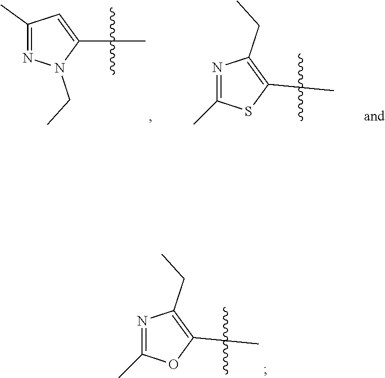

X² is N or CR^{X};
R^{X} is —OR¹ or —SR¹; and
R¹ is methyl, or -propylidene-OH.

The present disclosure further provides a compound of formula (III), or a deuterated compound, a stereoisomer, or a pharmaceutically acceptable salt thereof:

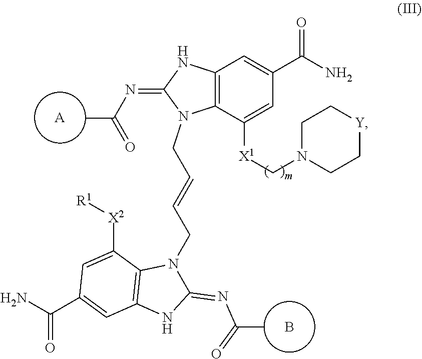

wherein ring A and ring B are independently

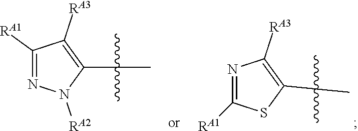

and at least one of the ring A and the ring B is

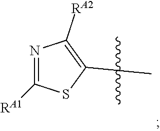

R^{A1} and R^{A2} are independently hydrogen, or —C₁₋₆ alkyl;
R^{A3} is selected from the group consisting of hydrogen, halogen and —C₁₋₆ alkyl;
X¹ and X² are independently O or S;
m is 1, 2, 3, 4, 5 or 6;
Y is O or NR^{Y};
R^{Y} is selected from the group consisting of —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆ alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in the alkylene group are adapted to be replaced with an oxygen atom;
R¹ is selected from the group consisting of —C₁₋₆ alkyl, —C₁₋₆ alkylene-NH₂ and —C₁₋₆ alkylene-NH—R²; and when Y is O, R¹ is not —C₁₋₆ alkyl;
R² is selected from the group consisting of —C₁₋₆ alkyl, —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in the alkylene group are adapted to be replaced with an oxygen atom.

In some embodiments, Y is NR^{Y}; and R¹ is —C₁₋₆ alkyl.

In some embodiments, the compound of formula (III) is represented by the formula (IVa) or formula (IVb):

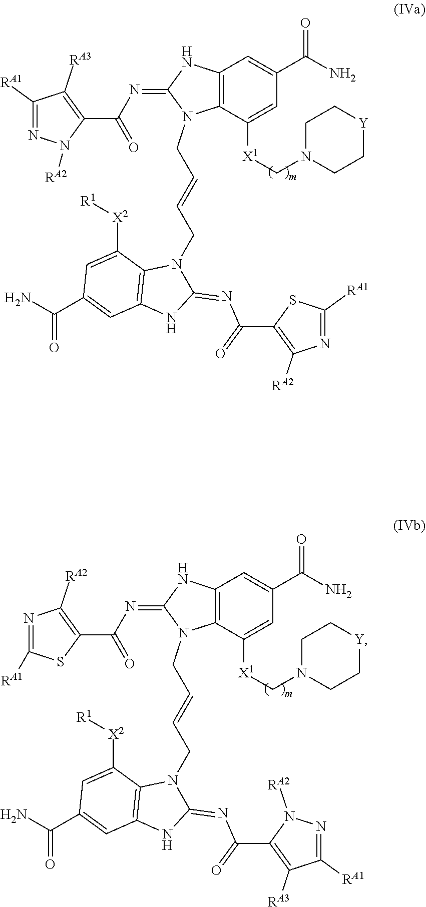

wherein R^{A1} is selected from the group consisting of methyl, ethyl, n-propyl and isopropyl;
R^{A2} is selected from the group consisting of methyl, ethyl, n-propyl and isopropyl;
R^{A3} is selected from the group consisting of hydrogen, fluorine, chlorine, methyl and ethyl;
X¹ is O or S;
X² is O or S;
m is 1, 2, 3, 4, 5 or 6;
R¹ is selected from the group consisting of methyl, ethyl, n-propyl and isopropyl;
Y is NR^{Y}; and
R^{Y} is selected from the group consisting of —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆ alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in the alkylene group are adapted to be replaced with an oxygen atom.

In some embodiments, R^{Y} is selected from the group consisting of

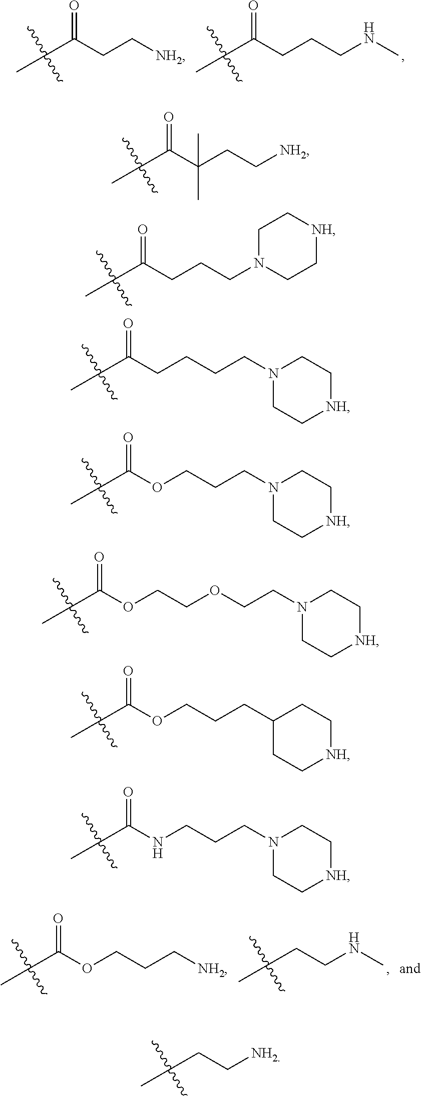

In some embodiments, Y is O; R¹ is —C₁₋₆ alkylene-NH₂ or —C₁₋₆ alkylene-NH—R².

In some embodiments, the compound of formula (III) is represented by the formula (IVc) or formula (IVd):

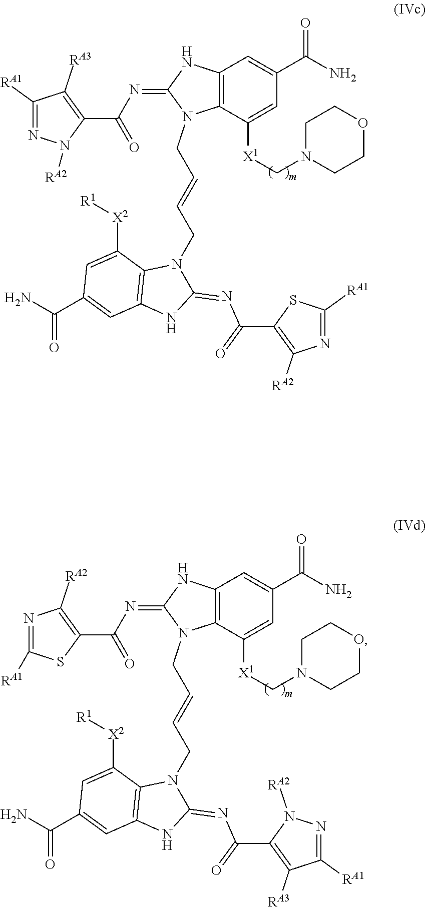

wherein R^{A1} is selected from the group consisting of methyl, ethyl, n-propyl and isopropyl;
R^{A2} is selected from the group consisting of methyl, ethyl, n-propyl and isopropyl;
R^{A3} is selected from the group consisting of hydrogen, fluorine, chlorine, methyl and ethyl;
X¹ is O or S;
X² is O or S;
m is 1, 2, 3, 4, 5 or 6;
R¹ is —C₁₋₆ alkylene-NH₂ or —C₁₋₆ alkylene-NH—R²;
R² is selected from the group consisting of —C₁₋₆ alkyl, —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in the alkylene group are adapted to be replaced with an oxygen atom.

In some embodiments, R¹ is

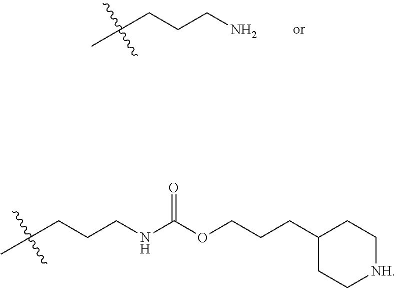 [SMILES: *CCCN ; *CCCNC(=O)OCCCC1CCNCC1]

In some embodiments, the compound of formula (II) or (III) is selected from the group consisting of:

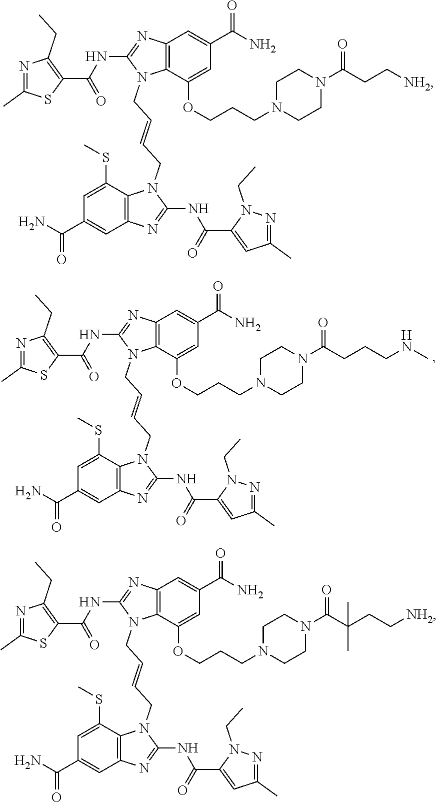

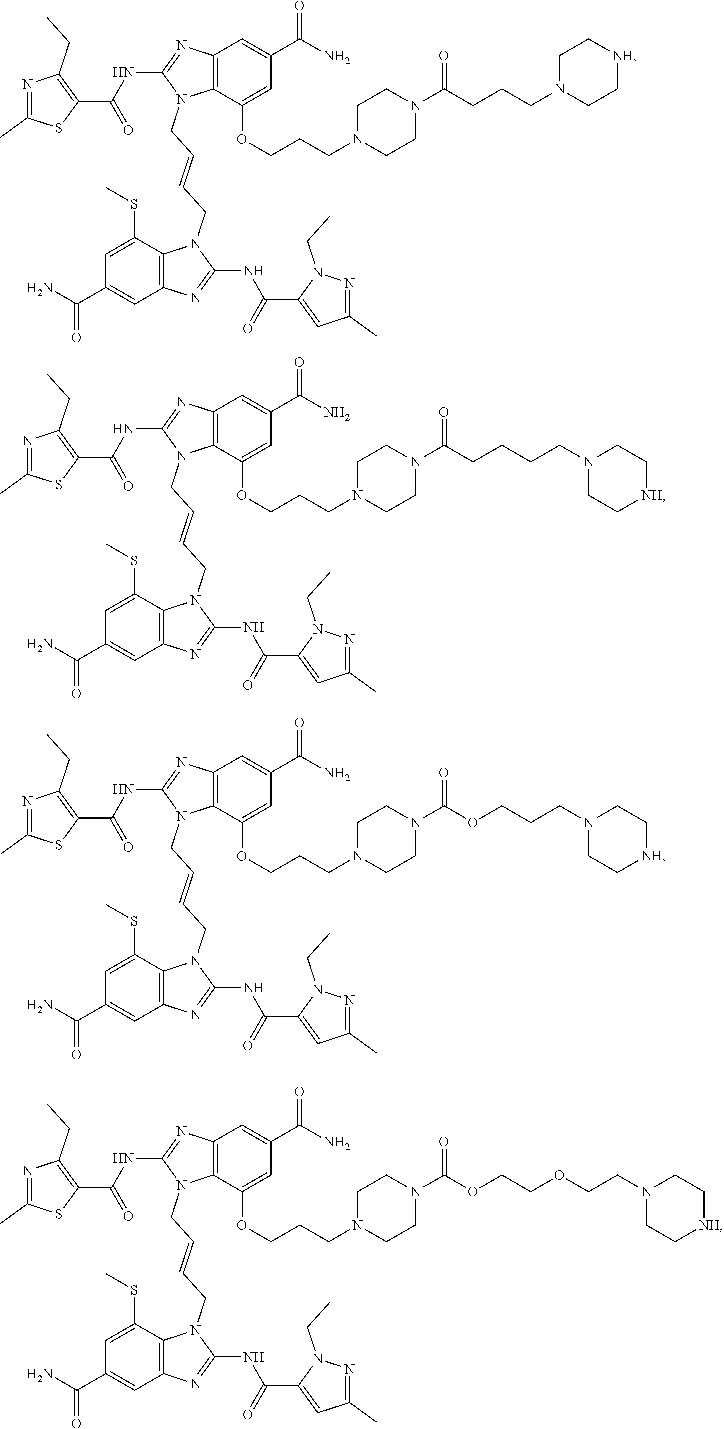

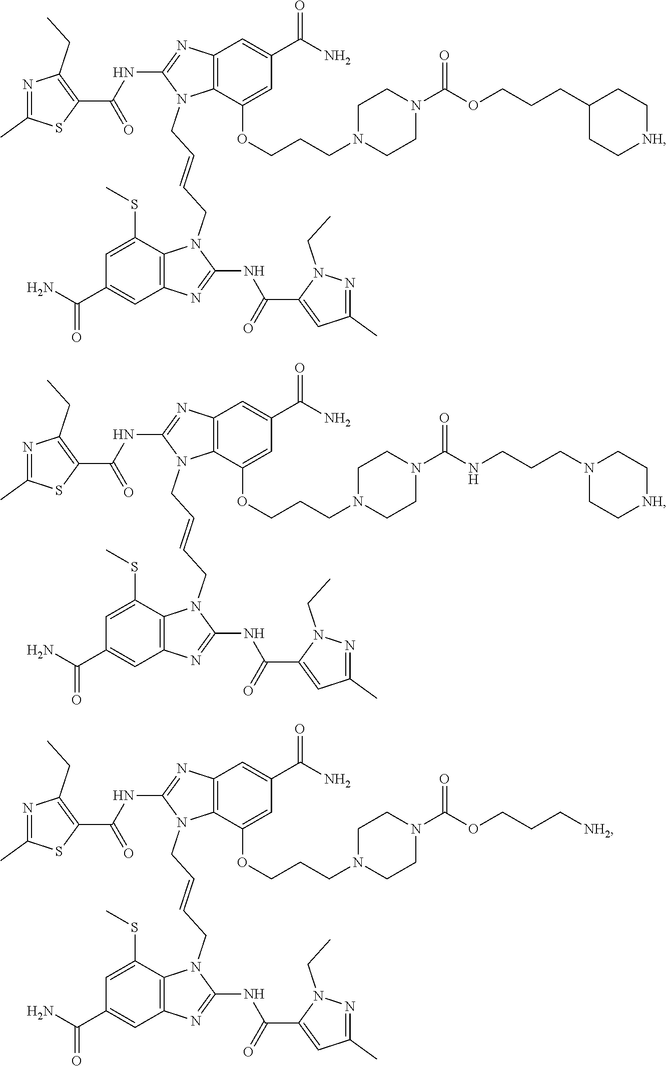

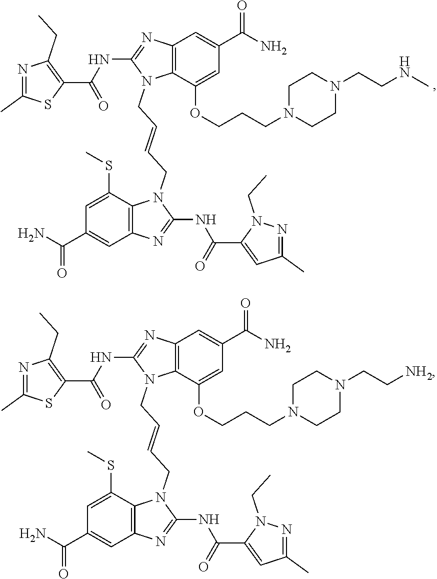

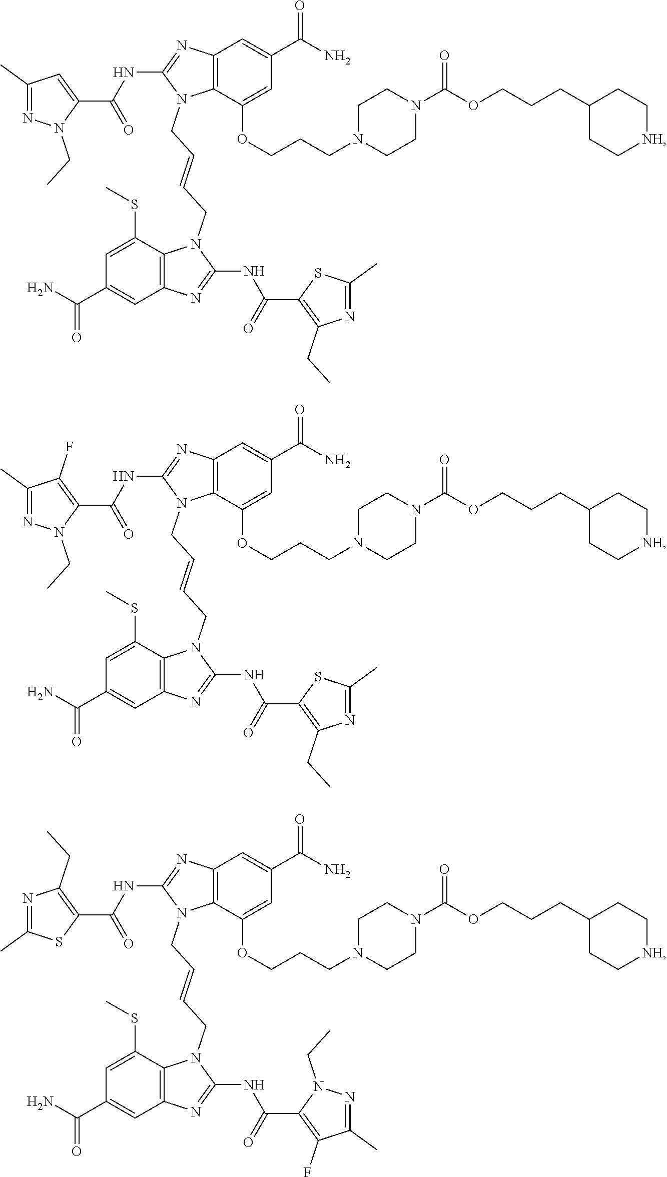

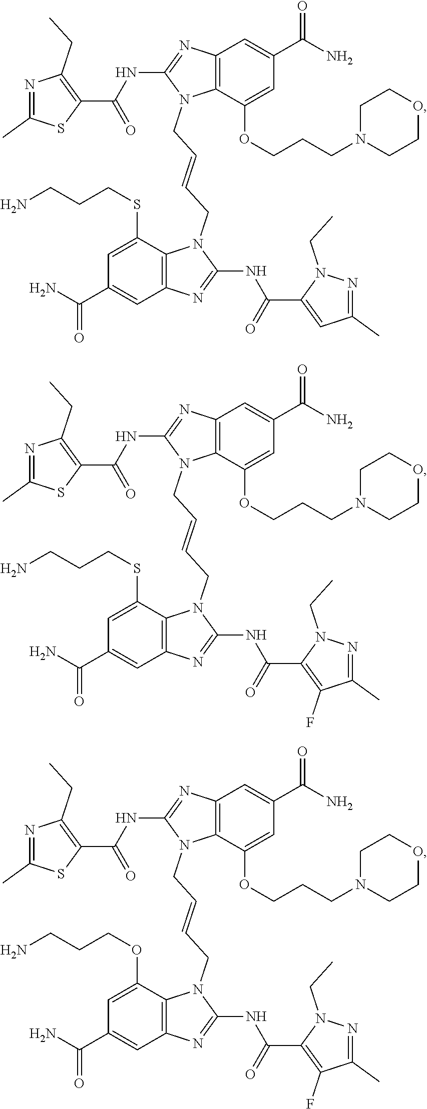

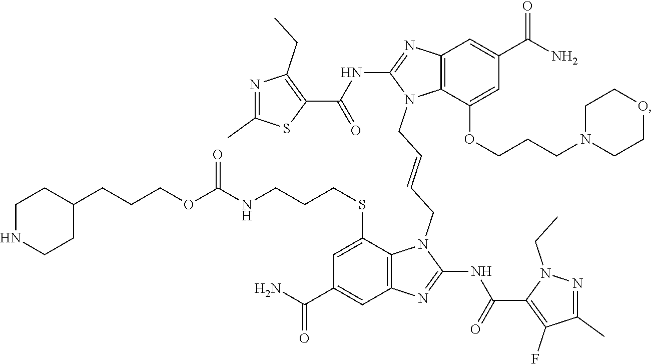 [SMILES: CCc1nc(C)sc1C(=O)Nc1nc2cc(C(N)=O)cc(OCCCN3CCOCC3)c2n1C/C=C/Cn1c(NC(=O)c2c(F)c(C)nn2CC)nc2cc(C(N)=O)cc(SCCCNC(=O)OCCCC3CCNCC3)c21]

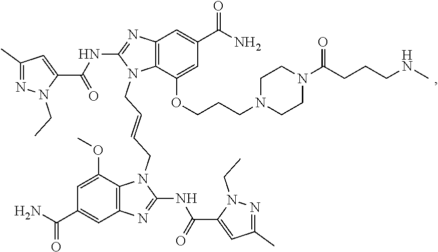

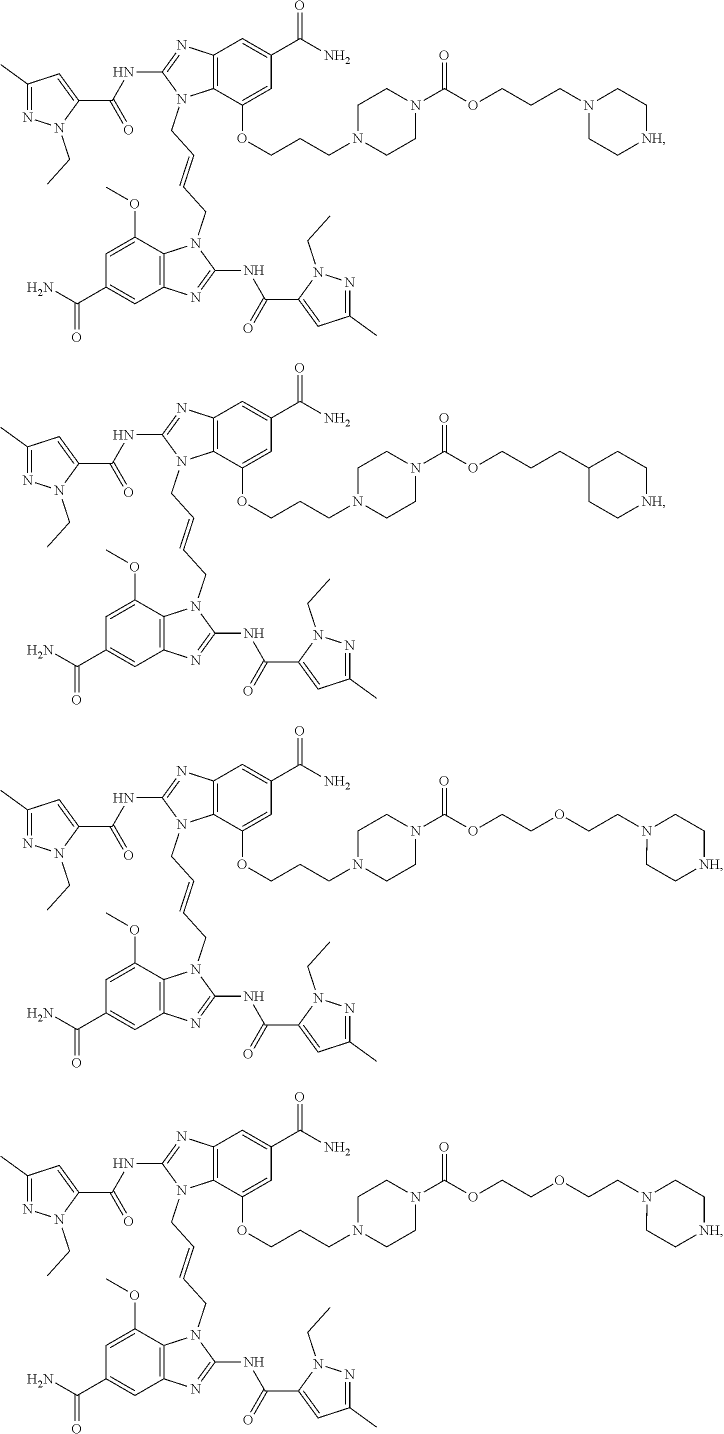

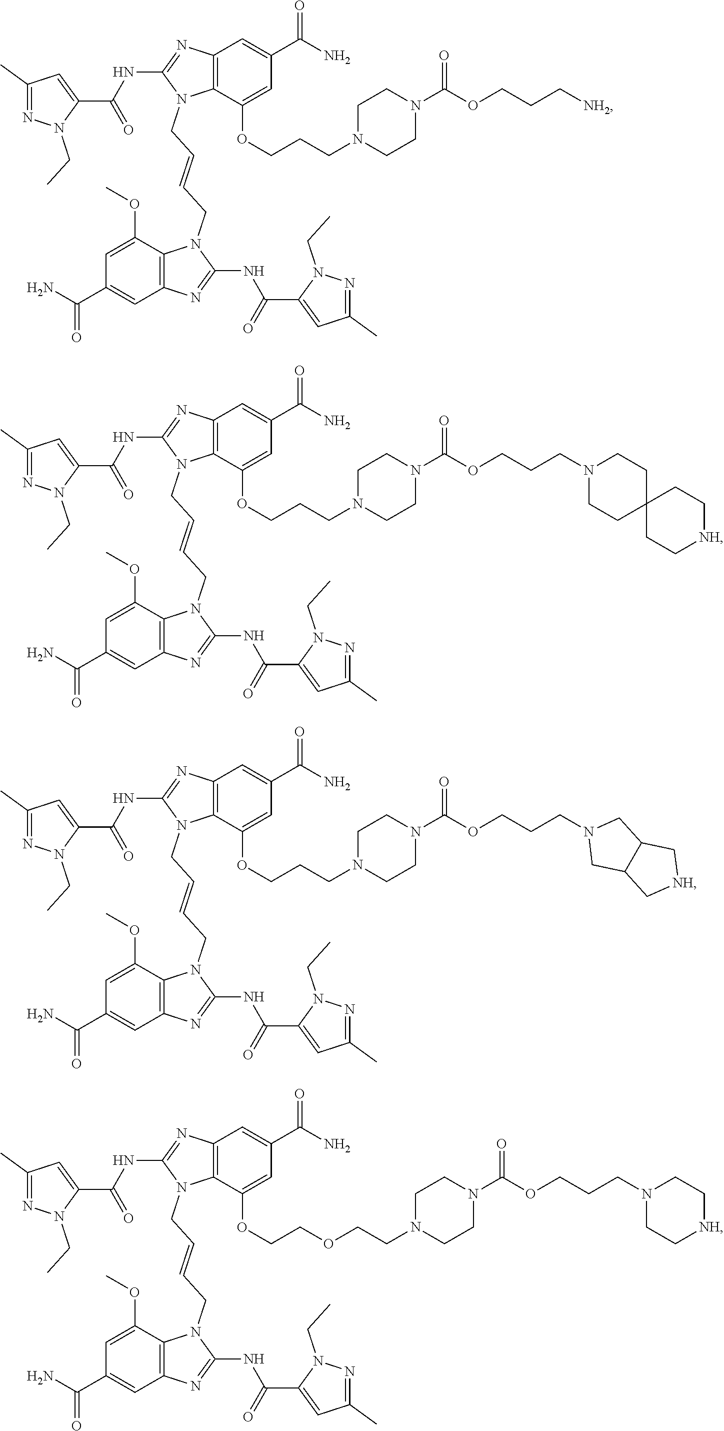

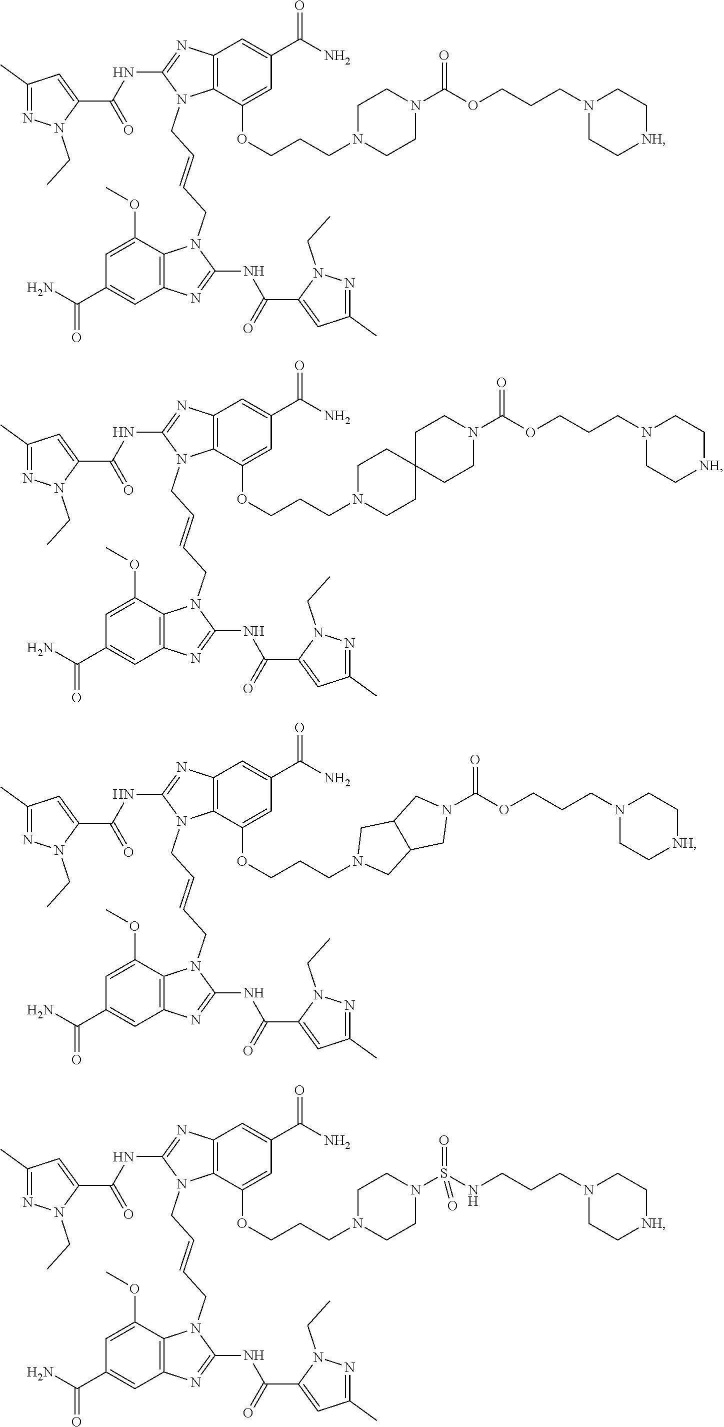

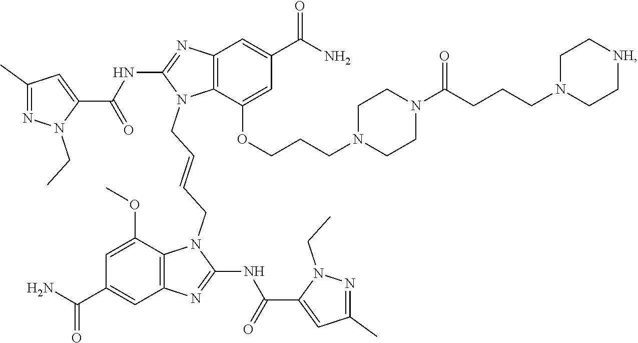 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCCN4CCNCC4)CC3)c21]

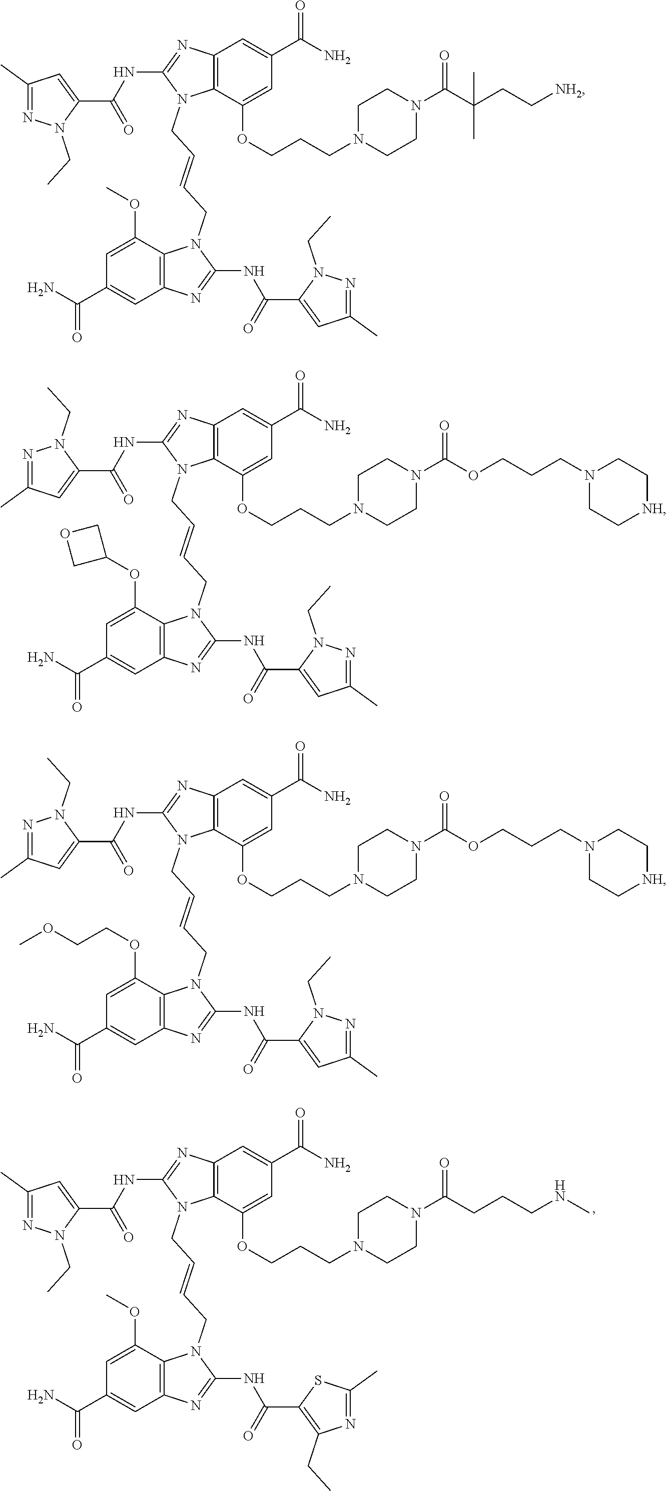

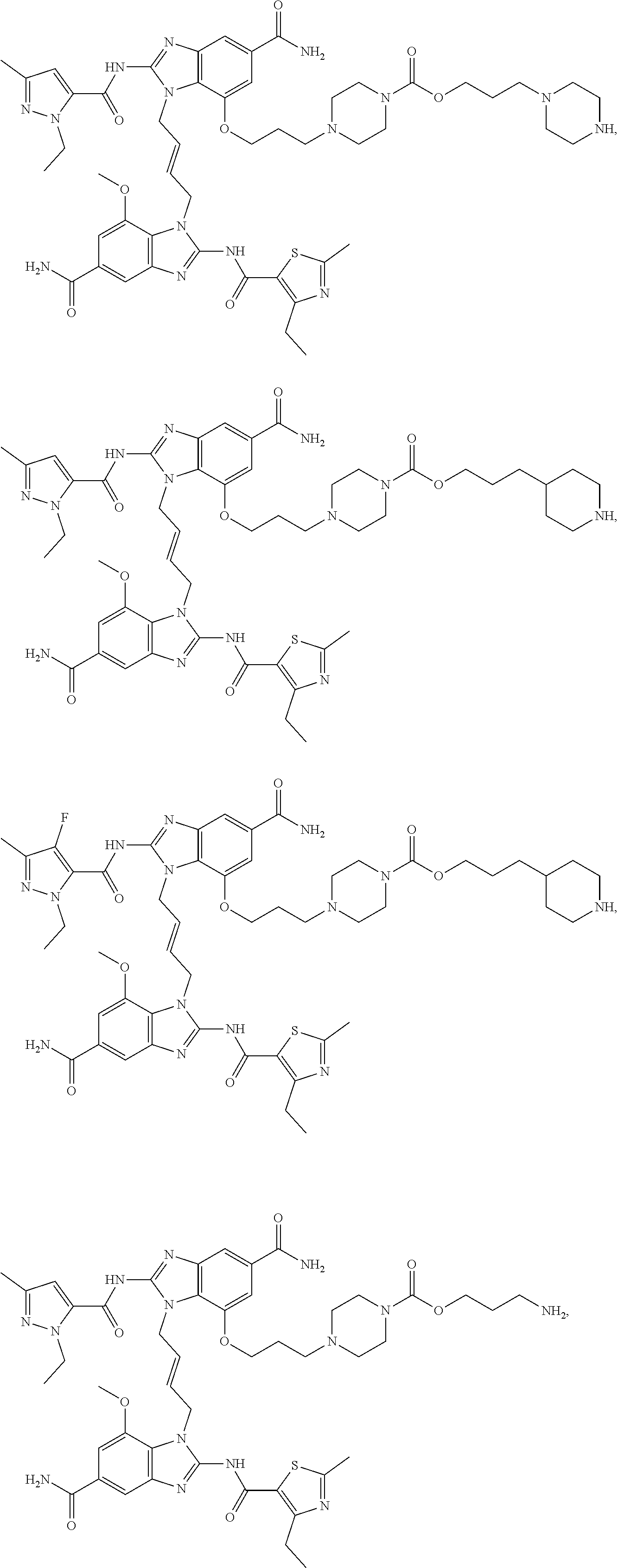

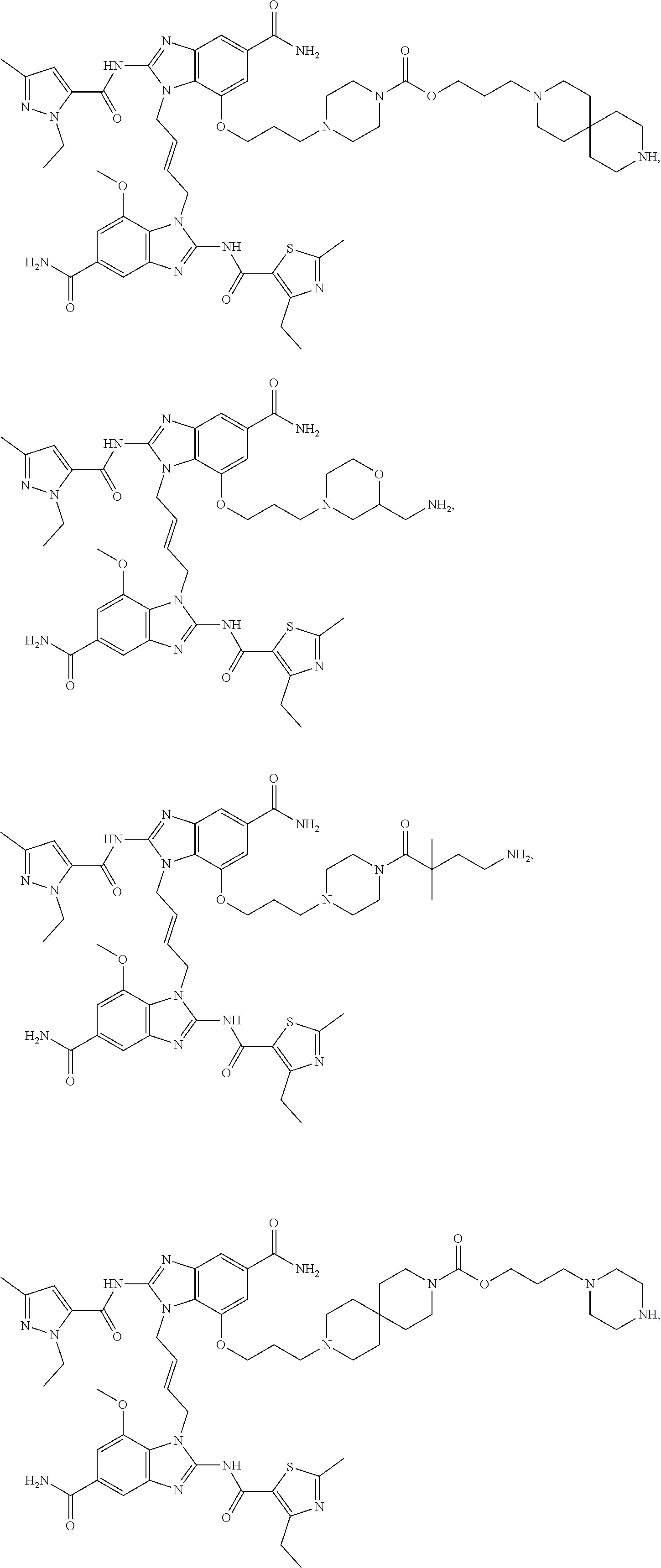

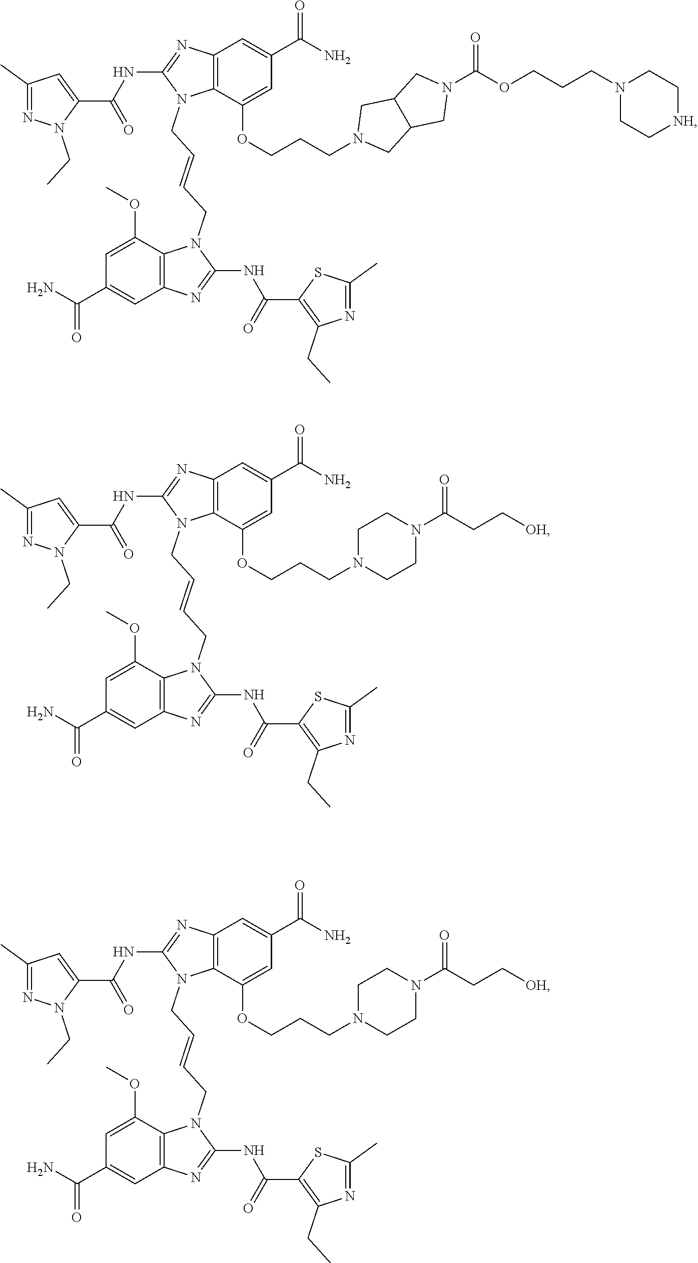 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4CN(C(=O)OCCCN5CCNCC5)CC4C3)c21 ; CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCO)CC3)c21 ; CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCO)CC3)c21]

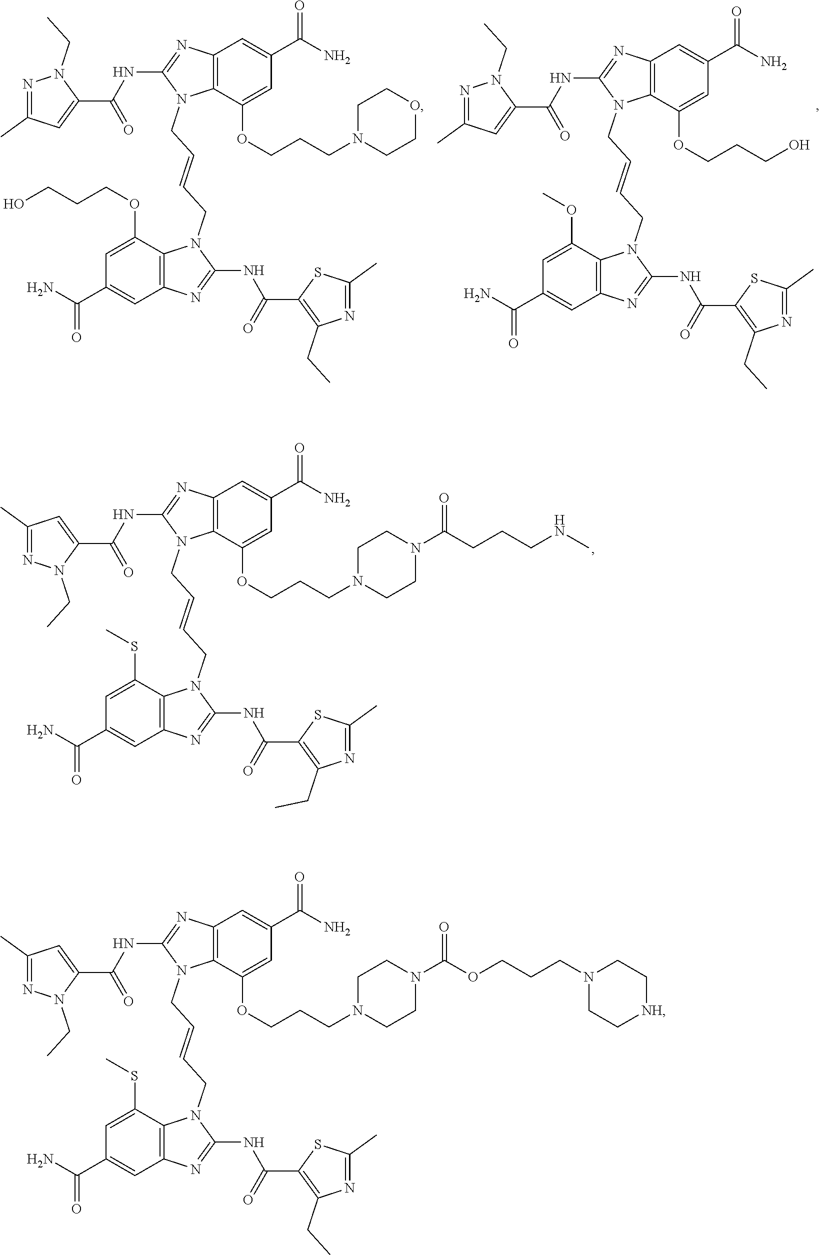

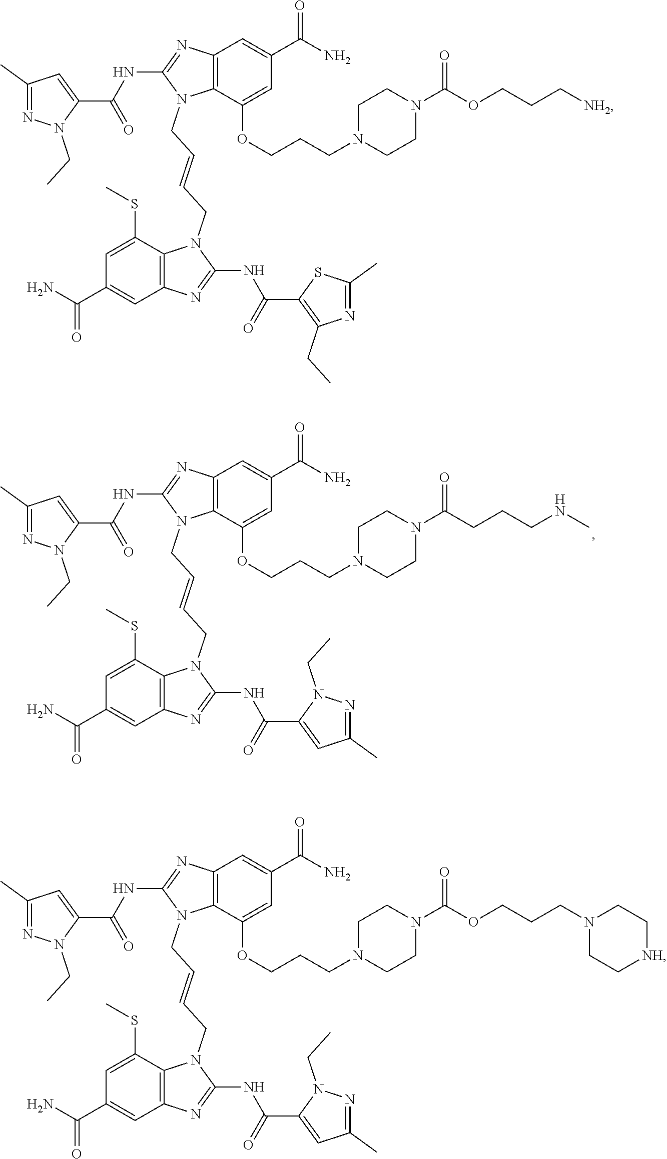

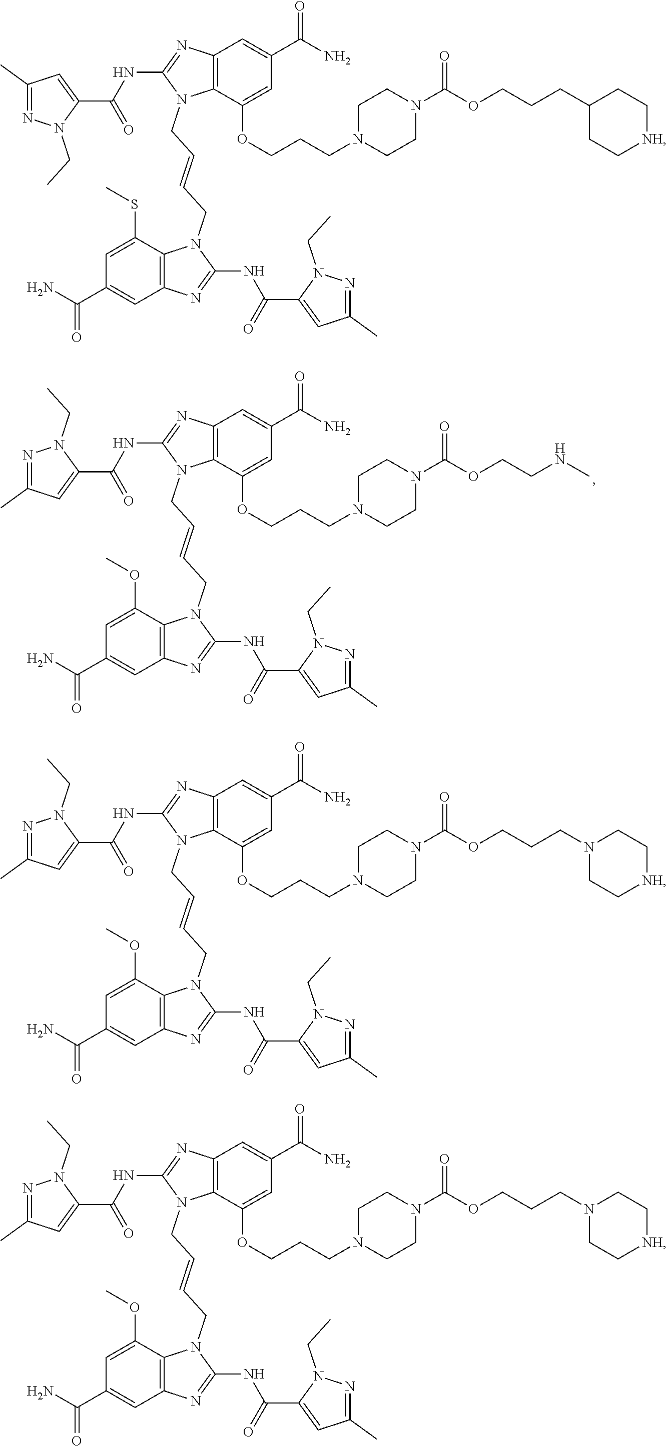

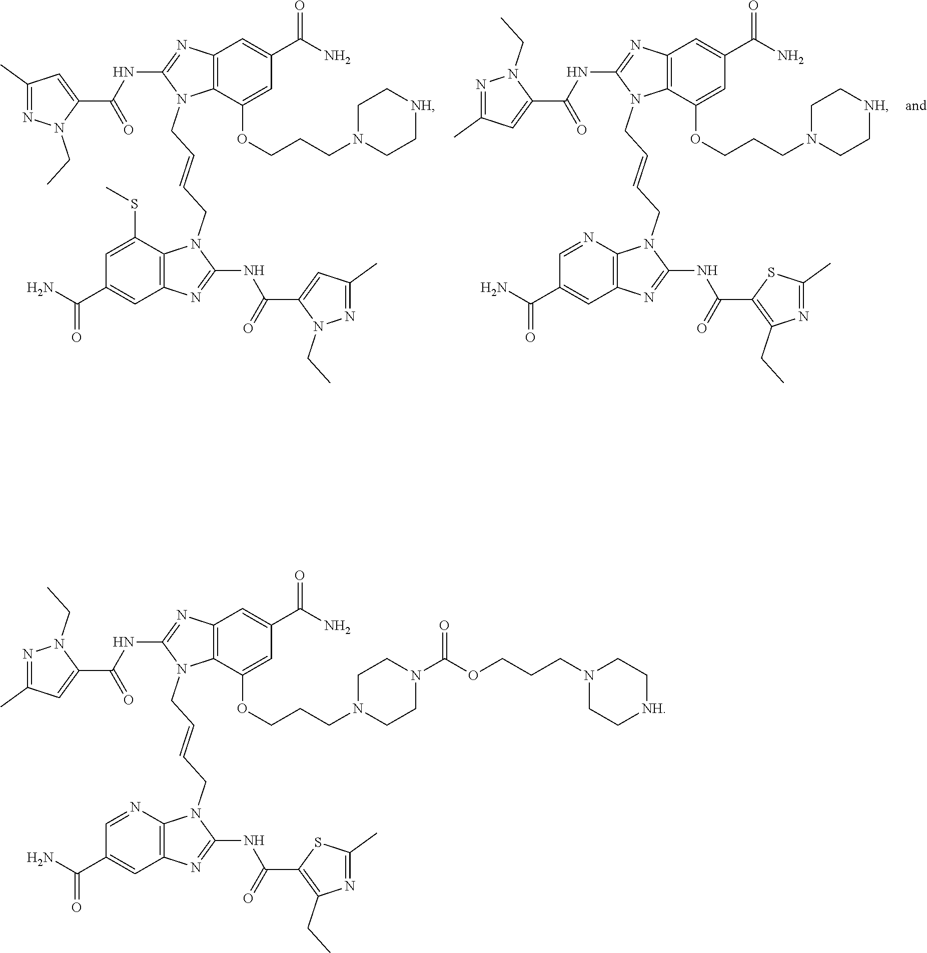

The present disclosure further provides an application of the aforementioned compound or a deuterated compound, a stereoisomer or a pharmaceutically acceptable salt thereof, or the aforementioned compound-linker conjugate in the preparation of antibody drug conjugates.

In some embodiments, the aforementioned compound, or a deuterated compound, a stereoisomer or a pharmaceutically acceptable salt thereof, or the aforementioned compound-linker conjugate is used as an intermediate in the preparation of antibody drug conjugates.

The present disclosure further provides an application of the aforementioned compound, or a deuterated compound, a stereoisomer, or a pharmaceutically acceptable salt thereof as a payload in the preparation of antibody drug conjugates.

The compound provided by the present disclosure can be coupled to the antibody via the linker with the amino group or imino group as a linking site. In some embodiments, the antibody drug conjugate prepared from the compound provided by the present disclosure can release the compound of the present disclosure in specific conditions, and maintain its biological activity.

The compounds provided herein and derivatives thereof can be named according to the IUPAC (International Union of Pure and Applied Chemistry) or CAS (Chemical Abstracts Service, Columbus, OH) nomenclature system.

### Terminology

Unless otherwise specified, the initial definition of group or term provided herein is applicable throughout the specification; and for those terms that are not specifically defined herein should be construed according to the disclosure and context.

“Substitution” means that the hydrogen atom in the molecule is replaced with other different atoms or groups; or the lone electron pair of an atom in the molecule is replaced by other atom or group, for example, the lone electron pair on the S atom can be replaced by the O atom to generate

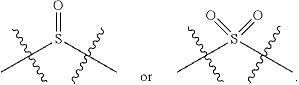

The minimum and maximum carbon atom number in a hydrocarbon group are expressed by a prefix, for example, the prefix C_{a-b} alkyl indicates any alkyl group containing “a-b” carbon atoms. Exemplarily, the “C₁₋₆ alkyl” refers to an alkyl group containing 1-6 carbon atoms.

“Alkyl” refers to a saturated hydrocarbon chain which has a dictated number of member atoms. The alkyl group can be straight-chain alkyl or branched alkyl. The representative branched alkyl group has one, two or three branches. The alkyl group may be optionally substituted with one or more substituents as defined herein. The alkyl group includes methyl, ethyl, propyl (n-propyl and isopropyl), butyl (n-butyl, isobutyl and tert-butyl), pentyl (n-pentyl, isopentyl and neopentyl) and hexyl. The alkyl group may also be a part of other groups, such as —O(C₁₋₆ alkyl).

“Alkylene” refers to a divalent saturated aliphatic hydrocarbon group with a dictated number of member atoms. C_{a-b} alkylene refers to an alkylene group having a-b carbon atoms. The alkylene group includes branched and straight-chain hydrocarbon groups. For example, the term “propylidene” can be exemplified by the following structure:

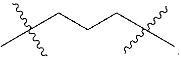 [SMILES: *CCC*]

Likewise, the term “dimethylbutylene” can be exemplified by any one of the following structures:

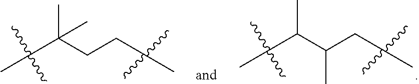

The term “unsaturated” in the present disclosure refers to the groups or molecules which have double bonds and/or triple bonds, including but not limited to carbon-carbon double bonds, carbon-carbon triple bonds, carbon-oxygen double bonds, carbon-sulfur double bonds and carbon-nitrogen triple bonds.

The “halogen” mentioned herein refers to fluorine, chlorine, bromine or iodine.

The “chemical bond” in the present disclosure refers to the direct linking of two parts via a chemical single bond.

The “halogenated alkyl” in the present disclosure means that one or more hydrogen atoms in the alkyl group are replaced with halogen, e.g., monofluoromethyl, difluoromethyl and trifluoromethyl.

“—OR”, “—NRR” and the analogical groups mentioned in the present disclosure indicates that the R group is linked to an oxygen or nitrogen atom via a single bond.

Regarding the “—C(O)R”, the oxygen atom is linked to a carbon atom or a sulfur atom via a double bond, and the R group is linked to an oxygen atom or a sulfur atom via a single bond.

As used herein,

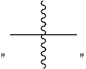

is used to describe the substituent position.

The “deuterated compound” in the present disclosure refers to the substitution of one or more hydrogen atoms in a molecule or group by deuterium atoms, where the proportion of deuterium atoms is greater than the natural abundance of deuterium.

The compounds of the present disclosure include their tautomers, and those skilled in the art can understand their possible tautomers according to the structural formulas of the compounds, for example, the two following structural formulas represent tautomers of the same compound:

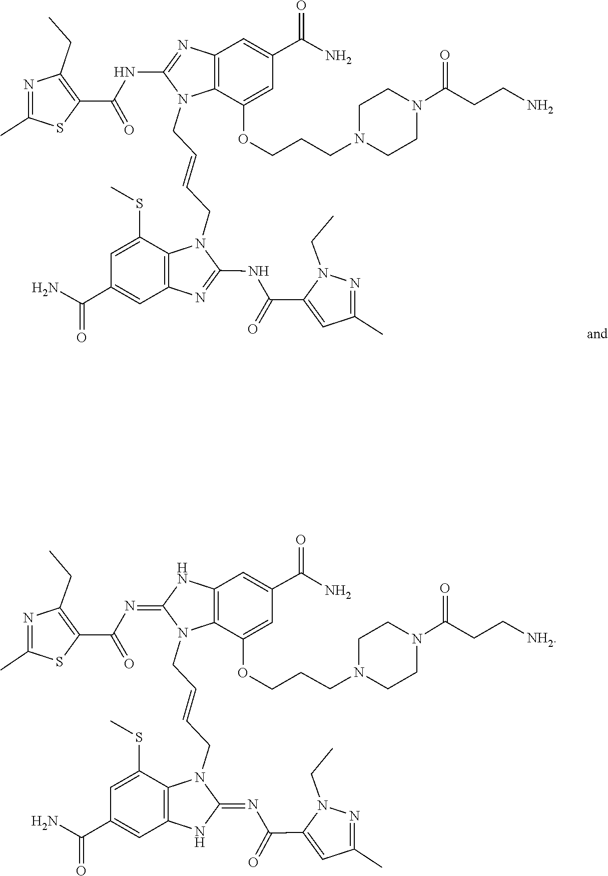 [SMILES: CCc1nc(C)sc1C(=O)Nc1nc2cc(C(N)=O)cc(OCCCN3CCN(C(=O)CCN)CC3)c2n1C/C=C/Cn1c(NC(=O)c2cc(C)nn2CC)nc2cc(C(N)=O)cc(SC)c21 ; CCc1nc(C)sc1C(=O)/N=c1/[nH]c2cc(C(N)=O)cc(OCCCN3CCN(C(=O)CCN)CC3)c2n1C/C=C/Cn1/c(=N/C(=O)c2cc(C)nn2CC)[nH]c2cc(C(N)=O)cc(SC)c21]

The term “pharmaceutically acceptable” means that a certain carrier, diluent, excipient, and/or the salt thereof typically has/have chemical or physical compatibility with other ingredients of a pharmaceutical preparation, and physiological compatibility with receptors.

The terms “salt” and “pharmaceutically acceptable salt” include acidic and/or basic salts formed by the aforementioned compounds or their stereoisomers with inorganic and/or organic acids and bases, zwitterionic salts (internal salts), and quaternary ammonium salts, such as alkyl ammonium salts. These salts can be directly obtained from the separation and purification of the compounds. The salts can also be obtained by mixing the aforementioned compounds, or stereoisomer thereof, with a certain amount of acid or base as appropriate (e.g., equivalent). The salts may be insoluble, and can be collected by filtration, evaporation or freeze drying. The salts described herein include but not limited to hydrochloride, sulfate, citrate, benzene sulfonate, hydrobromide, hydrofluoride, phosphate, acetate, propionate, succinate, oxalate, malate, fumarate, maleate, tartrate, and trifluoroacetate.

Apparently, according to the foregoing disclosure, various modifications, replacements or variations can be made by those skilled in the art without departing from the spirit of the present disclosure.

The disclosure will be further described in detail below with reference to the following examples. However, it is to be understood that the scope of the present disclosure is not limited to the following exemplary embodiments. All embodiments implemented in light of the content disclosed herein should be included within the scope of the present disclosure.

### BRIEF DESCRIPTION OF THE DRAWINGS

FIG. 1 is a ¹HNMR spectrogram of a compound prepared in Example 41 of the present disclosure;

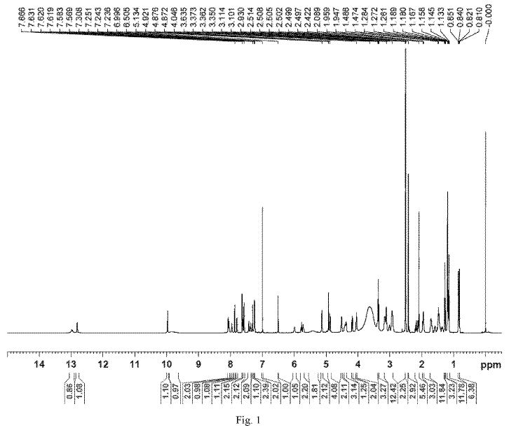

FIG. 2 is a ¹HNMR spectrogram of a compound prepared in Example 40 of the present disclosure;

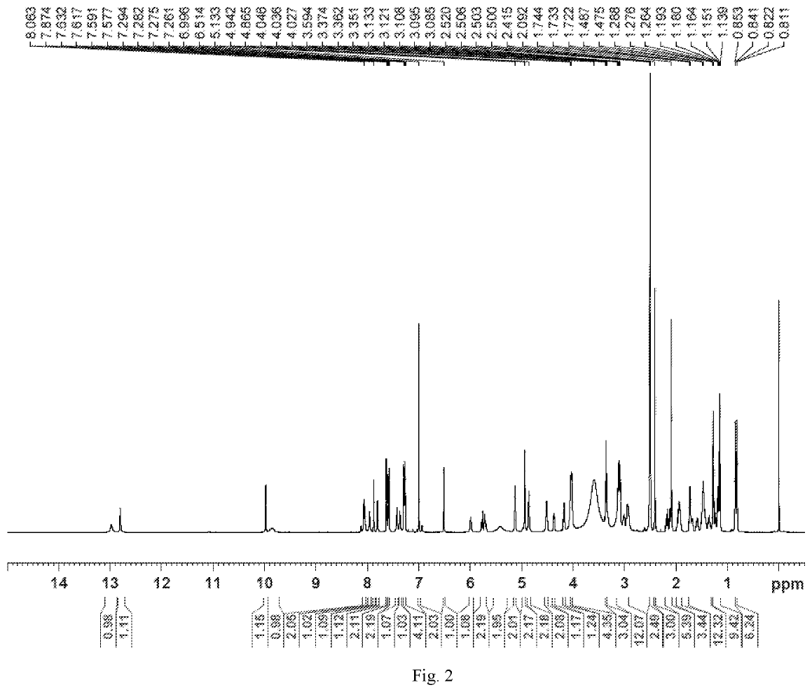

FIG. 3 is a ¹HNMR spectrogram of a compound prepared in Example 34 of the present disclosure;

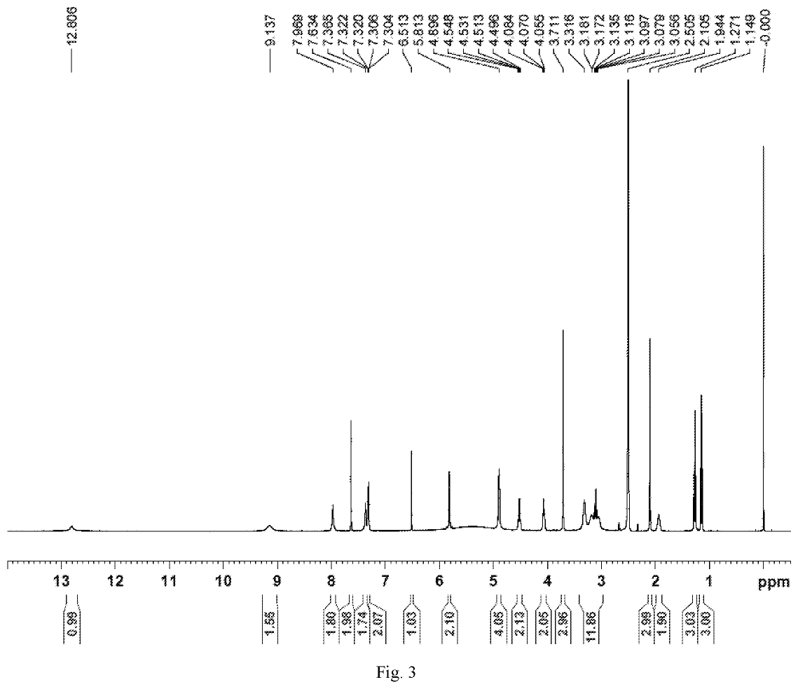

FIG. 4 is a ¹HNMR spectrogram of a compound prepared in Example 35 of the present disclosure;

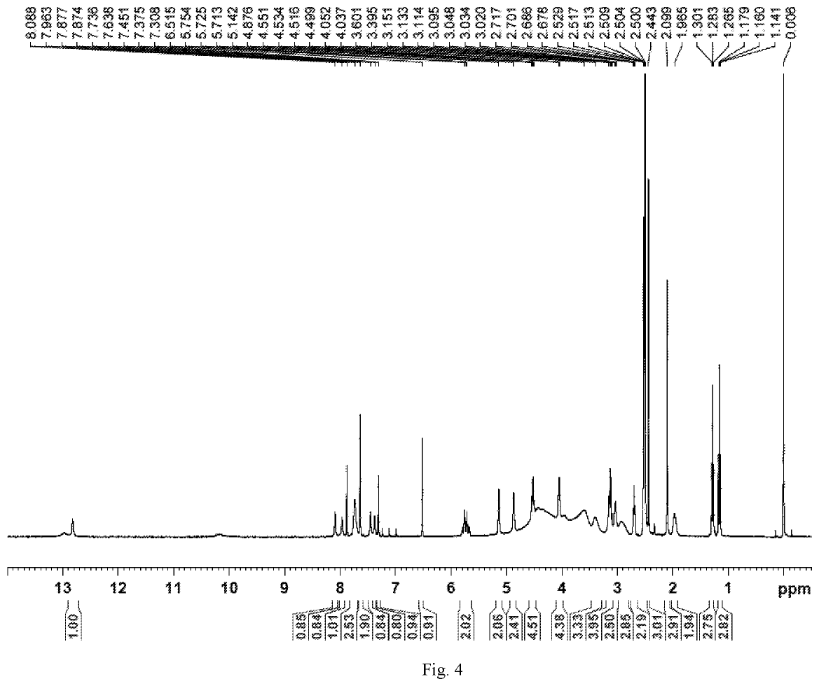

FIG. 5 is a ¹HNMR spectrogram of the compound prepared in Example 28 of the present disclosure; and

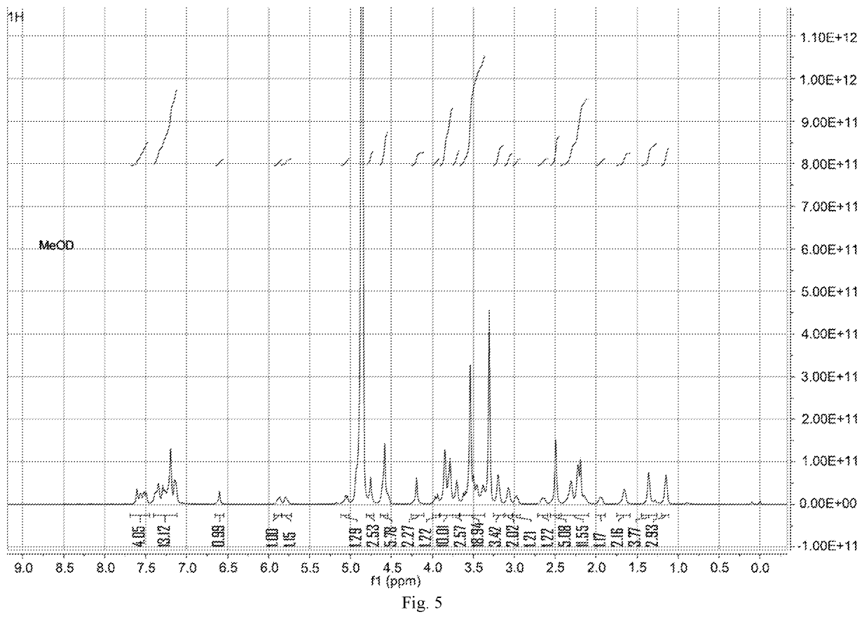

FIG. 6 is a ¹HNMR spectrogram of a compound prepared in Example 29 of the present disclosure.

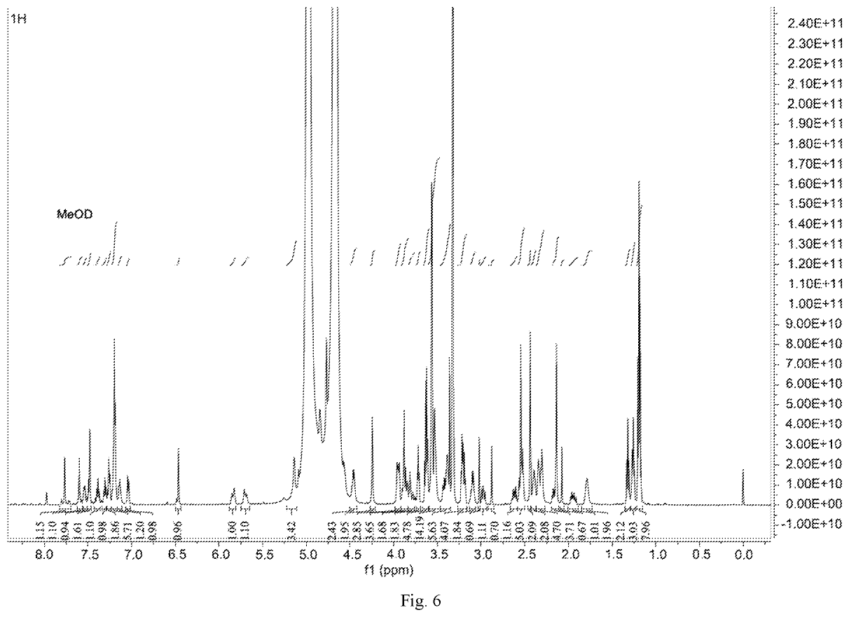

### DETAILED DESCRIPTION OF EMBODIMENTS

Compounds are structurally characterized by Nuclear Magnetic Resonance (NMR) Spectroscopy and Mass Spectroscopy (MS). The NMR shift (δ) is expressed in a unit of 10⁻⁶ (ppm). The NMR spectra are obtained by using a Nuclear Magnetic Resonance Spectrometer (Bruker AvanceIII 400 and Bruker Avance 300) with deuterated dimethyl sulfoxide (DMSO-d₆), deuterated chloroform (CDCl₃), or deuterated methanol (CD₃OD) as the solvent, and tetramethylsilane (TMS) as the internal standard.

LC-MS is carried out using Shimadzu LC-MS 2020 (ESI). HPLC is performed using a Shimadzu High-Performance Liquid Chromatograph (Shimadzu LC-20A). MPLC (Medium-Pressure Preparative Liquid Chromatography) is performed on a Gilson GX-281 reversed phase preparative chromatograph. Yantai Huanghai HSGF254 or Qingdao GF254 silica gel plates are used for thin layer chromatography, and the product size used in the thin layer chromatography was 0.4 mm to 0.5 mm. Typically, 200-300 mesh silica gel (Yantai Huanghai silica gel) was used as the carrier in column chromatography.

The known starting materials of the present disclosure can be synthesized according to the methods known in the prior art, or can be purchased from Energy Chemical LLC, Chengdu Kelong Chemical LLC, Shaoyuan Chemical Scientific LLC, or J&K Scientific LLC.

Unless otherwise specified, the reaction is carried out under a nitrogen atmosphere; the solution refers to an aqueous solution; the reaction temperature is room temperature; and M represents mol/L.

### Abbreviations

PE: petroleum ether; EA: ethyl acetate; DCM: dichloromethane; MeOH: methanol; DMF: N,N-dimethylformanide; DMSO: dimethylsulfoxide; DIAD: diisopropyl azodicarboxylate; DIPEA: diisopropylethylamine; Boc: tert-butyloxycarbonyl; TFA: trifluoroacetic acid; and HATU: O-(7-azabenzotriazol-1-yl)-N,N,N′,N′-tetramethyluronium hexafluorophosphate.

### Synthesis of Intermediate Compound M1: 1-ethyl-3-methyl-1H-pyrazole-5-carbonyl isothiocyanate

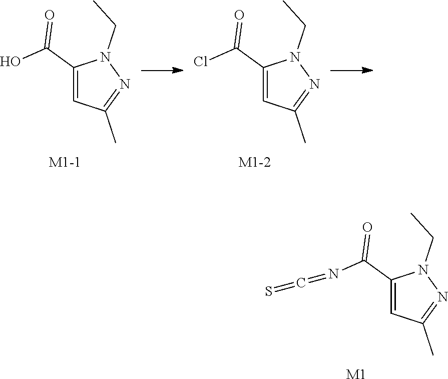

1-ethyl-3-methyl-1H-pyrazole-5-carboxylic acid (4.00 g, 25.9 mmol) was dispersed in dry DCM (80 mL), to which oxalyl chloride (3.9 g, 31.1 mmol) and a catalytic amount of DMF were added dropwise in an ice bath. The reaction mixture was reacted at room temperature for 1 h, and subjected to rotary evaporation under reduced pressure to remove the volatile matter. The crude product was added with DCM (20 mL) and subjected to rotary evaporation to remove the solvent, so as to give 1-ethyl-3-methyl-1H-pyrazole-5-carbonyl chloride (4.46 g, 100% yield), which was directly used in the next step.

1-ethyl-3-methyl-1H-pyrazole-5-carbonyl chloride (4.46 g, 25.9 mmol) was dissolved in dry acetone (20 mL) and added dropwise to a solution of potassium thiocyanate (5.0 g, 51.5 mmol) in acetone (100 mL) at 0° C. The reaction mixture was stirred at room temperature for 3 h and filtered to remove inorganic salts, and the filtrate was concentrated and purified by silica gel column chromatography (eluent: ethyl acetate/petroleum ether (v/v)=1/15) to give a clear brownish yellow liquid as 1-ethyl-3-methyl-1H-pyrazole-5-carbonyl isothiocyanate (4.0 g, 20.4 mmol, 78.7% yield). MS (ESI) m/z=196[M+H]⁺.

### Synthesis of Intermediate Compound M2: 4-ethyl-2-methylthiazole-5-carbonyl isothiocyanate

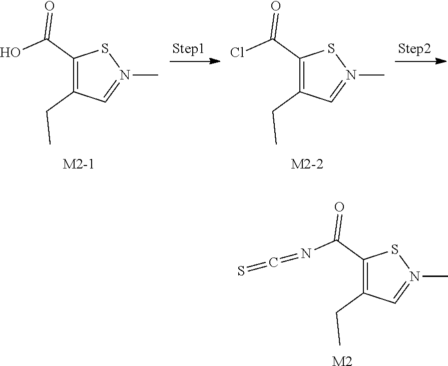

### (Step 1) Synthesis of 4-ethyl-2-methylthiazole-5-carbonyl chloride

4-ethyl-2-methylthiazole-5-carboxylic acid (2 g, 11.7 mmol) was dispersed in dry DCM (40 mL), into which oxalyl chloride (1.9 g, 15.1 mmol) and a catalytic amount of DMF were added dropwise in an ice bath. After reacting at room temperature for 1 h, the volatile matter was removed by rotary evaporation under reduced pressure. The crude product was added with DCM (20 mL) and subjected to rotary evaporation to remove the solvent, so as to give 4-ethyl-2-methylthiazole-5-carbonyl chloride (2.2 g, 100% yield), which was directly used in the next step.

### (Step 2) Synthesis of 4-ethyl-2-methylthiazole-5-carbonyl isothiocyanate

4-ethyl-2-methylthiazole-5-carbonyl chloride (2.2 g, 11.7 mmol) was dissolved in dry acetone (10 mL) and added dropwise to a solution of potassium thiocyanate (2.3 g, 23.4 mmol) in acetone (50 mL) at 0° C. The reaction mixture was stirred at room temperature for 3 h, and filtered to remove inorganic salts, and the filtrate was concentrated and purified by silica gel column chromatography (eluent: ethyl acetate/petroleum ether, v/v=1/15) to give a clear brownish yellow liquid as 4-ethyl-2-methylthiazole-5-carbonyl isothiocyanate (2.15 g, 10.2 mmol, 87% yield). MS (ESI) m/z=213[M+H]⁺.

### Synthesis of Intermediate Compound M3: 1-ethyl-4-fluoro-3-methyl-1H-pyrazole-5-carbonyl isothiocyanate

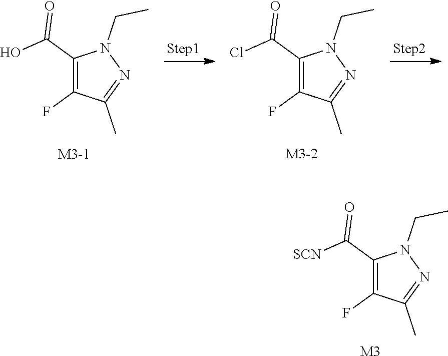

### (Step 1) Synthesis of 1-ethyl-4-fluoro-3-methyl-1H-pyrazole-5-carbonyl chloride

1-ethyl-4-fluoro-3-methyl-1H-pyrazole-5-carboxylic acid (2 g, 11.7 mmol) was dispersed in dry DCM (40 mL), into which oxalyl chloride (1.9 g, 15.1 mmol) and a catalytic amount of DMF were added dropwise in an ice bath. After reacting at room temperature for 1 h, the volatile matter was removed by rotary evaporation under reduced pressure. DCM (20 mL) was added to the crude product, and the solvent was removed by rotary evaporation to give 4-ethyl-2-methylthiazole-5-carbonyl chloride (2.2 g, 100% yield), which was directly used in the next step.

### (Step 2) Synthesis of 1-ethyl-4-fluoro-3-methyl-1H-pyrazole-5-carbonyl isothiocyanate

1-ethyl-4-fluoro-3-methyl-1H-pyrazole-5-carbonyl chloride (2.2 g, 11.7 mmol) was dissolved in dry acetone (10 mL) and added dropwise into a solution of potassium thiocyanate (2.3 g, 23.4 mmol) in acetone (50 mL) at 0° C., and the reaction mixture was stirred at room temperature for 3 h and filtered to remove inorganic salts. The filtrate was concentrated and purified by silica gel column chromatography (eluent: ethyl acetate/petroleum ether, v/v=1/15) to afford a clear brownish-yellow liquid as 4-ethyl-2-methylthiazole-5-carbonyl isothiocyanate (2.15 g, 10.2 mmol, 87% yield). MS (ESI) m/z=214[M+H]⁺

### Synthesis of Intermediate Compound M4: tert-butyl 4-(3-(2-chloro-5-(methoxycarbonyl)-3-nitrophenoxy)propyl)piperazine-1-carboxylate

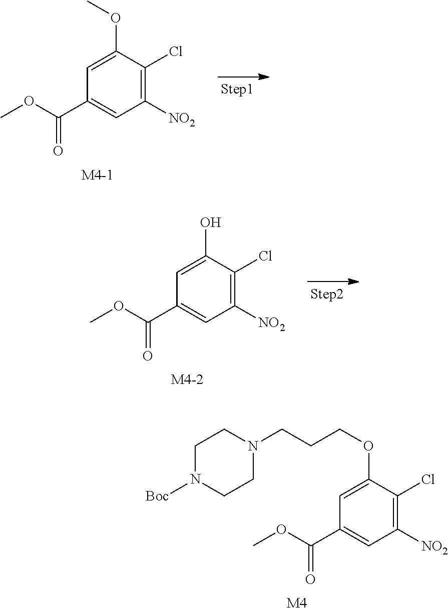

### (Step 1) Synthesis of methyl 4-chloro-3-hydroxy-5-nitrobenzoate

Methyl 4-chloro-3-methoxy-5-nitrobenzoate (10 g, 40.7 mmol) was dispersed in anhydrous dichloromethane (100 mL), into which boron tribromide (40.8 g, 162.8 mmol) was slowly added dropwise in an ice bath. After that, the reaction mixture was slowly heated to room temperature and reacted under stirring overnight. After the reaction was completed, methanol was slowly added dropwise in an ice bath to quench the reaction, and then the reaction mixture was dried by rotary evaporation, added with methanol (100 mL) and concentrated sulfuric acid (0.2 mL), and heated to 75° C. and stirred overnight. After cooled, the reaction mixture was concentrated under reduced pressure to remove the solvent, added with 150 mL of water, ultrasonically dispersed and filtered, and the resulting solids were collected, washed with water and dried to afford methyl 4-chloro-3-hydroxy-5-nitrobenzoate (8.89 g, 38.4 mmol). MS (ESI) m/z=232[M+H]⁺.

### (Step 2) Synthesis of tert-butyl 4-(3-(2-chloro-5-(methoxycarbonyl)-3-nitrophenoxy)propyl)piperazine-1-carboxylate

Methyl 4-chloro-3-hydroxy-5-nitrobenzoate (10 g, 47.2 mmol), tert-butyl 4-(3-hydroxypropyl) piperazine-1-carboxylate (10.6 g, 47.2 mmol) and triphenylphosphine (20.4 g, 77.7 mmol) were dissolved in anhydrous THE (200 mL) under the protection of nitrogen. The reaction mixture was dropwise added with DIAD (15.8 g, 77.7 mmol) in an ice bath, warmed up to room temperature and stirred for 16 h. After that, the reaction mixture was poured to water, and ethyl acetate was added for extraction. The organic phase was collected, washed with a saturated NaCl solution and dried with anhydrous magnesium sulfate, concentrated and purified by silica gel column chromatography (eluent: petroleum ether/ethyl acetate=2/1) to afford the target compound (13 g, 61% yield). MS (ESI) m/z=458[M+H]⁺

### Synthesis of Intermediate Compound M5: tert-butyl 4-(3-(2-chloro-5-(methoxycarbonyl)-3-nitrophenoxy)propyl)piperazine-1-carboxylate

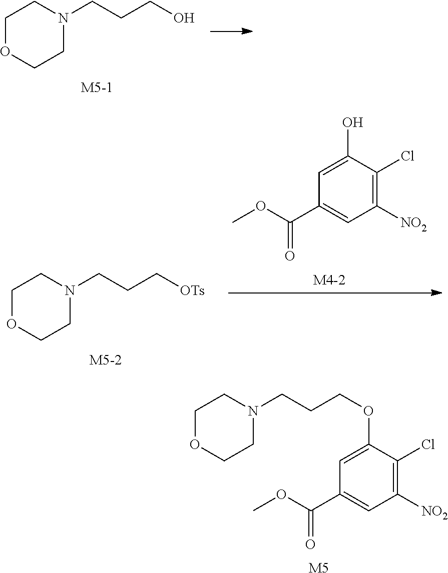

(Step 1) TEA (13.9 g, 137.8 mmol) and p-toluenesulfonyl chloride (14.5 g, 75.8 mmol) were added to a solution of 3-morpholinopropan-1-ol (10 g, 68.9 mmol) in dichloromethane (200 mL), and the mixture was reacted at room temperature for 2 h, and then added with water and dichloromethane for extraction. The organic phase was washed respectively with water and saturated NaCl solution, dried with anhydrous sodium sulfate, filtered and concentrated to afford 4-morpholinopropyl 4-methylbenzenesulfonate, which was directly used in the next step. MS (ESI) m/z=300[M+H]⁺

(Step 2) Methyl 4-chloro-3-hydroxy-5-nitrobenzoate (3.68 g, 15.93 mmol) was dissolved in DMF (50 mL), to which 4-morpholinopropyl 4-methylbenzenesulfonate (5.7 g, 19.11 mmol) and K₂CO₃ (4.4 g, 31.86 mmol) were added. The reaction mixture was stirred at 75° C. for 16 h, and filtered to remove the inorganic salt. The filtrate was distilled under reduced pressure to remove DMF, dissolved in ethyl acetate, washed respectively with water and saturated NaCl solution, dried with anhydrous magnesium sulfate, filtered and concentrated such that the remaining ethyl acetate had a volume of 30 mL. The obtained product was filtered to afford a yellow solid as the target compound (4.56 g, 80% yield). MS (ESI) m/z=359[M+H]⁺

### Intermediate Compound M6

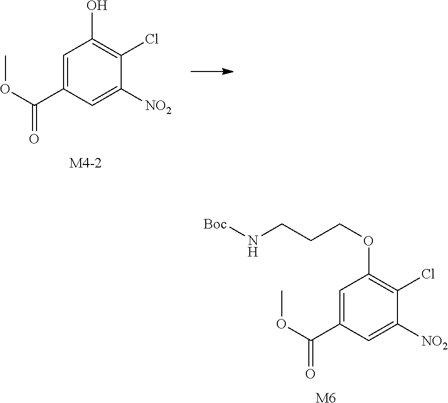

Compound 2b (761 mg, 4.35 mmol) and methyl 4-chloro-3-hydroxy-5-nitrobenzoate (1.0 g, 4.35 mmol) were dissolved in dry tetrahydrofuran (30 mL) at room temperature. Triphenylphosphine (1.71 g, 6.53 mmol) and diisopropyl azodicarboxylate (1.32 g, 6.53 mmol) were added after the replacement of air with nitrogen, and then the mixture was stirred and reacted for 15 h. After the reaction was completed, water and ethyl acetate were added for extraction (20 mL×3). The resulting organic phases were combined, dried by rotary evaporation and separated by column chromatography (eluent: ethyl acetate/petroleum ether=1/2, v/v) to afford the target compound M6 (1.31 g, 75% yield). MS (ESI) m/z=389[M+H]⁺

### Synthesis of Intermediate Compound M7: methyl 4-fluoro-3-(methylthio)-5-nitrobenzoate

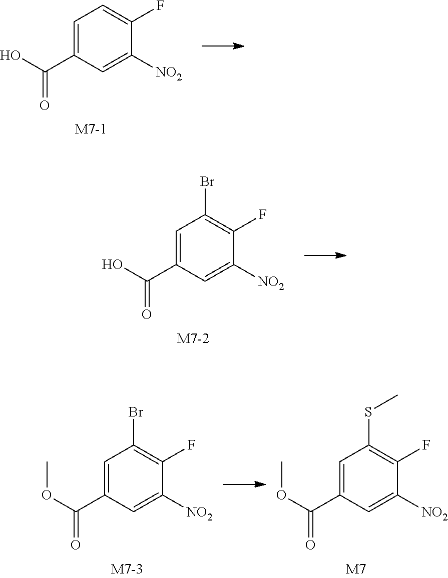 [SMILES: O=C(O)c1ccc(F)c([N+](=O)[O-])c1 ; O=C(O)c1cc(Br)c(F)c([N+](=O)[O-])c1 ; COC(=O)c1cc(Br)c(F)c([N+](=O)[O-])c1 ; COC(=O)c1cc(SC)c(F)c([N+](=O)[O-])c1 ; "M7"]

### (Step 1) Synthesis of 3-bromo-4-fluoro-5-nitrobenzoic acid

4-fluoro-3-nitrobenzoic acid (50 g, 270 mmol) was dispersed in concentrated sulfuric acid (200 mL), to which NBS (47.5 g, 270 mmol) was added. The reaction mixture was heated to 75° C. and stirred overnight. After cooled to room temperature, the reaction mixture was slowly poured into ice water under stirring, and light-yellow solids were precipitated. The resulting solids were collected by filtration and dried to afford the target compound (66 g).

### (Step 2) Synthesis of methyl 3-bromo-4-fluoro-5-nitrobenzoate

Thionyl chloride (44.5 g, 373.5 mmol) was added dropwise into a methanol (400 mL) solution of 3-bromo-4-fluoro-5-nitrobenzoic acid (66 g, 249 mmol) in an ice bath. The mixture was heated to 75° C. and stirred overnight. The reaction solution was concentrated to about 100 mL, and cooled to allow the solids continue to precipitate. The resulting solids were collected by filtration and dried to afford methyl 3-bromo-4-fluoro-5-nitrobenzoate (56 g).

### (Step 3) Synthesis of methyl 4-fluoro-3-(methylthio)-5-nitrobenzoate

S-methyl thioacetate (7.8 g, 86.4 mmol), Pd₂(dba)₃ (6.6 g, 7.2 mmol), xantphos (4.2 g, 7.2 mmol) and K₃PO₄ (45.8 g, 216 mmol) were sequentially added to a solution of methyl 3-bromo-4-fluoro-5-nitrobenzoate (20 g, 72 mmol) in toluene (330 mL) and tert-butanol (30 mL), and the reaction mixture was reacted at 110° C. overnight under the protection of nitrogen until the reaction was completed. The reaction mixture was cooled to room temperature, and filtered to remove the solids. The filtrate was then concentrated and purified and separated by a normal-phase column chromatography (eluent: PE/EA=5/1, v/v) to afford the target compound (7.9 g, 45% yield).

### Synthesis of Intermediate Compound M8: methyl 4-fluoro-3-((3-morpholinopropyl)thio)-5-nitrobenzoate

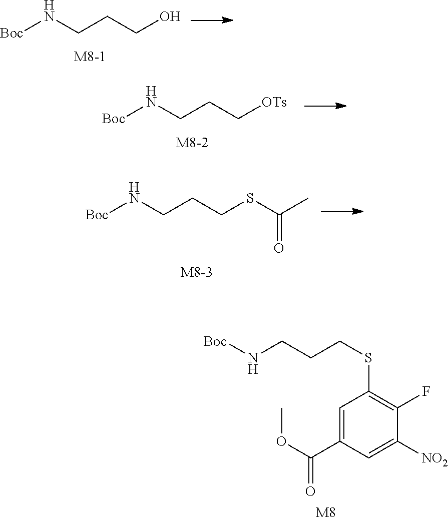

(Step 1) TEA (4.8 g, 48 mmol) and p-toluenesulfonyl chloride (4.6 g, 24 mmol) were added to a solution of tert-butyl N-(3-hydroxypropyl) carbamate (4.2 g, 24 mmol) in dichloromethane (50 mL), and the reaction mixture was reacted at room temperature for 2 h, and added with water and dichloromethane for extraction. The organic phase was washed respectively with water and saturated NaCl solution, dried with anhydrous sodium sulfate, filtered and concentrated to afford the compound M8-2 (6.3 g), which was directly used in the next step. MS (ESI) m/z=330[M+H]⁺

(Step 2) Potassium thioacetate (629 mg, 5.52 mmol) and potassium carbonate (761 mg, 5.52 mmol) were added to a solution of 3-(N-tert-butoxycarbonyl)propyl 4-methylbenzenesulfonate (1.4 g, 4.25 mmol) in DMF (50 mL), and the reaction mixture was stirred at room temperature overnight. The inorganic salt was removed by filtration, and the solvent was removed by rotary evaporation to give a crude product, which was purified by silica gel column chromatography (eluent: PE/EA=1/1) to afford the target compound M8-3 (0.8 g, 80% yield).

### (Step 3) Synthesis of methyl 4-fluoro-3-((3-morolinopropyl)thio)-5-nitrobenzoate

The compound M8-3 (0.8 g, 3.4 mmol), Pd₂(dba)₃ (0.31 g, 0.34 mmol), xantphos (0.19 g, 0.34 mmol) and K₃PO₄ (2.16 g, 10.2 mmol) were added sequentially to a solution of methyl 3-bromo-4-fluoro-5-nitrobenzoate (0.94 g, 3.4 mmol) in toluene (20 mL) and tert-butanol (2 mL), and the reaction mixture was reacted at 110° C. overnight under the protection of nitrogen until the reaction was completed. The reaction mixture was cooled to room temperature, and the solids were removed by filtration. Then the reaction mixture was concentrated to give a crude product, which was purified and separated by silica gel column chromatography (eluent: PE/EA=3/1, v/v) to afford the target compound M8 (0.66 g, 50% yield). MS (ESI) m/z=389[M+H]⁺

### Synthesis of Intermediate Compound M9: N-tert-butoxycarbonyl-4-3-((4-nitrooxy)carbonyl)oxypropylpiperazine

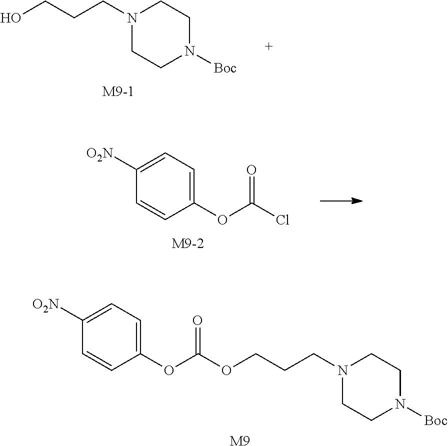 [SMILES: CC(C)(C)OC(=O)N1CCN(CCCO)CC1 ; O=C(Cl)Oc1ccc([N+](=O)[O-])cc1 ; CC(C)(C)OC(=O)N1CCN(CCCOC(=O)Oc2ccc([N+](=O)[O-])cc2)CC1 ; "M9"]

Triethylamine (248 mg, 2.46 mmol) and chloroformic acid-4-nitrobenzene ester (412 mg, 2.05 mmol) were added to a solution of 4-(3-hydroxypropyl)tert-butoxycarbonylpiperazine (500 mg, 2.05 mmol) in THE in an ice bath. The mixture was then reacted at room temperature for 2 h, and dried by rotary evaporation to give a crude product, which was purified by silica gel column chromatography to afford the target compound (570 mg, 68% yield). MS (ESI) m/z=410[M+H]⁺

The intermediate compounds M10 to M13 were synthesized by replacing 4-(3-hydroxypropyl) tert-butoxycarbonyl piperazine in the synthesis of the intermediate compound M9 with the corresponding alcohol/amine (listed in the following table)

| | | m/z |
|---|---|---|
| Alcohol/amine | Intermediate | [M + H]⁺ |
| 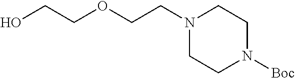 [SMILES: CC(C)(C)OC(=O)N1CCN(CCOCCO)CC1] | 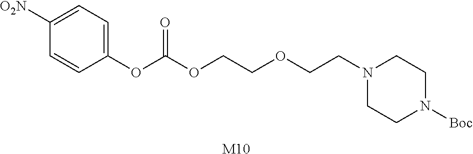 [SMILES: CC(C)(C)OC(=O)N1CCN(CCOCCOC(=O)Oc2ccc([N+](=O)[O-])cc2)CC1 ; "M10"] | 440 |
| 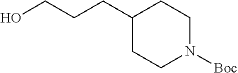 [SMILES: CC(C)(C)OC(=O)N1CCC(CCCO)CC1] | 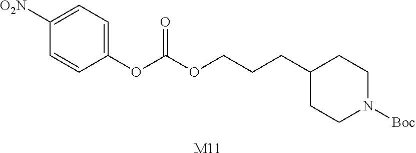 [SMILES: CC(C)(C)OC(=O)N1CCC(CCCOC(=O)Oc2ccc([N+](=O)[O-])cc2)CC1 ; "M11"] | 409 |
| 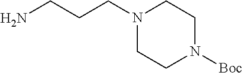 [SMILES: CC(C)(C)OC(=O)N1CCN(CCCN)CC1] | 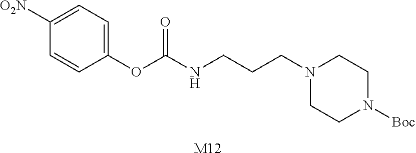 [SMILES: CC(C)(C)OC(=O)N1CCN(CCCNC(=O)Oc2ccc([N+](=O)[O-])cc2)CC1 ; "M12"] | 409 |
| 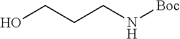 [SMILES: CC(C)(C)OC(=O)NCCCO] | 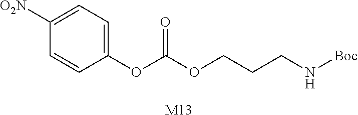 | 341 |

### Example 1

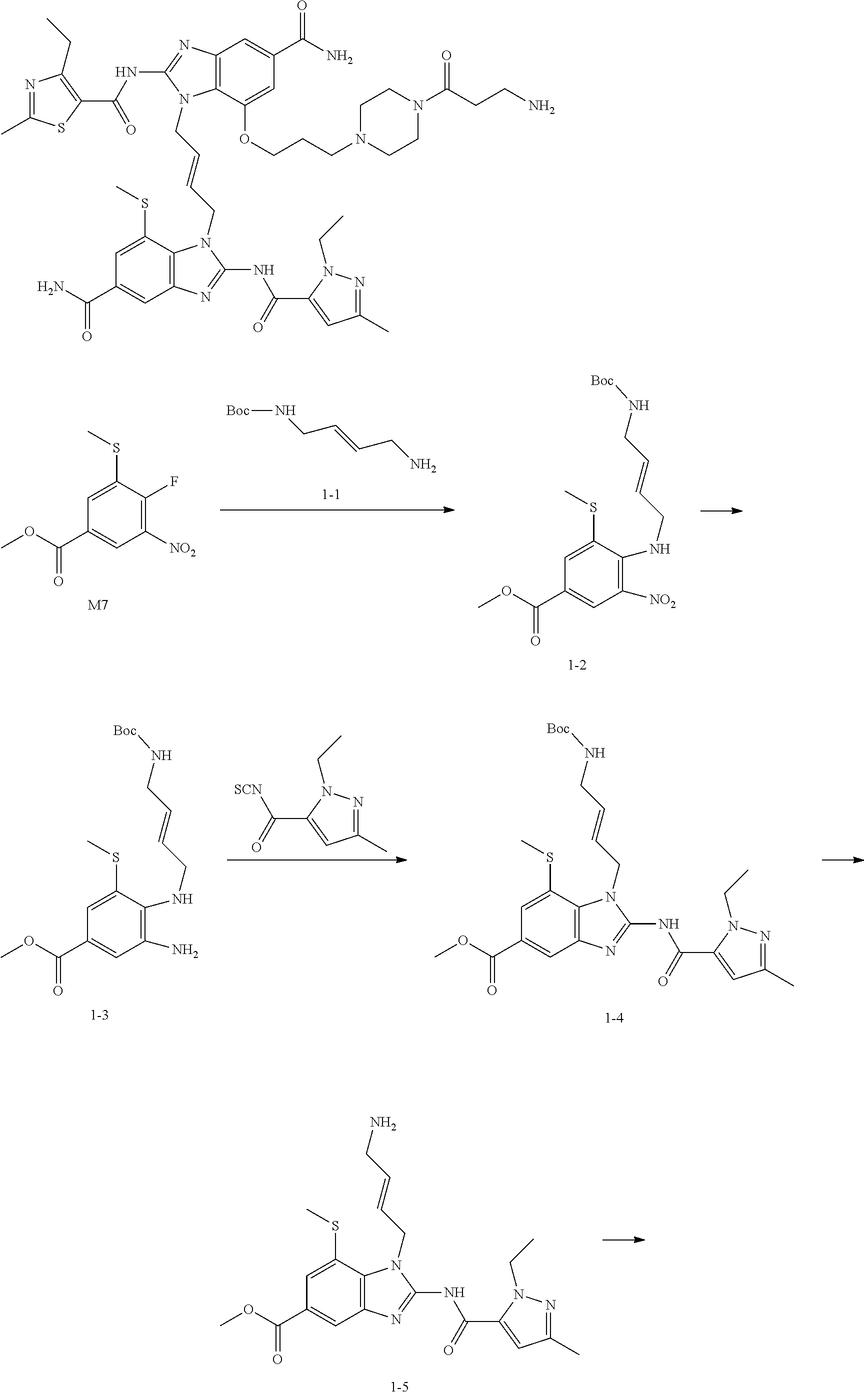

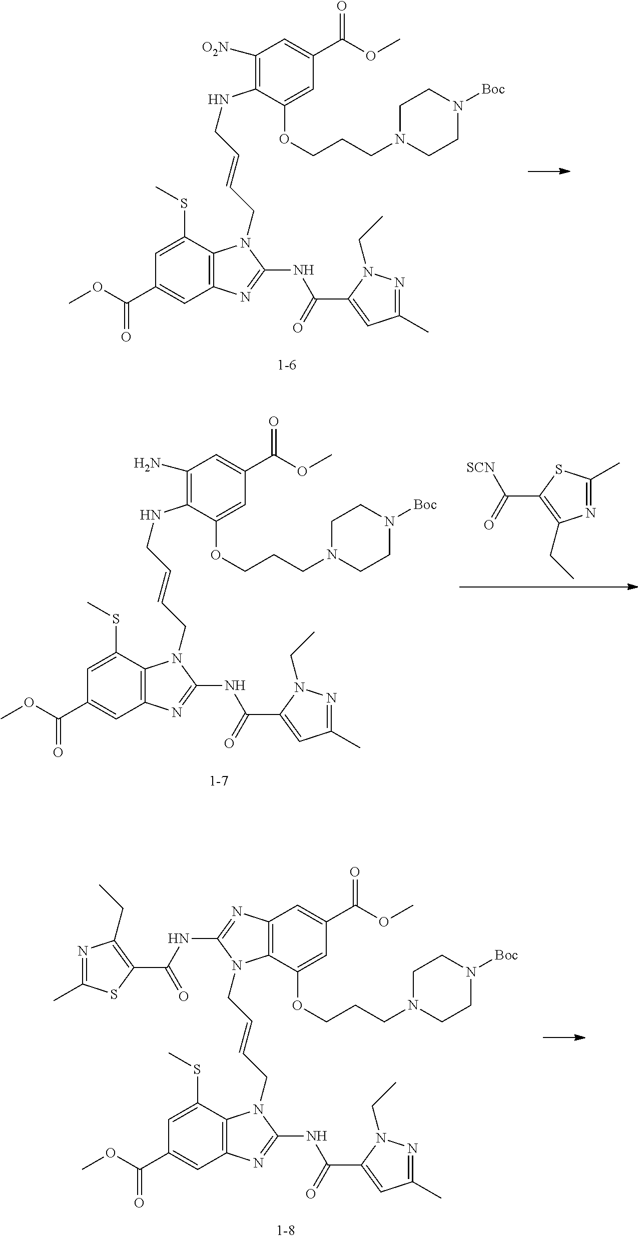

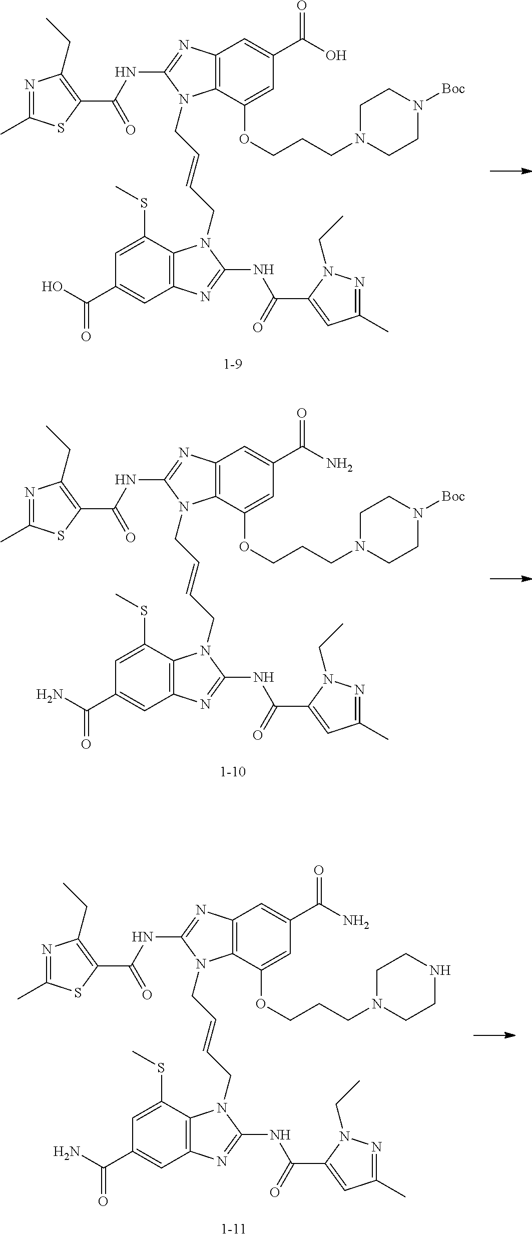 [SMILES: CCc1nc(C)sc1C(=O)Nc1nc2cc(C(=O)O)cc(OCCCN3CCN(C(=O)OC(C)(C)C)CC3)c2n1C/C=C/Cn1c(NC(=O)c2cc(C)nn2CC)nc2cc(C(=O)O)cc(SC)c21 ; CCc1nc(C)sc1C(=O)Nc1nc2cc(C(N)=O)cc(OCCCN3CCN(C(=O)OC(C)(C)C)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCNCC3)c2n1C/C=C/Cn1c(NC(=O)c2cc(C)nn2CC)nc2cc(C(N)=O)cc(SC)c21]

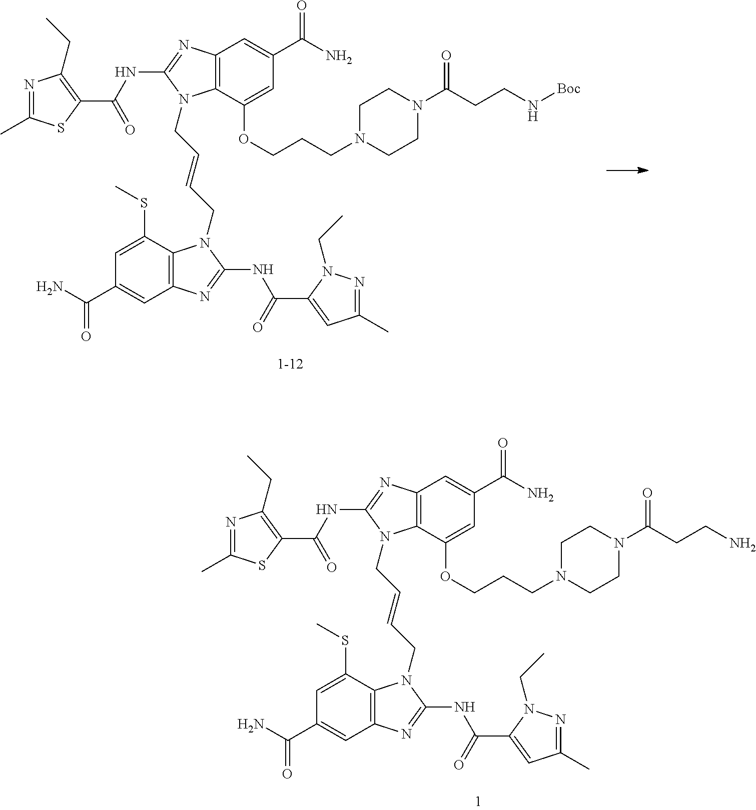 [SMILES: CCc1nc(C)sc1C(=O)Nc1nc2cc(C(N)=O)cc(OCCCN3CCN(C(=O)CCNC(=O)OC(C)(C)C)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)CCN)CC3)c2n1C/C=C/Cn1c(NC(=O)c2cc(C)nn2CC)nc2cc(C(N)=O)cc(SC)c21]

(Step 1) DIPEA (3.1 g, 24.4 mmol) and compound 1-1 (2.3 g, 12.2 mmol) were added to a DMF solution (40 mL) of the compound M7 (3 g, 12.2 mmol), and the reaction mixture was reacted at room temperature for 2 h, and then dried by rotary evaporation to give a crude product, which was purified by silica gel column chromatography (PE/EA=2/1) to afford the target compound 1-2 (4.6 g). MS (ESI) m/z=412[M+H]⁺

(Step 2) Aqueous ammonia (20 mL) was added to a methanol (120 mL) solution of the compound 1-2 (4.38 g, 10.65 mmol) in an ice bath. 10 minutes later, an aqueous solution of sodium dithionite (9.3 g, 53.25 mmol) was added, and the reaction mixture was warmed up to room temperature and reacted for 1 h. Methanol was removed by rotary evaporation, and water and ethyl acetate were added for extraction. The resulting organic phase was washed with water and saturated NaCl solution, and then concentrated to give a crude product, which was then purified by silica gel column chromatography (PE/EA=3/1) to afford the target compound 1-3 (2.7 g). MS (ESI) m/z=382[M+H]⁺

(Step 3) The compound M1 (1.38 g, 7.1 mmol) was added to a DMF solution (50 mL) of the compound 1-3 (2.7 g, 7.1 mmol) in an ice bath. After 20 minutes, DIPEA (2.75 g, 21.3 mmol) and HATU (3.24 g, 8.52 mmol) were added, and the reaction mixture was reacted at room temperature overnight. The reaction mixture was poured into water, and solids were precipitated, collected by filtration and dried to afford the target compound 1-4 (3.1 g). MS (ESI) m/z=543[M+H]⁺

(Step 4) An ethyl acetate solution (4 M, 20 mL) of hydrogen chloride was added to the compound 1-4 (1.8 g, 3.3 mmol), and the reaction mixture was reacted at room temperature for 1 h, and dried by rotary evaporation to afford the target compound 1-5 (1.57 g, hydrochloride). MS (ESI) m/z=443[M+H]⁺

(Step 5) The compound 1-5 (1.57 g, 3.3 mmol) and the compound M4 (1.5 g, 3.3 mmol) were added to n-butanol (20 mL), to which DIPEA (2.1 g, 16.5 mmol) was added. The reaction mixture was heated to 120° C. and reacted for 18 h. The solvent was removed by rotary evaporation, and then the residue was purified by silica gel column chromatography (DCM/MeOH=60/1) to afford the target compound 1-6 (1.46 g). MS (ESI) m/z=864[M+H]⁺

(Step 6) Aqueous ammonia (4 mL) was added to a methanol (10 mL) and THF (5 mL) solution of the compound 1-6 (1.46 g, 1.69 mmol) in an ice bath. After 10 minutes, an aqueous solution of sodium dithionite (1.47 g, 8.45 mmol) was added, and the reaction mixture was warmed up to room temperature and reacted for 1 h. Methanol and THF were removed by rotary evaporation, and water and ethyl acetate were added for extraction. The resulting organic phase was washed with water and saturated NaCl solution, and concentrated to give a crude product, which was then purified by silica gel column chromatography (DCM/MeOH=20/1) to afford the target compound 1-7 (0.91 g). MS (ESI) m/z=834 [M+H]⁺

(Step 7) The compound M2 (0.23 g, 1.08 mmol) was added to a DMF solution (10 mL) of the compound 1-7 (0.9 g, 1.08 mmol) in an ice bath. After 20 minutes, DIPEA (0.42 g, 3.24 mmol) and HATU (0.49 g, 1.3 mmol) were added, and the reaction mixture was reacted at room temperature overnight, and then poured into water. The solid precipitates were collected by filtration and dried to afford the target compound 1-8 (1.0 g). MS (ESI) m/z=1012[M+H]⁺

(Step 8) Lithium hydroxide hydrate (0.41 g, 9.8 mmol) was added to a methanol (10 mL) and water (2 mL) solution of the compound 1-8 (1.0 g, 0.98 mmol), and the reaction mixture was heated to 70° C. and reacted for 12 h. Methanol was removed by rotary evaporation, and then 5 mL of water was added. Dilute hydrochloric acid (1 M) was added in an ice bath until the solids no longer precipitated, and the resulting solids were collected by filtration and dried to afford the target compound 1-9 (0.82 g). MS (ESI) m/z=984[M+H]⁺

(Step 9) The compound 1-9 (0.82 g, 0.83 mmol) was dissolved in DMF (10 mL), to which HATU (0.69 g, 1.83 mmol) and DIPEA (0.54 g, 4.2 mmol) were added. After 0.5 h, ammonium bicarbonate (0.65 g, 8.3 mmol) was added, and the reaction mixture was stirred at room temperature for 2 h, and added with water to precipitate solids. The resulting solids were collected by filtration, washed with pure water and dried to afford the compound 1-10 (0.72 g). MS (ESI) m/z=982[M+H]⁺

(Step 10) Trifluoroacetic acid (5 mL) was added to a dichloromethane solution (5 mL) of the compound 1-10 (0.72 g, 0.73 mmol). The reaction mixture was reacted at room temperature for 1 h, and dried by rotary evaporation to afford the target compound 1-11 (1 g, with a content of 64% (containing trifluoroacetic acid)). MS (ESI) m/z=882[M+H]⁺

(Step 11) DIPEA (26 mg, 0.204 mmol) and HATU (26 mg, 0.068 mmol) were added to a DMF (2 mL) solution of N-Boc-3-aminopropanoic acid (13 mg, 0.068 mmol). After 10 minutes, the reaction mixture was added with the compound 1-11 (50 mg, 0.057 mmol), reacted at room temperature for 3 h, dried by rotary evaporation, and dissolved with dichloromethane. The organic phase was washed with water and dried by rotary evaporation to afford the target compound 1-12 (70 mg, 80% purity), which was directly used in the next step of reaction. MS (ESI) m/z=1053[M+H]⁺

(Step 12) Trifluoroacetic acid (3 mL) was added to a dichloromethane (3 mL) solution of the compound 1-12 (70 mg, 0.053 mmol), and the reaction mixture was reacted at room temperature for 1 h, which was then dried by rotary evaporation to give a crude product. The resulting crude product was purified by preparative HPLC to afford the target compound 1 (16 mg). MS (ESI) m/z=953[M+H]⁺

¹H NMR (400 MHz, DMSO, D₂O) δ 7.82 (d, J=1.5 Hz, 1H), 7.60 (dd, J=3.9, 1.2 Hz, 2H), 7.28 (s, 1H), 6.48 (s, 1H), 5.89-5.53 (m, 2H), 5.12 (s, 2H), 4.85 (s, 2H), 4.48 (q, J=6.9 Hz, 3H), 4.48 (q, J=6.9 Hz, 3H), 4.05 (s, 2H), 3.23-2.94 (m, 9H), 2.69 (t, J=6.3 Hz, 3H), 2.41 (s, 3H), 2.07 (s, 3H), 1.98 (m, 2H), 1.24 (t, J=7.1 Hz, 3H), 1.11 (t, J=7.5 Hz, 3H).

The target compounds of Examples 2, 3, 4, and 5 were synthesized basically in accordance with the synthesis method of Example 1 except that the N-Boc-3-aminopropionic acid in Step 11 was replaced with the corresponding carboxylic acid (namely, N-methyl-N-Boc-4-aminobutyric acid, N-Boc-2,2-dimethyl 4-aminobutanoic acid, 4-(4-(tert-butoxycarbonyl)piperazin-1-yl)butanoic acid, 5-(4-(tert-butoxycarbonyl)piperazin-1-yl) pentanoic acid, listed in the following table).

| Example | Carboxylic acid | Target product | Characterization data |
|---|---|---|---|
| 2 | 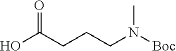 | 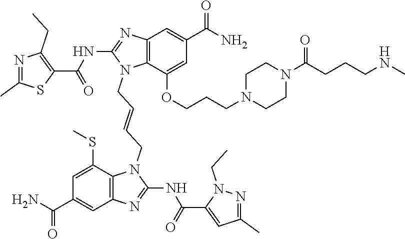 | MS (ESI) m/z = 981 [M + H]⁺ ¹H NMR (400 MHz, DMSO-D₂O) δ 7.84 (s, 1H), 7.62 (d, J = 5.8 Hz, 2H), 7.30 (s, 1H), 6.50 (s, 1H), 5.72 (dt, J = 20.6, 15.7 Hz, 2H), 5.14 (s, 2H), 4.88 (s, 2H), 4.50 (d, J = 7.1 Hz, 3H), 4.06 (s, 3H), 3.23-3.13 (m, 3H), 3.10 (m, 3H), 2.97-2.85 (m, 4H), 2.46 (t, J = 7.1 Hz, 2H), 2.43 (s, 3H), 2.10 (s, 3H), 1.99 (m, 2H), 1.89-1.76 (m, 2H), 1.26 (t, J = 7.0 Hz, 3H), 1.14 (t, J = 7.5 Hz, 3H). |
| 3 | 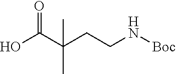 | 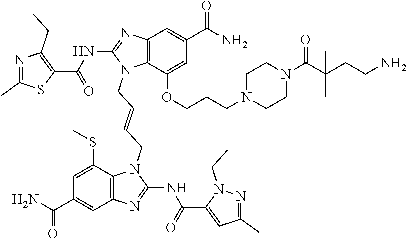 [SMILES: CCc1nc(C)sc1C(=O)Nc1nc2cc(C(N)=O)cc(OCCCN3CCN(C(=O)C(C)(C)CCN)CC3)c2n1C/C=C/Cn1c(NC(=O)c2cc(C)nn2CC)nc2cc(C(N)=O)cc(SC)c21] | MS (ESI) m/z = 994 [M + H]⁺ ¹H NMR (400 MHz, DMSO-D₂O) δ 7.83 (s, 1H), 7.61 (d, J = 8.2 Hz, 2H), 7.30 (s, 1H), 6.50 (s, 1H), 5.97-5.59 (m, 2H), 5.15 (s, 2H), 4.89 (s, 2H), 4.50 (d, J = 6.7 Hz, 3H), 4.08 (s, 2H), 3.27-3.13 (m, 4H), 3.09 (dd, J = 14.4, 7.0 Hz, 3H), 2.88- 2.77 (m, 2H), 2.42 (s, 3H), 2.10 (s, 3H), 2.02 (s, 2H), 1.88-1.74 (m, 2H), 1.38-1.20 (m, 9H), 1.13 (t, J = 7.4 Hz, 3H). |
| 4 | 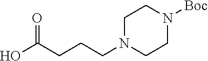 [SMILES: CC(C)(C)OC(=O)N1CCN(CCCC(=O)O)CC1] | 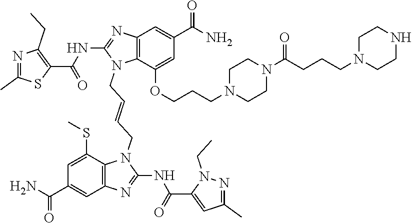 [SMILES: CCc1nc(C)sc1C(=O)Nc1nc2cc(C(N)=O)cc(OCCCN3CCN(C(=O)CCCN4CCNCC4)CC3)c2n1C/C=C/Cn1c(NC(=O)c2cc(C)nn2CC)nc2cc(C(N)=O)cc(SC)c21] | MS (ESI) m/z = 1036 [M + H]⁺ ¹HNMR (400M, DMSO- D₂O) δ 7.86 (s, 1H), 7.58- 7.65 (m, 2H), 7.31 (s, 1H), 6.52 (s, 1H), 5.61-5.79 (m, 2H), 5.09-5.16 (m, 2H), 4.81-4.90 (m, 2H), 4.45- 4.53 (m, 2H), 4.00- 4.07 (m, 2H), 3.35- 3.45 (m, 6H), 3.04- 3.20 (m, 7H), 2.81- 3.00 (m, 2H), 2.43- 2.48 (m, 2H), 2.41 (s, 3H), 2.08 (s, 3H), 1.82-2.02 (m, 4H), 1.25(t, J = 7.16 Hz, 3H), 1.13(t, J = 7.48 Hz, 3H). |
| 5 | 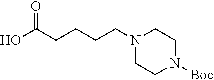 [SMILES: CC(C)(C)OC(=O)N1CCN(CCCCC(=O)O)CC1] | 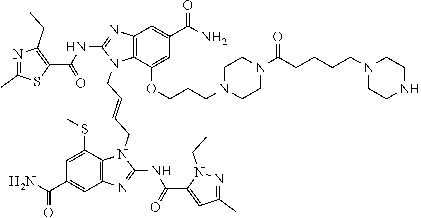 [SMILES: CCc1nc(C)sc1C(=O)Nc1nc2cc(C(N)=O)cc(OCCCN3CCN(C(=O)CCCCN4CCNCC4)CC3)c2n1C/C=C/Cn1c(NC(=O)c2cc(C)nn2CC)nc2cc(C(N)=O)cc(SC)c21] | MS (ESI) m/z = 1050 [M + H]⁺ ¹HNMR (400M, DMSO) δ 12.83 (s, br, 2H) 8.08 (s, 1H) 7.96 (s, 1H) 7.86 (s, 1H), 7.58-7.65 (m, 2H), 7.42 (s, 1H) 7.36 (s, 1H) 7.31 (s, 1H), 6.52 (S, 1H), 5.61-5.79 (m, 2H), 5.09- 5.16 (m, 2H), 4.81- 4.90 (m, 2H), 4.45- 4.53 (m, 2H), 4.00- 4.07 (m, 2H), 3.35- 3.45 (m, 6H), 3.04- 3.20 (m, 7H), 2.81- 3.00 (m, 2H), 2.43- 2.48 (m, 2H), 2.41 (s, 3H), 2.08 (s, 3H), 1.82-2.02 (m, 2H), 1.65-1.69 (m, 2H), 1.55-1.63 (m, 2H), 1.28 (t, J = 7.16 Hz, 3H), 1.16 (t, J = |
| | | | 7.48 Hz, 3H). |

### Example 6

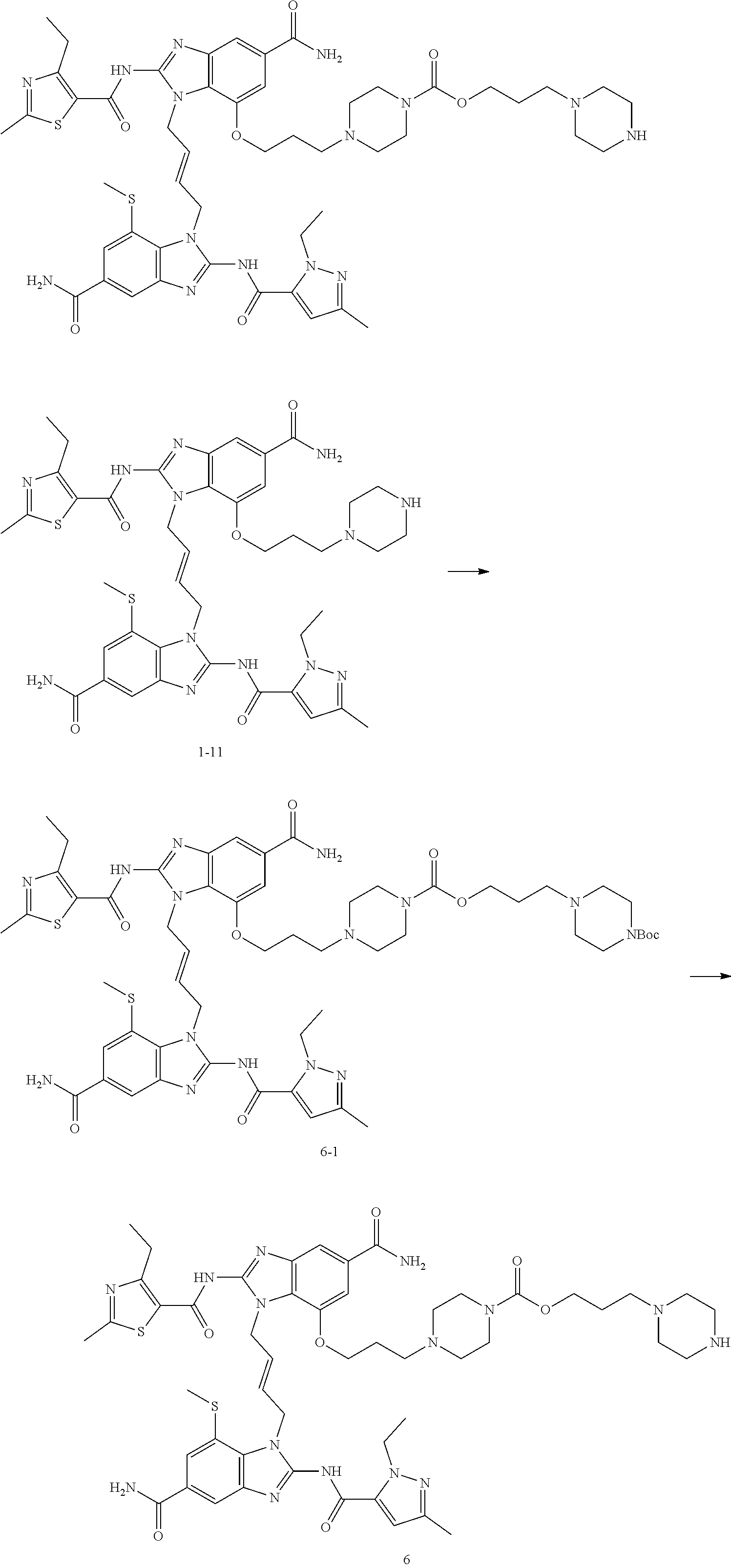

(Step 1) DIPEA (22 mg, 0.171 mmol) and the compound M9 (23 mg, 0.057 mmol) were added to a DMF (2 mL) solution of the compound 1-11 (50 mg, 0.057 mmol). The reaction mixture was reacted at room temperature for 1 h, dried by rotary evaporation, and dissolved with dichloromethane. The organic phase was washed with water, dehydrated and dried by rotary evaporation to afford the target compound 6-1 (65 mg, 80% purity), which was directly used in the next step of reaction. MS (ESI) m/z=1152[M+H]⁺

(Step 2) Trifluoroacetic acid (3 mL) was added to a dichloromethane (3 mL) solution of the compound 6-1 (65 mg, 0.045 mmol). The reaction mixture was reacted at room temperature for 1 h and then dried by rotary evaporation to give a crude product. The resulting crude product was purified by preparative HPLC to afford the target compound 6 (25 mg). MS (ESI) m/z=1052[M+H]⁺

¹H NMR (400 MHz, DMSO-d₆) δ12.91-12.95 (in, 2H), 7.83 (s, 1H), 7.57-7.63 (m, 2H), 7.26 (m, 1H), 6.45 (s, 1H), 5.63-5.80 (m, 2H), 5.07-5.17 (m, 2H), 4.81-4.89 (m, 2H), 4.45-4.53 (m, 2H), 3.99-4.16 (m, 4H), 3.26-3.43 (m, 10H), 3.04-3.21 (m, 8H), 2.40 (s, 3H), 2.07 (s, 3H), 1.93-2.02 (m, 4H), 1.25 (t, J=7.16 Hz, 3H), 1.12 (t, J=7.48 Hz, 3H).

The target compounds of Examples 7, 8, 9, and 10 were synthesized basically in accordance with the synthesis method of Example 6 except that the intermediate M9 used in Step 1 was replaced with the corresponding active ester (namely M10, M5, M12, and M13, listed in the following table).

| Example | Active ester | Example | Characterization data |
|---|---|---|---|
| 7 | 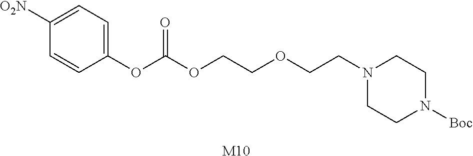 [SMILES: CC(C)(C)OC(=O)N1CCN(CCOCCOC(=O)Oc2ccc([N+](=O)[O-])cc2)CC1 ; "M10"] | 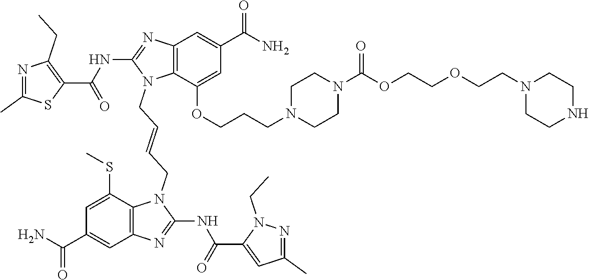 [SMILES: CCc1nc(C)sc1C(=O)Nc1nc2cc(C(N)=O)cc(OCCCN3CCN(C(=O)OCCOCCN4CCNCC4)CC3)c2n1C/C=C/Cn1c(NC(=O)c2cc(C)nn2CC)nc2cc(C(N)=O)cc(SC)c21] | MS (ESI) m/z = 1082 [M + H]⁺ ¹H NMR (400M, DMSO-d6) δ12.65- 12.69 (m, 2H), 7.84 (s, 1H), 7.57-7.64 (m, 2H), 7.25-7.30 (m, 1H), 6.49 (s, 1H), 5.63- 5.81 (m, 2H), 5.08- 5.16 (m, 2H), 4.81- 4.90 (m, 2H), 4.45- 4.55 (m, 2H), 4.14- 4.28 (m, 2H), 4.02- 4.06 (m, 2H), 3.73- 3.78 (m, 2H), 3.67- 3.72 (m, 2H), 3.34- 3.46 (m, 8H), 3.26- 3.34 (m, 2H), 3.05- 3.18 (m, 6H), 2.40 (s, 3H), 2.08 (s, 3H), 1.93- 2.01 (m, 2H), 1.25 (t, J = |
| | | | 7.16 Hz, 3H), 1.12 (t, J = |
| | | | 7.48 Hz, 3H). |
| 8 | 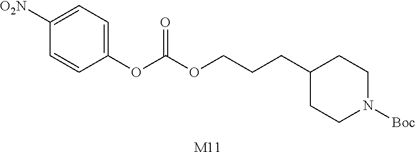 [SMILES: CC(C)(C)OC(=O)N1CCC(CCCOC(=O)Oc2ccc([N+](=O)[O-])cc2)CC1 ; "M11"] | 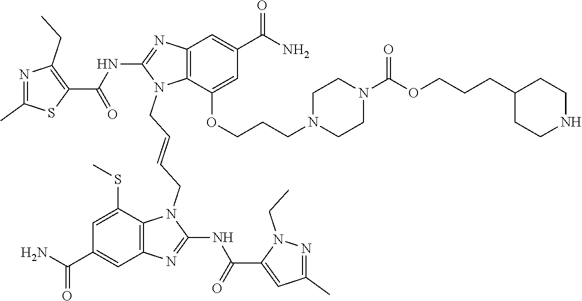 [SMILES: CCc1nc(C)sc1C(=O)Nc1nc2cc(C(N)=O)cc(OCCCN3CCN(C(=O)OCCCC4CCNCC4)CC3)c2n1C/C=C/Cn1c(NC(=O)c2cc(C)nn2CC)nc2cc(C(N)=O)cc(SC)c21] | MS (ESI) m/z = 1051 [M + H]⁺ ¹HNMR (400M, DMSO) δ 12.85 (s, br, 2H) 8.05 (s, 1H) 7.96 (s, 1H) 7.87 (s, 1H), 7.60 (s, 1H), 7.57 (s, 1H), 7.42 (s, 1H) 7.36 (s, 1H) 7.30 (s, 1H), 6.52 (s, 1H), 5.61- 5.79 (m, 2H), 5.09- 5.16 (m, 2H), 4.81- 4.90 (m, 2H), 4.45- 4.53 (m, 2H), 4.00- 4.07 (m, 2H), 3.04- 3.20 (m, 7H), 2.81- 3.00 (m, 2H), 2.43- 2.48 (m, 2H), 2.41 (s, 3H), 2.10 (s, 3H), 1.93- 1.97 (m, 2H), 1.78- 1.82 (m, 2H), 1.58- 1.63 (m, 3H), 1.24- |
| | | | 1.30 (m, 5H), 1.11 (t, J = |
| | | | 7.48 Hz, 3H). |
| 9 | 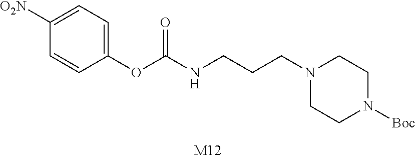 [SMILES: CC(C)(C)OC(=O)N1CCN(CCCNC(=O)Oc2ccc([N+](=O)[O-])cc2)CC1 ; "M12"] | 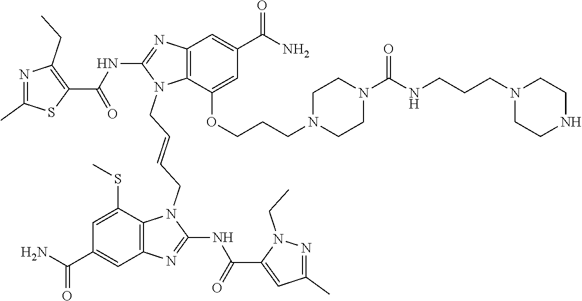 [SMILES: CCc1nc(C)sc1C(=O)Nc1nc2cc(C(N)=O)cc(OCCCN3CCN(C(=O)NCCCN4CCNCC4)CC3)c2n1C/C=C/Cn1c(NC(=O)c2cc(C)nn2CC)nc2cc(C(N)=O)cc(SC)c21] | MS (ESI) m/z = 1051 [M + H]⁺ ¹HNMR (400M, DMSO) δ 12.85 (s, br, 2H) 8.07 (s, 1H) 7.96 (s, 1H) 7.86 (s, 1H), 7.62 (s, 1H), 7.64 (s, 1H), 7.43 (s, 1H) 7.36 (s, 1H) 7.31 (s, 1H), 6.51 (s, 1H), 5.71- 5.77 (m, 2H), 5.09- 5.16 (m, 2H), 4.81- 4.90 (m, 2H), 4.45- 4.53 (m, 2H), 4.00- 4.07 (m, 2H), 3.04- 3.13 (m, 9H), 2.87- 2.94 (m, 2H), 2.43(s, 3H), 2.09 (s, 3H), 1.91- 1.99 (m, 2H), 1.78- 1.81 (m, 2H), 1.27 (t, J = 7.20 Hz, 5H), 1.15 (t, J = 7.48 Hz, 3H). |
| 10 | 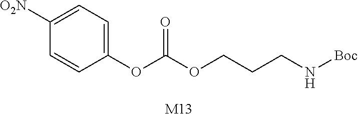 | 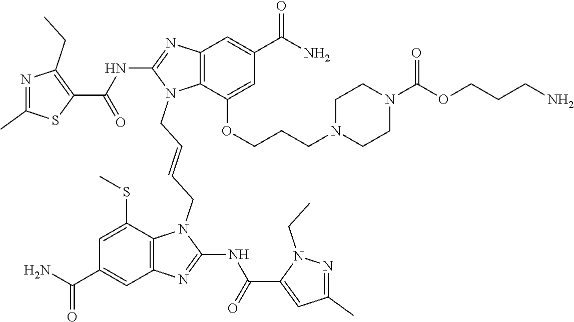 [SMILES: CCc1nc(C)sc1C(=O)Nc1nc2cc(C(N)=O)cc(OCCCN3CCN(C(=O)OCCCN)CC3)c2n1C/C=C/Cn1c(NC(=O)c2cc(C)nn2CC)nc2cc(C(N)=O)cc(SC)c21] | MS (ESI) m/z = 983 [M + H]⁺ ¹HNMR (400M, DMSO) δ 12.81 (s, br, 2H) 8.07 (s, 1H) 7.95 (s, 1H) 7.86 (s, 1H), 7.62 (s, 1H), 7.64 (s, 1H), 7.43 (s, 1H) 7.36 (s, 1H) 7.31 (s, 1H), 6.51 (s, 1H), 5.70- 5.75 (m, 2H), 5.13 (s, 2H), 4.86 (s, 2H), 4.52- 4.54 (m, 2H), 4.03- 4.20 (m, 4H), 3.30- 3.45 (m, 2H), 3.08- 3.16 (m, 6H), 2.88- 2.93 (m, 4H), 2.42 (s, 3H), 2.09 (s, 3H), 1.87- 1.94 (m, 4H), 1.28 (t, J = 7.20 Hz, 5H), 1.15 (t, J = 7.48 Hz, 3H). |

### Example 11

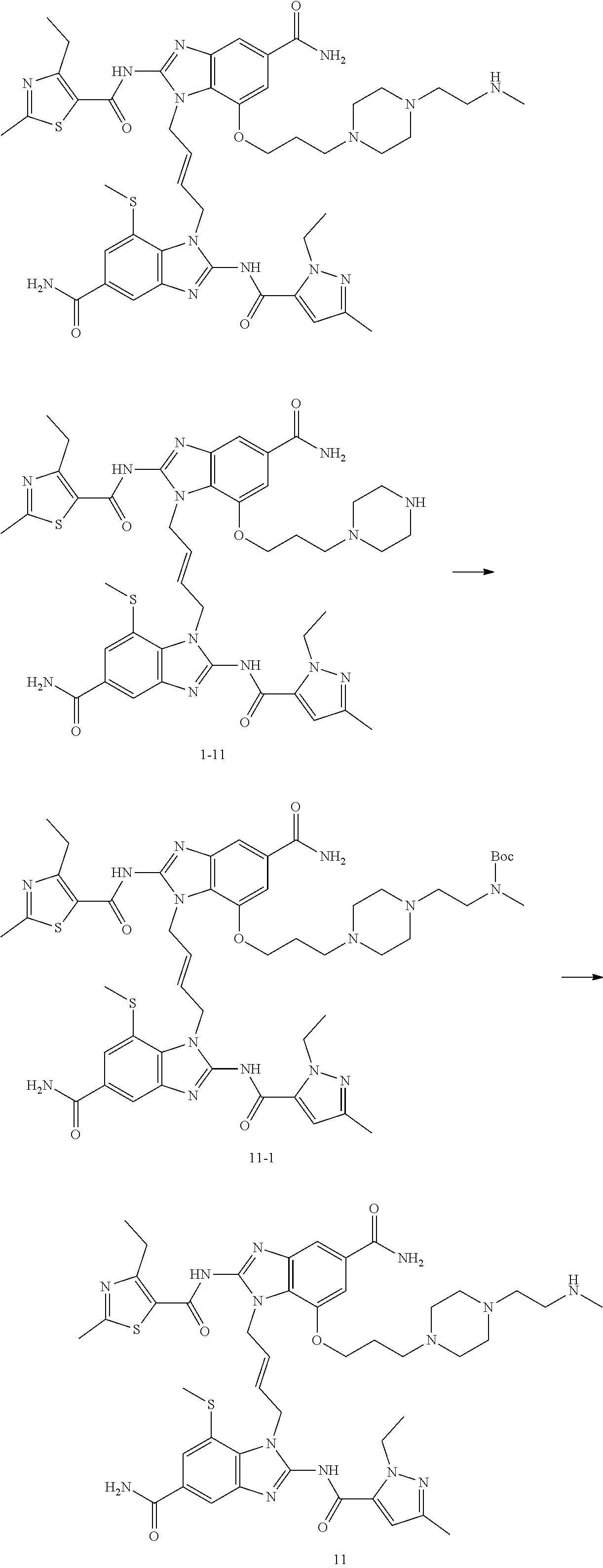

(Step 1) The compound 1-11 (50 mg, 0.057 mmol) was dissolved in a mixture of methanol (1 mL) and tetrahydrofuran (2 mL), to which N-tert-butoxycarbonyl-N-methyl-2-aminoacetaldehyde (29 mg, 0.171 mmol) was added. After 0.5 h, acetic acid (34 mg, 0.57 mmol) and sodium cyanoborohydride (11 mg, 0.171 mmol) were added. The reaction mixture was heated to 60° C., reacted for 2 h, dried by rotary evaporation, and dissolved with dichloromethane. The organic phase was washed with water, dehydrated and dried by rotary evaporation to afford the target compound 11-1 (58 mg), which was directly used in the next step of reaction. MS (ESI) m/z=1039[M+H]⁺

(Step 2) Trifluoroacetic acid (3 mL) was added to a dichloromethane (3 mL) solution of the compound 11-1 (58 mg, 0.055 mmol). The reaction mixture was reacted at room temperature for 1 h and then dried by rotary evaporation to give a crude product. The resulting crude product was purified by preparative HPLC to afford the target compound 11 (33 mg). MS (ESI) m/z=939[M+H]⁺

¹H NMR (400 MHz, DMSO-d₆) δ 12.81-12.85 (m, 2H), 8.22-8.31 (m, 1H), 8.05-8.10 (m, 1H), 7.94-7.99 (m, 1H), 7.84-7.88 (m, 1H), 7.64 (s, 2H), 7.42-7.47 (m, 1H), 7.36-7.41 (m, 1H), 7.29-7.34 (m, 1H), 6.52 (s, 1H), 5.72-5.85 (m, 2H), 5.12-5.17 (m, 2H), 4.81-4.97 (m, 2H), 4.48-4.56 (m, 2H), 4.06-4.11 (m, 2H), 3.33-3.42 (m, 2H), 3.10-3.17 (m, 4H), 3.02-3.08 (m, 2H), 2.92-3.01 (m, 2H), 2.82-2.91 (m, 2H), 2.59-2.65 (m, 5H), 2.53 (s, 3H), 2.44 (s, 3H), 2.29-2.37 (m, 2H), 2.10 (s, 3H), 1.91-2.04 (m, 2H), 1.29 (t, J=7.6 Hz, 3H), 1.16 (t, J=7.6 Hz, 3H).

### Example 12

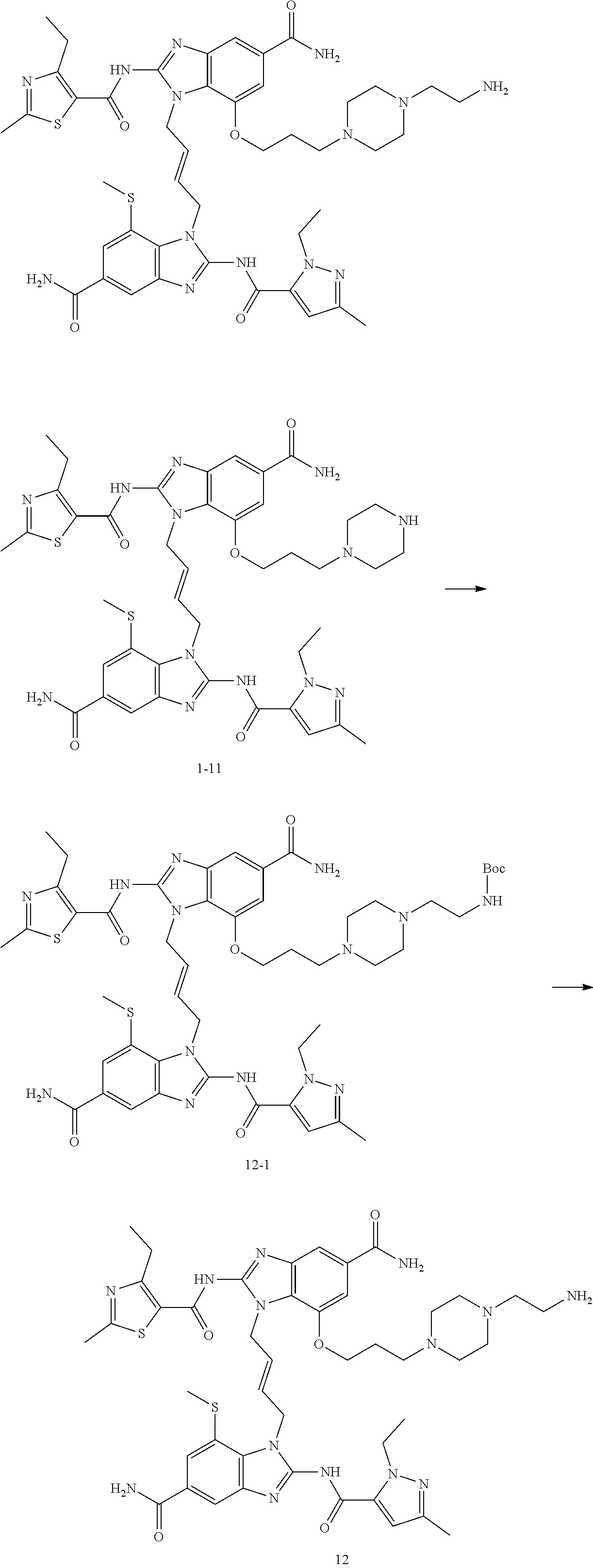 [SMILES: CCc1nc(C)sc1C(=O)Nc1nc2cc(C(N)=O)cc(OCCCN3CCN(CCN)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCNCC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(CCNC(=O)OC(C)(C)C)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(CCN)CC3)c2n1C/C=C/Cn1c(NC(=O)c2cc(C)nn2CC)nc2cc(C(N)=O)cc(SC)c21]

(Step 1) The compound 1-11 (50 mg, 0.057 mmol) was dissolved in a mixed solution of methanol (1 mL) and tetrahydrofuran (2 mL), to which N-tert-butoxycarbonyl-2-aminoacetaldehyde (27 mg, 0.171 mmol) was added. After 0.5 h, acetic acid (34 mg, 0.57 mmol) and sodium cyanoborohydride (11 mg, 0.171 mmol) were added. The reaction mixture was heated to 60° C., reacted for 2 h, dried by rotary evaporation, and dissolved with dichloromethane. The organic phase was washed with water, dehydrated and dried by rotary evaporation to afford the target compound 12-1 (45 mg), which was directly used in the next step of reaction. MS (ESI) m/z=1025[M+H]⁺

(Step 2) Trifluoroacetic acid (3 mL) was added to a dichloromethane (3 mL) solution of the compound 12-1 (45 mg, 0.044 mmol). The reaction mixture was reacted at room temperature for 1 h and then dried by rotary evaporation to give a crude product. The resulting crude product was purified by preparative HPLC to afford the target compound 12 (25 mg). MS (ESI) m/z=925[M+H]⁺

¹H NMR (400M, DMSO-d6, D₂O) δ 7.80 (s, 1H), 7.57-7.62 (m, 2H), 7.26 (s, 1H), 6.47 (s, 1H), 5.61-5.83 (m, 2H), 5.09-5.17 (m, 2H), 4.82-4.91 (m, 2H), 4.43-4.52 (m, 2H), 4.02-4.11 (m, 2H), 3.26-3.40 (m, 2H), 3.01-3.17 (m, 5H), 2.76-2.97 (m, 6H), 2.55-2.62 (m, 2H), 2.40 (s, 3H), 2.07 (s, 3H), 1.94-2.03 (m, 2H), 1.24 (t, J=7.16 Hz, 3H), 1.11 (t, J=7.48 Hz, 3H).

### Example 13

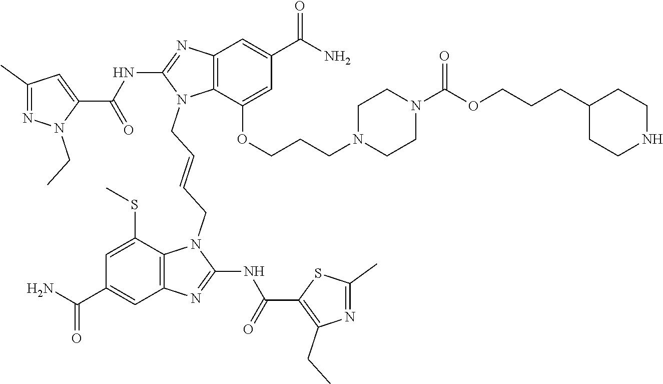 [SMILES: CCc1nc(C)sc1C(=O)Nc1nc2cc(C(N)=O)cc(SC)c2n1C/C=C/Cn1c(NC(=O)c2cc(C)nn2CC)nc2cc(C(N)=O)cc(OCCCN3CCN(C(=O)OCCCC4CCNCC4)CC3)c21]

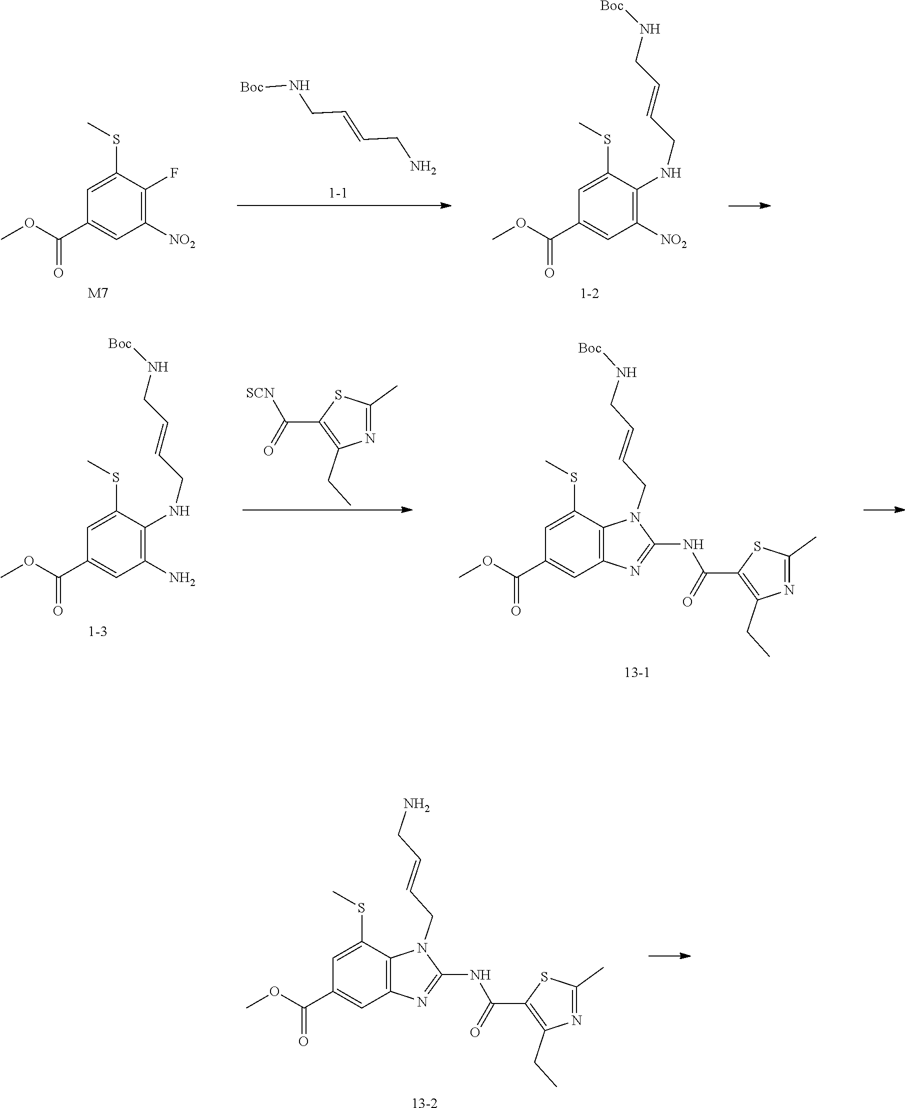

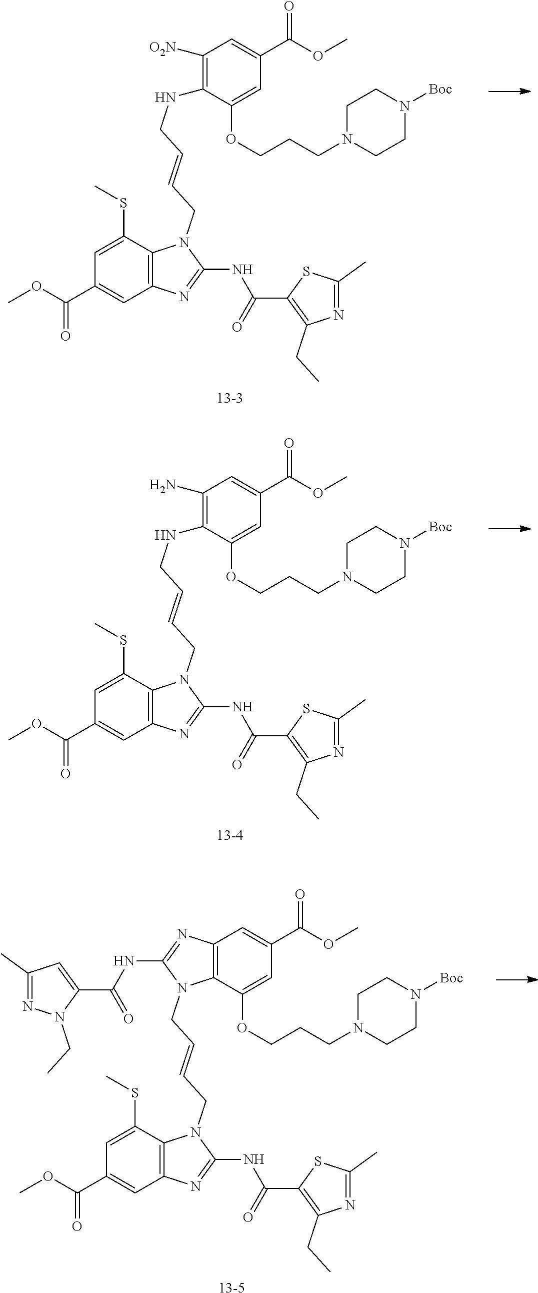 [SMILES: CCc1nc(C)sc1C(=O)Nc1nc2cc(C(=O)OC)cc(SC)c2n1C/C=C/CNc1c(OCCCN2CCN(C(=O)OC(C)(C)C)CC2)cc(C(=O)OC)cc1[N+](=O)[O-] ; "13-3" ; CCc1nc(C)sc1C(=O)Nc1nc2cc(C(=O)OC)cc(SC)c2n1C/C=C/CNc1c(N)cc(C(=O)OC)cc1OCCCN1CCN(C(=O)OC(C)(C)C)CC1 ; "13-4" ; CCc1nc(C)sc1C(=O)Nc1nc2cc(C(=O)OC)cc(SC)c2n1C/C=C/Cn1c(NC(=O)c2cc(C)nn2CC)nc2cc(C(=O)OC)cc(OCCCN3CCN(C(=O)OC(C)(C)C)CC3)c21 ; "13-5"]

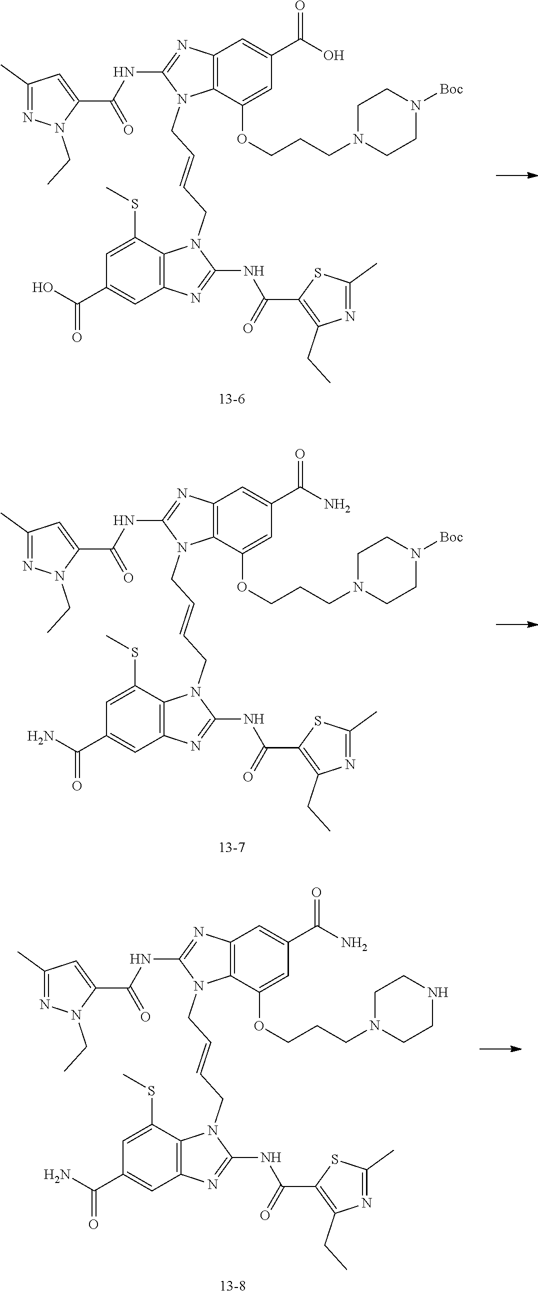 [SMILES: CCc1nc(C)sc1C(=O)Nc1nc2cc(C(=O)O)cc(SC)c2n1C/C=C/Cn1c(NC(=O)c2cc(C)nn2CC)nc2cc(C(=O)O)cc(OCCCN3CCN(C(=O)OC(C)(C)C)CC3)c21 ; "13-6" ; CCc1nc(C)sc1C(=O)Nc1nc2cc(C(N)=O)cc(SC)c2n1C/C=C/Cn1c(NC(=O)c2cc(C)nn2CC)nc2cc(C(N)=O)cc(OCCCN3CCN(C(=O)OC(C)(C)C)CC3)c21 ; "13-7" ; CCc1nc(C)sc1C(=O)Nc1nc2cc(C(N)=O)cc(SC)c2n1C/C=C/Cn1c(NC(=O)c2cc(C)nn2CC)nc2cc(C(N)=O)cc(OCCCN3CCNCC3)c21 ; "13-8"]

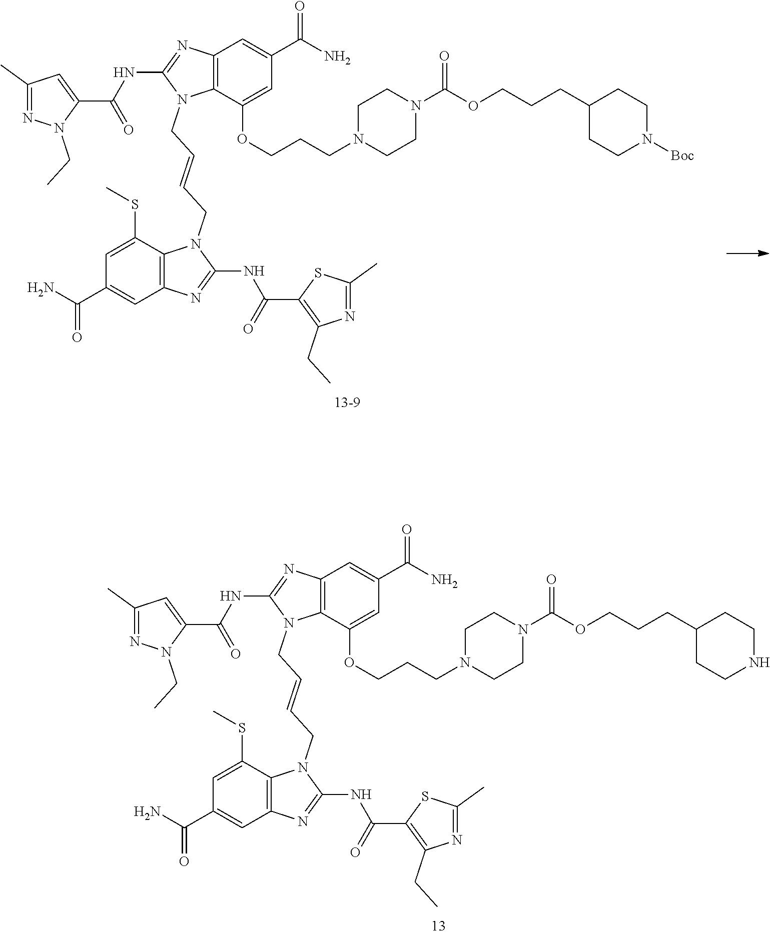 [SMILES: CCc1nc(C)sc1C(=O)Nc1nc2cc(C(N)=O)cc(SC)c2n1C/C=C/Cn1c(NC(=O)c2cc(C)nn2CC)nc2cc(C(N)=O)cc(OCCCN3CCN(C(=O)OCCCC4CCN(C(=O)OC(C)(C)C)CC4)CC3)c21 ; CCc1nc(C)sc1C(=O)Nc1nc2cc(C(N)=O)cc(SC)c2n1C/C=C/Cn1c(NC(=O)c2cc(C)nn2CC)nc2cc(C(N)=O)cc(OCCCN3CCN(C(=O)OCCCC4CCNCC4)CC3)c21]

The synthesis method of Example 13 was basically the same as that of Example 8 except that the intermediate M1 used in Step 3 was replaced with the intermediate M2, and the intermediate M2 used in Step 7 was replaced with the intermediate M1, so as to afford the target compound 13 (11.4 mg). MS (ESI) m/z=1051 [M+H]⁺

¹H NMR (400 MHz, DMSO-D₂O) δ 7.66-7.58 (m, 2H), 5.83 (m 2H), 5.08 (s, 2H), 4.94 (s, 2H), 4.58-4.50 (m, 2H), 4.01-3.91 (m, 6H), 3.22 (d, J=25.8 Hz, 9H), 3.09 (q, J=7.4 Hz, 4H), 2.78 (t, J=12.6 Hz, 3H), 2.48 (s, 4H), 2.44 (s, 4H), 2.31-2.22 (m, 2H), 2.13 (s, 4H), 1.85-1.72 (m, 3H), 1.72-1.62 (m, 3H), 1.58 (d, J=7.6 Hz, 4H), 1.29 (t, J=7.1 Hz, 5H), 1.18-1.10 (m, 5H).

### Example 14

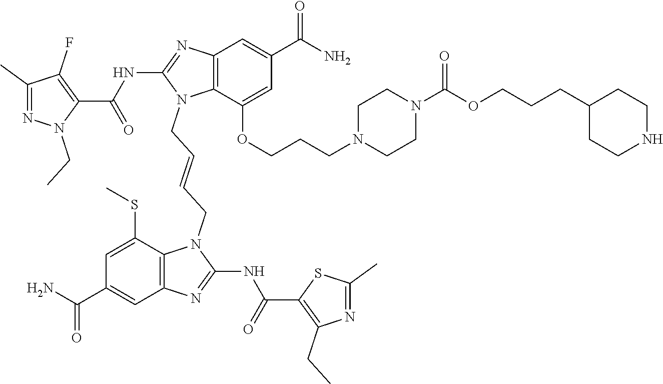 [SMILES: CCc1nc(C)sc1C(=O)Nc1nc2cc(C(N)=O)cc(SC)c2n1C/C=C/Cn1c(NC(=O)c2c(F)c(C)nn2CC)nc2cc(C(N)=O)cc(OCCCN3CCN(C(=O)OCCCC4CCNCC4)CC3)c21]

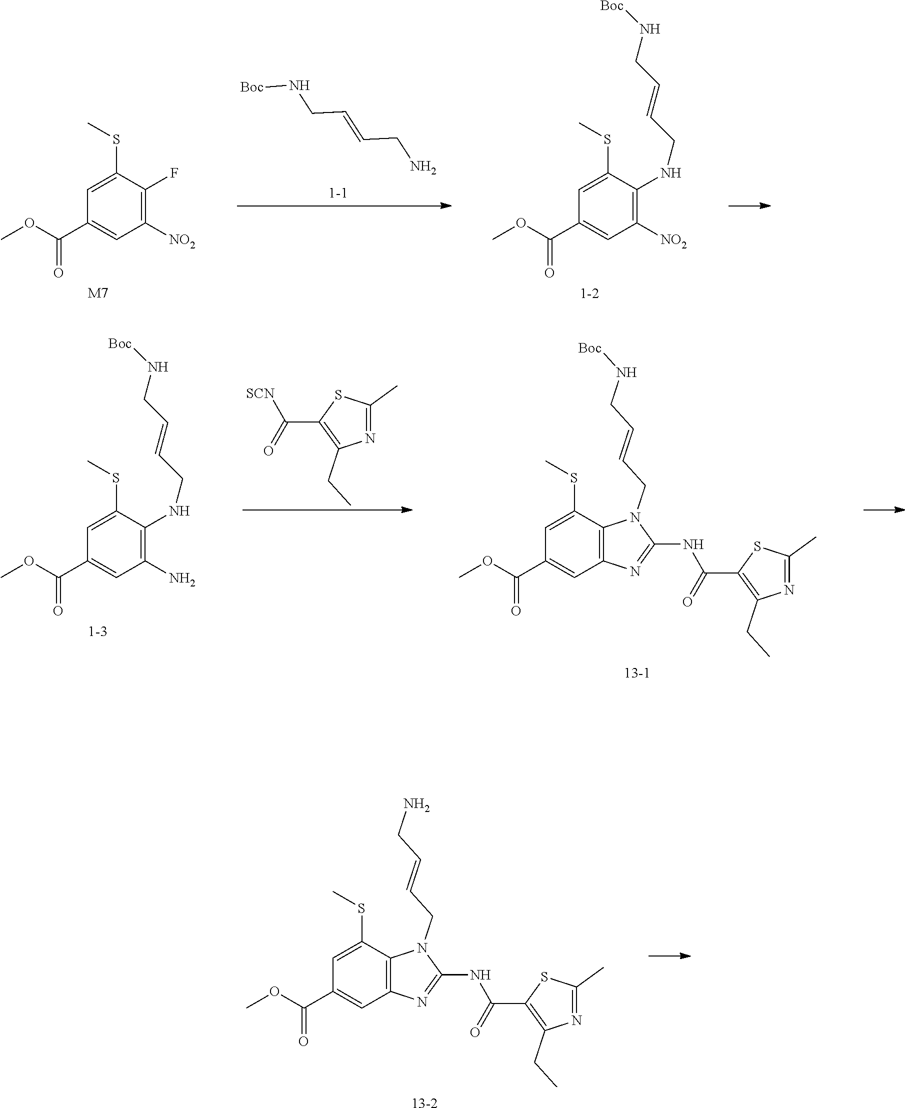

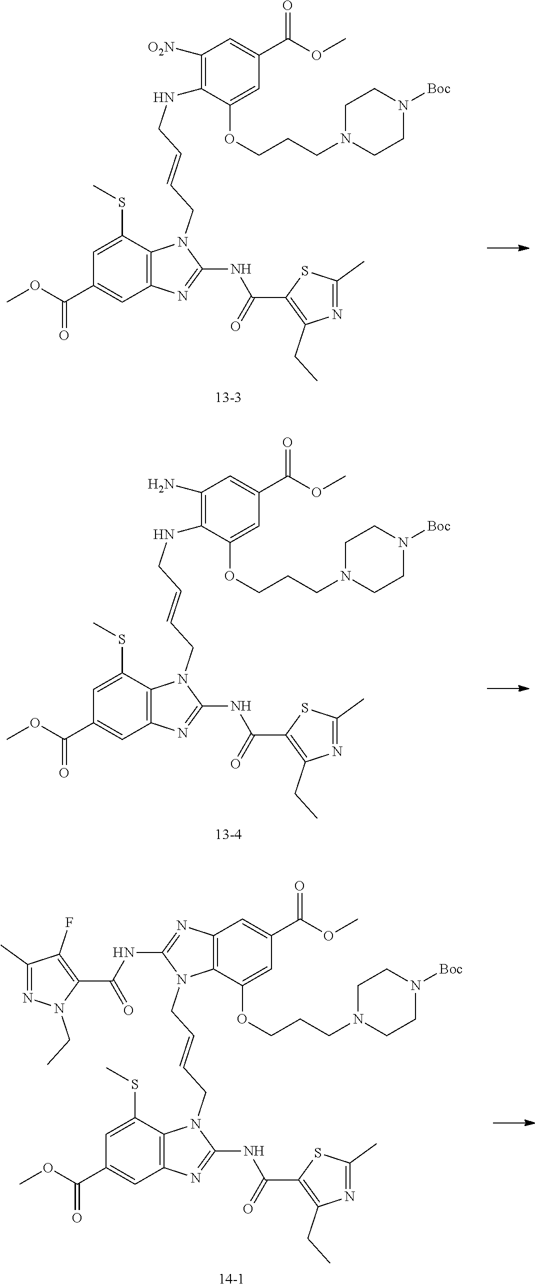 [SMILES: CCc1nc(C)sc1C(=O)Nc1nc2cc(C(=O)OC)cc(SC)c2n1C/C=C/CNc1c(OCCCN2CCN(C(=O)OC(C)(C)C)CC2)cc(C(=O)OC)cc1[N+](=O)[O-] ; "13-3" ; CCc1nc(C)sc1C(=O)Nc1nc2cc(C(=O)OC)cc(SC)c2n1C/C=C/CNc1c(N)cc(C(=O)OC)cc1OCCCN1CCN(C(=O)OC(C)(C)C)CC1 ; "13-4" ; CCc1nc(C)sc1C(=O)Nc1nc2cc(C(=O)OC)cc(SC)c2n1C/C=C/Cn1c(NC(=O)c2c(F)c(C)nn2CC)nc2cc(C(=O)OC)cc(OCCCN3CCN(C(=O)OC(C)(C)C)CC3)c21 ; "14-1"]

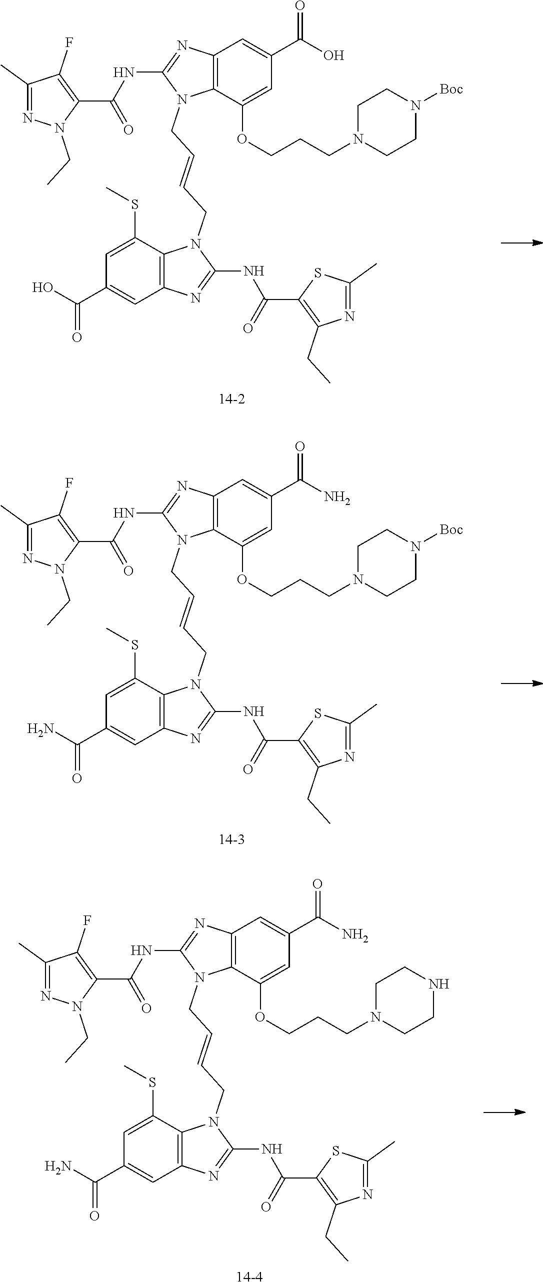 [SMILES: CCc1nc(C)sc1C(=O)Nc1nc2cc(C(=O)O)cc(SC)c2n1C/C=C/Cn1c(NC(=O)c2c(F)c(C)nn2CC)nc2cc(C(=O)O)cc(OCCCN3CCN(C(=O)OC(C)(C)C)CC3)c21 ; "14-2" ; CCc1nc(C)sc1C(=O)Nc1nc2cc(C(N)=O)cc(SC)c2n1C/C=C/Cn1c(NC(=O)c2c(F)c(C)nn2CC)nc2cc(C(N)=O)cc(OCCCN3CCN(C(=O)OC(C)(C)C)CC3)c21 ; "14-3" ; CCc1nc(C)sc1C(=O)Nc1nc2cc(C(N)=O)cc(SC)c2n1C/C=C/Cn1c(NC(=O)c2c(F)c(C)nn2CC)nc2cc(C(N)=O)cc(OCCCN3CCNCC3)c21 ; "14-4"]

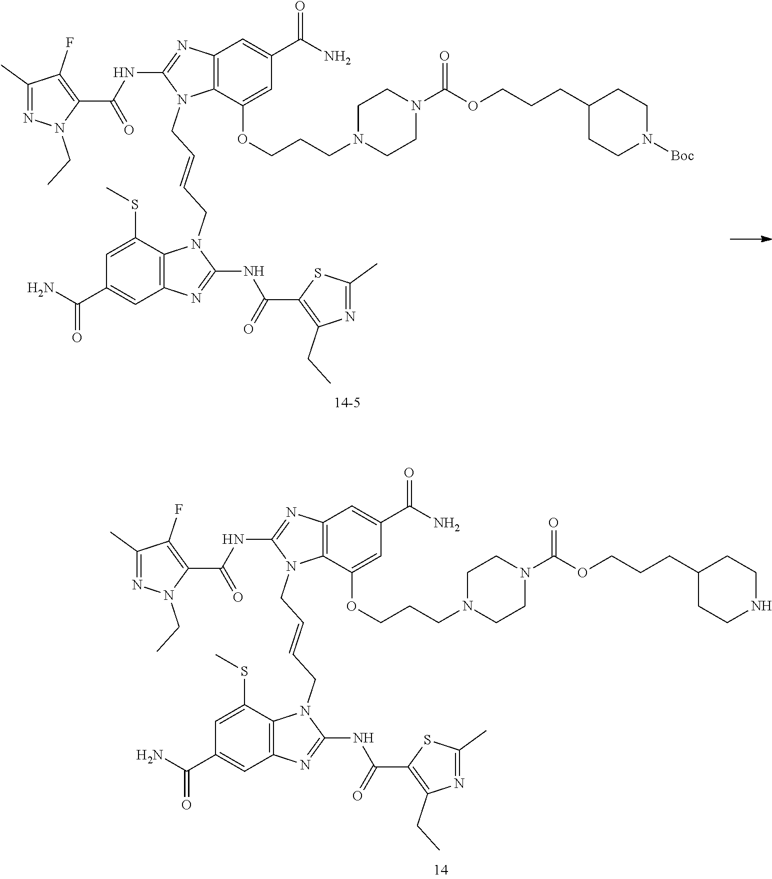 [SMILES: CCc1nc(C)sc1C(=O)Nc1nc2cc(C(N)=O)cc(SC)c2n1C/C=C/Cn1c(NC(=O)c2c(F)c(C)nn2CC)nc2cc(C(N)=O)cc(OCCCN3CCN(C(=O)OCCCC4CCN(C(=O)OC(C)(C)C)CC4)CC3)c21 ; CCc1nc(C)sc1C(=O)Nc1nc2cc(C(N)=O)cc(SC)c2n1C/C=C/Cn1c(NC(=O)c2c(F)c(C)nn2CC)nc2cc(C(N)=O)cc(OCCCN3CCN(C(=O)OCCCC4CCNCC4)CC3)c21]

The synthesis method of Example 14 was basically the same as that of Example 8 except that the intermediate M1 used in Step 3 was replaced with the intermediate M2, and the intermediate M2 used in Step 7 was replaced with M3, so as to afford the target compound 14 (12 mg). MS (ESI) m/z=1069[M+H]⁺

¹H NMR (400 MHz, DMSO-D₂O) δ 7.83 (s, 1H) 7.61 (d, J=1.7 Hz, 2H), 7.25 (s, 1H) 5.85 (m, 2H), 5.08 (s, 2H), 4.93 (s, 2H), 4.54-4.41 (m, 2H), 4.03-3.88 (m, 4H), 3.33-3.16 (m, 13H), 3.13-3.02 (m, 4H), 2.84-2.68 (m, 2H), 2.48 (s, 5H), 2.44 (s, 4H), 2.32-2.23 (m, 3H), 2.17 (s, 5H), 2.10 (s, 4H), 1.83-1.74 (m, 2H), 1.62-1.43 (m, 4H), 1.26 (t, J=7.1 Hz, 6H), 1.13 (t, J=7.5 Hz, 5H).

### Example 15

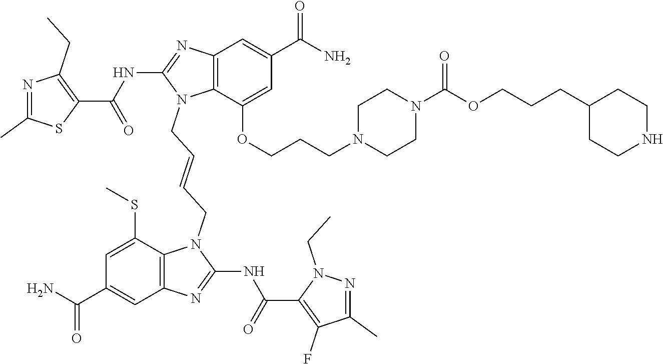 [SMILES: CCc1nc(C)sc1C(=O)Nc1nc2cc(C(N)=O)cc(OCCCN3CCN(C(=O)OCCCC4CCNCC4)CC3)c2n1C/C=C/Cn1c(NC(=O)c2c(F)c(C)nn2CC)nc2cc(C(N)=O)cc(SC)c21]

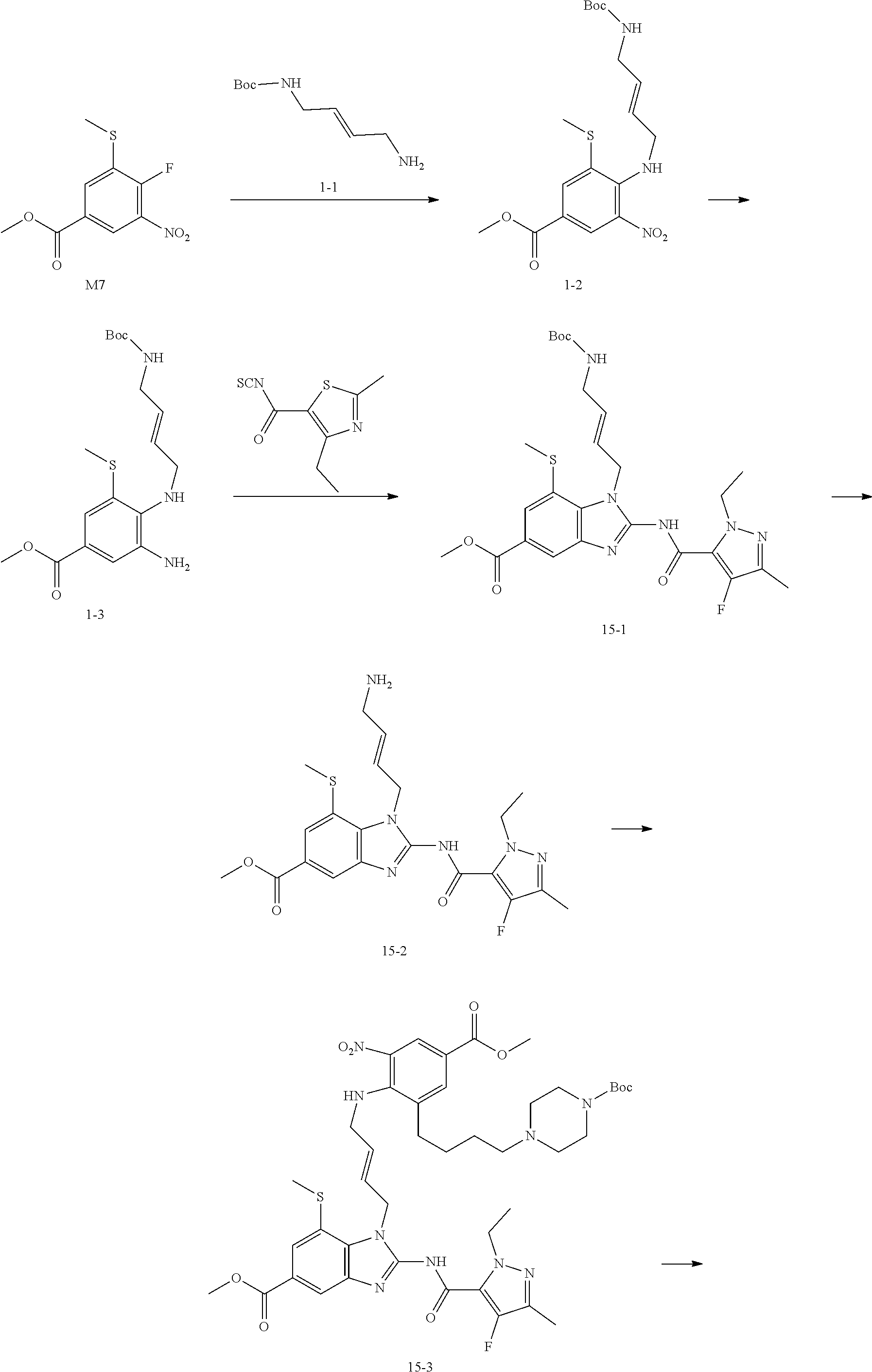

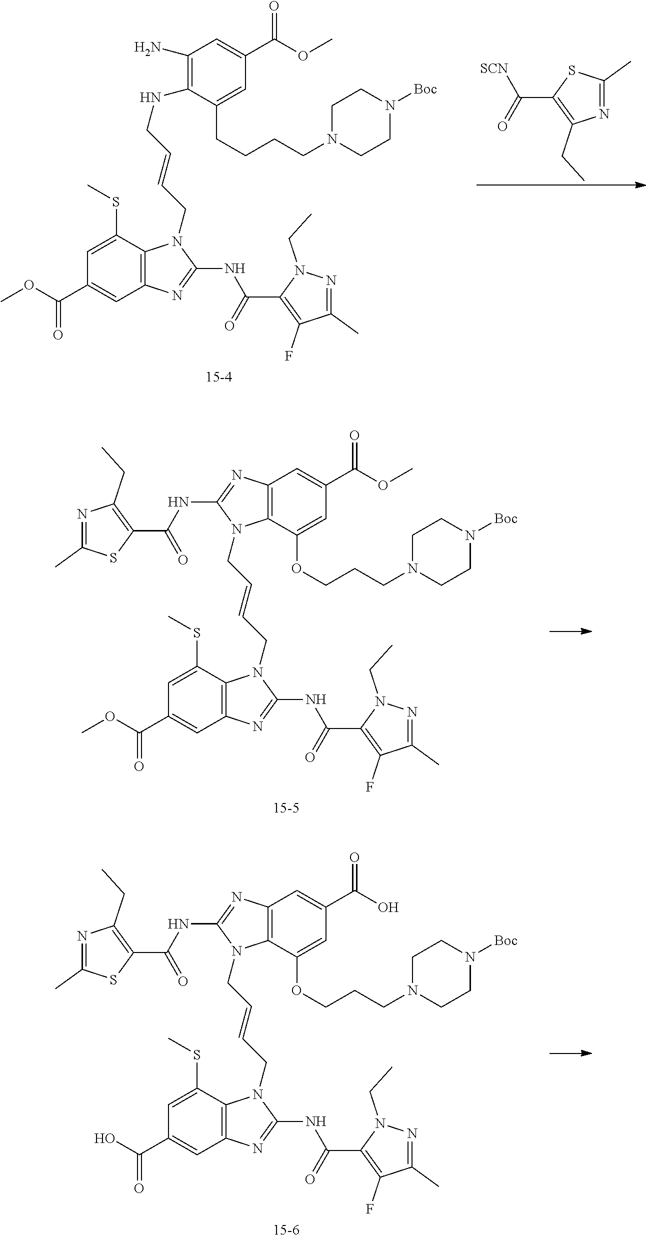

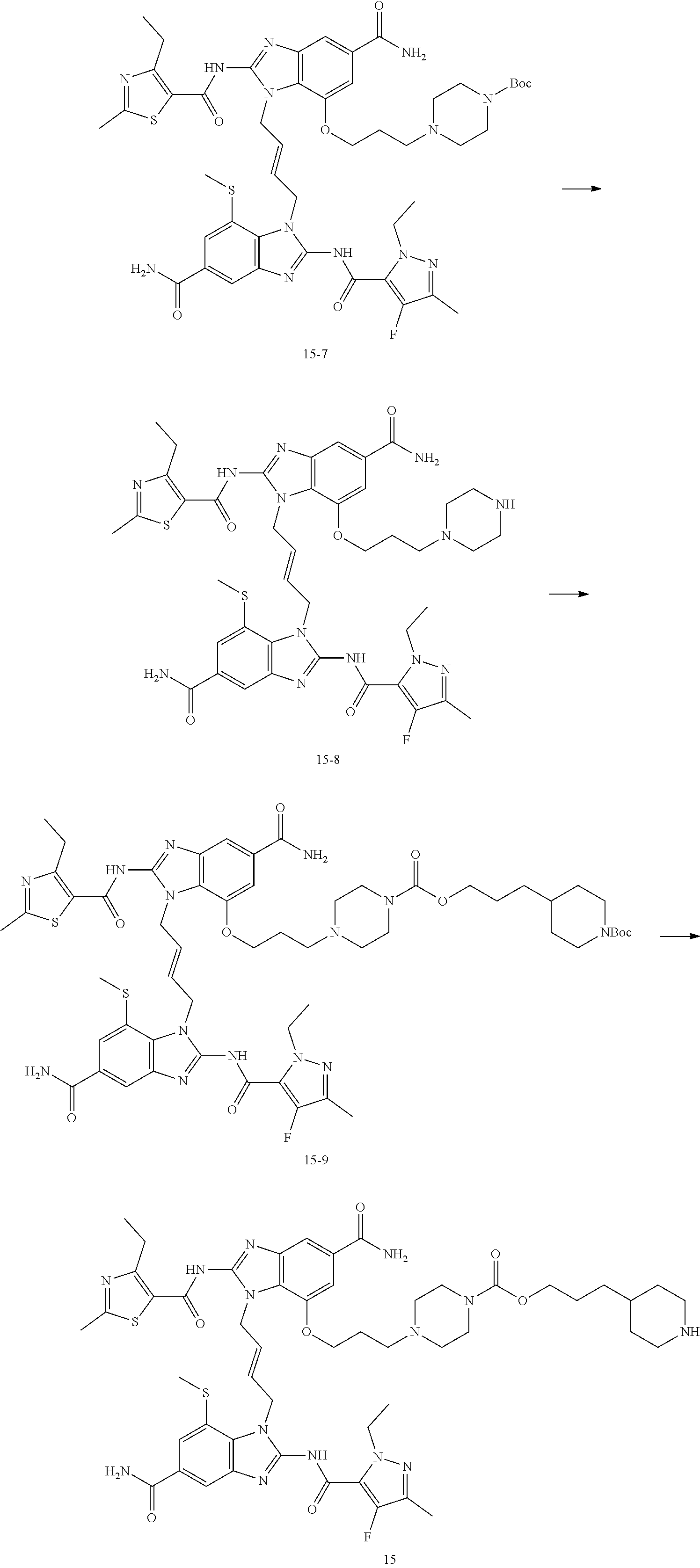

The synthesis method of Example 15 was basically the same as that of Example 8 except that the intermediate M1 used in Step 3 was replaced with the intermediate M3, so as to afford the target compound 15 (3.5 mg). MS (ESI) m/z=1069[M+H]⁺

¹H NMR (400 MHz, DMSO-D₂O) δ 7.84 (s, 1H), 7.61 (d, J=6.0, 1.4 Hz, 2H), 7.28 (s, 1H), 5.81 (m, 2H), 5.13 (s, 2H), 4.88 (s, 2H), 4.50-4.37 (m, 2H), 4.14-3.92 (m, 5H), 3.31-3.20 (m, 4H), 3.14-3.03 (m, 3H), 2.90-2.75 (m, 4H), 2.43 (s, 4H), 2.07 (s, 4H), 1.95-1.75 (m, 5H), 1.63-1.45 (m, 3H), 1.33-1.21 (m, 8H), 1.13 (t, J=7.5 Hz, 4H).

### Example 16

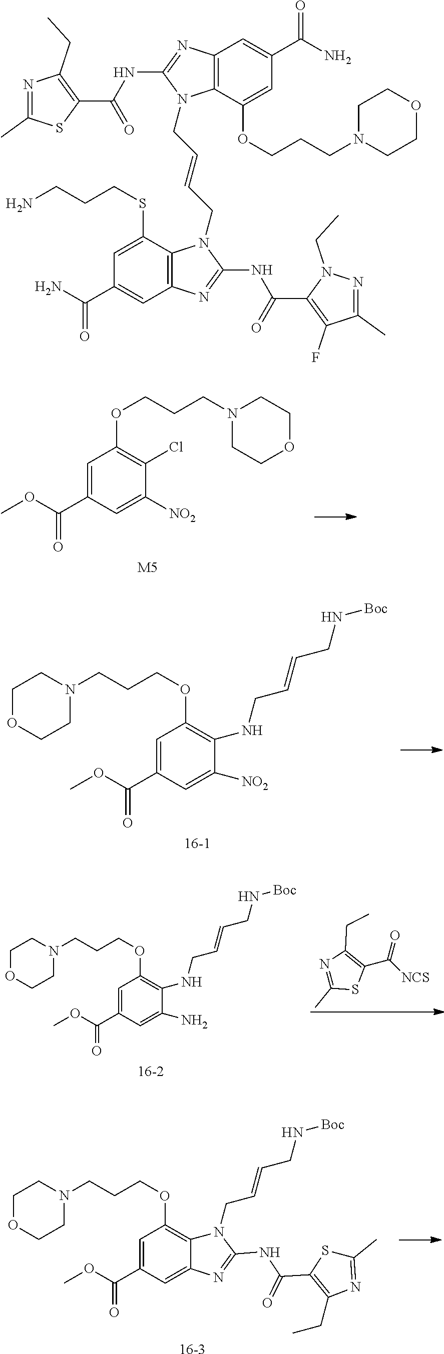

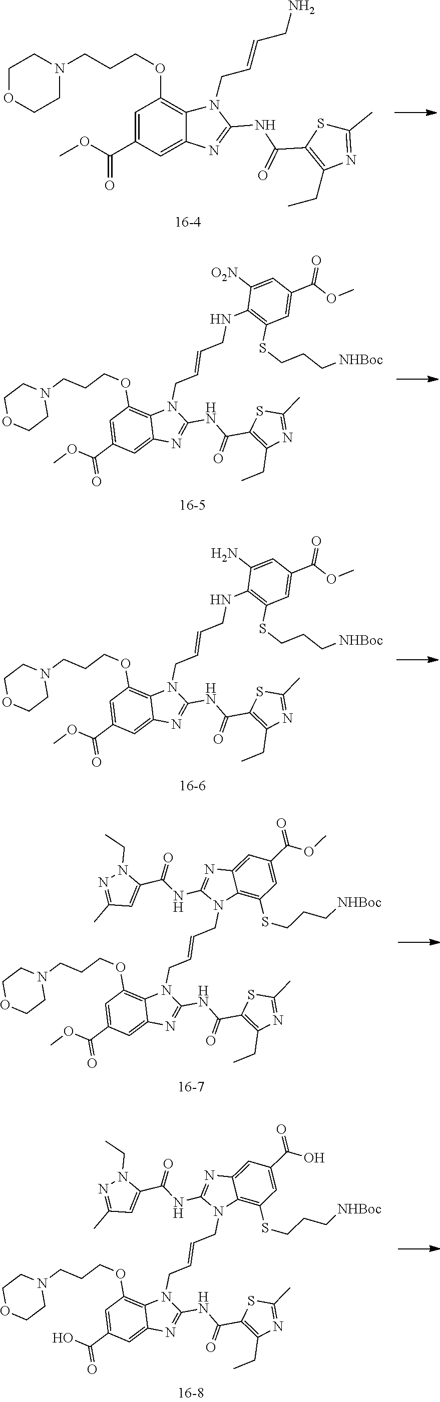

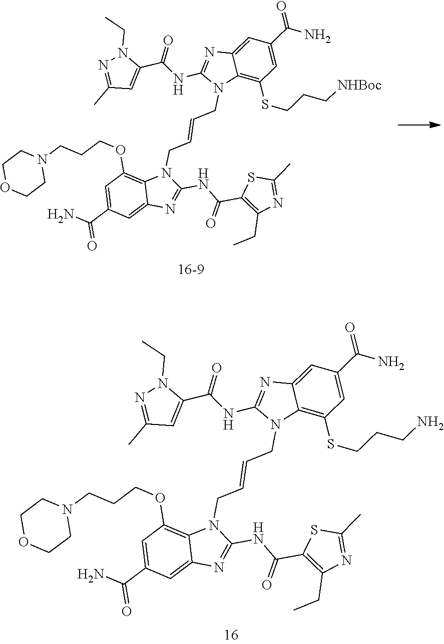 [SMILES: CCc1nc(C)sc1C(=O)Nc1nc2cc(C(N)=O)cc(OCCCN3CCOCC3)c2n1C/C=C/Cn1c(NC(=O)c2cc(C)nn2CC)nc2cc(C(N)=O)cc(SCCCNC(=O)OC(C)(C)C)c21 ; CCc1nc(C)sc1C(=O)Nc1nc2cc(C(N)=O)cc(OCCCN3CCOCC3)c2n1C/C=C/Cn1c(NC(=O)c2cc(C)nn2CC)nc2cc(C(N)=O)cc(SCCCN)c21]

(Step 1) Methyl 4-chloro-3-(3-morpholinopropoxy)-5-nitrobenzoate (6.8 g, 19 mmol) was dispersed in n-butanol (100 mL), to which tert-butyl (E)-(4-aminobut-2-en-1-yl) carbamate (3.5 g, 19 mmol) and DIPEA (12.3 g, 95 mmol) were added. The reaction mixture was heated to 120° C., stirred for 18 h, dried by rotary evaporation dissolved in ethyl acetate, and added with an appropriate amount of dilute hydrochloric acid (0.5 M) in an ice bath for adjusting the pH to neutrality. The separated organic phase was washed respectively with water and saturated NaCl solution, dehydrated and dried by rotary evaporation to afford the compound 16-1 (7.3 g, 76% yield). MS(ESI) m/z=509[M+H]⁺

(Step 2) Ammonia water (15 mL) was added to a methanol (100 mL) solution of the compound 16-1 (7.3 g, 14.4 mmol) in an ice bath. After 10 minutes, an aqueous solution of sodium dithionite (12.5 g, 71.9 mmol) was added, and the reaction mixture was warmed up slowly to room temperature and reacted for 2 h. Inorganic salts were removed by filtration, and ethyl acetate was added for extraction. The organic phase was washed with saturated NaCl solution, dehydrated with anhydrous magnesium sulfate, dried by rotary evaporation and purified by silica gel column chromatography (eluent: PE/EA=1/2) to afford the compound 16-2 (4.5 g, 65% yield). MS(ESI) m/z=479.3[M+H]⁺

(Step 3) 4-ethyl-2-methylthiazole-5-carbonyl isothiocyanate (0.96 g, 4.52 mmol) was added to a DMF (30 mL) solution of the compound 16-2 (1.8 g, 3.76 mmol) in an ice bath. The reaction mixture was reacted for 0.5 h, added with DIPEA (1.46 g, 11.3 mmol) and HATU (1.72 g, 4.52 mmol), warmed up to room temperature and reacted for 12 h. Then the reaction solution was slowly poured into water, and yellow solids were precipitated, collected by filtration and dried to afford the compound 16-3 (1.9 g, 77% yield). MS(ESI) m/z=657[M+H]⁺

(Step 4) TFA (5 mL) was added to a DCM (10 mL) solution of the compound 4d (1.9 g, 2.9 mmol) in an ice bath, and then the reaction solution was warmed up to room temperature and reacted for 2 h. The solvent was removed by rotary evaporation under reduced pressure. After ethyl acetate was added, the free TFA was removed by rotary evaporation under reduced pressure to afford the compound 16-4 (1.6 g, 99% yield). MS(ESI) m/z=557[M+H]⁺

(Step 5) DIPEA (387 mg, 3 mmol) and M8 (388 mg, 1 mmol) were added to a DMF (10 mL) solution of methyl (E)-1-(4-aminobut-2-en-1-yl)-2-(4-ethyl-2-methylthiazole-5-carboxamido)-7-(3-morpholinopropoxy)-1H-benzo[d] imidazole-5-carboxylate (556 mg, 1 mmol), and the reaction mixture was reacted at room temperature for 3 h. The reaction solution was poured into water, and ethyl acetate was added for extraction. The organic phase was collected, washed with saturated NaCl solution, dehydrated with anhydrous sodium sulfate and concentrated to afford the compound 16-5 (740 mg, 80% yield). MS(ESI) m/z=925[M+H]⁺

(Step 6) Ammonia water (1 mL) was added to a methanol (10 mL) solution of the compound 16-5 (740 mg, 0.8 mmol) in an ice bath. After 10 minutes, an aqueous solution of sodium dithionite (696 mg, 4.0 mmol) was added, the reaction solution was warmed up slowly to room temperature and reacted for 2 h. Inorganic salts were removed by filtration, and ethyl acetate was added for extraction. The organic phase was washed with saturated NaCl solution and then dehydrated with anhydrous magnesium sulfate. The resulting residue was then dried by rotary evaporation to give a crude product. The crude product was purified by silica gel column chromatography (eluent: petroleum ether/ethyl acetate=1/1) to afford the compound 16-6 (520 mg, 73% yield). MS(ESI) m/z=895[M+H]⁺

(Step 7) The compound M1 (46 mg, 0.232 mmol) was added to a DMF (5 mL) solution of the compound 16-6 (145 mg, 0.162 mmol) in an ice bath. After reacted for 0.5 h, DIPEA (62 mg, 0.486 mmol) and HATU (73 mg, 0.194 mmol) were added, and then the reaction solution was warmed up to room temperature and reacted for 12 h. The reaction solution was slowly poured into water, and solids were precipitated, collected by filtration and dried to afford the compound 16-7 (144 mg, 85% yield). MS(ESI) m/z=1056[M+H]⁺

(Step 8) The compound 16-7 (144 mg, 0.137 mmol) was dissolved in a mixed solution of methanol, tetrahydrofuran and water (9 mL, volume ratio: 1/1/1), and hydrated lithium hydroxide (57 mg, 1.36 mmol) was added. The reaction solution was heated up to 75° C. and reacted overnight. The organic solvent was removed by rotary evaporation, and the resulting residue was cooled to room temperature, and added with dilute hydrochloric acid (1 M) in an ice bath until the solids no longer precipitate. The resulting solids were collected by filtration and dried to afford the compound 16-8 (98 mg, 70% yield). MS(ESI) m/z=1028[M+H]⁺

(Step 9) The compound 16-8 (98 mg, 0.096 mmol) was dissolved in DMF (3 mL), to which HATU (87 mg, 0.23 mmol) and DIPEA (62 mg, 0.48 mmol) were added. After 0.5 h, ammonium bicarbonate (38 mg, 0.48 mmol) was added, and the reaction mixture was stirred at room temperature for 2 h, subjected to rotary evaporation, and dissolved with dichloromethane. The organic phase was washed with water, dehydrated and dried by rotary evaporation to afford the target compound 16-9 (80 mg), which was directly used in the next step of reaction. MS(ESI) m/z=1026[M+H]⁺

(Step 10) Trifluoroacetic acid (3 mL) was added to a dichloromethane (3 mL) solution of the compound 16-9 (80 mg, 0.078 mmol), and the reaction mixture was reacted at room temperature for 1 h and dried by rotary evaporation to give a crude product. The resulting crude product was purified by preparative HPLC to afford the target compound 16 (28 mg). MS(ESI) m/z=926[M+H]⁺

¹HNMR (400M, DMSO) δ 12.85 (s, br, 2H) 8.05 (s, 1H) 7.95-8.00 (m, 2H) 7.75 (s, 1H), 7.62 (s, 1H), 7.48 (s, 1H), 7.40 (s, 1H) 7.28 (s, 1H) 6.53 (s, 1H), 5.69-5.75 (m, 2H), 5.20 (s, 2H), 4.85 (s, 2H), 4.51-4.54 (m, 2H), 3.91-4.02 (m, 4H), 3.55-3.70 (m, 2H), 3.20-3.35 (m, 2H), 3.05-3.19 (m, 4H), 2.85-3.04 (m, 4H), 2.70-2.83 (m, 2H), 2.55 (s, 3H), 2.10 (s, 3H), 1.88-1.90 (m, 2H), 1.64-1.68 (m, 2H), 1.28 (t, J=7.20 Hz, 5H), 1.17 (t, J=7.48 Hz, 3H).

### Example 17

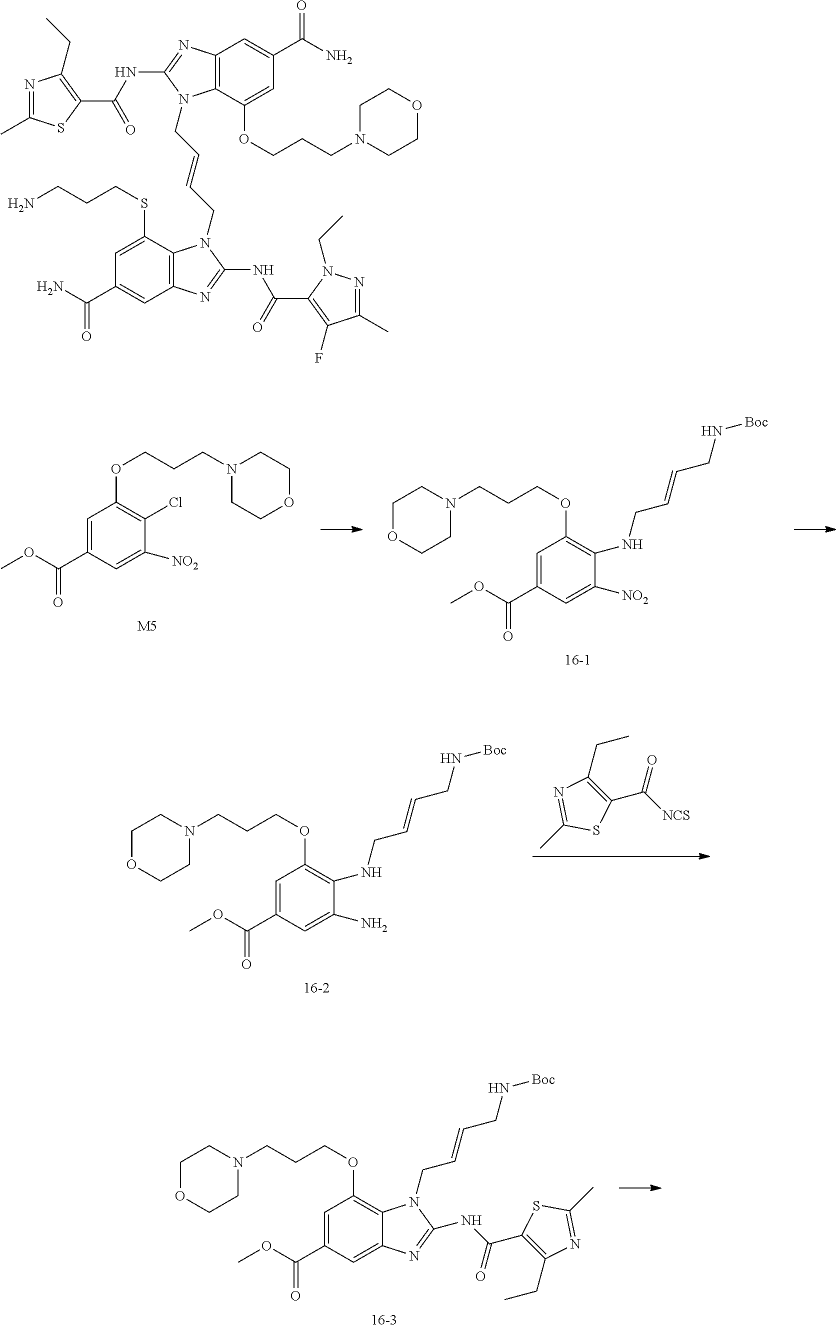

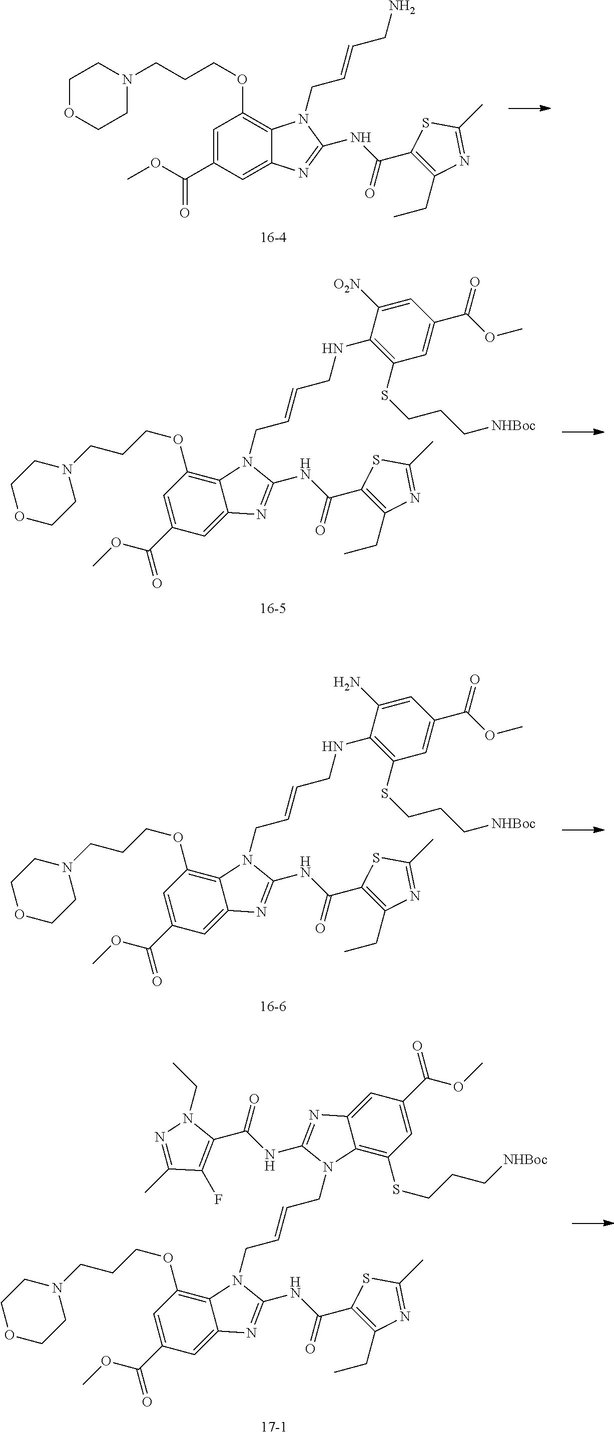

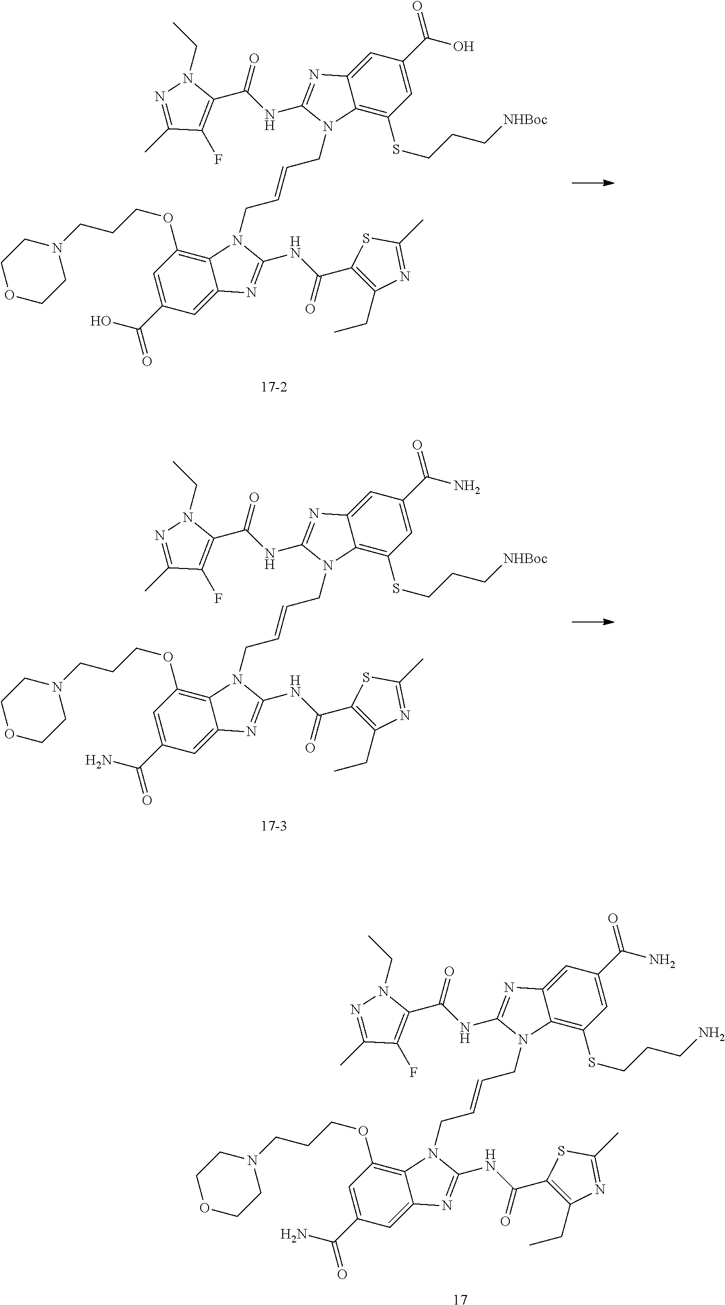 [SMILES: CCc1nc(C)sc1C(=O)Nc1nc2cc(C(=O)O)cc(OCCCN3CCOCC3)c2n1C/C=C/Cn1c(NC(=O)c2c(F)c(C)nn2CC)nc2cc(C(=O)O)cc(SCCCNC(=O)OC(C)(C)C)c21 ; "17-2" ; CCc1nc(C)sc1C(=O)Nc1nc2cc(C(N)=O)cc(OCCCN3CCOCC3)c2n1C/C=C/Cn1c(NC(=O)c2c(F)c(C)nn2CC)nc2cc(C(N)=O)cc(SCCCNC(=O)OC(C)(C)C)c21 ; "17-3" ; CCc1nc(C)sc1C(=O)Nc1nc2cc(C(N)=O)cc(OCCCN3CCOCC3)c2n1C/C=C/Cn1c(NC(=O)c2c(F)c(C)nn2CC)nc2cc(C(N)=O)cc(SCCCN)c21 ; "17"]

The synthesis method of Example 17 was basically the same as that of Example 16 except that the intermediate M1 used in Step 7 was replaced with the intermediate M3, so as to afford the target compound 17 (100 mg). MS(ESI) m/z=944[M+H]⁺

¹HNMR (400M, DMSO) δ 12.85 (s, br, 2H) 8.05 (s, 1H) 7.95-8.00 (m, 2H) 7.75 (s, 1H), 7.62 (s, 1H), 7.48 (s, 1H), 7.40 (s, 1H) 7.28 (s, 1H), 5.69-5.75 (m, 2H), 5.20 (s, 2H), 4.85 (s, 2H), 4.51-4.54 (m, 2H), 3.91-4.02 (m, 4H), 3.55-3.70 (m, 2H), 3.20-3.35 (m, 2H), 3.05-3.19 (m, 4H), 2.85-3.04 (m, 4H), 2.70-2.83 (m, 2H), 2.55 (s, 3H), 2.10 (s, 3H), 1.88-1.90 (m, 2H), 1.64-1.68 (m, 2H), 1.28 (t, J=7.20 Hz, 5H), 1.17 (t, J=7.48 Hz, 3H).

### Example 18

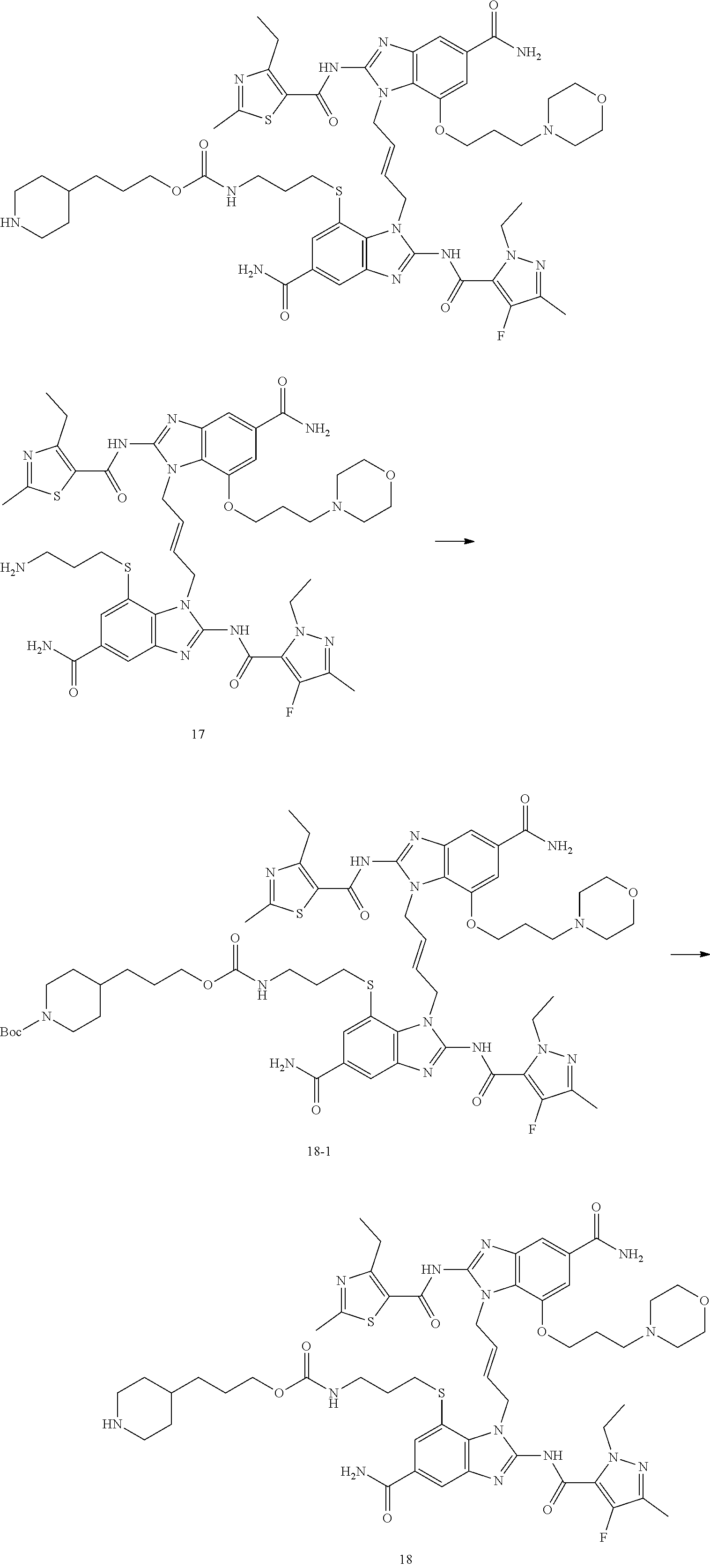

(Step 1) DIPEA (20 mg, 0.159 mmol) and the compound M11 (21 mg, 0.053 mmol) were added to a DMF (2 mL) solution of the compound 17 (50 mg, 0.053 mmol). The reaction mixture was reacted at room temperature for 1 h, dried by rotary evaporation, and dissolved with dichloromethane. The organic phase was washed with water, dehydrated and dried by rotary evaporation to afford the target compound 18-1 (40 mg), which was directly used in the next step of reaction. MS(ESI) m/z=1213[M+H]⁺

(Step 2) Trifluoroacetic acid (3 mL) was added to a dichloromethane (3 mL) solution of the compound 18-1 (40 mg, 0.033 mmol), and the reaction mixture was reacted at room temperature for 1 h and dried by rotary evaporation to give a crude product. The resulting crude product was purified by preparative HPLC to afford the target compound 18 (3.5 mg). MS(ESI) m/z=1113[M+H]⁺

¹HNMR (400M, DMSO) δ 13.09 (s, 1H) 12.80 (s, 1H) 8.50 (s, 1H) 8.20 (s, 1H) 8.05 (s, 1H) 7.90-8.00 (m, 2H) 7.75 (s, 1H), 7.65 (s, 1H), 7.48 (s, 1H), 7.35 (s, 1H) 7.30 (s, 1H), 7.02-7.10 (m, 1H) 5.70-5.85 (m, 2H), 5.20 (s, 2H), 4.85 (s, 2H), 4.40-4.50 (m, 2H), 3.85-4.05 (m, 5H), 3.10-3.30 (m, 6H), 2.85-3.05 (m, 4H), 2.75-2.85 (m, 2H), 2.08 (s, 3H), 1.88-2.00 (m, 2H), 1.65-1.80 (m, 2H), 1.35-1.60 (m, 5H), 1.15-1.25 (m, 10H).

### Example 19

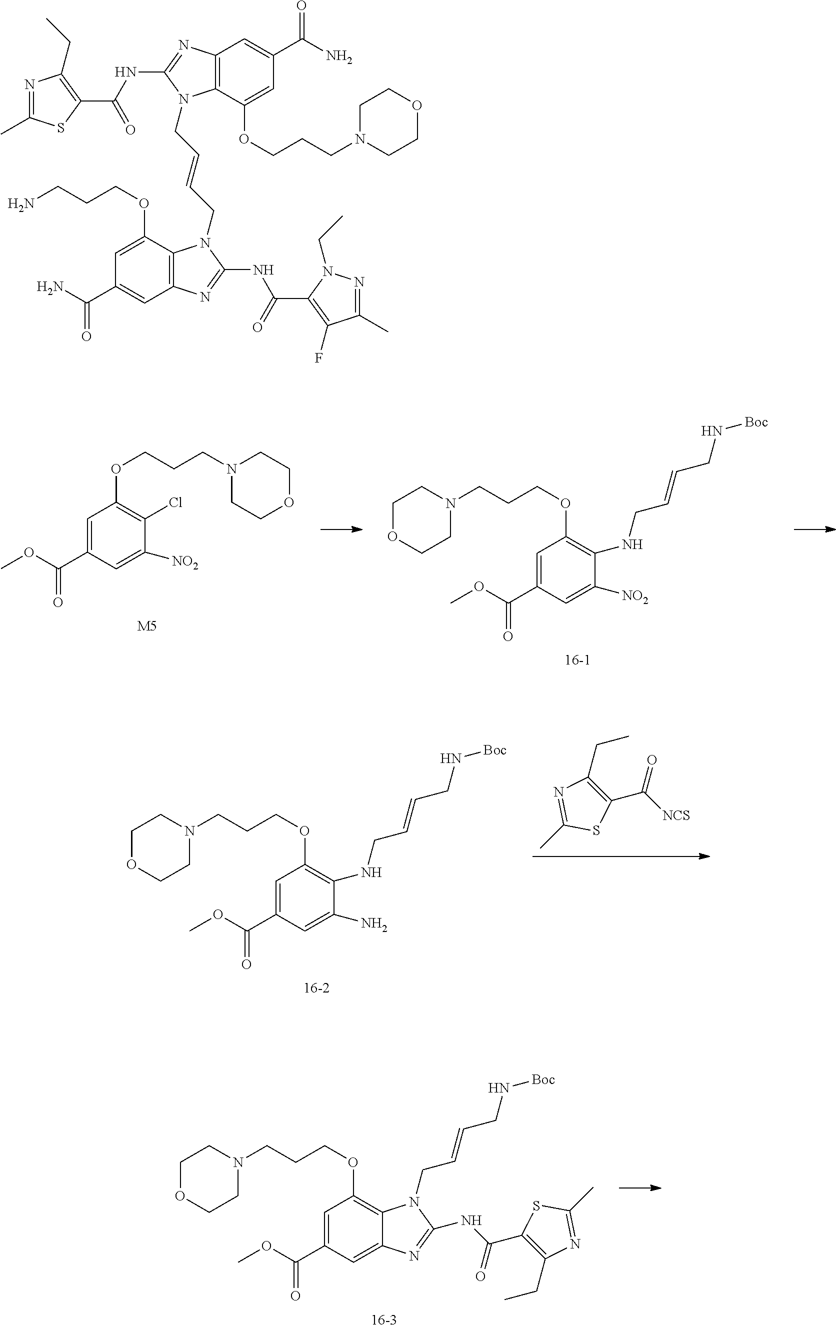

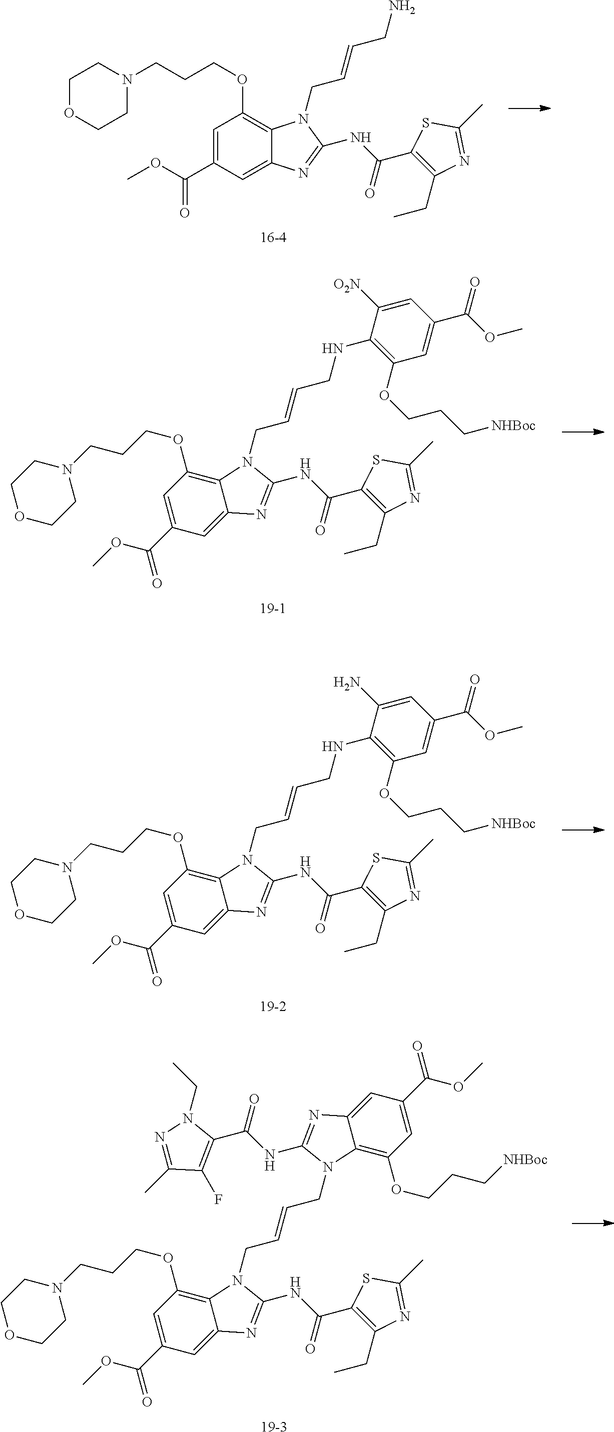

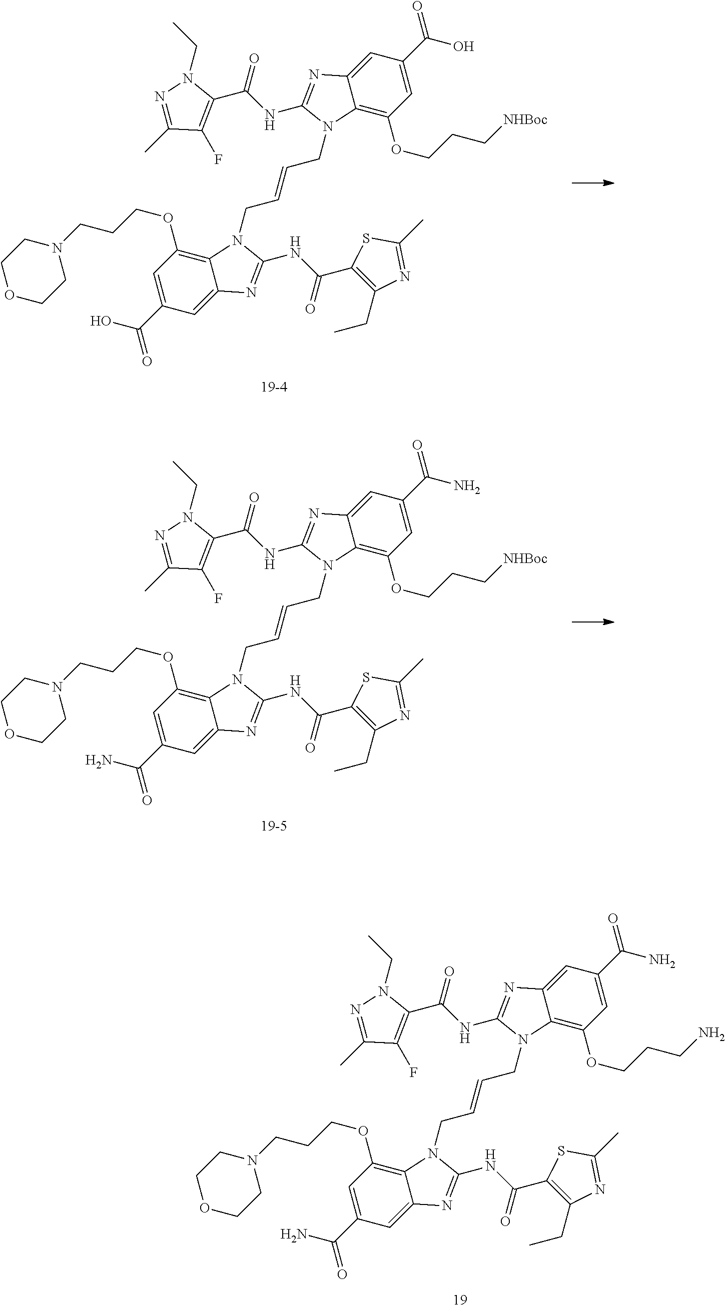 [SMILES: CCc1nc(C)sc1C(=O)Nc1nc2cc(C(=O)O)cc(OCCCN3CCOCC3)c2n1C/C=C/Cn1c(NC(=O)c2c(F)c(C)nn2CC)nc2cc(C(=O)O)cc(OCCCNC(=O)OC(C)(C)C)c21 ; "19-4" ; CCc1nc(C)sc1C(=O)Nc1nc2cc(C(N)=O)cc(OCCCN3CCOCC3)c2n1C/C=C/Cn1c(NC(=O)c2c(F)c(C)nn2CC)nc2cc(C(N)=O)cc(OCCCNC(=O)OC(C)(C)C)c21 ; "19-5" ; CCc1nc(C)sc1C(=O)Nc1nc2cc(C(N)=O)cc(OCCCN3CCOCC3)c2n1C/C=C/Cn1c(NC(=O)c2c(F)c(C)nn2CC)nc2cc(C(N)=O)cc(OCCCN)c21 ; "19"]

The synthesis method of Example 19 was basically the same as that of Example 16 except that the intermediate M8 used in Step 5 was replaced with the intermediate M6, and the intermediate M1 used in Step 7 was replaced with the intermediate M3, so as to afford the target compound 19 (15 mg). MS(ESI) m/z=928[M+H]⁺

¹HNMR (400M, DMSO) δ 12.88 (s, br, 2H) 8.05 (s, 1H) 7.95 (s, 1H) 7.75 (s, 1H), 7.70 (s, 2H), 7.66 (s, 1H), 7.64 (s, 1H), 7.45 (s, 1H), 7.40 (s, 1H) 7.31 (s, 1H) 7.20 (s, 1H) 6.55 (s, 1H), 5.60-5.50 (m, 2H), 4.80-4.95 (m, 4H), 4.55-4.65 (m, 2H), 3.85-4.02 (m, 4H), 3.05-3.25 (m, 6H), 2.90-3.00 (m, 2H), 2.80-2.88 (m, 2H), 2.17 (s, 3H), 1.66-1.80 (m, 4H), 1.31 (t, J=7.20 Hz, 5H), 1.16 (t, J=7.48 Hz, 3H).

### Example 20

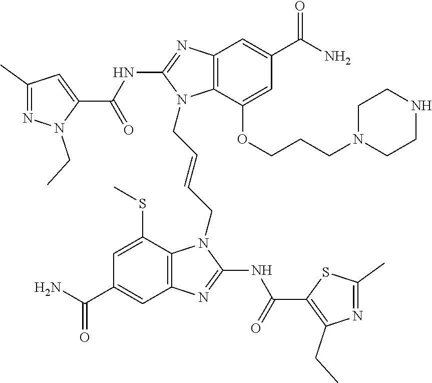 [SMILES: CCc1nc(C)sc1C(=O)Nc1nc2cc(C(N)=O)cc(SC)c2n1C/C=C/Cn1c(NC(=O)c2cc(C)nn2CC)nc2cc(C(N)=O)cc(OCCCN3CCNCC3)c21]

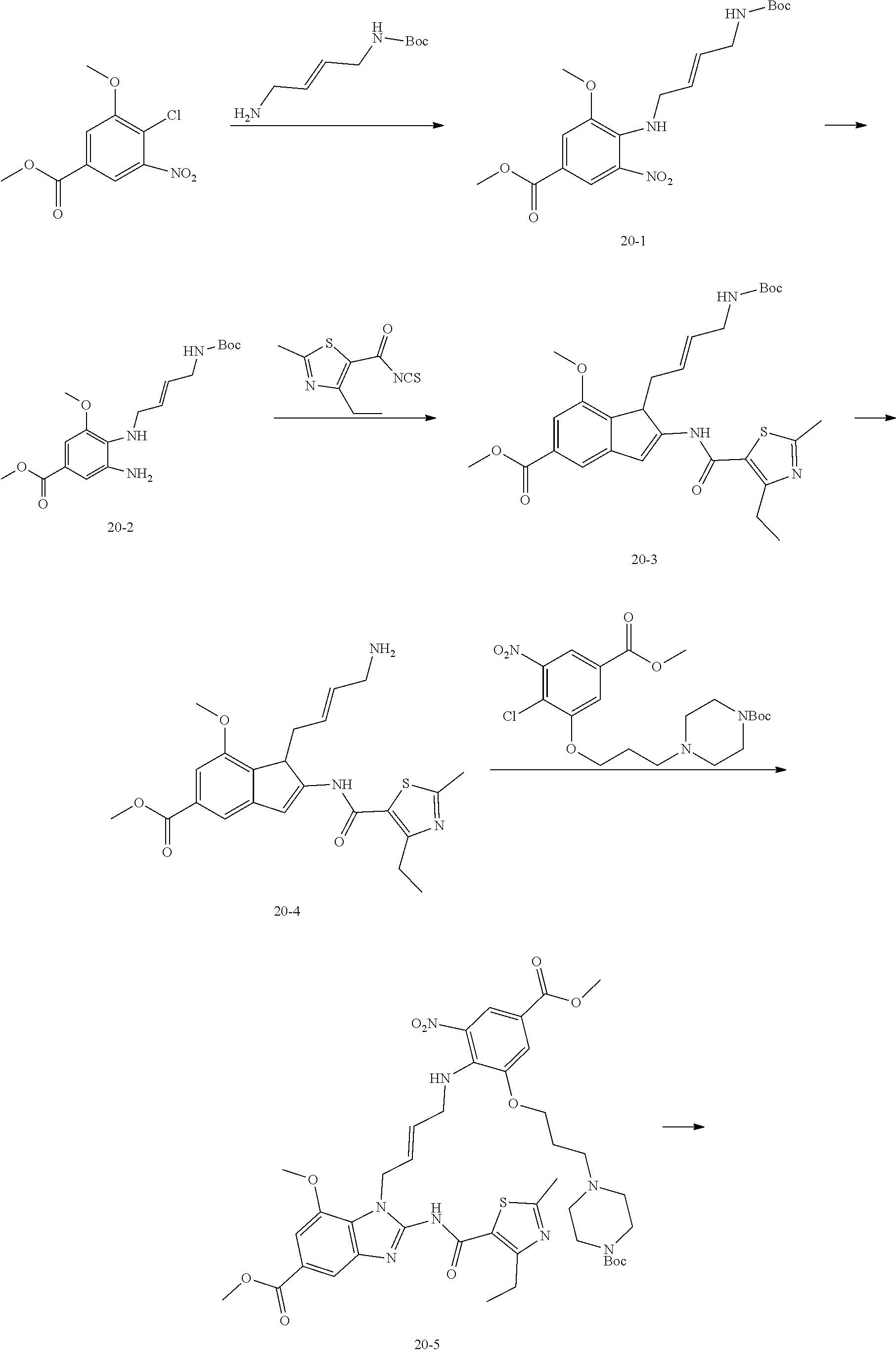

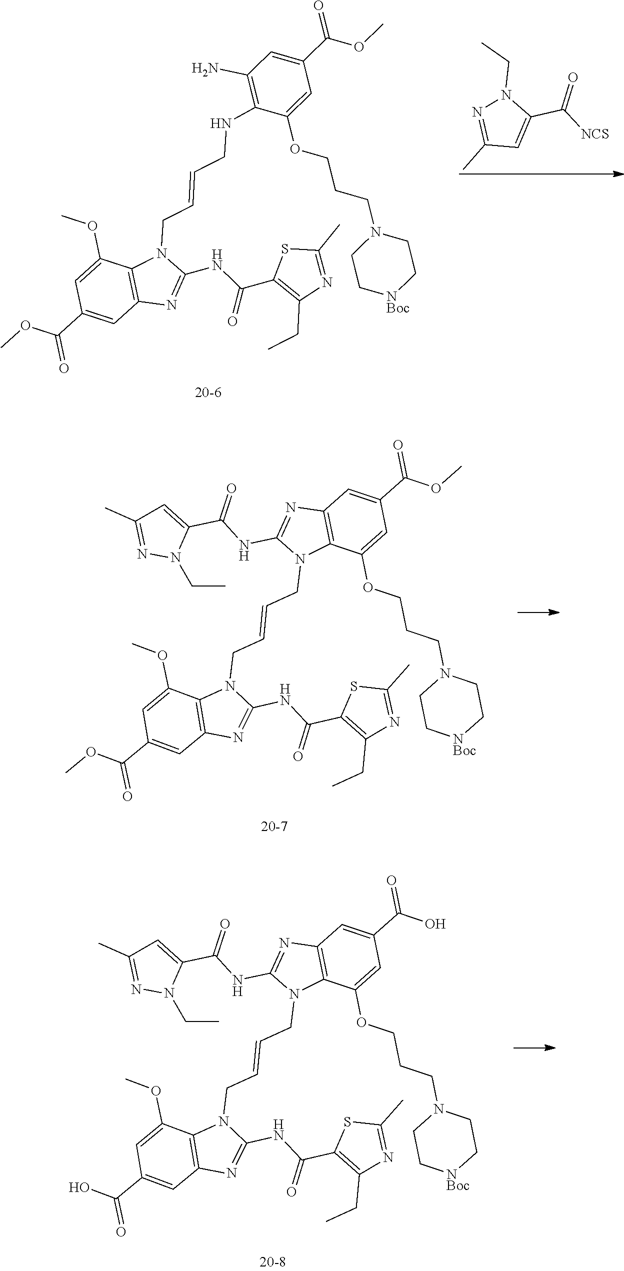

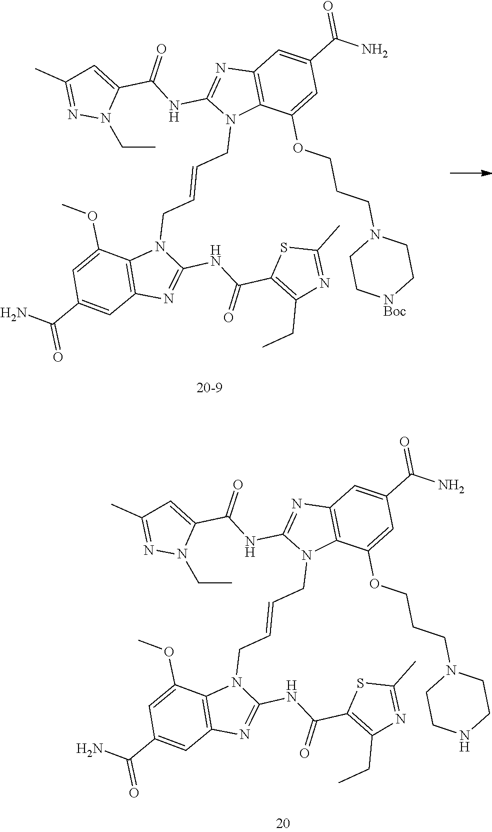 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OC(C)(C)C)CC3)c21 ; CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCNCC3)c21]

(Step 1) Methyl 4-chloro-3-methoxy-5-nitrobenzoate (30 g, 122 mmol) was dispersed in n-butanol (500 mL), to which tert-butyl (E)-(4-aminobut-2-en-1-yl) carbamate (22.8 g, 122 mmol) and DIPEA (78.9 g, 609 mmol) were added. The reaction solution was heated to 120° C., stirred for 18 h, distilled under reduced pressure, dissolved in ethyl acetate, and added with an appropriate amount of dilute hydrochloric acid (0.5 M) in an ice bath for adjusting the pH to neutrality. The separated organic phase was washed respectively with water and saturated NaCl solution, dehydrated with anhydrous magnesium sulfate and dried by rotary evaporation to afford the compound 20-1 (48.3 g, 99% yield). MS(ESI) m/z=396[M+H]

(Step 2) Ammonia water (120 mL) was added to a methanol (400 mL) solution of the compound 20-1 (48.3 g, 122 mmol) in an ice bath. After 10 minutes, an aqueous solution of sodium dithionite (106.3 g, 611 mmol) was added, and the reaction solution was warmed up slowly to room temperature and reacted for 2 h. Inorganic salts were removed by filtration, and ethyl acetate was added for extraction. The organic phase was washed with saturated NaCl solution, dehydrated with anhydrous magnesium sulfate and dried by rotary evaporation to give a crude product. The obtained crude product was purified by silica gel column chromatography (eluent: petroleum ether/ethyl acetate=1/1) to afford the compound 20-2 (28.6 g, 64% yield). MS(ESI) m/z=366[M+H]

(Step 3) The compound M2 (8.7 g, 41.1 mmol) was added to a DMF (200 mL) solution of the compound 20-2 (15 g, 41.1 mmol) in an ice bath. After reacted for 0.5 h, DIPEA (15.9 g, 123.3 mmol) and HATU (18.8 g, 49.3 mmol) were added, and then the reaction solution was warmed up to room temperature and reacted for 12 h. The reaction solution was slowly poured into water, and yellow solids were precipitated, which were then collected by filtration. The resulting solids were dried to afford the compound 20-3 (14.4 g, 64% yield). MS(ESI) m/z=544[M+H]

(Step 4) TFA (6 mL) was added to a DCM (200 mL) solution of the compound 20-3 (6 g, 11 mmol) in an ice bath, and then the reaction solution was warmed up to room temperature and reacted for 2 h. The solvent was removed by rotary evaporation under reduced pressure. After ethyl acetate was added, the free TFA was removed by rotary evaporation under reduced pressure to afford the compound 20-4 (4.9 g, 99% yield). MS(ESI) m/z=444[M+H]

(Step 5) The compound 20-4 (1 g, 2.2 mmol) was dispersed in n-butanol (20 mL), to which the intermediate M5 (1.05 g, 2.2 mmol) and DIPEA (1.42 g, 11 mmol) were added. The reaction solution was heated to 120° C., stirred for 18 h, distilled under reduced pressure, dissolved in ethyl acetate, washed respectively with water and saturated NaCl solution, dehydrated with anhydrous magnesium sulfate, dried by rotary evaporation and purified by silica gel column chromatography (eluent: petroleum ether/ethyl acetate=1/2) to afford the compound 20-5 (1.71 g, 90% yield). MS(ESI) m/z=865[M+H]

(Step 6) Ammonia water (120 mL) was added to a methanol (400 mL) solution of the compound 20-5 (1 g, 1.16 mmol) in an ice bath. After 10 minutes, an aqueous solution of sodium dithionite (1.01 g, 5.8 mmol) was added, and the reaction solution was warmed up slowly to room temperature and reacted for 2 h. Inorganic salts were removed by filtration, and ethyl acetate was added for extraction. The organic phase was washed with saturated NaCl solution, dehydrated with anhydrous magnesium sulfate and dried by rotary evaporation to afford the compound 20-6 (0.6 g, 62% yield). MS(ESI) m/z=835[M+H]

(Step 7) The compound M1 (0.14 g, 0.72 mmol) was added to a DMF (10 mL) solution of the compound 20-6 (0.6 g, 0.72 mmol) in an ice bath. After reacted for 0.5 h, DIPEA (0.28 g, 2.16 mmol) and HATU (0.32 g, 0.86 mmol) were added, and then the reaction solution was warmed up to room temperature and reacted for 12 h. The reaction solution was slowly poured into water, and yellow solids were precipitated, which were then collected by filtration. The resulting solids were dried to afford the compound 20-7 (0.56 g, 80% yield). MS(ESI) m/z=996[M+H]

(Step 8) The compound 20-7 (0.56 g, 0.57 mmol) was dissolved in a mixed solution (10 mL) of methanol, tetrahydrofuran and water (volume ratio: 1/1/1), to which hydrated lithium hydroxide (0.24 g, 5.7 mmol) was added. The reaction solution was heated to 75° C. and stirred overnight. The organic solvent was removed by rotary evaporation, and then the resulting residue was cooled to room temperature, and adjusted with dilute hydrochloric acid (1 M) in an ice bath such that the solids no longer precipitated. The resulting solids were collected by filtration and dried to afford the compound 20-8 (0.49 g, 90% yield). MS(ESI) m/z=968[M+H]

(Step 9) The compound 20-8 (0.49 g, 0.51 mmol) was dissolved in DMF (5 mL), to which HATU (0.46 g, 1.22 mmol) and DIPEA (0.33 g, 2.55 mmol) were added. After 0.5 h, ammonium bicarbonate (0.2 g, 2.55 mmol) was added, and the reaction mixture was stirred at room temperature for 2 h, concentrated, and dissolved with dichloromethane. The organic phase was washed with water, dehydrated and dried by rotary evaporation to afford the target compound 20-9 (0.33 g), which was directly used in the next step of reaction. MS(ESI) m/z=966[M+H]

(Step 10) Trifluoroacetic acid (5 mL) was added to a dichloromethane (5 mL) solution of the compound 20-9 (0.33 mg, 0.34 mmol), and the reaction mixture was reacted at room temperature for 1 h and dried by rotary evaporation to give a crude product. The obtained crude product was purified by preparative HPLC to afford the target compound 20 (150 mg). MS(ESI) m/z=866[M+H]

¹H NMR (400 MHz, DMSO) δ 12.85 (s, 1H), 12.79 (s, 1H), 8.53 (s, br, 1H), 7.99 (s, 1H), 7.94 (s, 1H), 7.63 (s, 2H), 7.32-7.38 (m, 3H), 7.26 (s, 1H), 6.54 (s, 1H), 5.94-5.70 (m, 2H), 4.92 (d, J=4.5 Hz, 2H), 4.85 (d, J=4.9 Hz, 3H), 4.55 (d, J=7.2 Hz, 3H), 3.96 (s, 2H), 3.77 (s, 3H), 2.95-3.20 (m, 8H), 2.09 (s, 3H), 1.63-1.75 (s, 2H), 1.29 (t, J=7.1 Hz, 3H), 1.15 (t, J=7.5 Hz, 3H).

### Example 21

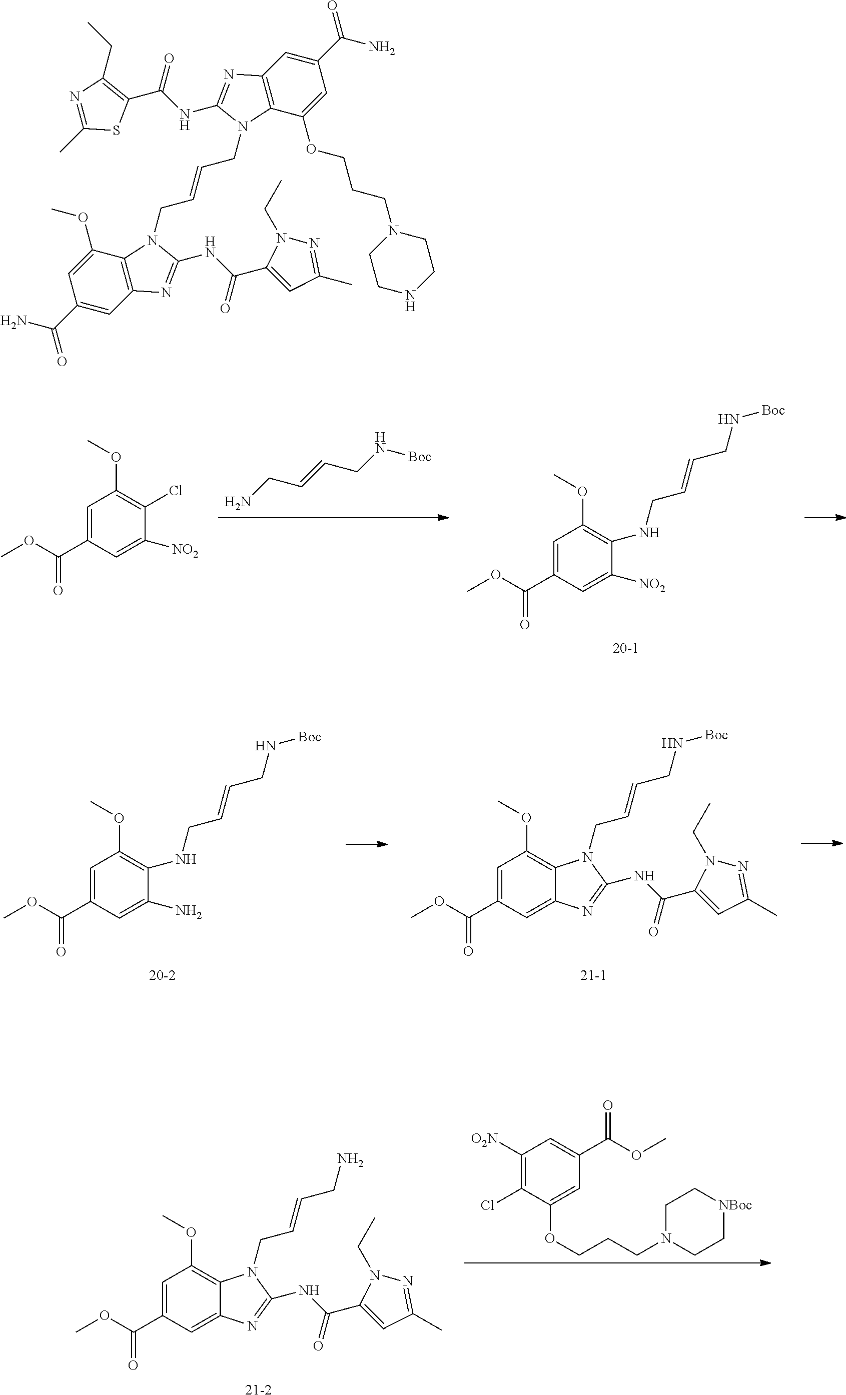

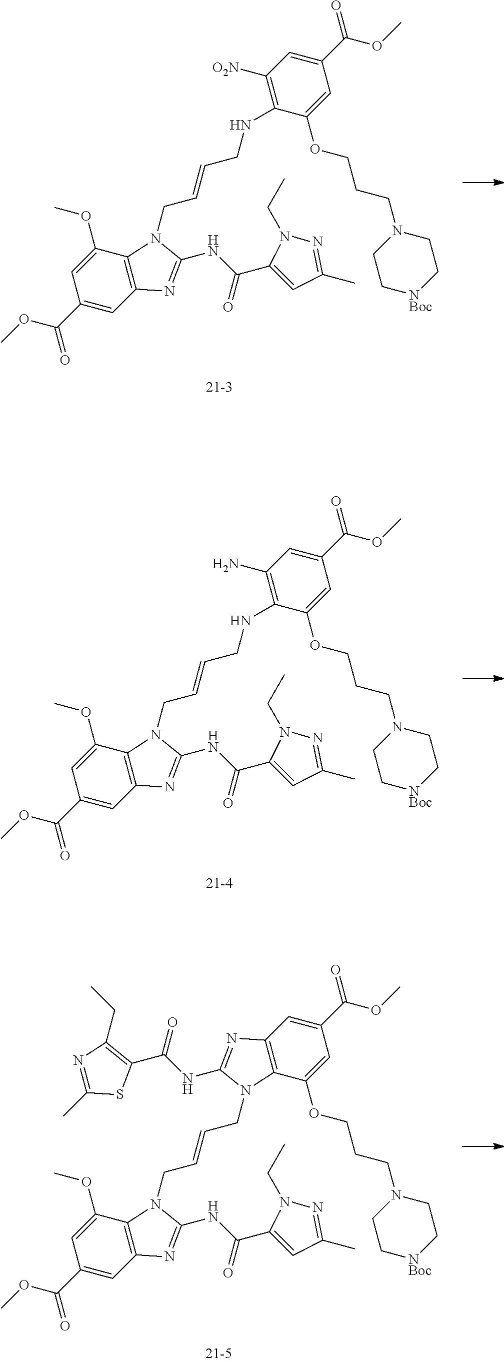 [SMILES: CCn1nc(C)cc1C(=O)Nc1nc2cc(C(=O)OC)cc(OC)c2n1C/C=C/CNc1c(OCCCN2CCN(C(=O)OC(C)(C)C)CC2)cc(C(=O)OC)cc1[N+](=O)[O-] ; CCn1nc(C)cc1C(=O)Nc1nc2cc(C(=O)OC)cc(OC)c2n1C/C=C/CNc1c(N)cc(C(=O)OC)cc1OCCCN1CCN(C(=O)OC(C)(C)C)CC1 ; CCc1nc(C)sc1C(=O)Nc1nc2cc(C(=O)OC)cc(OCCCN3CCN(C(=O)OC(C)(C)C)CC3)c2n1C/C=C/Cn1c(NC(=O)c2cc(C)nn2CC)nc2cc(C(=O)OC)cc(OC)c21]

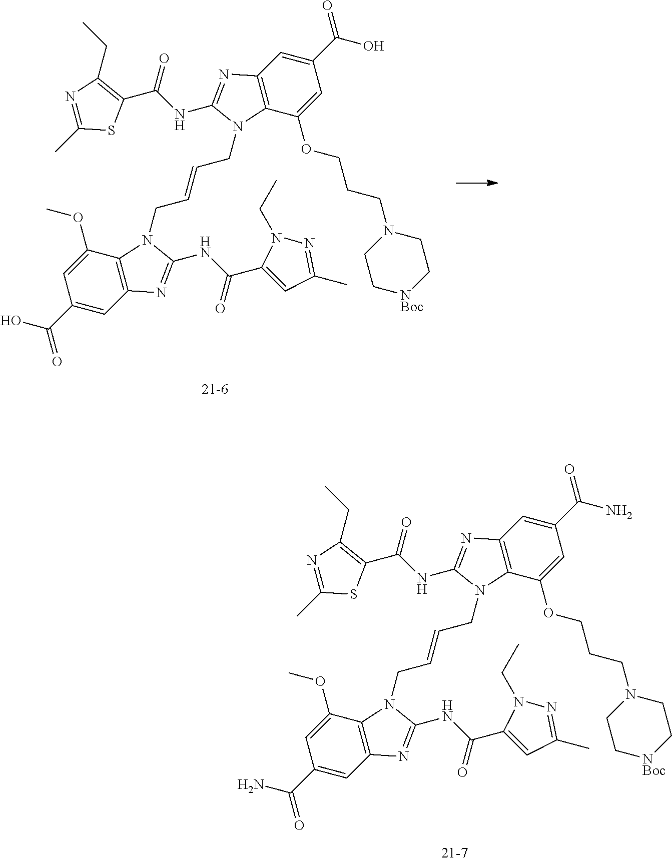 [SMILES: CCc1nc(C)sc1C(=O)Nc1nc2cc(C(=O)O)cc(OCCCN3CCN(C(=O)OC(C)(C)C)CC3)c2n1C/C=C/Cn1c(NC(=O)c2cc(C)nn2CC)nc2cc(C(=O)O)cc(OC)c21 ; CCc1nc(C)sc1C(=O)Nc1nc2cc(C(N)=O)cc(OCCCN3CCN(C(=O)OC(C)(C)C)CC3)c2n1C/C=C/Cn1c(NC(=O)c2cc(C)nn2CC)nc2cc(C(N)=O)cc(OC)c21]

The synthesis method of Example 21 was basically the same as that of Example 20 except that the intermediate M2 used in Step 3 was replaced with the intermediate M1, and the intermediate M1 used in Step 7 was replaced with the intermediate M2, so as to afford the target compound 21 (50 mg). MS(ESI) m/z=866[M+H]⁺

¹H NMR (400 MHz, DMSO) δ 12.82 (s, 1H), 9.57 (s, 2H), 7.99 (s, 1H), 7.64 (d, J=3.9 Hz, 2H), 7.35-7.40 (m, 2H), 7.33 (s, 1H), 6.52 (s, 1H), 5.75-5.85 (m, 2H), 4.88-4.95 (m, 4H), 4.51 (q, J=7.1 Hz, 2H), 4.10-4.15 (m, 2H), 3.72 (s, 3H), 3.23-3.30 (m, 6H), 3.05-3.13 (m, 2H), 2.54 (s, 3H), 2.08-2.15 (m, 5H), 1.27 (t, J=7.1 Hz, 3H), 1.15 (t, J=7.5 Hz, 3H).

### Example 22

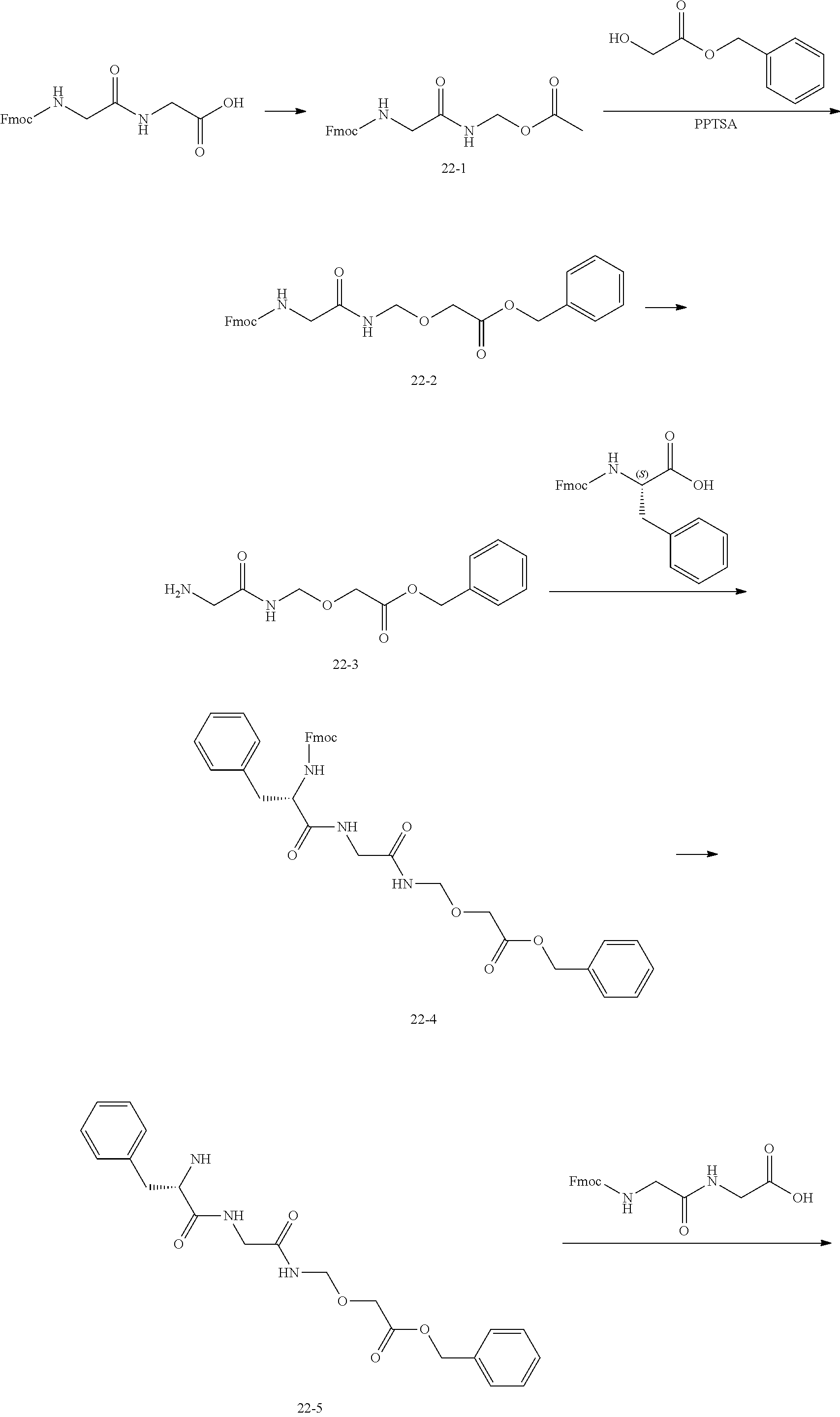

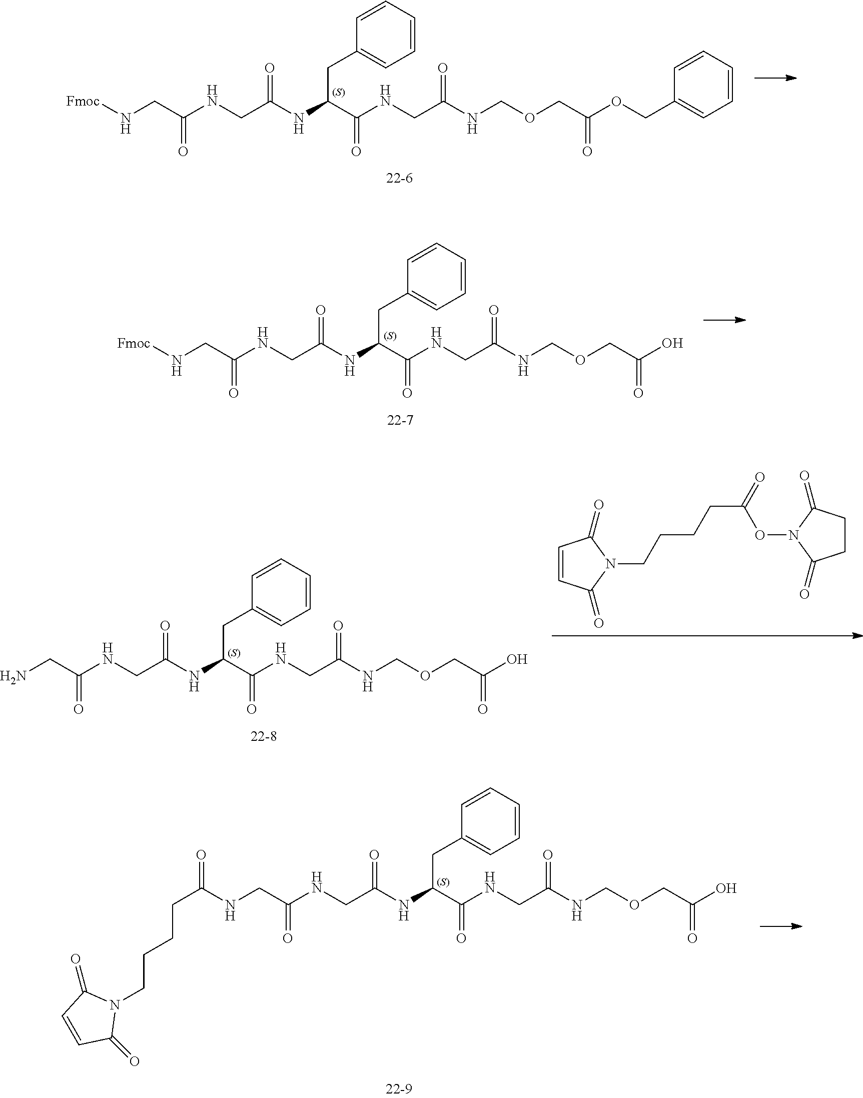

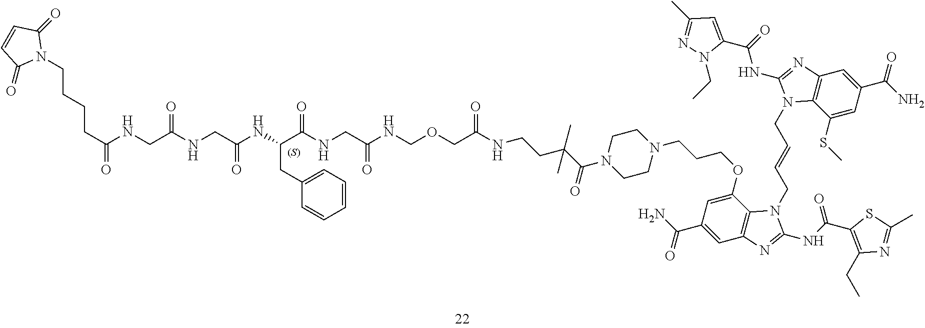

(Step 1) N-fluorenylmethoxycarbonyl-glycyl-glycine (15 g, 102.64 mmol) and Pb(OAc)₄ (91.02 g, 205.28 mmol) were added into a three-necked flask, in which the air was replaced with nitrogen three times. A mixed solution of tetrahydrofuran and toluene (500 mL, 1/1) was added. The reaction mixture was heated to 85° C. under the protection of nitrogen, reacted for 3 h, cooled to room temperature, and filtered. The obtained filtrate was dried by rotary evaporation and purified by normal-phase column chromatography (PE/EA (v/v)=10/1 to 1/1) to afford the compound 22-1 (15 g, 96% yield).

MS(ESI) m/z=369[M+H]⁺

(Step 2) Pyridine p-toluenesulfonate (5.22 g, 20.86 mmol) was added to a dichloromethane (40 mL) solution of the compound 22-1 (3.66 g, 9.94 mmol) and benzyl 2-hydroxyacetate (16.51 g, 99.4 mmol). The reaction mixture was heated to 45° C., reacted for 18 h, dried by rotary evaporation, purified by normal-phase column chromatography (PE/EA (v/v)=10/1 to 1/10), and then purified again by reverse-phase column chromatography to afford the compound 22-2 (3.5 g, 75% yield).

MS(ESI) m/z=475[M+H]⁺

(Step 3) Piperidine (87 mg, 1.02 mmol) was added to an acetonitrile (5 mL) solution of the compound 22-2 (243 mg, 0.51 mmol), and the reaction mixture was reacted at room temperature for 2 h, added with petroleum ether for extraction of impurities (3*3 mL), and dried by rotary evaporation to afford the compound 22-3, which was directly used in the next step of reaction.

MS(ESI) m/z=253[M+H]⁺

(Step 4) HATU (320 mg, 0.84 mmol), DIPEA (144 mg, 1.12 mmol) and the compound 22-3 (128 mg, 0.51 mmol) were added to a dichloromethane (5 mL) solution of N-Fmoc phenylalanine (217 mg, 0.56 mmol). The reaction mixture was reacted at room temperature for 2 h, diluted with dichloromethane, washed respectively with water and saturated NaCl solution, dried by rotary evaporation and purified by normal-phase column chromatography (PE/EA (v/v)=2/1) to afford the target compound 22-4 (221 mg, 70% yield).

MS(ESI) m/z=622[M+H]⁺

(Step 5) Piperidine (60 mg, 0.7 mmol) was added to an acetonitrile (5 mL) solution of the compound 22-4 (221 mg, 0.35 mmol), and the reaction mixture was reacted at room temperature for 2 h, added with petroleum ether for extraction of impurities (3*3 mL), and dried by rotary evaporation to afford the compound 22-5, which was directly used in the next step of reaction.

MS(ESI) m/z=400[M+H]⁺

(Step 6) HATU (194 mg, 0.51 mmol), DIPEA (88 mg, 0.68 mmol) and the compound 22-5 (136 mg, 0.34 mmol) were added to a DMF (5 mL) solution of N-Fremoxycarbonyl-glycyl-glycine (180 mg, 0.51 mmol). The reaction mixture was reacted in an ice bath for 2 h, dried by rotary evaporation and purified by reverse-phase column chromatography (ACN/H₂O: 5%-95%) to afford the compound 22-6 (176 mg, 70% yield).

MS(ESI) m/z=736[M+H]⁺

(Step 7) 10% palladium on carbon (Pd/C) (26 mg) was added to a methanol (10 mL) solution of the compound 22-6 (176 mg, 0.24 mmol), and the air in the reaction system was replaced with hydrogen three times and the hydrogen atmosphere was maintained overnight. After filtration, the obtained filtrate was dried by rotary evaporation to afford the compound 22-7 (148 mg, 95% yield).

MS(ESI) m/z=646[M+H]⁺

(Step 8) Piperidine (39 mg, 0.46 mmol) was added to an acetonitrile (5 mL) solution of the compound 22-7 (148 mg, 0.23 mmol), and the reaction mixture was reacted at room temperature for 2 h, added with petroleum ether for extraction of impurities (3*3 mL), and dried by rotary evaporation to afford the compound 22-8, which was directly used in the next step of reaction.

MS(ESI) m/z=424[M+H]⁺

(Step 9) DIPEA (50 mg, 0.39 mmol) and 5-maleimidovalericacid-NHS (83 mg, 0.28 mmol) were added into a DMF (3 mL) solution of the compound 22-8 (97 mg, 0.23 mmol) in an ice bath, and then the reaction mixture was warmed up to room temperature, reacted for 1 h and purified by reverse-phase MPLC to afford the compound 22-9 (84 mg, 62% yield).

MS(ESI) m/z=603[M+H]⁺

(Step 10) DMTMM (47 mg, 0.16 mmol) was added to a DMF (2 mL) solution of the compound 22-9 (84 mg, 0.14 mmol) in an ice bath. After 5 minutes, the compound 3 (139 mg, 0.14 mmol) and DIPEA (45 mg, 0.35 mmol) were added. The reaction mixture was reacted at room temperature for 1 h, and purified by preparative HPLC to afford the target compound 22 (60 mg, 27% yield).

MS(ESI) m/z=1579[M+H]⁺

### Examples 23-27

The target compounds of Examples 23, 24, 25, 26 and 27 were synthesized basically in accordance with the synthesis method of Example 22 except that the compound 3 used in Step 10 was replaced with the corresponding amine in the following table.

| Example | Amine | Target product |
|---|---|---|
| 23 | Intermediate 1-11 of compound 1 | 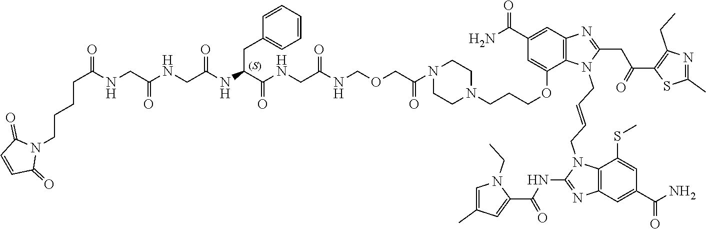 |
| | | MS (ESI) m/z = 1466.5 [M + H]⁺ |
| 24 | Compound 20 | 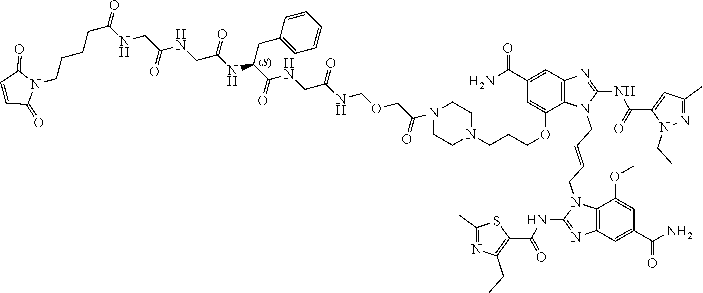 |
| | | MS (ESI) m/z = 1466.5 [M + H]+ |
| 25 | Compound 1 | 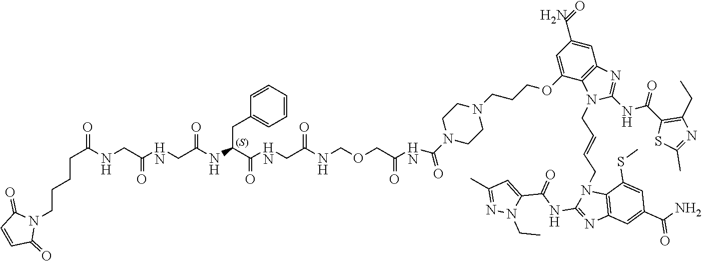 |
| | | MS (ESI) m/z = 1537.6 [M + H]⁺ |
| 26 | Compound 10 | 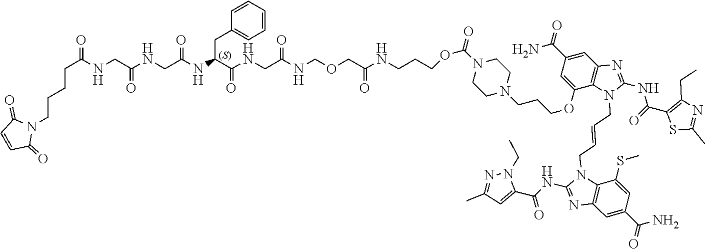 |
| | | MS (ESI) m/z = 1567.2 [M + H]⁺ |
| 27 | Compound 21 | 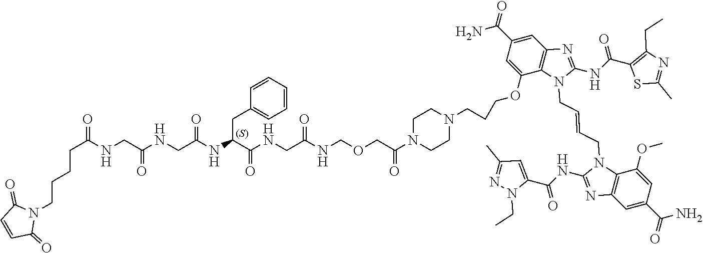 |
| | | MS (ESI) m/z = 1450.5 [M + H]⁺ |

### Example 28

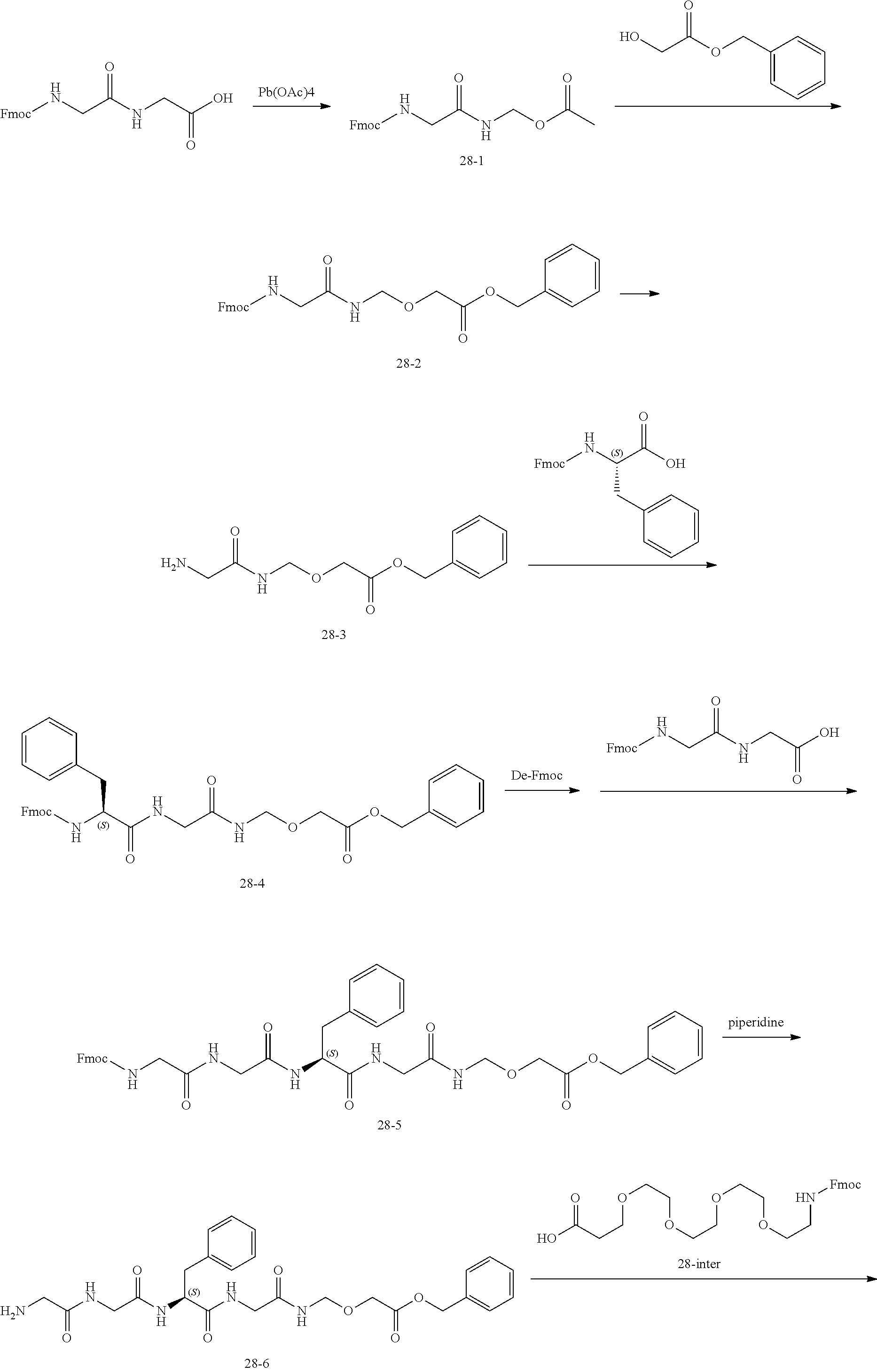

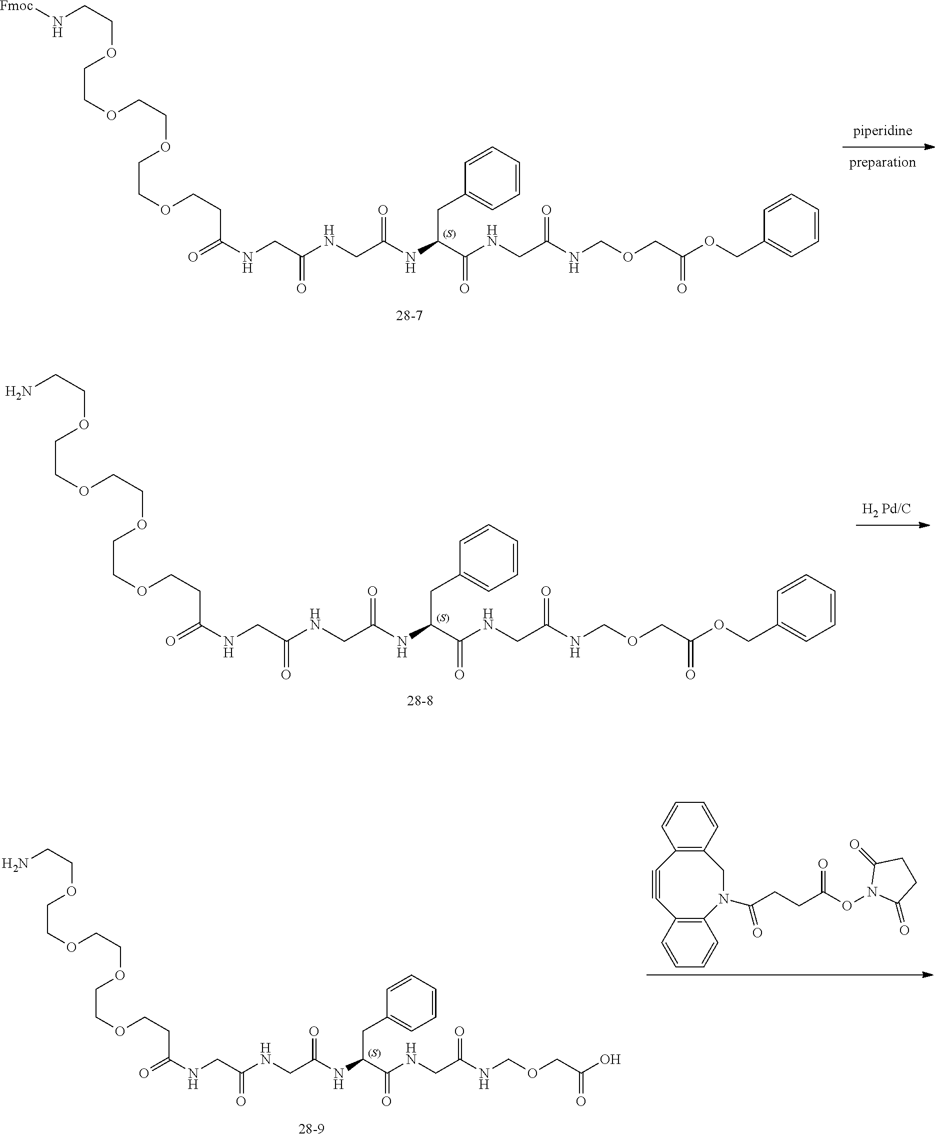

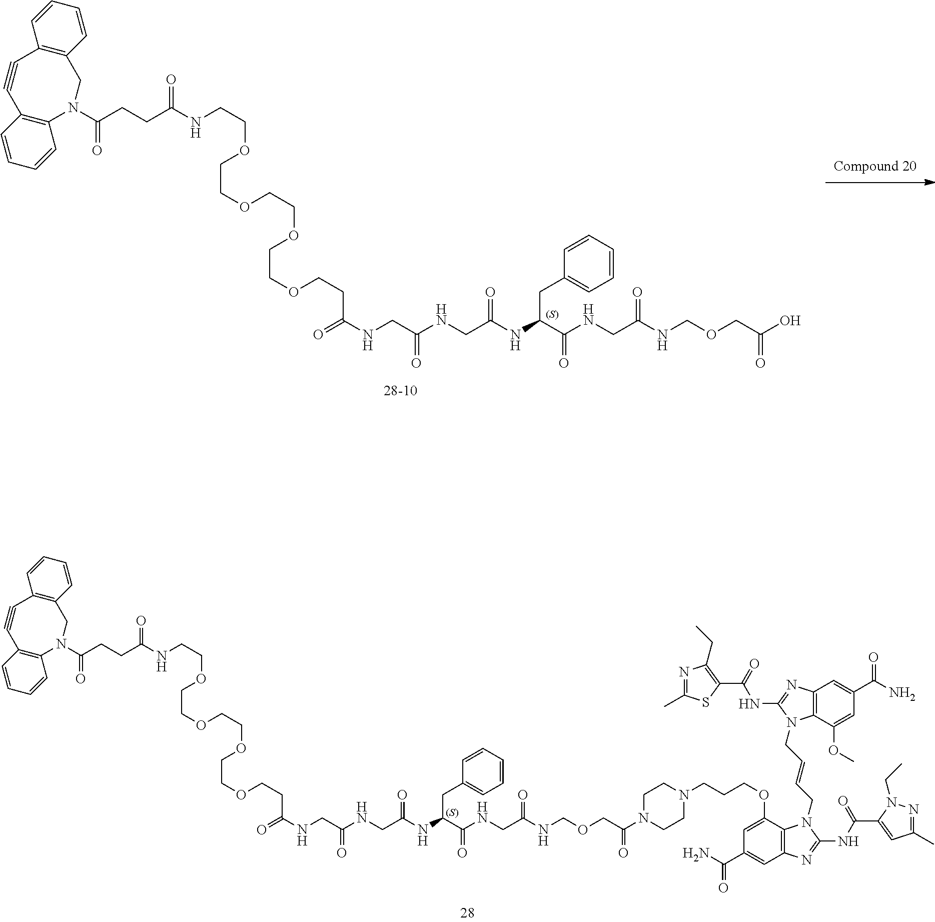

(Step 1) N-fluorenylmethoxycarbonyl-glycyl-glycine (15 g, 102.64 mmol, 1 eq.) and Pb(OAc)₄ (91.02 g, 205.28 mmol, 2 eq.) were added into a three-necked flask, to which THE and toluene were added under the protection of nitrogen. The reaction mixture was heated to 85° C., reacted under stirring for 3 h, cooled to room temperature and filtered. The filter cake was collected, and washed with 15 mL of DCM, and then the filtrate was concentrated to give a crude product. The resulting crude product was separated and purified by silica gel column chromatography (eluent: EA/PE=1:10 to 1:1, v/v) to afford the intermediate compound 28-1 (15 g, 40.7 mmol, 96% yield).

MS (ESI) m/z=369.2 [M+H]⁺

(Step 2) The intermediate 28-1 (3.68 g, 9.94 mmol, 1 eq.) and benzyl glycolate (16.51 g, 99.35 mmol, 10 eq.) were dissolved in DCM, to which piperazine (bis) p-toluenesulfonate (PPTSA) (5.22 g, 20.86 mmol, 2.1 eq.) was added. The reaction mixture was heated to 45° C., reacted under stirring overnight and dried by evaporation to give a crude product which was separated by silica gel column chromatography (EA:PE=10% to 100%, v/v) to remove benzyl glycolate and PPTSA. The obtained crude product contained the intermediate 28-1 and the product 28-2, which were further separated and purified by C18 column chromatography (formic acid-water:acetonitrile=95:5 to 5:95, v/v) to afford the intermediate compound 28-2 (3.56 g, 7.49 mmol, 75% yield).

MS (ESI) m/z=475.2 [M+H]⁺

(Step 3) Hexahydropyridine (86.84 mg, 1.02 mmol, 2 eq.) was added to a CH₃CN solution of the compound 28-2 (243 mg, 509.95 μmol, 1 eq.), and the reaction mixture was reacted under stirring at 25° C. for 2 h, subjected to extraction with PE three times to remove by-products and hexahydropyridine, and concentrated to afford the intermediate 28-3, which was directly used in the next step of reaction without further purification.

MS (ESI) m/z=253.2 [M+H]⁺

(Step 4) HATU (266.62 mg, 701.64 μmol, 1.5 eq.), DIPEA (120.91 mg, 935.52 mol, 162.95 μL, 2 eq.) and the intermediate 28-3 (118 mg, 467.7 μmol, 1 eq.) were added to a DCM solution of Fmoc-L-phenylalanine (199.35 mg, 514.54 μmol, 1.1 eq.), and the reaction mixture was reacted under stirring at room temperature for 2 h, concentrated, separated and purified by silica gel column chromatography to afford the intermediate 28-4 (248 mg, 0.4 mmol, two-step yield: 78%).

MS (ESI) m/z=622.2 [M+H]⁺

(Step 5) Hexahydropiperidine (63.01 mg, 739.93 μmol, 2 eq.) was added to a CH₃CN solution of the intermediate 28-4 (230 mg, 369.97 μmol, 1 eq.), and the reaction mixture was reacted under stirring at room temperature for 2 h, subjected to extraction with PE three times to remove by-products and hexahydropiperidine and concentrated to give a crude product, which was directly used in the next step of reaction without further purification.

HATU (195.50 mg, 514.47 μmol, 1.5 eq.) and DIPEA (88.65 mg, 685.96 μmol, 119.48 μL, 2 eq.) were added to a DMF solution of N-fluorenylmethoxycarbonyl-glycyl-glycine (182.31 mg, 514.47 μmol, 1.5 eq.) at 0° C. The reaction mixture was stirred for 5 min, added with the crude product (137 mg, 342.98 μmol, 1 eq.) was added, and then reacted at 0° C. under stirring for 2 h. After the reaction was completed, part of the solvent was removed by rotary evaporation, and the residue was separated and purified by mHPLC (H₂O/CH₃CN=95:5 to 5:95, v/v) to afford the intermediate 28-5 (190 mg, 0.26 mmol, 70% yield).

MS (ESI) m/z=736.2 [M+H]⁺

(Step 6) Hexahydropyridine (243.53 mg, 2.86 mmol, 293.5 μL, 2 eq.) was added to a MeOH (10 mL) solution of the intermediate 28-5 (1.05 g, 1.43 mmol, 1 eq.), and the reaction mixture was stirred at room temperature for 2 h, and then directly used in the next step of reaction without further purification.

(Step 7) HATU (994.51 mg, 2.62 mmol, 1.5 eq.) and DIPEA (450.99 mg, 3.49 mmol, 607.81 μL, 2 eq.) were added to a DMF solution of 28-inter (1.02 g, 2.09 mmol, 1.2 eq.) at 0° C., and the reaction mixture was reacted under stirring for 5 min, added with the crude product (896 mg, 1.74 mmol, 1 eq.) from the step 6, stirred at 0° C. for 2 h, dried by evaporation, and separated by mHPLC to afford the intermediate 28-7 (613 mg, 0.62 mmol, two-step yield: 45%).

MS (ESI) m/z=984.2 [M+H]⁺

(Step 8) Hexahydropyridine (190.55 mg, 2.24 mmol, 230 μL, 2 eq.) was added to a DMF (5 mL) solution of the intermediate 28-7 (1.1 g, 1.12 mmol, 1 eq.), and the reaction mixture was reacted under stirring at room temperature for 2 h and concentrated to give a crude product 28-8, which was directly used in the next step of reaction without further purification.

(Step 9) The crude product 28-8 (1.1 g, 1.45 mmol) and Pd/C (200 mg, 1.65 mmol) were added to MeOH (15 mL) to produce a suspension, which was vacuumized and then stirred at room temperature for 2 h under the protection of hydrogen. After the reaction was completed, the reaction solution was filtered, and the obtained filtrate was concentrated to give a crude product, which was separated and purified by mHPLC to afford the intermediate 28-9 (225 mg, 0.33 mmol, two-step yield: 30%).

MS (ESI) m/z=671.2 [M+H]⁺

(Step 10) Dibenzocyclooctyne-N-hydroxysuccinimidyl ester (158.39 mg, 393.61 μmol, 1.2 eq.) and DIPEA (105.98 mg, 820.03 μmol, 142.83 μL, 2.5 eq.) were added to a DMF (2 mL) solution of the intermediate 28-9 (220 mg, 328.01 μmol, 1 eq.) at 0° C., and the reaction mixture was reacted under stirring at 0° C. for 1 h. After the reaction was completed, the reaction solution was separated and purified by mHPLC to afford the intermediate 28-10 (188 mg, 0.19 mmol, 60% yield of).

MS (ESI) m/z=958.2 [M+H]⁺

(Step 11) The intermediate 28-10 (21.9 mg, 22.86 μmol, 230 μL, 1.1 eq.) and DMT-MM (8.05 mg, 29.09 μmol, 290 μL, 1.4 eq.) were mixed at 0° C., and the mixture was stirred for 5 minutes at 0° C. Compound 20 (18.00 mg, 20.78 μmol, 210 μL, 1 eq.) and DIPEA (6.71 mg, 51.95 μmol, 9.05 μL, 2.5 eq.) were then added, and the reaction mixture solution was kept stirring and reacted at 0° C. for 0.5 h. After the reaction was completed, the obtained crude product was separated and purified by Pre-HPLC to afford the compound 25 (21 mg, 0.012 mmol, 53% yield).

MS (ESI) m/z=1805.5 [M+H]⁺

### Examples 29-33

In accordance with the synthesis method of Example 28, the compound 20 used in Step 11 was replaced with the corresponding amine in the following table, and the rest of reagents and operations remained unchanged to afford the corresponding target compounds of Examples 29, 30, 31, 32 and 33 in the table.

| Example | Amine | Structure |
|---|---|---|
| 29 | intermediate 1-11 | 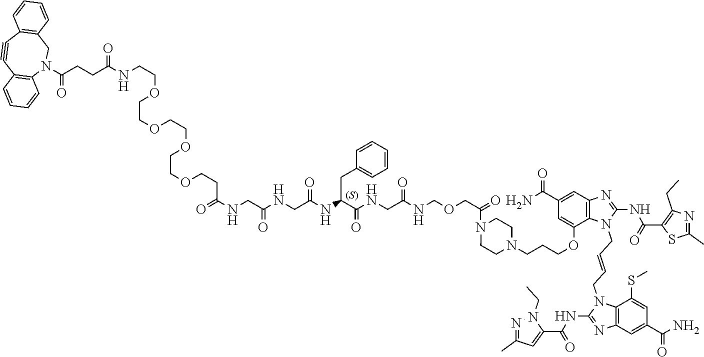 |
| | | MS (ESI) m/z = 1819.8 [M − H]⁺ |
| 30 | Compound 3 | 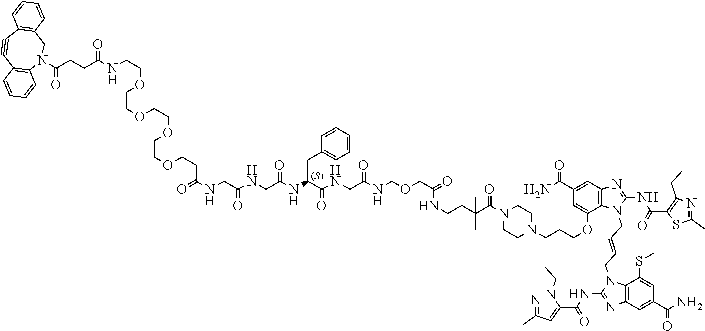 |
| | | MS (ESI) m/z = 1934.5 [M + H]⁺ |
| 31 | Compound 1 | 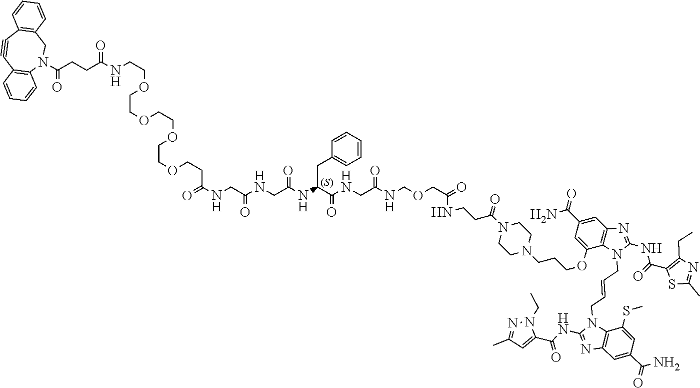 |
| | | MS (ESI) m/z = 1892.5 [M + H]⁺ |
| 32 | Compound 10 | 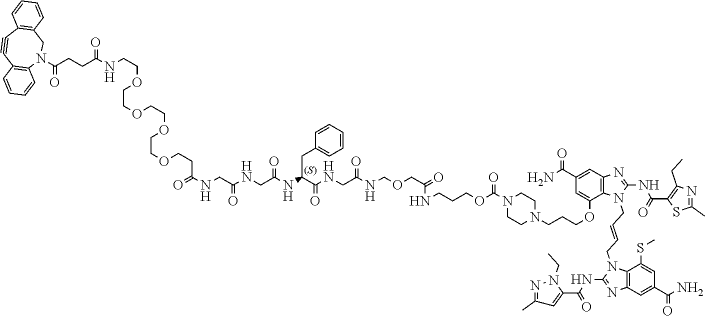 |
| | | MS (ESI) m/z = 1922.6 [M + H]⁺ |
| 33 | Compound 21 | 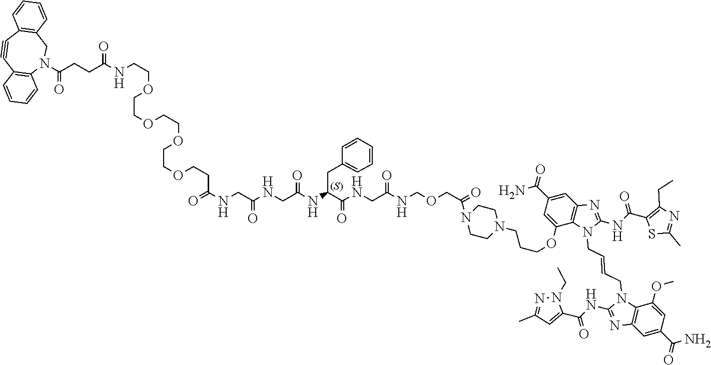 |
| | | MS (ESI) m/z = 1085.6 [M + H]⁺ |

### Example 34

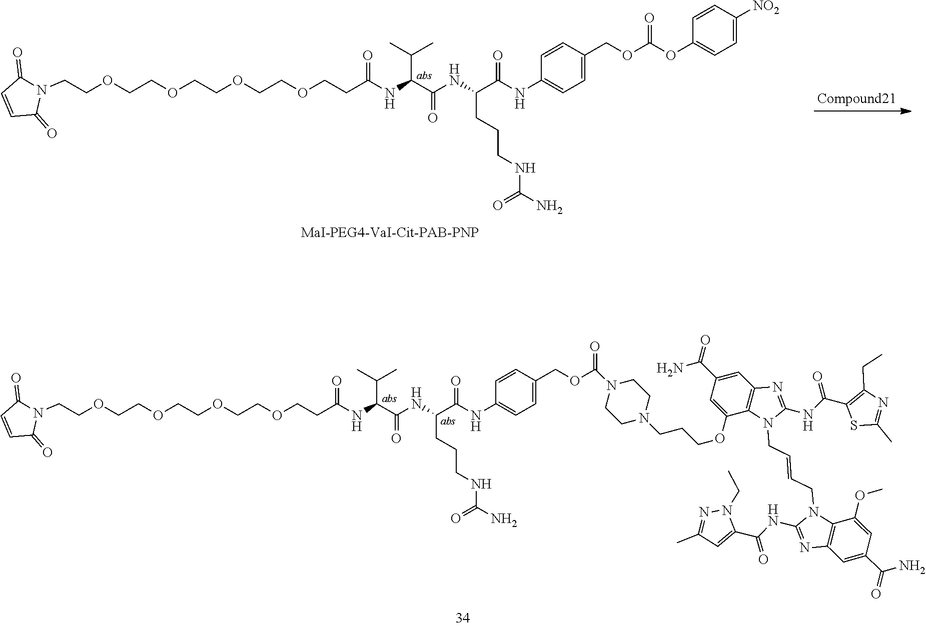 [SMILES: CC(C)[C@H](NC(=O)CCOCCOCCOCCOCCN1C(=O)C=CC1=O)C(=O)N[C@@H](CCCNC(N)=O)C(=O)Nc1ccc(COC(=O)Oc2ccc([N+](=O)[O-])cc2)cc1 ; CCc1nc(C)sc1C(=O)Nc1nc2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(OC)c21]

DIPEA (2.46 mg, 19.05 μmol) and the compound 21 (7.43 mg) were added to a DMF (300 uL) solution of Mal-PEG₄-Val-Cit-PAB-PNP (7.5 mg, 8.6 μmol) at room temperature, and the mixture solution was stirred and reacted at room temperature for 2 h. After the reaction was completed, the reaction solution was separated and purified by Pre-HPLC (C18 column, mobile phase: CH₃CN/H₂O=3/7 to 95/5, v/v, the product was collected at the CH₃CN/H₂O ratio of 55/45) to afford a white solid as the compound 34 (3.02 mg, 22% yield).

MS (ESI) m/z=1599.5 [M+H]⁺

### Examples 35-39

In accordance with the synthesis method of Example 34, the compound 21 used in the step was replaced with the corresponding amine in the following table, and the rest of reagents and operations remained unchanged to afford the corresponding target compounds of Examples 35, 36, 37, 38 and 39 in the table.

| Example | Amine | Compound |
|---|---|---|
| 35 | Compound 1 | 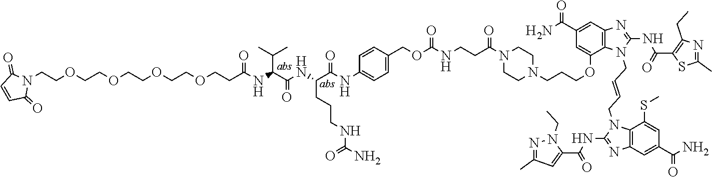 [SMILES: CCc1nc(C)sc1C(=O)Nc1nc2cc(C(N)=O)cc(OCCCN3CCN(C(=O)CCNC(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(SC)c21] |
| | | MS (ESI) m/z = 843.8 [(M + 2H)/2]⁺. |
| 36 | Compound 20 | 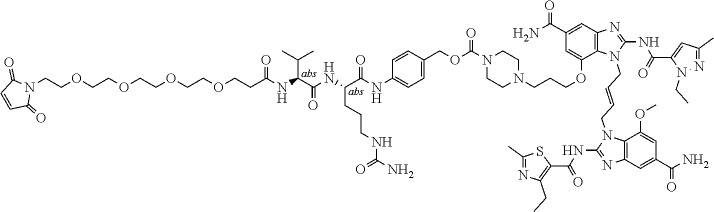 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c21] |
| | | MS (ESI) m/z = 1598.7 [M + H]⁺. |
| 37 | intermediate 1-11 | 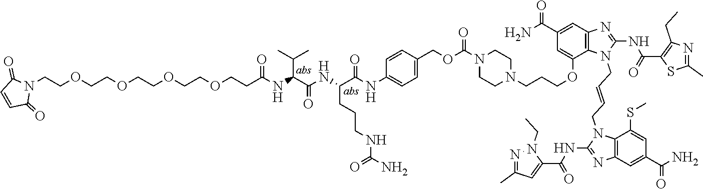 [SMILES: CCc1nc(C)sc1C(=O)Nc1nc2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(SC)c21] |
| | | MS (ESI) m/z = 1614.7 [M + H]⁺ |
| 38 | Compound 3 | 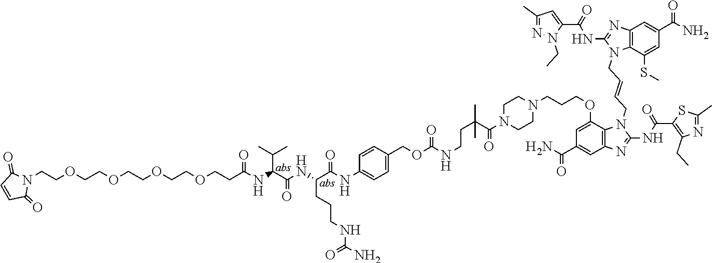 [SMILES: CCc1nc(C)sc1C(=O)Nc1nc2cc(C(N)=O)cc(OCCCN3CCN(C(=O)C(C)(C)CCNC(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(SC)c21] |
| | | MS (ESI) m/z = 1727.7 [M + H]⁺ |
| 39 | Compound 10 | 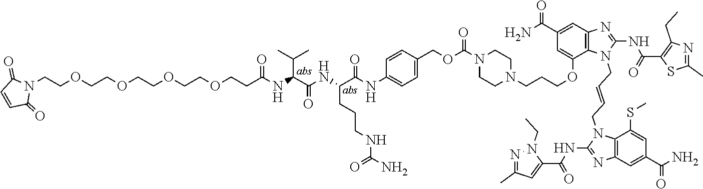 [SMILES: CCc1nc(C)sc1C(=O)Nc1nc2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(SC)c21] |
| | | MS (ESI) m/z = 1715.7 [M + H]⁺ |

### Example 40

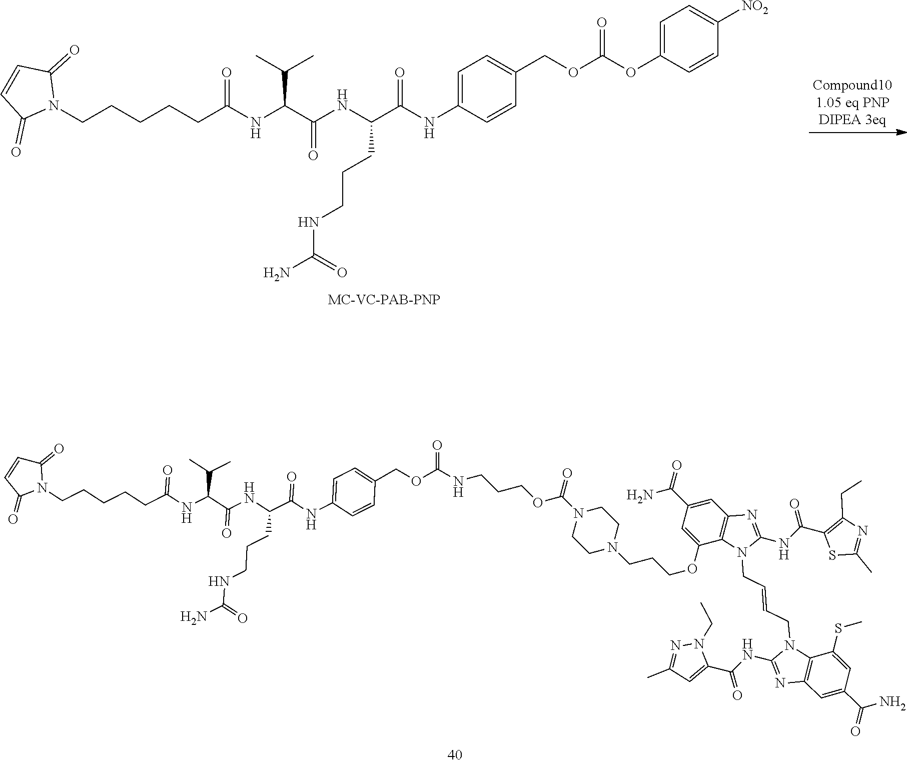 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CCc1nc(C)sc1C(=O)Nc1nc2cc(C(N)=O)cc(OCCCN3CCN(C(=O)OCCCNC(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21]

p-nitrophenol (PNP) (2 mg, 0.009 mmol) and DIPEA (3.5 mg, 0.027 mmol) were added to a DMF solution of MC-VC-PAB-PNP (7.4 mg, 0.01 mmol) and the compound 10 (9 mg, 0.009 mmol) at room temperature, and the mixture solution was stirred at room temperature for 2 h. After the reaction was completed, the reaction solution was directly separated and purified by Pre-HPLC (C18 column, mobile phase CH₃CN/H₂O=3/7 to 95/5, v/v) to afford the compound 40 (4.5 mg, 0.0025 mmol, 28% yield). MS (ESI) m/z=1593.6 [M+H]⁺.

### Examples 41-45

In accordance with the synthesis method of Example 40, the compound 3 used in the step was replaced with the corresponding amine in the following table, and the rest of reagents and operations remained unchanged to afford the corresponding target compounds of Examples 41, 42, 43, 44 and 45 in the table.

| Example | Amine | Compound |
|---|---|---|
| 41 | Compound 3 | 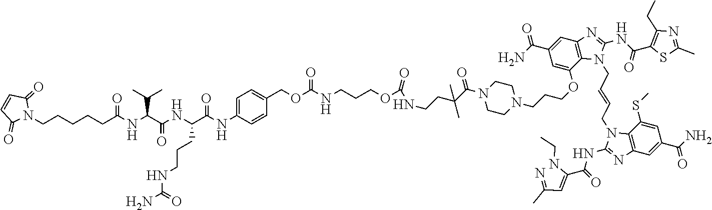 [SMILES: CCc1nc(C)sc1C(=O)Nc1nc2cc(C(N)=O)cc(OCCCN3CCN(C(=O)C(C)(C)CCNC(=O)OCCCNC(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21] |
| | | MS (ESI) m/z = 1694.5 [M + H]⁺ |
| 42 | Compound 21 | 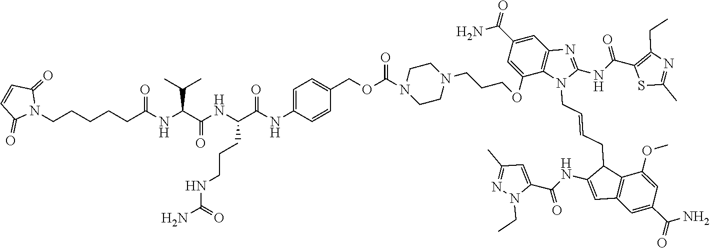 [SMILES: CCc1nc(C)sc1C(=O)Nc1nc2cc(C(N)=O)cc(OCCCN3CCN(C(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/CC1C(NC(=O)c2cc(C)nn2CC)=Cc2cc(C(N)=O)cc(OC)c21] |
| | | MS (ESI) m/z = 1464.2 [M + H]⁺ |
| 43 | Compound 1 | 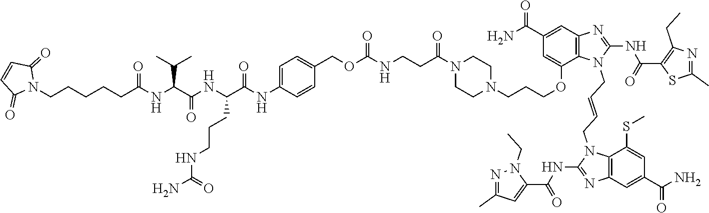 [SMILES: CCc1nc(C)sc1C(=O)Nc1nc2cc(C(N)=O)cc(OCCCN3CCN(C(=O)CCNC(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21] |
| | | MS (ESI) m/z = 1551.1 [M + H]⁺ |
| 44 | Compound 20 | 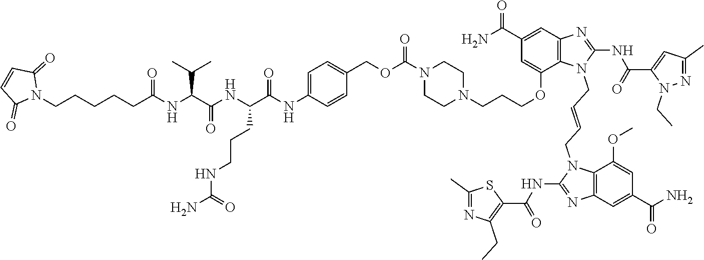 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)[C@H](CCCNC(N)=O)NC(=O)[C@@H](NC(=O)CCCCCN5C(=O)C=CC5=O)C(C)C)cc4)CC3)c21] |
| | | MS (ESI) m/z = 1464.2 [M + H]⁺ |
| 45 | intermediate 1-11 | 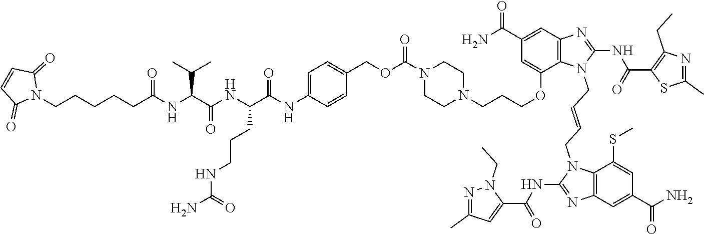 [SMILES: CCc1nc(C)sc1C(=O)Nc1nc2cc(C(N)=O)cc(OCCCN3CCN(C(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21] |
| | | MS (ESI) m/z = 1480.2 [M + H]⁺ |

### Example 46

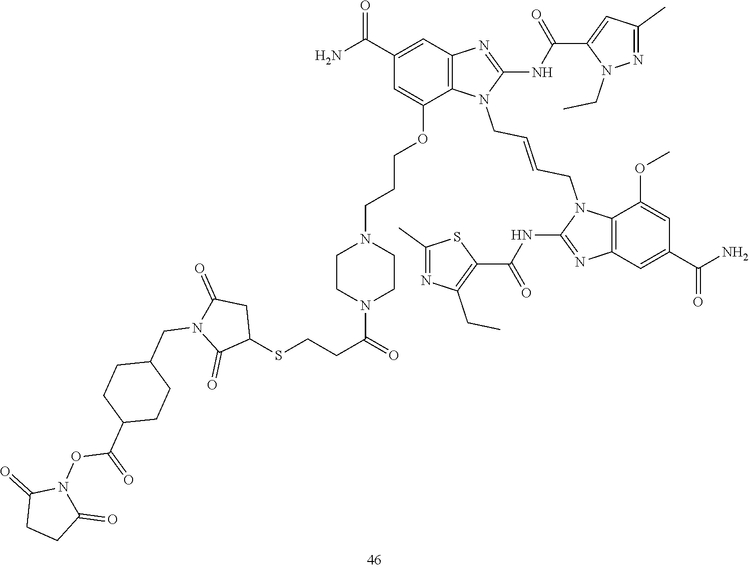 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCSC4CC(=O)N(CC5CCC(C(=O)ON6C(=O)CCC6=O)CC5)C4=O)CC3)c21]

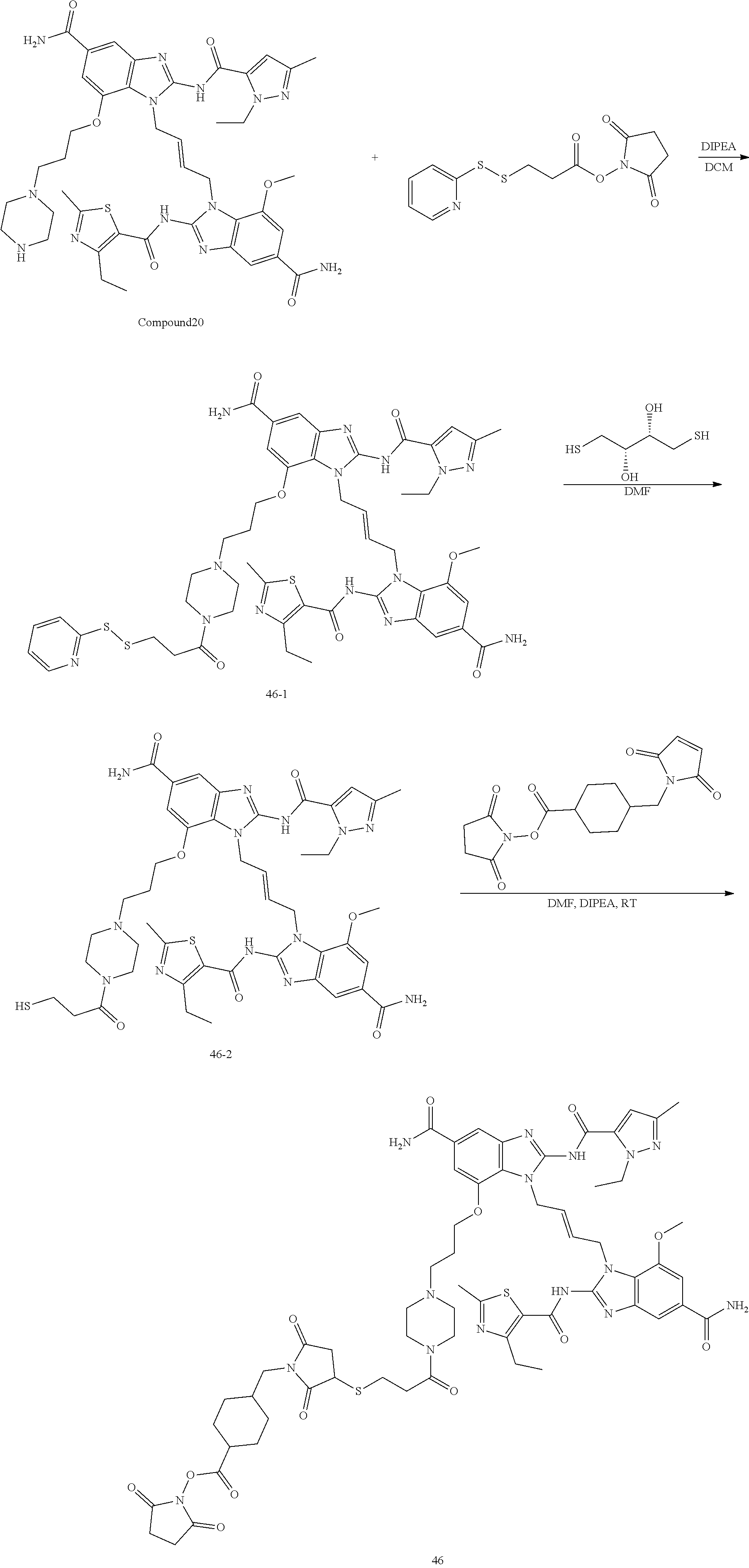

(Step 1) 3-(2-pyridyldithio) propionic acid N-hydroxysuccinimide ester (32 mg, 0.1 mmol) and DIPEA (36 mg, 0.28 mmol) were added to a DCM/THF solution of compound 20 (80 mg, 0.09 mmol) at room temperature, and the mixture solution was stirred at room temperature for 5 h. After the reaction was completed, the solvent was removed by rotary evaporation to give a crude product. The obtained crude product was separated and purified by silica gel column chromatography to afford the intermediate 46-1 (68 mg, 0.06 mmol, 70% yield). MS (ESI) m/z=1065.2[M+H]⁺

(Step 2) Dithiothreitol (20 mg, 0.13 mmol) was added to a DMF (3 mL) solution of intermediate 46-1 (68 mg, 0.06 mmol), and the reaction mixture was stirred at room temperature for 6 h. After the reaction was completed, water was added to the reaction solution for dilution, and DCM/MeOH was added for extraction. The organic phase combined was washed with water and saturated NaCl solution to give a crude product. The obtained crude product was separated and purified by silica gel column chromatography to afford the intermediate 46-2 (39 mg, 0.04 mmol, 65% yield). MS (ESI) m/z=955.2[M+H]⁺

(Step 3) 4-(N-maleimidomethyl) cyclohexane-1-carboxylic acid succinimidyl ester (21 mg, 0.06 mmol) and DIPEA (16 mg, 0.12 mmol) were added to a DMF (2 mL) solution of the intermediate 46-2 (39 mg, 0.04 mmol), and the mixture solution was stirred at room temperature for 6 h. After the reaction was completed, water was added to the reaction solution for dilution, and DCM/MeOH was added for extraction. The organic phase combined was concentrated to give a crude product. The obtained crude product was then separated and purified by Pre-HPLC to afford the compound 46 (10 mg, 0.008 mmol, 20% yield). MS (ESI) m/z=1289.2[M+H]⁺

### Synthesis of Examples 47, 48, 49, 50 and 51

In accordance with the synthesis method of Example 46, the compound 20 used in step 1 was replaced with the corresponding amine in the following table, and the rest of reagents and operations remained unchanged to afford the corresponding target compounds of Examples 47, 48, 49, 50 and 51 in the table.

| Example | Amine | Compound |
|---|---|---|
| 47 | Intermediate 1-11 | 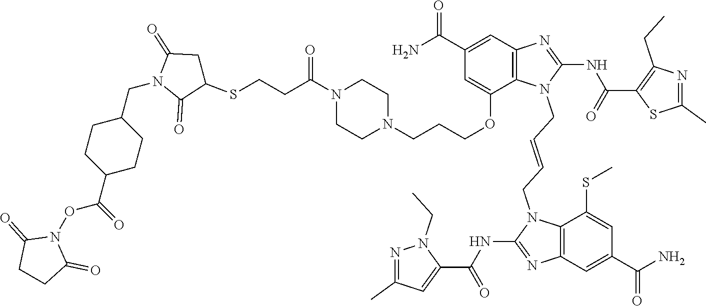 [SMILES: CCc1nc(C)sc1C(=O)Nc1nc2cc(C(N)=O)cc(OCCCN3CCN(C(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21] |
| | | MS (ESI) m/z = 1304.2 [M + H]⁺ |
| 48 | Compound 3 | 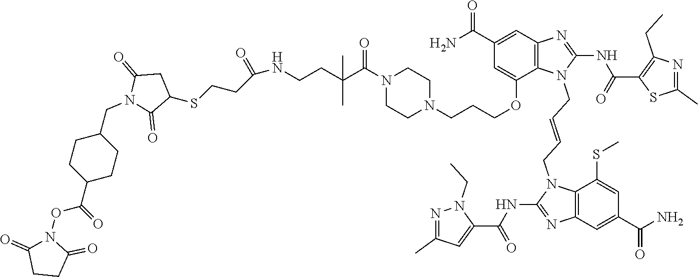 [SMILES: CCc1nc(C)sc1C(=O)Nc1nc2cc(C(N)=O)cc(OCCCN3CCN(C(=O)C(C)(C)CCNC(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21] |
| | | MS (ESI) m/z = 1417.5 [M + H]⁺ |
| 49 | Compound 1 | 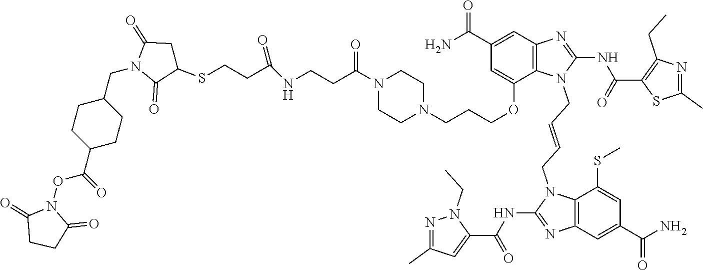 [SMILES: CCc1nc(C)sc1C(=O)Nc1nc2cc(C(N)=O)cc(OCCCN3CCN(C(=O)CCNC(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21] |
| | | MS (ESI) m/z = 1375.5 [M + H]⁺ |
| 50 | Compound 10 | 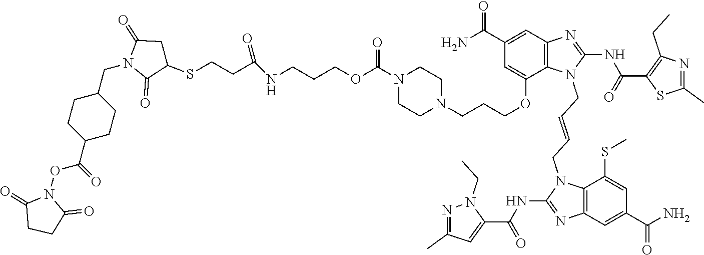 [SMILES: CCc1nc(C)sc1C(=O)Nc1nc2cc(C(N)=O)cc(OCCCN3CCN(C(=O)OCCCNC(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21] |
| | | MS (ESI) m/z = 1405.5 [M + H]⁺ |
| 51 | Compound 21 | 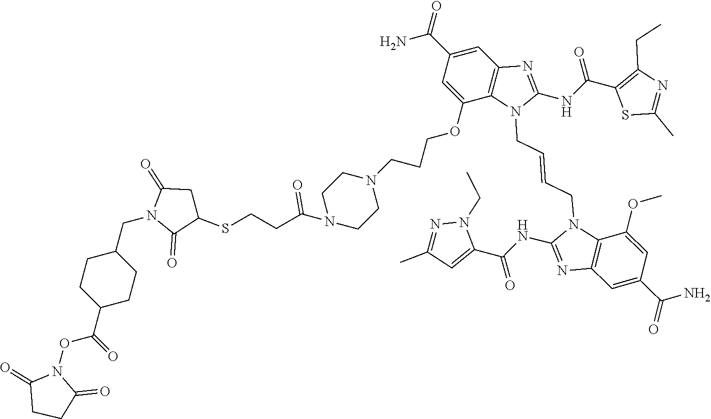 [SMILES: CCc1nc(C)sc1C(=O)Nc1nc2cc(C(N)=O)cc(OCCCN3CCN(C(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(OC)c21] |
| | | MS (ESI) m/z = 1288.4 [M + H]⁺ |

### Examples 52 and 53

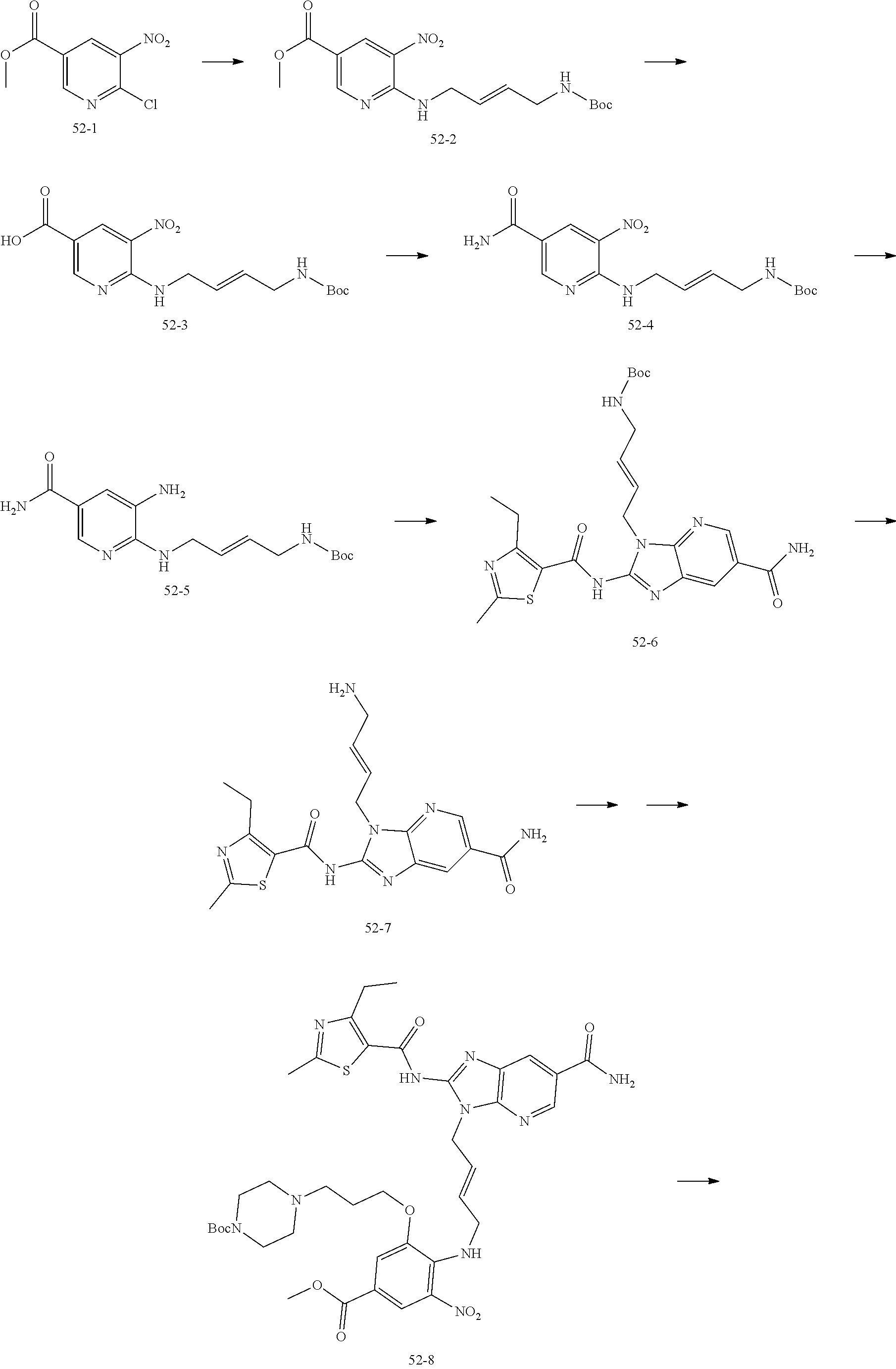

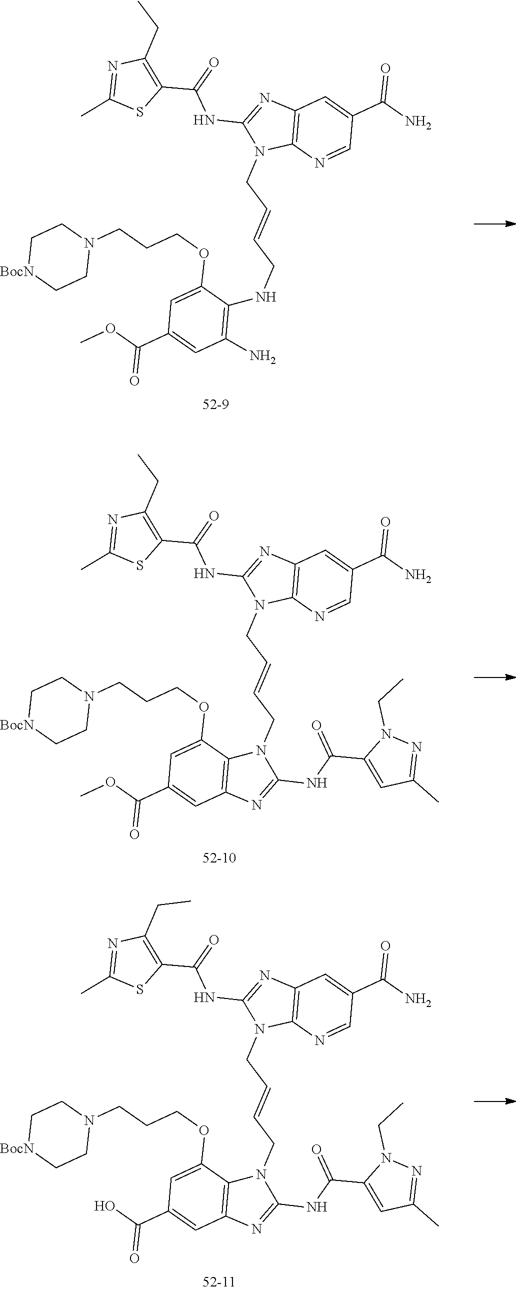 [SMILES: CCc1nc(C)sc1C(=O)Nc1nc2cc(C(N)=O)cnc2n1C/C=C/CNc1c(N)cc(C(=O)OC)cc1OCCCN1CCN(C(=O)OC(C)(C)C)CC1 ; CCc1nc(C)sc1C(=O)Nc1nc2cc(C(N)=O)cnc2n1C/C=C/Cn1c(NC(=O)c2cc(C)nn2CC)nc2cc(C(=O)OC)cc(OCCCN3CCN(C(=O)OC(C)(C)C)CC3)c21 ; CCc1nc(C)sc1C(=O)Nc1nc2cc(C(N)=O)cnc2n1C/C=C/Cn1c(NC(=O)c2cc(C)nn2CC)nc2cc(C(=O)O)cc(OCCCN3CCN(C(=O)OC(C)(C)C)CC3)c21]

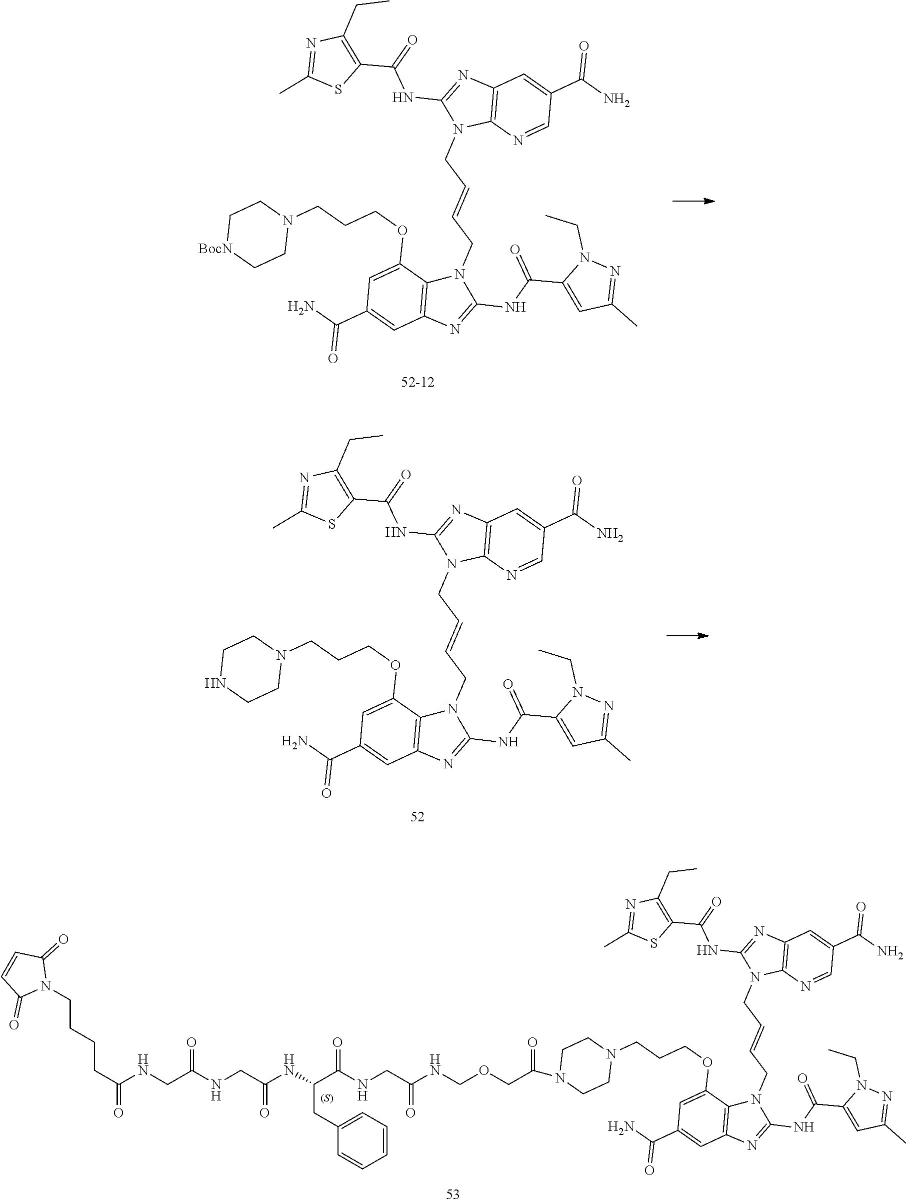

(Step 1) Methyl 6-chloro-5-nitronicotinate (1000 mg, 4.6 mmol) and tert-butyl (4-aminobut-2-en-1-yl)carbamate (1026 mg, 4.6 mmol) were dissolved in DMF (20 mL) at room temperature, to which triethylamine (1400 mg, 13.8 mmol) was added dropwise. The reaction mixture was stirred at room temperature for 2 h. After the reaction was completed, the reaction mixture was diluted with water, and ethyl acetate (20 mL×3) was added for extraction. The organic phase was washed with water (10 mL×2) and then dehydrated with anhydrous sodium sulfate. After the rotary evaporation under reduced pressure, the resulting residue was separated and purified by silica gel column chromatography (eluent: PE/EA=1/5 to 1/1) to afford the compound 52-2 (1500 mg, orange solid, 89% yield). MS (ESI) m/z=367 [M+H]⁺.

(Step 2) The compound 52-2 (1500 mg, 4.09 mmol) was dissolved in a mixed solvent of THE (4 mL) and methanol (4 mL), and an aqueous solution of lithium hydroxide (860 mg, 20.5 mmol) was added at room temperature. The mixture solution was stirred at room temperature for 1 h. And then 1 N hydrochloric acid was added to the reaction solution for adjusting the pH to precipitate solids. After suction filtration, the filter cake was washed with water and dried to afford the compound 52-3 (1300 mg, white solids, 90% yield).

MS (ESI) m/z=353 [M+H]⁺.

(Step 3) HATU (1406 mg, 3.7 mmol) was added to a DMF (15 mL) solution of compound 52-3 (1300 mg, 3.69 mmol) in an ice bath, and the mixture solution was stirred for 0.2 h. And then ammonium chloride (900 mg, 18.45 mmol) and N, N-diisopropylethylamine (1439 mg, 11.07 mmol) were added sequentially, the mixture solution was kept stirring and reacted at room temperature for 3 h. Water was added to the reaction solution for dilution, and ethyl acetate (20 mL×3) was added for extraction. The organic phase was washed with water (10 mL×2) and then dehydrated with anhydrous sodium sulfate. After rotary evaporation under reduced pressure, the resulting residue was separated and purified by silica gel column chromatography (eluent: PE/EA=1/5 to 1/1) to afford the compound 52-4 (1030 mg, white solids, yield of 80%).

MS (ESI) m/z=351.4 [M+H]⁺.

(Step 4) The compound 52-4 (670 mg, 1.91 mmol) was dissolved in methanol (15 mL) and cooled to 0° C., and then ammonia water (1.62 mL, 11.80 mmol) and an aqueous solution (6 mL) of sodium dithionite (1.22 g, 7.01 mmol) were added sequentially. The reaction mixture was stirred at 0° C. for 1 h, and the color of the reaction solution changed from orange red to white. Methanol was removed from the reaction solution by rotary evaporation. Water was then added for dilution and ethyl acetate (30 mL×4) was added for extraction. The separated organic phase was washed with saturated NaCl solution (20 mL×2) and then dehydrated with anhydrous sodium sulfate. The resulting residue was dried by rotary evaporation to afford the compound 52-5 (White solids, 320 mg, 62% yield). MS (ESI) m/z=321.4 [M+H]⁺.

(Step 5) 4-ethyl-2-methylthiazole-5-carbonyl isothiocyanate (210 mg, 0.99 mmol) was added to a DMF (10 mL) solution of the compound 52-5 (320 mg, 0.99 mmol) in an ice bath, and the mixture was stirred at room temperature for 0.5 h. HATU (376 mg, 0.99 mmol) and N,N-diisopropylethylamine (130 mg, 1.00 mmol) were then added to the reaction solution, and the reaction mixture was reacted under stirring at room temperature for 3 h. Water was added to the reaction solution for dilution. The mixture was filtered to collect white solids, which were then washed with water (5 mL×3) and separated by column chromatography (eluent acetonitrile/water=1/3, v/v) to afford the compound 52-6 (404 mg, white solids, 82% yield). MS (ESI) m/z=499.20[M+H]⁺.

(Step 6) Compound 52-6 (404 mg, 0.81 mmol) was dissolved in dichloromethane (10 mL), to which trifluoroacetic acid (5 mL) was added dropwise. After stirred at room temperature for 30 minutes, the reaction solution was dried by rotary evaporation to afford the compound 52-7 (320 mg, light yellow solids, 99% yield). MS (ESI) m/z=399.5[M+H]⁺.

(Step 7) Compound 52-7 (320 mg, 0.8 mmol) was dispersed in n-butanol (15 mL), and 4-(3-(2-chloro-5-(methoxycarbonyl)-3-nitrophenoxy)propyl)piperazine-1-carboxylate tert-butyl ester (365 mg, 0.8 mmol) and DIPEA (1.03 g, 8 mmol) were added. The reaction solution was heated up to 120° C. and stirred for 18 h. The mixture was then distilled under reduced pressure to give a crude product which was then dissolved in ethyl acetate. The above product was washed with water and saturated NaCl solution respectively and then dehydrated with anhydrous magnesium sulfate. The resulting residue was dried by rotary evaporation and then purified by silica gel column chromatography (eluent: petroleum ether/ethyl acetate=1/2) to afford the compound 52-8 (287 mg, 44% yield). MS(ESI) m/z=820.9 [M+H]⁺

(Step 8) Ammonia water (0.5 mL) was added to a methanol (10 mL) solution of the compound 52-8 (287 mg, 0.35 mmol) in an ice bath. After reacted for 10 minutes, an aqueous solution of sodium dithionite (311 mg, 1.8 mmol) was added, and the mixture was warmed up slowly to room temperature and reacted for 2 h. Inorganic salts were removed by filtration, and ethyl acetate was added for extraction. The organic phase was washed with saturated NaCl solution and then dehydrated with anhydrous magnesium sulfate. The resulting residue was dried by rotary evaporation to afford a crude product as the compound 52-9 (269 mg, 96% yield). MS(ESI) m/z=790.9 [M+H]⁺

(Step 9) 1-ethyl-3-methyl-1H-pyrazole-5-formyl isothiocyanate (66 mg, 0.34 mmol) was added to a DMF (3 mL) solution of the compound 52-9 (269 mg, 0.34 mmol). After reacted for 0.5 h, HATU (155 mg, 0.41 mmol) and DIPEA (88 mg, 0.68 mmol) were added, and then the mixture was warmed up to room temperature and reacted for 12 h. The reaction solution was slowly poured into water, and yellow solids were precipitated, which were filtered and dried to afford the compound 52-10 (304 mg, 94% yield). MS(ESI) m/z=952.1 [M+H]⁺

(Step 10) The compound 52-10 (304 mg, 0.32 mmol) was dissolved in a mixed solution (5.5 mL) of methanol, tetrahydrofuran and water (volume ratio: 1/1/0.5), and lithium hydroxide (134 mg, 3.2 mmol) was added. The reaction solution was heated to 75° C. and stirred overnight. The organic solvent was removed by rotary evaporation, and the obtained crude product was dissolved in water. Ethyl acetate was added for extraction, and the resulting aqueous phase was separated, and then dilute hydrochloric acid (1 M) was added in an ice bath for adjustment such that the solids were no longer precipitated. The obtained solids were filtered and dried to afford the compound 52-11 (197 mg, 67% yield). MS(ESI) m/z=938.1[M+H]⁺

(Step 11) The compound 52-11 (187 mg, 0.2 mmol) was dissolved in DMF (4 mL), and HATU (114 mg, 0.3 mmol) and DIPEA (103 mg, 0.8 mmol) were added. After 0.5 h, ammonium bicarbonate (47.4 mg, 0.6 mmol) was added and the mixture was stirred at room temperature for 2 h. Water was added to the reaction solution to precipitate solids, which were collected by filtration, washed with pure water and dried to afford the compound 52-12 (168 mg, 90% yield). MS(ESI) m/z=937.1 [M+H]⁺

(Step 12) TFA (3 mL) was added to a DCM (5 mL) solution of compound 52-12 (168 mg, 0.18 mmol) in an ice bath, and the mixture was warmed up slowly to room temperature. After reacted for 1 h, the reaction solvent was dried by rotary evaporation. The free TFA was removed under reduced pressure with an oil pump to afford the compound 52 (187 mg, 80% purity, containing TFA salt). MS(ESI) m/z=837.1 [M+H]⁺

(Step 13) DMTMM (35 mg, 0.12 mmol) was added to a DMF (2 mL) solution of compound 22-9 (61 mg, 0.1 mmol) in an ice bath. After 5 minutes, compound 52 (105 mg, 0.1 mmol, 80% content) and DIPEA (39 mg, 0.3 mmol) were added. The mixture was reacted at room temperature for 1 h. The reaction solution was purified by preparative HPLC to afford the target compound 53 (37 mg, yield of 26%).

MS(ESI) m/z=1421.6[M+H]⁺

The beneficial effect of the present disclosure will be illustrated with reference to the following experimental examples.

### Experimental Example 1 Activation Effect of the Compounds on the STING Signaling Pathway in THP1

In this experiment, the function of sting agonists was evaluated by detecting the changes of IFN-β and CXCL10 (IP10) cytokines which were produced by the stimulation of the compounds to human peripheral blood mononuclear cell line THP1 cell (Shanghai Cell Bank). On the first day of the experiment, ELISA plates were coated according to the instructions of IFN-β (R&D, #DY814-05) and IP10 (BD, #550926) ELISA detection kits. The compound was dissolved in DMSO as a stock solution, which was diluted with culture medium to a 2× working concentration, and then added to a 96-well plate at 100 μL per well; THP1 cells in the logarithmic growth phase were counted and diluted to a concentration of 2*10⁶/mL, which was then added at 100 μL per well to the aforementioned 96-well plate containing the compounds. After mixed, the above cells in the 96-well plate were incubated at 37° C. and 5% CO₂ for 18 h. On the second day, the above cell culture supernatant was aspirated at 100 μL per well, and then their OD450 values were determined using the IFN-β and IP10 ELISA detection kits respectively. The obtained data was converted to the concentration of IFN-β and IP10 according to the standard curve, and the EC50 values were calculated using GraphPad 5.0 to fit the dose-effect curve. The test results were shown in the following table, where the EC₅₀ value of each compound was classified according to the following instructions:
“+” indicated that the value of EC₅₀ was greater than 1 μM;
“++” indicated that the value of EC₅₀ was less than 1 μM and greater than 100 nM;

| | IFN-β |
|---|---|
| Example | EC₅₀ (μM) |
| 1 | ++ |
| 2 | ++ |
| 3 | ++ |
| 6 | ++ |
| 8 | ++ |
| 10 | ++ |
| 11 | + |
| 12 | + |
| 13 | ++ |
| 14 | ++ |
| 15 | ++ |
| 16 | + |
| 17 | + |
| 18 | + |
| 19 | + |

### Experimental Example 2 Activation Effect of the Compounds on the STING Signaling Pathway in THP1-Blue™ ISG

In this experiment, THP1-Blue™ ISG (interferon-stimulated genes) cell line (InvivoGen, catalog number: thp-isg) was used to evaluate the activating of interferon signaling pathway after the treatment by the compounds. THP1-Blue™ ISG cells were cultured using RPMI1640 medium (containing 10% heat-inactivated fetal bovine serum, 100 μg/ml Zeocin™, InvivoGen, catalog number: ant-zn-1). On the first day of the experiment, the compound was dissolved in DMSO as a stock solution, which was then diluted with culture medium to 2× working concentration and then 100 μL per well of the above dilution was added to a 96-well plate; THP1 cells in the logarithmic growth phase were counted and diluted to a concentration of 1*10⁶ cells/mL, which was then added at 100 μL per well (1*10⁵ cells/well) to the aforementioned 96-well plate containing the compounds. After mixed, the above cells in the 96-well plate were incubated at 37° C. with 5% CO₂ for 18 h. On the second day, the above cell culture supernatant was aspirated at 20 μL per well, which was added to a new 96-well plate. And then 180 μL per well of QUANTI-Blue™ solution (InvivoGen, catalog number: rep-qbs1) was added. The above cells in the 96-well plate were incubated at 37° C. for 1 h. OD (620-655 nm) value was read on a Microplate Reader, and the EC₅₀ value was calculated by using GraphPad to fit the dose-effect curve. 50 μL of Celltiter Glo solution (Promega, Cat. No. G756B) was added to the remaining cell culture plates. After incubated for 10 minutes, the luminescence value was measured on a Microplate Reader, and the 50% cytotoxic concentration (CC₅₀) was calculated.
“+” indicated that the value of EC₅₀ was greater than 1 μM;
“++” indicated that the value of EC₅₀ was less than 1 μM and greater than 100 nM;
“+++” indicated that the value of EC₅₀ was less than 100 nM.

| Example | EC₅₀ (μM) | CC₅₀ (μM) |
|---|---|---|
| 1 | +++ | >50 |
| 2 | +++ | >50 |
| 3 | +++ | >50 |
| 10 | +++ | >50 |
| 13 | +++ | >50 |
| 14 | +++ | >50 |
| 20 | ++ | >50 |
| 21 | +++ | NT |
| 40 | +++ | >50 |
| 41 | +++ | >50 |

### Experimental Example 3 Activation Effect of the Compounds on the STING Signaling Pathway in PBMC

The following experiments were performed to determine the activation effect of the compound on the STING signaling pathway in PBMC cells, which mainly using ELISA to detect the secretion of IL-6, TNF-, IFN-, and IFN- in the cell culture supernatant. At the same time, CTG (CellTiter-Glo) was used to detect the survival rate of the cells, and to calculate the half cytotoxic dose of cells CC₅₀.

The frozen PBMC cells were placed in a 37° C. water bath. After the ice was completely melted, the cells were transferred to 3 ml of RPMI 1640 complete culture medium (containing 10% FBS), and then centrifuged at 1500 rpm for 3 minutes. After removing the supernatant, the cells were re-suspended for counting. 200 μL of 50,000 PBMC cells were added in a 96-well plate, and the compounds to be tested were diluted with different gradient concentrations. After 6 h, 150 μL of the cell supernatant was aspirated, and the concentrations of IL-6, TNF-, IFN-, and IFN- were measured using ELISA kits, and the curves of compound concentration and cytokine release were drawn to calculate the values of EC₅₀. 50 μL of CTG solution was added to the remaining cell culture plate. After incubated for 10 minutes, the fluorescence values were determined on a Microplate Reader, and the values of CC₅₀ were calculated.
“+” indicated that the value of EC₅₀ was greater than 1 μM;
“++” indicated that the value of EC₅₀ was less than 1 μM and greater than 100 nM;
“+++” indicated that the value of EC₅₀ was less than 100 nM;
“NT” indicated that the compound was not tested.

| | IL-6 EC₅₀ | TNF-α EC₅₀ | IFN-β EC₅₀ | CC₅₀ |
|---|---|---|---|---|
| Example | (μM) | (μM) | (μM) | (μM) |
| 1 | ++ | ++ | ++ | >25 |
| 2 | ++ | ++ | ++ | >40 |
| 3 | ++ | ++ | ++ | >40 |
| 10 | ++ | ++ | ++ | >40 |
| 13 | +++ | +++ | ++ | >50 |
| 14 | ++ | +++ | ++ | >50 |
| 20 | ++ | ++ | ++ | >50 |
| 21 | ++ | +++ | ++ | NT |
| 40 | +++ | +++ | NT | NT |
| 41 | ++ | ++ | NT | NT |

The above experiments demonstrate that the compounds and the compound-link chain conjugates of the present disclosure have higher STING activation activity, and are suitable as payloads of antibody-drug conjugates.

What is claimed is:

1. A compound-linker conjugate of formula (I): 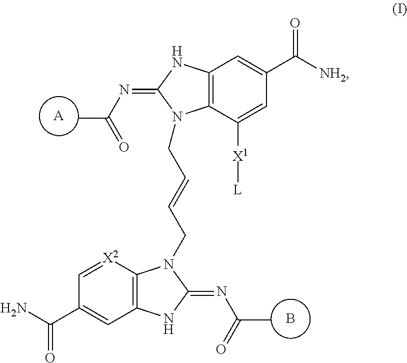
wherein ring A and ring B are each independently 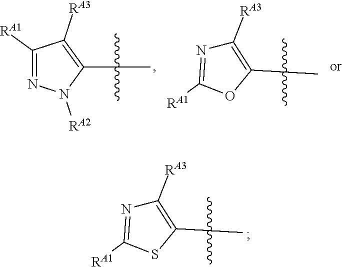
R^{A1} and R^{A3} are each independently selected from the group consisting of hydrogen, halogen, —CN, unsubstituted —C₁₋₆ alkyl and halogenated —C₁₋₆ alkyl;
R^{A2} is selected from the group consisting of hydrogen, unsubstituted —C₁₋₆ alkyl and halogenated —C₁₋₆ alkyl;
X¹ is O or S;
X² is N or CR^{X};
R^{X} is selected from the group consisting of hydrogen, halogen, —CN, unsubstituted —C₁₋₆ alkyl, halogenated —C₁₋₆ alkyl, —R¹, —OR¹, —SR¹ and —NR¹R^{1′};
R¹ and R^{1′} are each independently selected from the group consisting of hydrogen, —C₁₋₈ alkyl, —C₁₋₈ alkylene-NR²R^{2′}, —C₁₋₈ alkylene-OR², —C₀₋₈ alkylene-(3-10 membered cycloalkyl), —C₀₋₈ alkylene-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in an alkylene group are adapted to be replaced with an oxygen atom;
R² and R^{2′} are each independently selected from the group consisting of hydrogen, —C₁₋₆ alkyl, —C₀₋₈ alkylene-(3-10 membered cycloalkyl), —C₀₋₈ alkyl-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in an alkylene group are adapted to be replaced with an oxygen atom;
L is selected from -(L¹)_{q}-W;
q is an integer selected from 1-100;
L¹ is each independently selected from the group consisting of CRR, C(O), O, S, S(O), S(O)₂, NR, —CR═CR—, —C≡C—, P(O)R, P(O)OR, 3-10 membered cycloalkane, 3-10 membered heterocycloalkane, 5-10 membered aromatic ring, 5-10 membered aromatic heterocyclic ring, 5-12 membered spiro ring, 5-12 membered spiro heterocyclic ring, 5-12 membered bridged ring and 5-12 membered bridged heterocyclic ring; wherein cycloalkane, heterocycloalkane, aromatic ring, aromatic heterocyclic ring, spiro ring, spiro heterocyclic ring, bridged ring, and bridged heterocyclic ring are unsubstituted or substituted by one, two or three R^{L1};
R^{L1} is each independently selected from the group consisting of hydrogen, halogen, ═O, cyano, nitro, unsubstituted —C₁₋₆ alkyl, halogenated —C₁₋₆ alkyl, —OR, —NRR, —C₀₋₄ alkylene-(3-10 membered cycloalkyl) and —C₀₋₄ alkylene-(3-10 membered heterocycloalkyl);
R is each independently selected from the group consisting of hydrogen, halogen, cyano, unsubstituted —C₁₋₆ alkyl, unsubstituted —C₂₋₆ alkenyl, unsubstituted —C₂₋₆ alkynyl, halogenated —C₁₋₆ alkyl, halogenated —C₂₋₆ alkenyl, halogenated —C₂₋₆ alkynyl, —C₀₋₄ alkylene-OR^{R1}, —C₀₋₄ alkylene-OC(O)R^{R1}, —C₀₋₄ alkylene-SR^{R1}, —C₀₋₄ alkylene-S(O)₂R^{R1}, —C₀₋₄ alkylene-S(O)R^{R1}, —C₀₋₄ alkylene-S(O)₂NR^{R1}R^{R2}, —C₀₋₄ alkylene-S(O)NR^{R1}R^{R2}, —C₀₋₄ alkylene-S(O)(NH)R^{R1}, —C₀₋₄ alkylene-S(O)(NH)NR^{R1}R^{R2}, —C₀₋₄ alkylene-C(O)R^{R1}, —C₀₋₄ alkylene-C(O)OR^{R1}, —C₀₋₄ alkylene-C(O)NR^{R1}R^{R2}, —C₀₋₄ alkylene-NR^{R1}R^{R2}, —C₀₋₄ alkylene-NR^{R1}C(O)R^{R2}, C₀₋₄ alkylene-NR^{R1}S(O)₂R^{R2}, C₀₋₄ alkylene-NR^{R1}S(O)R^{R2}, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring);
R^{R1} and R^{R2} are independently selected from the group consisting of hydrogen, unsubstituted —C₁₋₆ alkyl, unsubstituted —C₂₋₆ alkenyl, unsubstituted —C₂₋₆ alkynyl, —OH, —NH₂, halogenated —C₁₋₆ alkyl, halogenated —C₂₋₆ alkenyl, halogenated —C₂₋₆ alkynyl, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring); and
W is selected from the group consisting of

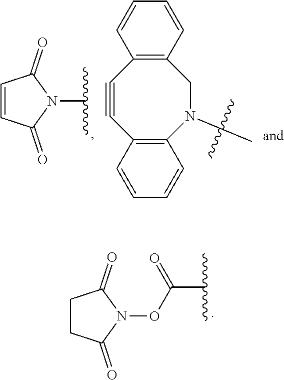

2. The compound-linker conjugate of claim 1, wherein L is -M-L^{A}-L^{B}-W;
M is selected from the group consisting of —C₁₋₈ alkylene-, —C₀₋₈ alkylene-(3-10 membered cycloalkyl)-, —C₀₋₈ alkylene-(3-10 membered heterocycloalkyl)-, —C₀₋₈ alkylene-(5-12 membered spirocycloalkyl)-, —C₀₋₈ alkylene-(5-12 membered spiroheterocycloalkyl)-, —C₀₋₈ alkylene-(5-12 membered bridged cycloalkyl)- and —C₀₋₈ alkylene-(5-12 membered bridged heterocycloalkyl)-; wherein one or two carbon atoms in an alkylene group are adapted to be replaced with an oxygen atom;
L^{A} is selected from the group consisting of —C(O)—C₁₋₈ alkylene-NH—, —C(O)—C₁₋₈ alkylene-N(C₁₋₆ alkyl)-, —C(O)O—C₁₋₈ alkylene-NH—, —C(O)O—C₁₋₈ alkylene-N(C₁₋₆ alkyl)-, —C(O)NH—C₁₋₈ alkylene-NH—, —C(O)NH—C₁₋₈ alkylene-N(C₁₋₆ alkyl)-, —C₁₋₈ alkylene-NH—, —C₁₋₈ alkylene-N(C₁₋₆ alkyl)-, —C(O)—C₁₋₈ alkylene-(3-10 membered cycloalkyl)-, —C(O)O—C₁₋₈ alkylene-(3-10 membered cycloalkyl)-, —C(O)NH—C₁₋₈ alkylene-(3-10 membered cycloalkyl)-, —C₁₋₈ alkylene-(3-10 membered cycloalkyl)-, —C(O)—C₁₋₈ alkylene-(3-10 membered heterocycloalkyl)-, —C(O)O—C₁₋₈ alkylene-(3-10 membered heterocycloalkyl)-, —C(O)NH—C₁₋₈ alkylene-(3-10 membered heterocycloalkyl)-, —C₁₋₈ alkylene-(3-10 membered heterocycloalkyl)-, —C(O)—C₁₋₈ alkylene-(5-12 membered spirocycloalkyl)-, —C(O)O—C₁₋₈ alkylene-(5-12 membered spirocycloalkyl)-, —C(O)NH—C₁₋₈ alkylene-(5-12 membered spirocycloalkyl)-, —C₁₋₈ alkylene-(5-12 membered spirocycloalkyl)-, —C(O)—C₁₋₈ alkylene-(5-12 membered spiroheterocycloalkyl)-, —C(O)O—C₁₋₈ alkylene-(5-12 membered spiroheterocycloalkyl)-, —C(O)NH—C₁₋₈ alkylene-(5-12 membered spiroheterocycloalkyl)-, —C₁₋₈ alkylene-(5-12 membered spiroheterocycloalkyl), —C(O)—C₁₋₈ alkylene-(5-12 membered bridged cycloalkyl)-, —C(O)O—C₁₋₈ alkylene-(5-12 membered bridged cycloalkyl)-, —C(O)NH—C₁₋₈ alkylene-(5-12 membered bridged cycloalkyl)-, —C₁₋₈ alkylene-(5-12 membered bridged cycloalkyl)-, —C(O)—C₁₋₈ alkylene-(5-12 membered bridged heterocycloalkyl)-, —C(O)O—C₁₋₈ alkylene-(5-12 membered bridged heterocycloalkyl)-, —C(O)NH—C₁₋₈ alkylene-(5-12 membered bridged heterocycloalkyl)-, —C₁₋₈ alkylene-(5-12 membered bridged heterocycloalkyl)-, —C(O)—C₁₋₈ alkylene-O—, —C(O)O—C₁₋₈ alkylene-O—, —C(O)NH—C₁₋₈ alkylene-O—, —C₁₋₈ alkylene-O—, —S(O)₂—C₁₋₈ alkylene-NH—, —S(O)₂—C₁₋₈ alkylene-N(C₁₋₆ alkyl)-, —S(O)₂O—C₁₋₈ alkylene-NH—, —S(O)₂O—C₁₋₈ alkylene-N(C₁₋₆ alkyl)-, —S(O)₂NH—C₁₋₈ alkylene-NH—, —S(O)₂NH—C₁₋₈ alkylene-N(C₁₋₆ alkyl)-, —S(O)₂—C₁₋₈ alkylene-(3-10 membered cycloalkyl)-, —S(O)₂O—C₁₋₈ alkylene-(3-10 membered cycloalkyl)-, —S(O)₂NH—C₁₋₈ alkylene-(3-10 membered cycloalkyl)-, —S(O)₂—C₁₋₈ alkylene-(3-10 membered heterocycloalkyl)-, —S(O)₂O—C₁₋₈ alkylene-(3-10 membered heterocycloalkyl)-, —S(O)₂NH—C₁₋₈ alkylene-(3-10 membered heterocycloalkyl)-, —S(O)₂—C₁₋₈ alkylene-(5-12 membered spirocycloalkyl)-, —S(O)₂O—C₁₋₈ alkylene-(5-12 membered spirocycloalkyl)-, —S(O)₂NH—C₁₋₈ alkylene-(5-12 membered spirocycloalkyl)-, —S(O)₂—C₁₋₈ alkylene-(5-12 membered spiroheterocycloalkyl)-, —S(O)₂O—C₁₋₈ alkylene-(5-12 membered spiroheterocycloalkyl)-, —S(O)₂NH—C₁₋₈ alkylene-(5-12 membered spiroheterocycloalkyl)-, —S(O)₂—C₁₋₈ alkylene-(5-12 membered bridged cycloalkyl)-, —S(O)₂O—C₁₋₈ alkylene-(5-12 membered bridged cycloalkyl)-, —S(O)₂NH—C₁₋₈ alkylene-(5-12 membered bridged cycloalkyl)-, —S(O)₂—C₁₋₈ alkylene-(5-12 membered bridged heterocycloalkyl)-, —S(O)₂O—C₁₋₈ alkylene-(5-12 membered bridged heterocycloalkyl)-, —S(O)₂NH—C₁₋₈ alkylene-(5-12 membered bridged heterocycloalkyl)-, —S(O)₂—C₁₋₈ alkylene-O—, —S(O)₂O—C₁₋₈ alkylene-O—, —S(O)₂NH—C₁₋₈ alkylene-O— and a chemical bond; wherein one or two carbon atoms in an alkylene group are adapted to be replaced with an oxygen atom;
L^{B} is -(L¹)ₚ-;
p is an integer selected from 1 to 50;
L¹ is each independently selected from the group consisting of CRR, C(O), O, S, S(O), S(O)₂, NR, —CR═CR—, —C≡C—, P(O)R, P(O)OR, 3-10 membered cycloalkane, 3-10 membered heterocycloalkane, 5-10 membered aromatic ring, 5-10 membered aromatic heterocyclic ring, 5-12 membered spiro ring, 5-12 membered spiro heterocyclic ring, 5-12 membered bridged ring and 5-12 membered bridged heterocyclic ring; wherein cycloalkane, heterocycloalkane, aromatic ring, aromatic heterocyclic ring, spiro ring, spiro heterocyclic ring, bridged ring and bridged heterocyclic ring are unsubstituted or substituted by one, two or three R^{L1};
R^{L1} is each independently selected from the group consisting of hydrogen, halogen, ═O, cyano, nitro, unsubstituted —C₁₋₆ alkyl, halogenated —C₁₋₆ alkyl, —OR, —NRR, —C₀₋₄ alkylene-(3-10 membered cycloalkyl) and —C₀₋₄ alkylene-(3-10 membered heterocycloalkyl);
R is each independently selected from the group consisting of hydrogen, halogen, cyano, unsubstituted —C₁₋₆ alkyl, unsubstituted —C₂₋₆ alkenyl, unsubstituted —C₂₋₆ alkynyl, halogenated —C₁₋₆ alkyl, halogenated —C₂₋₆ alkenyl, halogenated —C₂₋₆ alkynyl, —C₀₋₄ alkylene-OR^{R1}, —C₀₋₄ alkylene-OC(O)R^{R1}, —C₀₋₄ alkylene-SR^{R1}, —C₀₋₄ alkylene-S(O)₂R^{R1}, —C₀₋₄ alkylene-S(O)R^{R1}, —C₀₋₄ alkylene-S(O)₂NR^{R1}R^{R2}, —C₀₋₄ alkylene-S(O)NR^{R1}R^{R2}, —C₀₋₄ alkylene-S(O)(NH)R^{R1}, —C₀₋₄ alkylene-S(O)(NH)NR^{R1}R^{R2}, —C₀₋₄ alkylene-C(O)R^{R1}, —C₀₋₄ alkylene-C(O)OR^{R1}, —C₀₋₄ alkylene-C(O)NR^{R1}R^{R2}, —C₀₋₄ alkylene-NR^{R1}R^{R2}, —C₀₋₄ alkylene-NR^{R1}C(O)R^{R2}, C₀₋₄ alkylene-NR^{R1}S(O)₂R^{R2}, C₀₋₄ alkylene-NR^{R1}S(O)R^{R2}, —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring);
R^{R1} and R^{R2} are independently selected from the group consisting of hydrogen, unsubstituted —C₁₋₆ alkyl, unsubstituted —C₂₋₆ alkenyl, unsubstituted —C₂₋₆ alkynyl, —OH, —NH₂, halogenated —C₁₋₆ alkyl, halogenated —C₂₋₆ alkenyl, halogenated —C₂₋₆ alkynyl —C₀₋₄ alkylene-(3-10 membered carbocyclic group), —C₀₋₄ alkylene-(4-10 membered heterocycloalkyl), —C₀₋₄ alkylene-(6-10 membered aromatic ring) and —C₀₋₄ alkylene-(5-10 membered aromatic heterocyclic ring);
W is selected from the group consisting of

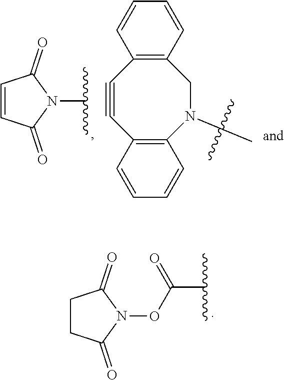

3. The compound-linker conjugate of claim 2, wherein M is selected from the group consisting of

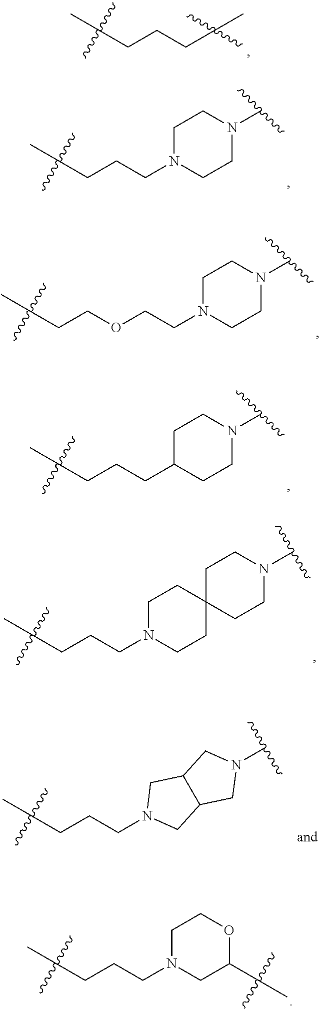

4. The compound-linker conjugate of claim 2, wherein L^{A} is selected from the group consisting of 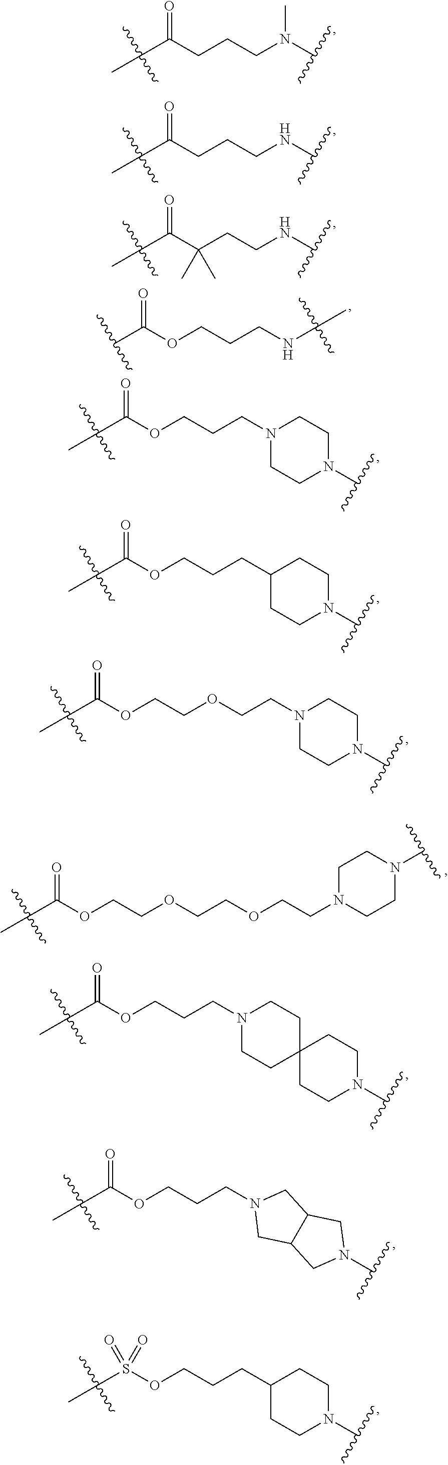 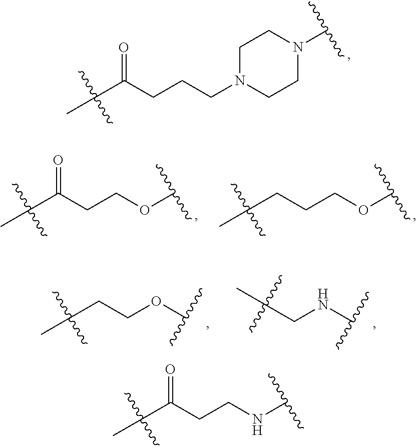
and a chemical bond.

5. The compound-linker conjugate of claim 2, wherein -L^{B}-W is selected from the group consisting of

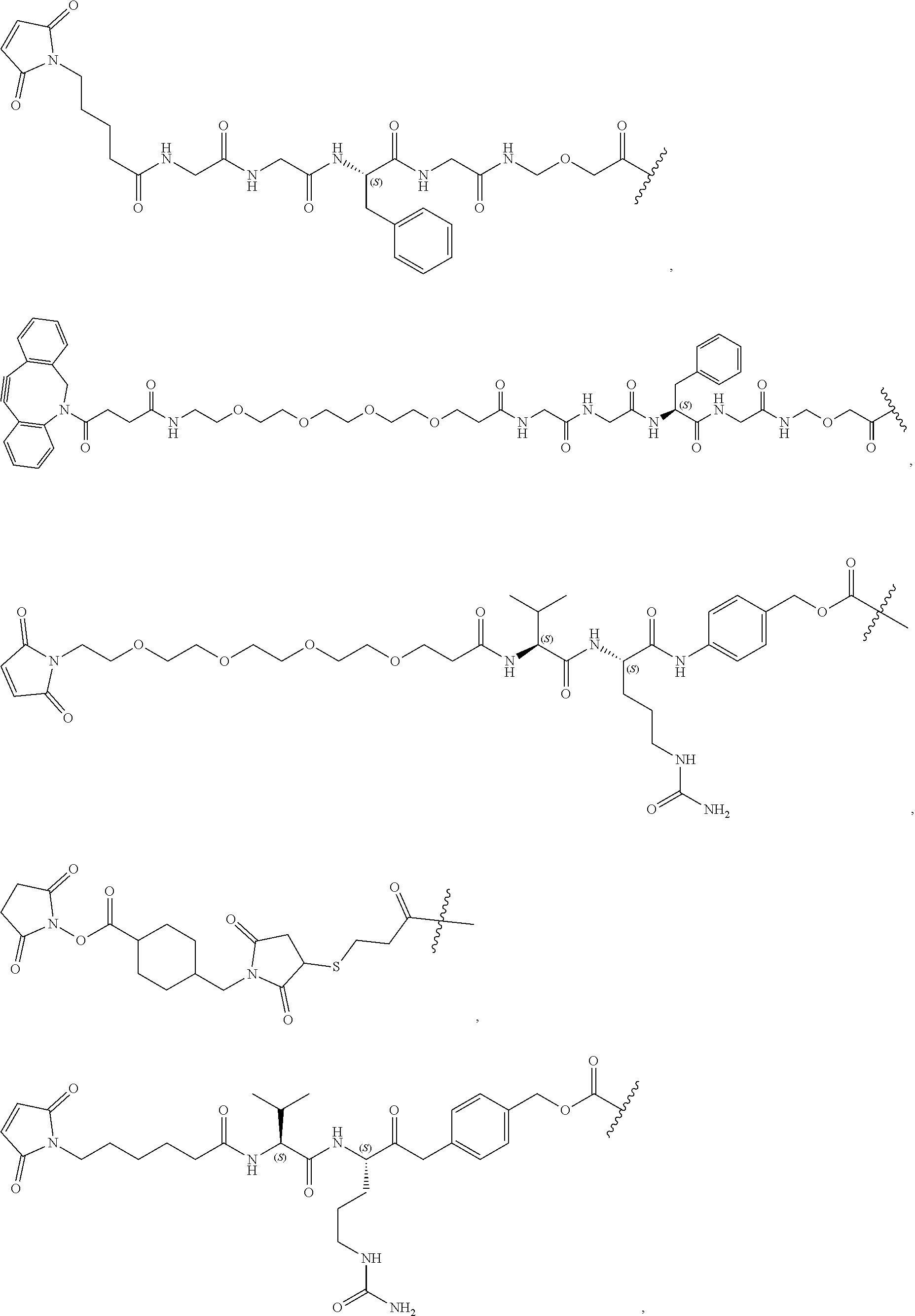

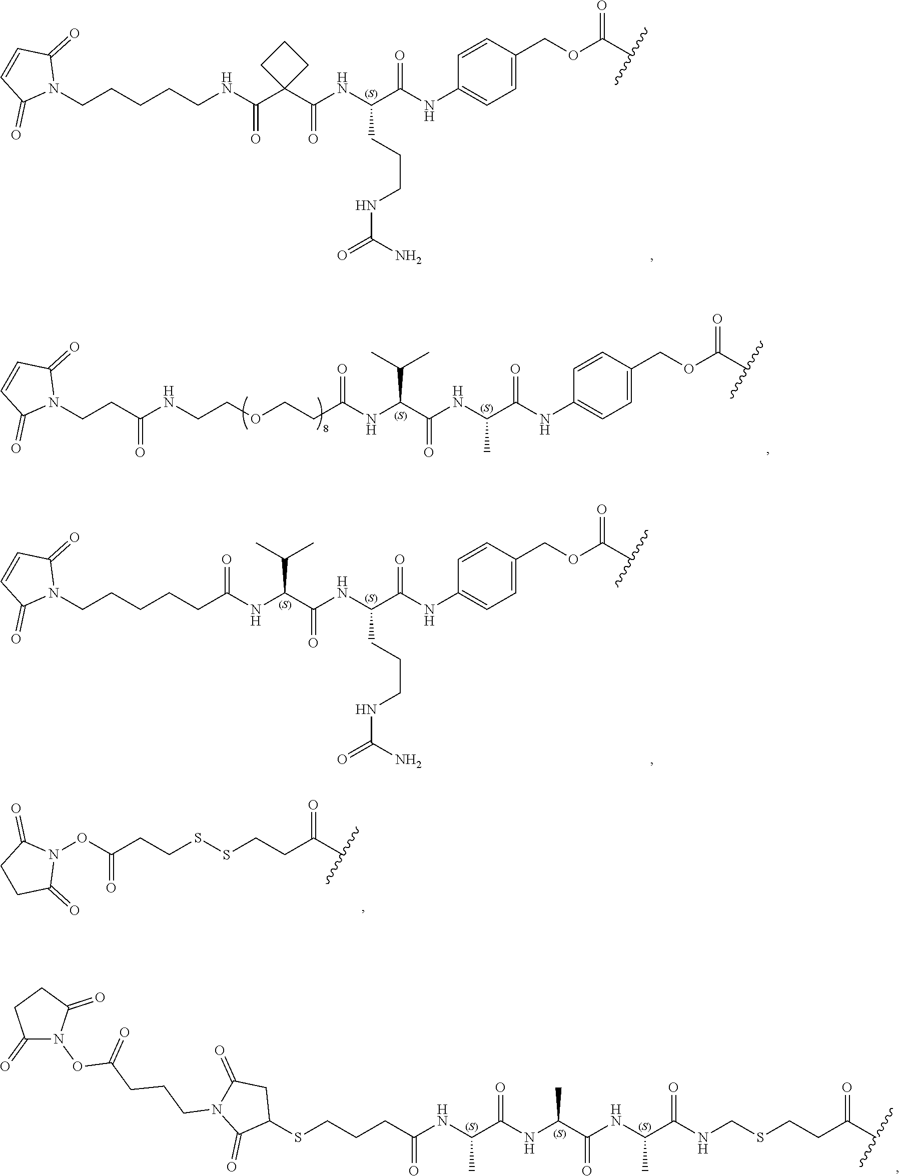

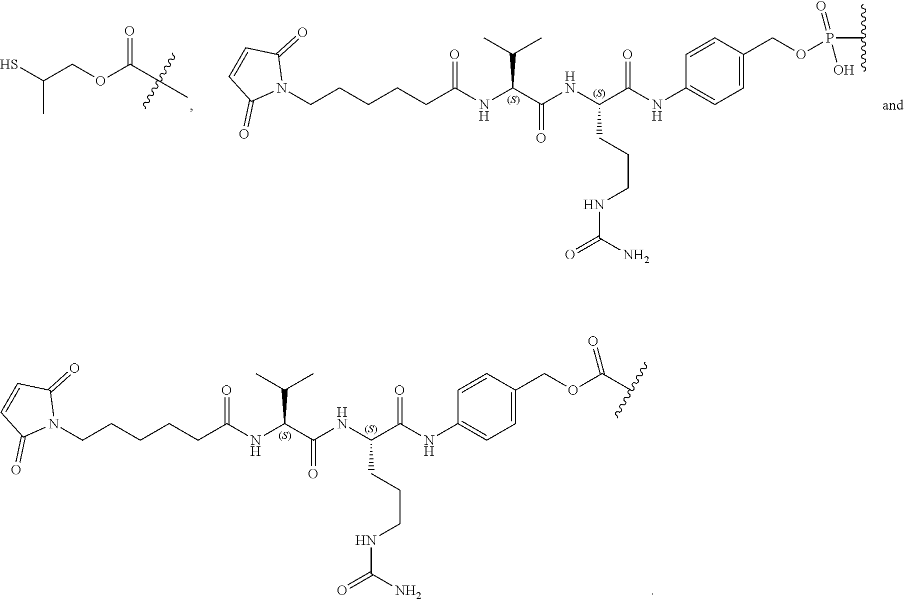

6. The compound-linker conjugate of claim 1, wherein the ring A and the ring B are independently selected from the group consisting of

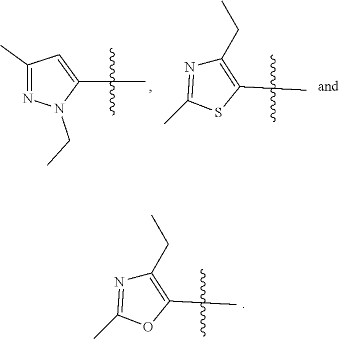

7. The compound-linker conjugate of claim 1, wherein X² is N or CR^{X};
R^{X} is —OR¹ or —SR¹; and
R¹ is methyl, or -propylidene-OH.

8. The compound-linker conjugate of claim 1, wherein the compound-linker conjugate is selected from the group consisting of:

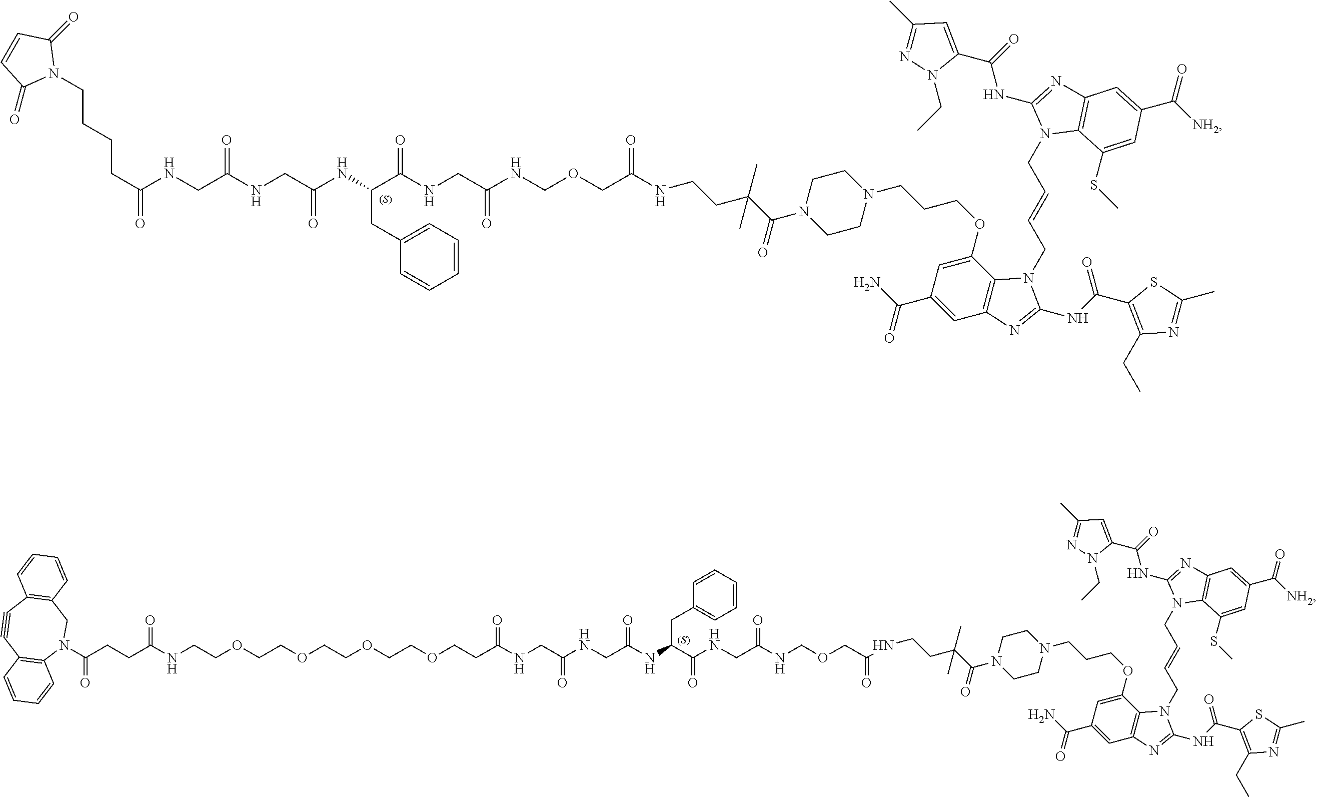

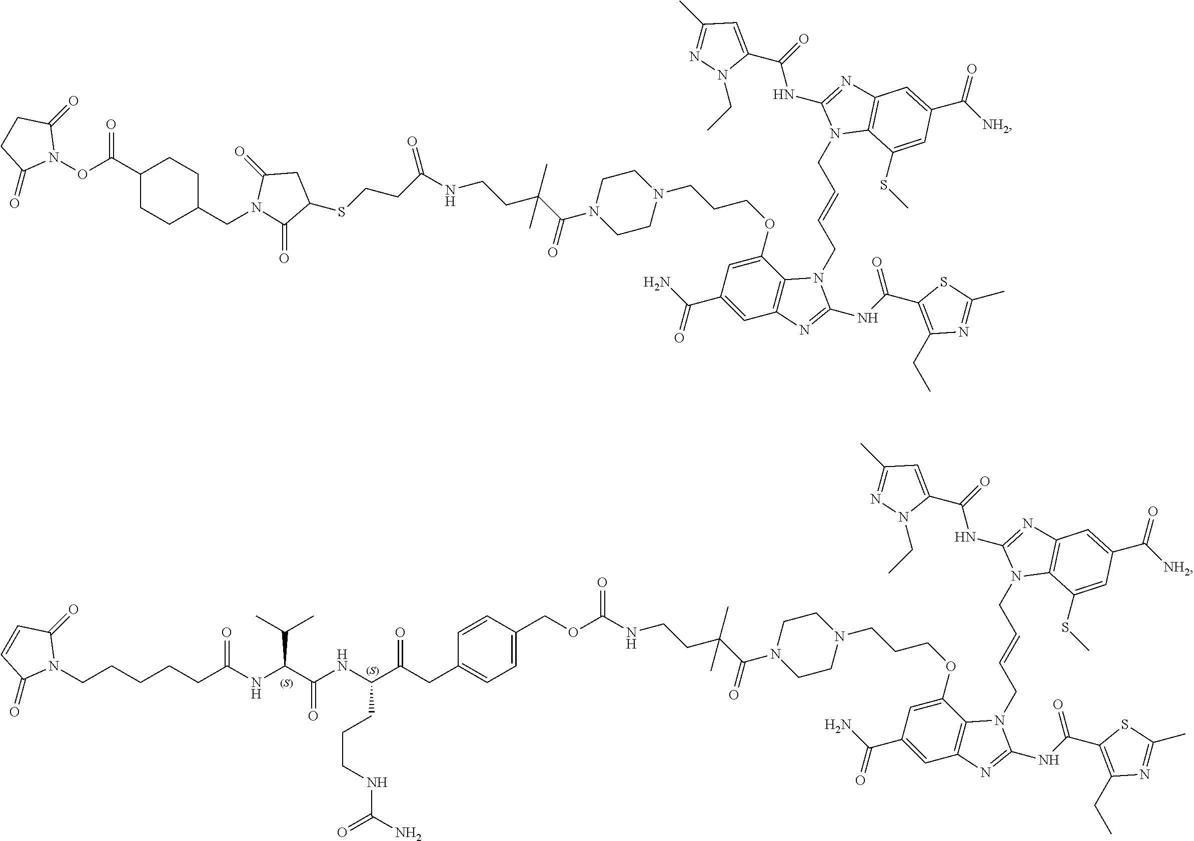

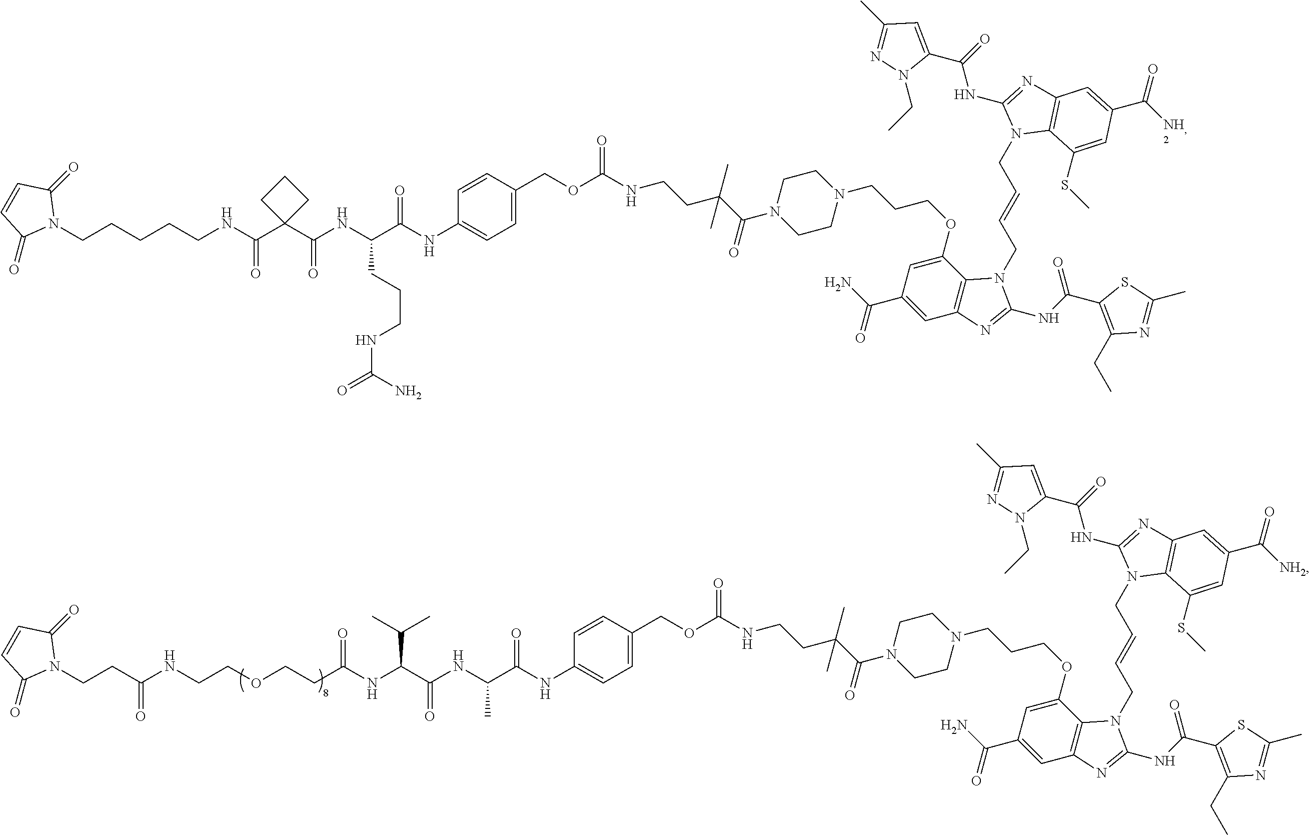

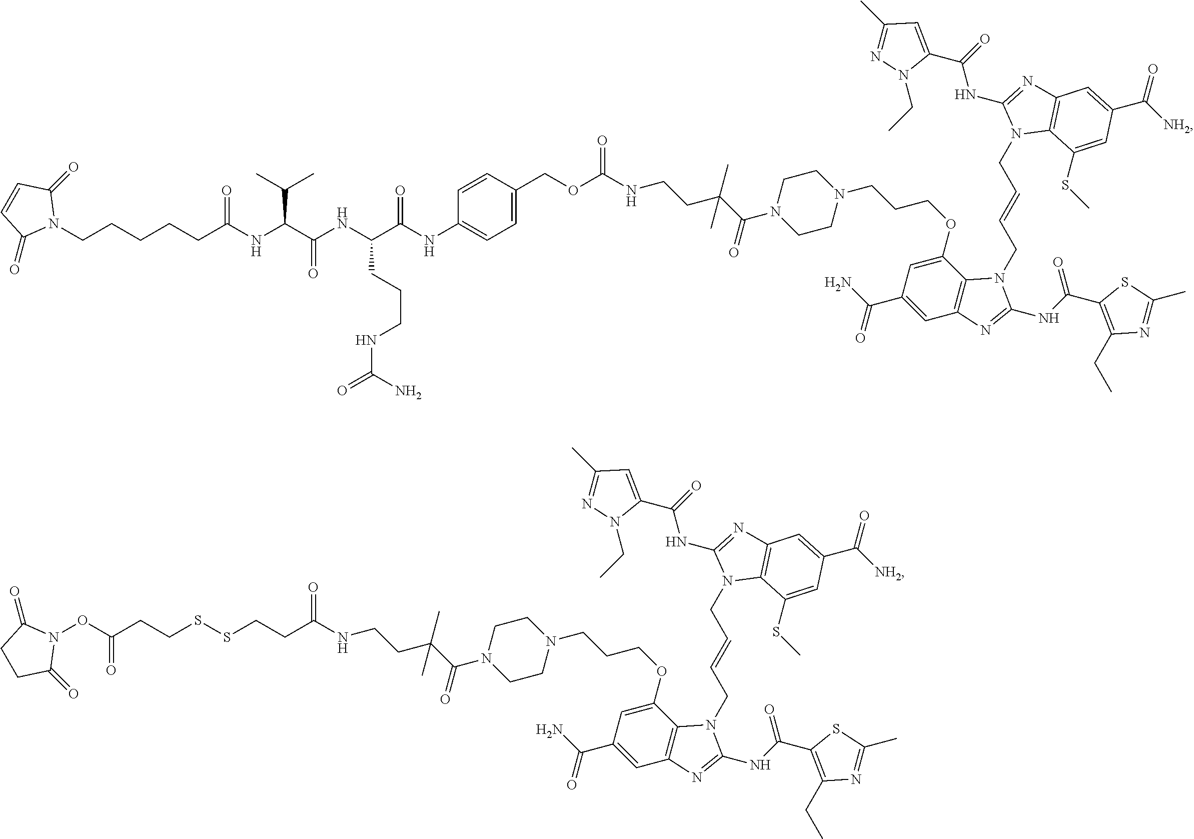 [SMILES: CCc1nc(C)sc1C(=O)Nc1nc2cc(C(N)=O)cc(OCCCN3CCN(C(=O)C(C)(C)CCNC(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)C(C)(C)CCNC(=O)CCSSCCC(=O)ON4C(=O)CCC4=O)CC3)c2n1C/C=C/Cn1c(NC(=O)c2cc(C)nn2CC)nc2cc(C(N)=O)cc(SC)c21]

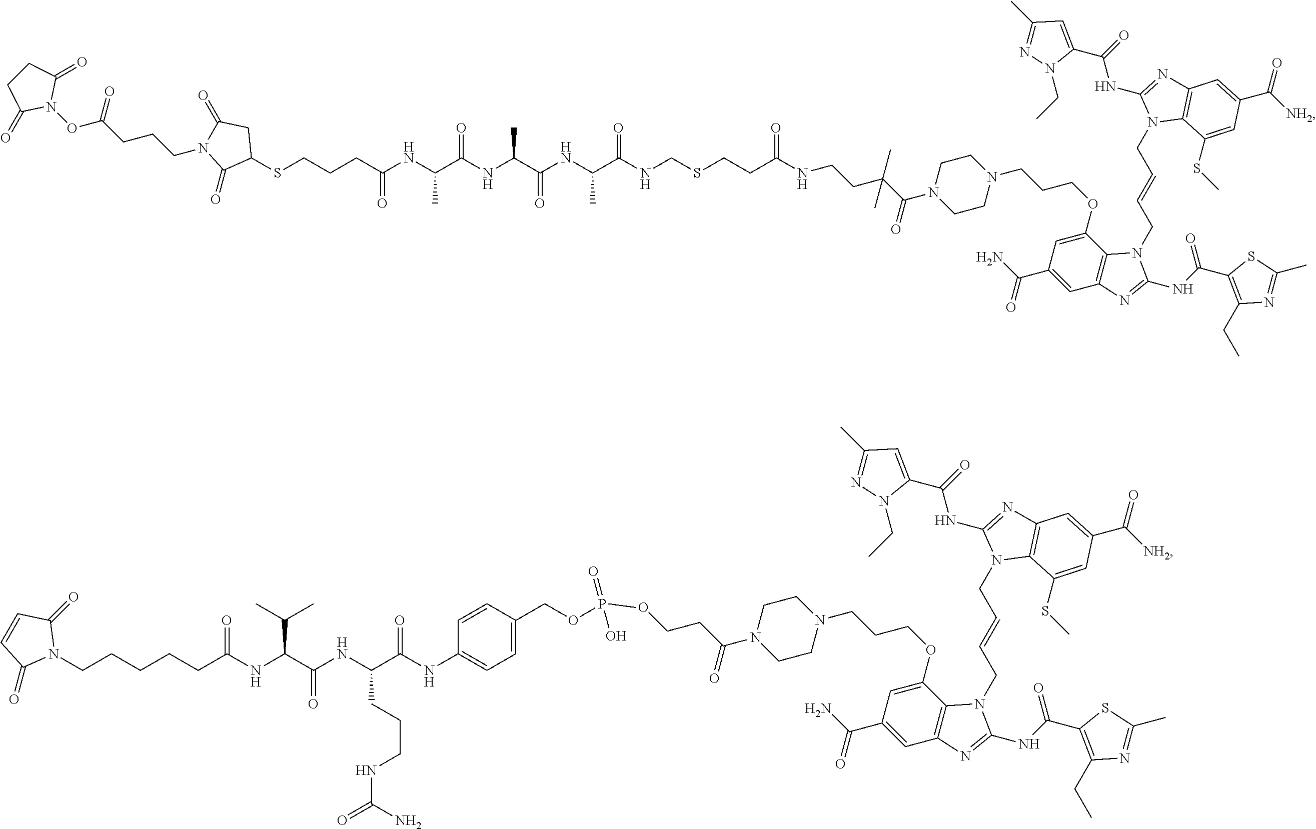 [SMILES: CCc1nc(C)sc1C(=O)Nc1nc2cc(C(N)=O)cc(OCCCN3CCN(C(=O)C(C)(C)CCNC(=O)CCSCNC(=O)[C@H](C)NC(=O)[C@H](C)NC(=O)[C@H](C)NC(=O)CCCSC4CC(=O)N(CCCC(=O)ON5C(=O)CCC5=O)C4=O)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)CCOP(=O)(O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21]

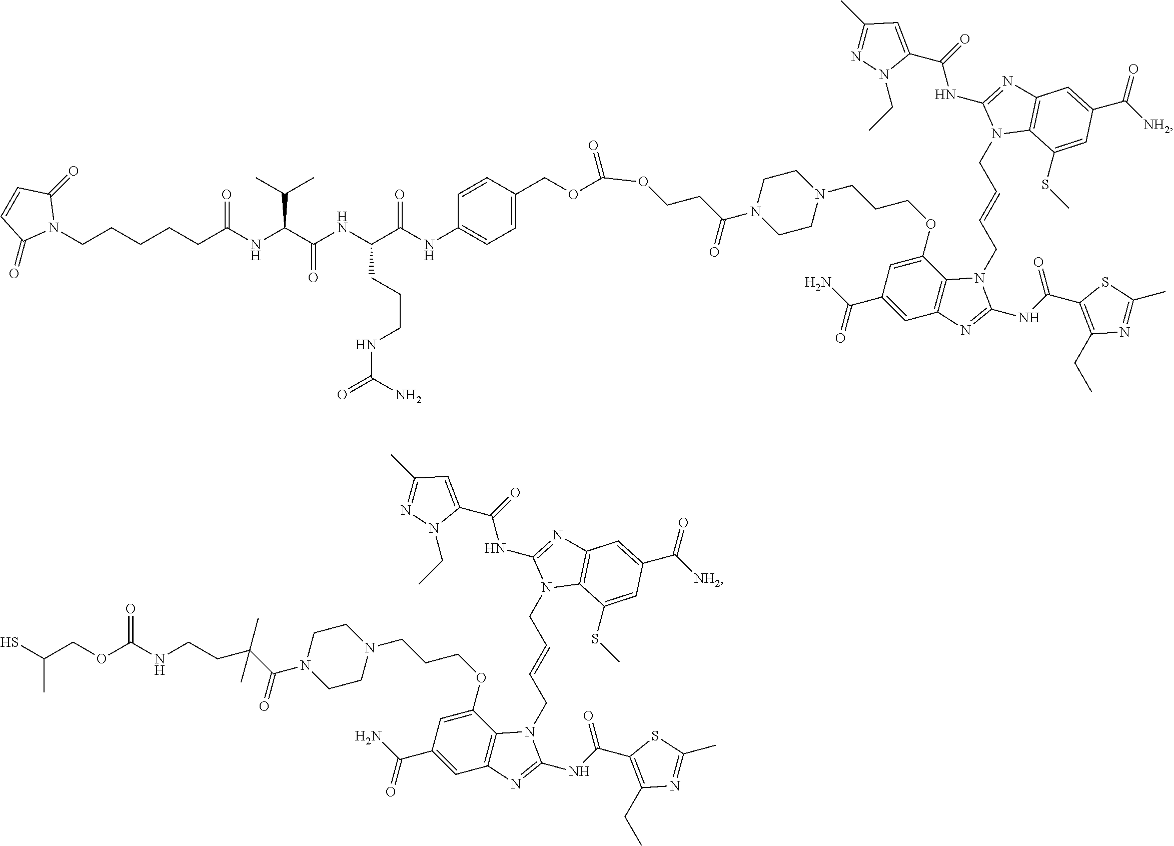 [SMILES: CCc1nc(C)sc1C(=O)Nc1nc2cc(C(N)=O)cc(OCCCN3CCN(C(=O)CCOC(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)C(C)(C)CCNC(=O)OCC(C)S)CC3)c2n1C/C=C/Cn1c(NC(=O)c2cc(C)nn2CC)nc2cc(C(N)=O)cc(SC)c21]

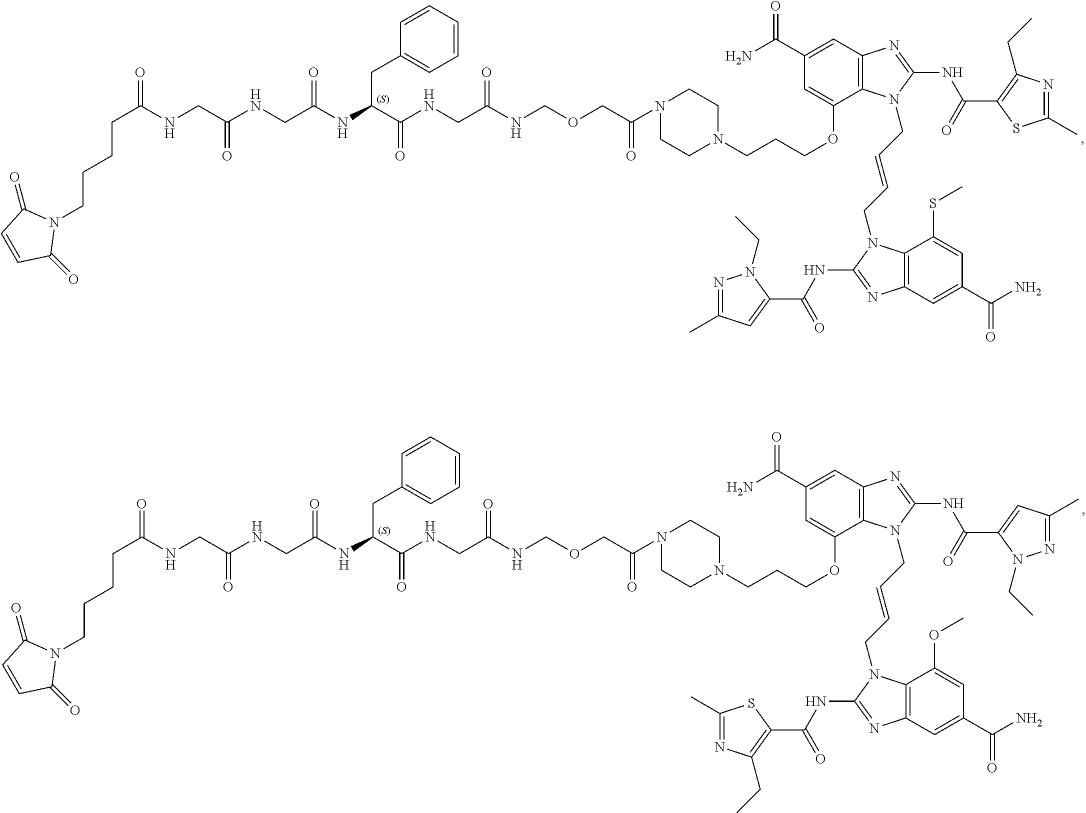

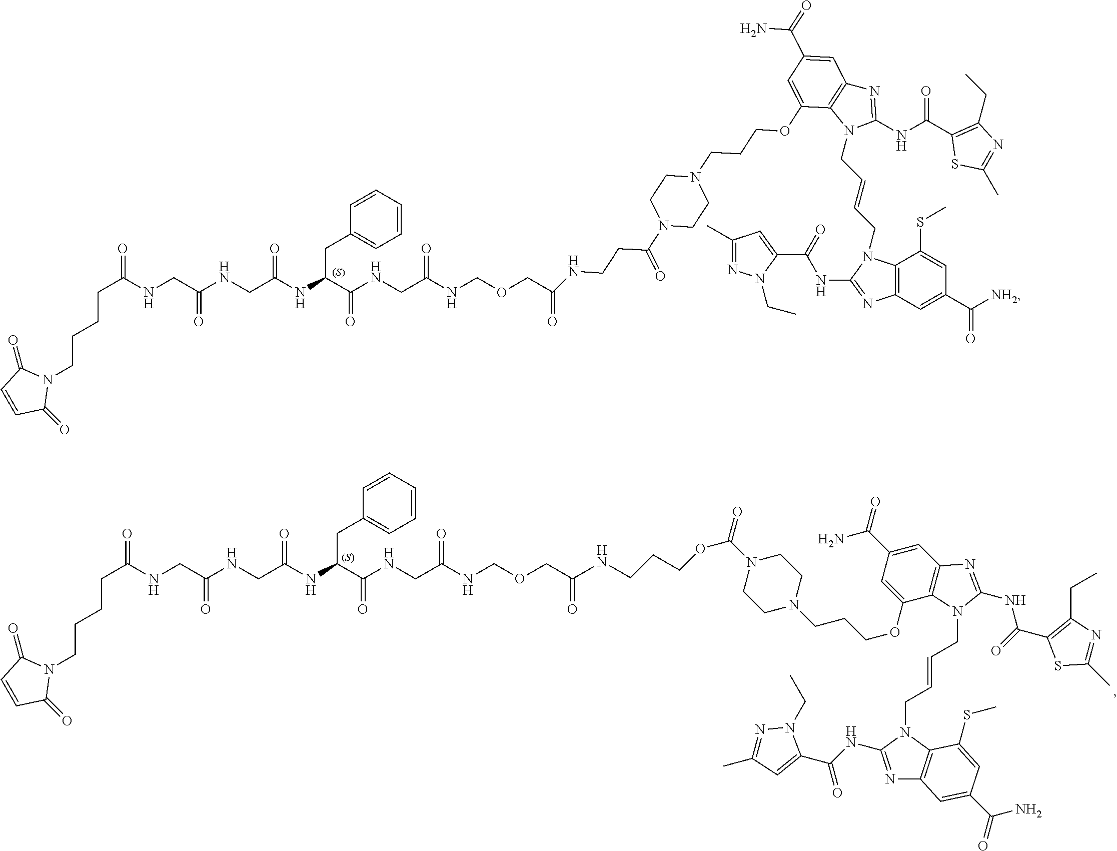

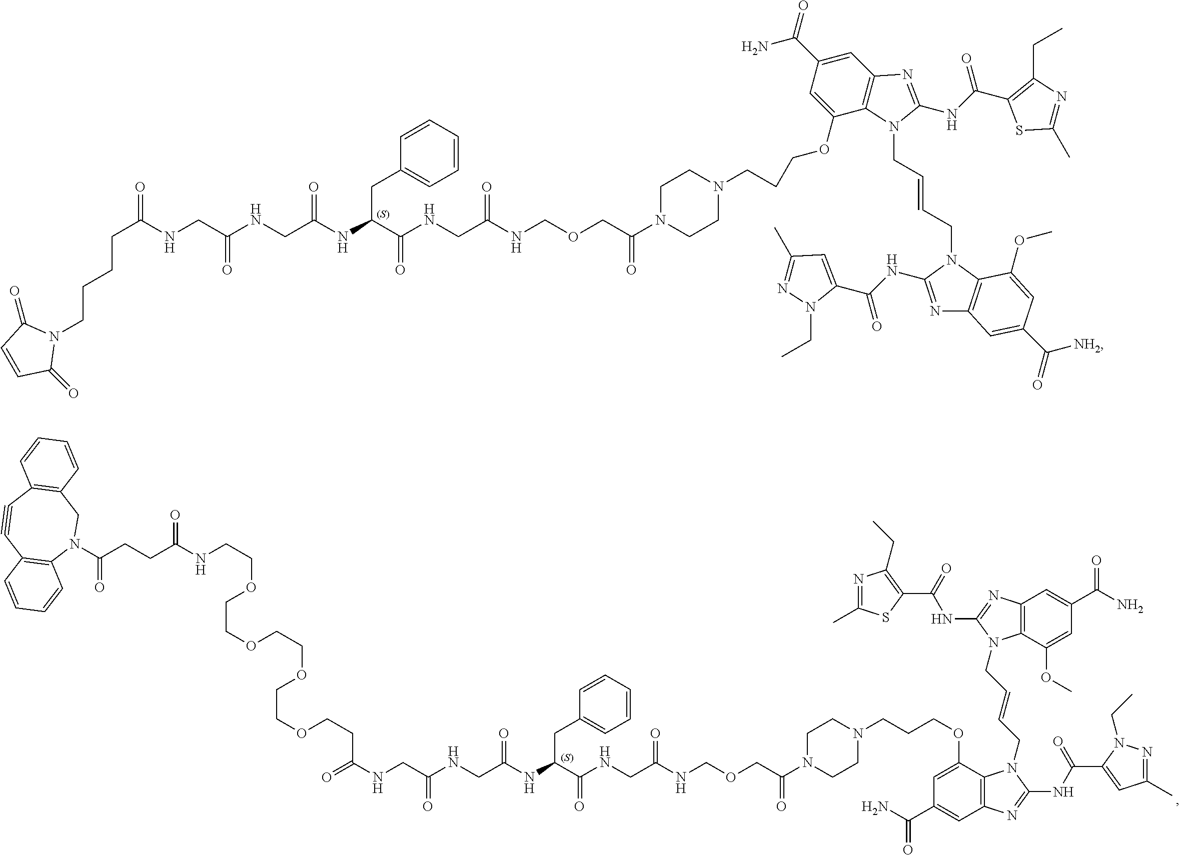

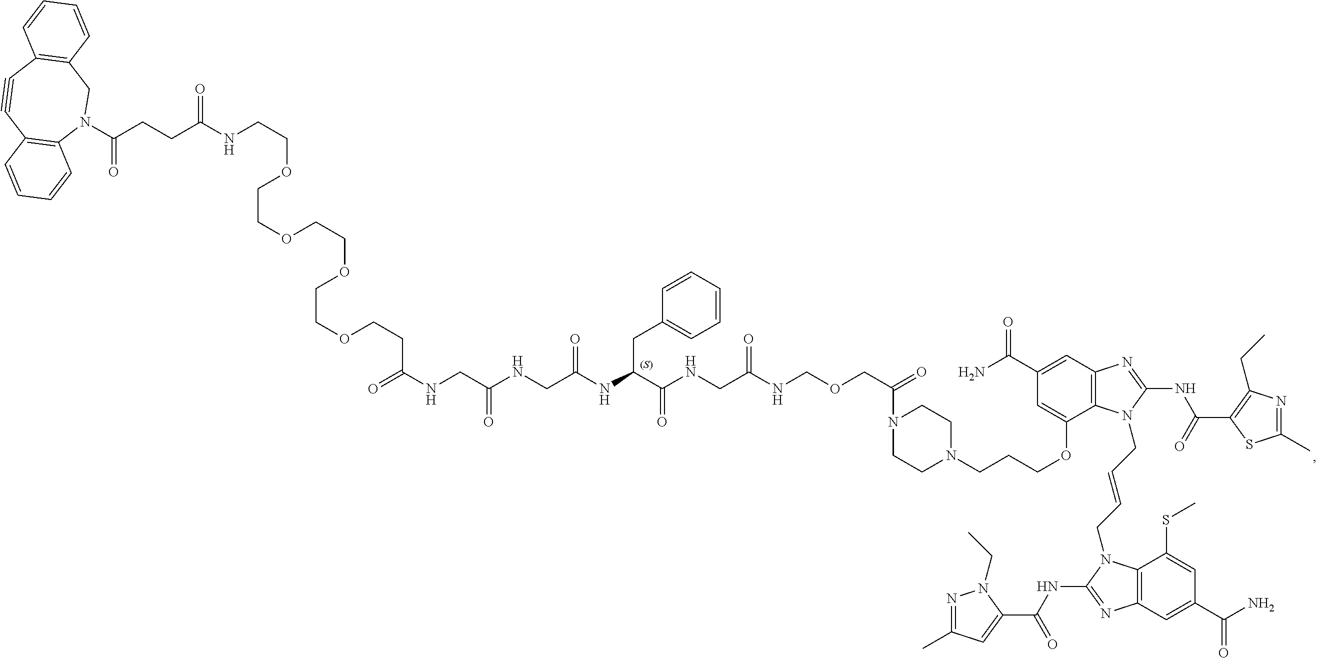

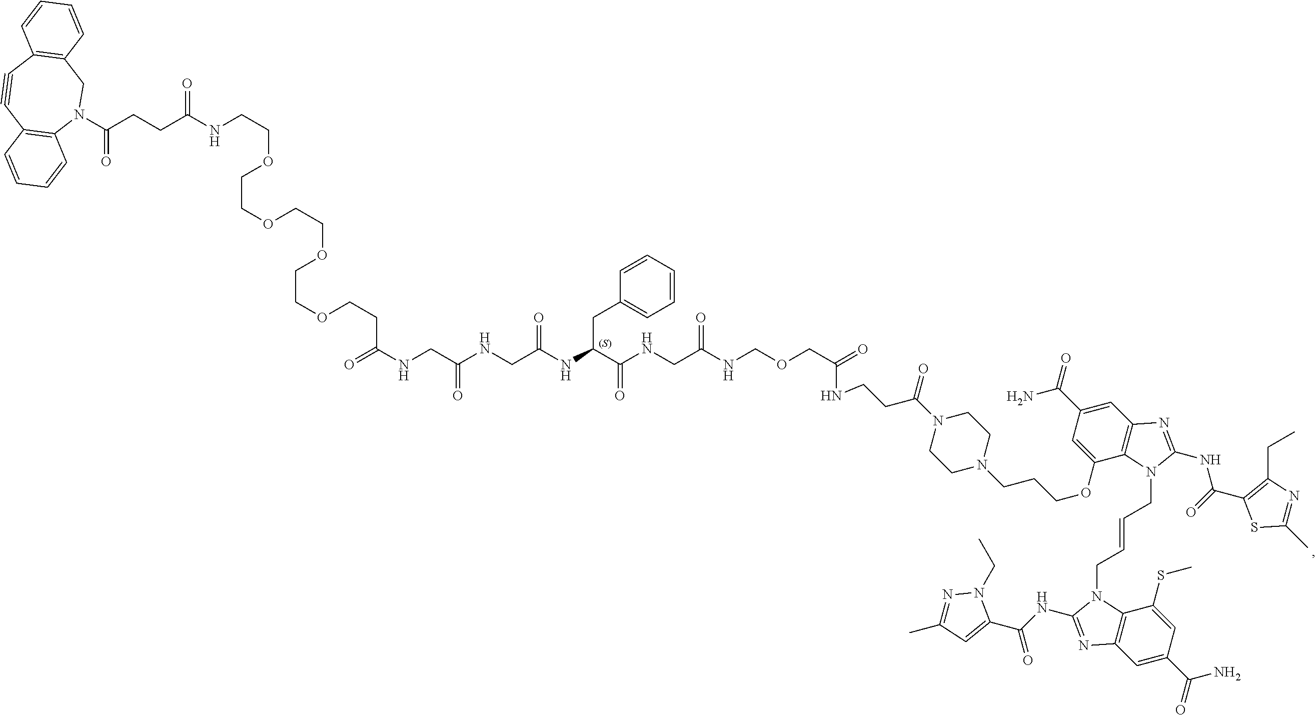

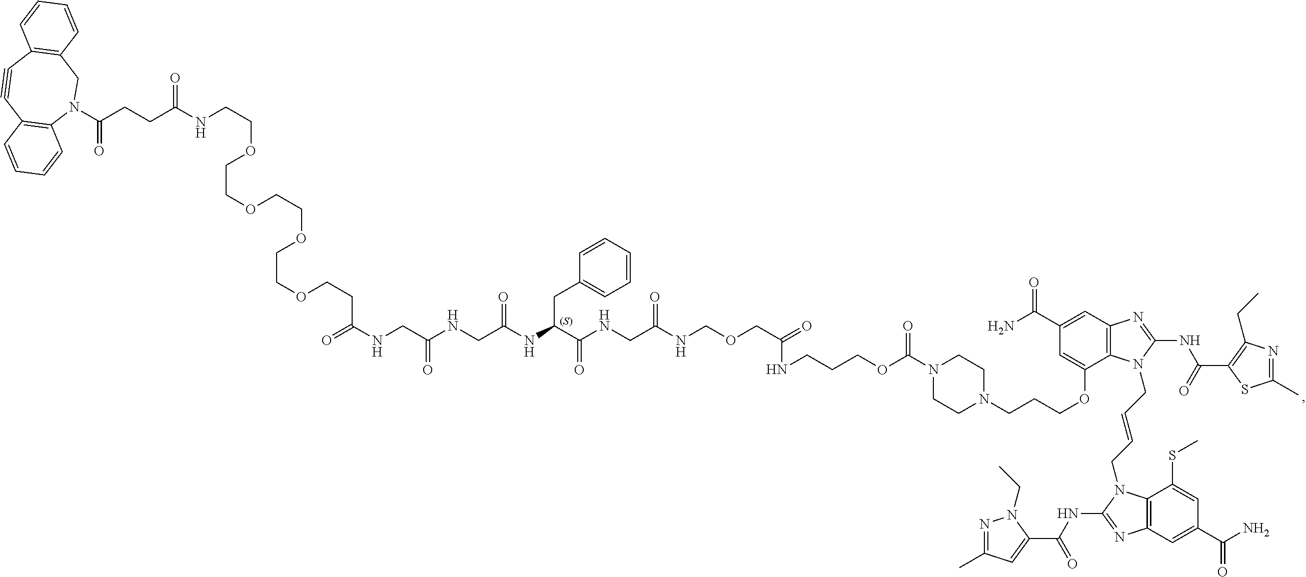

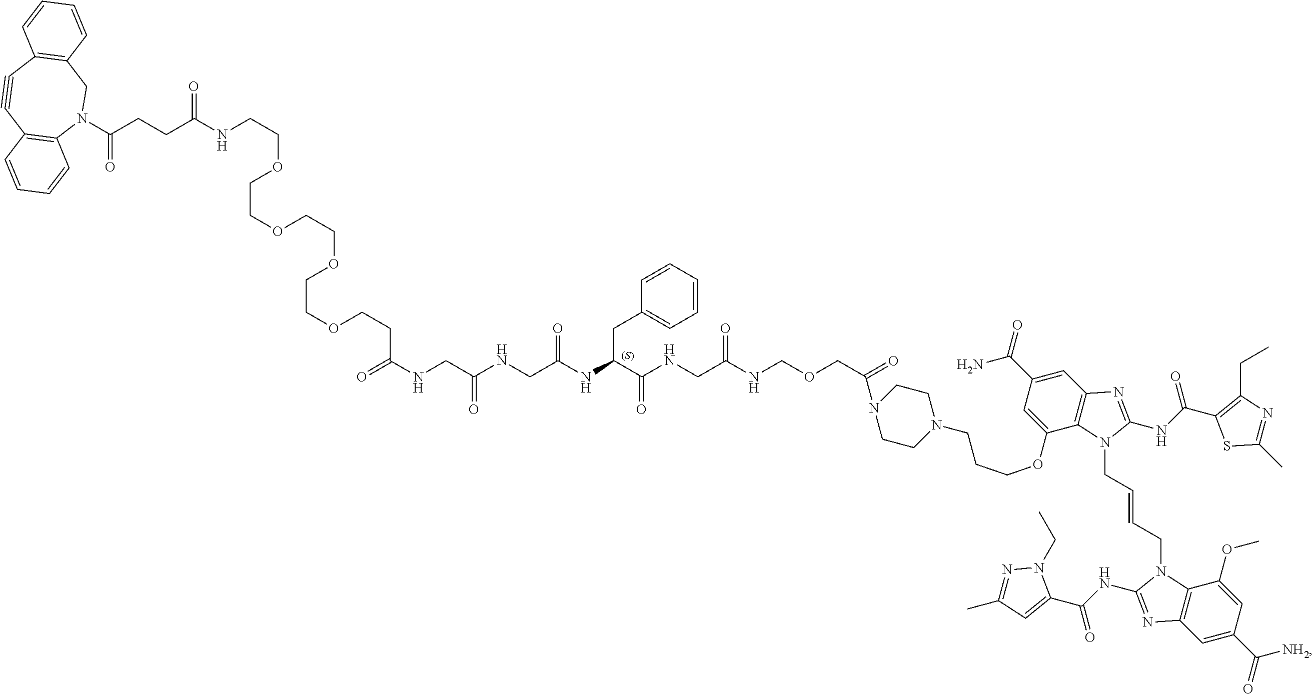

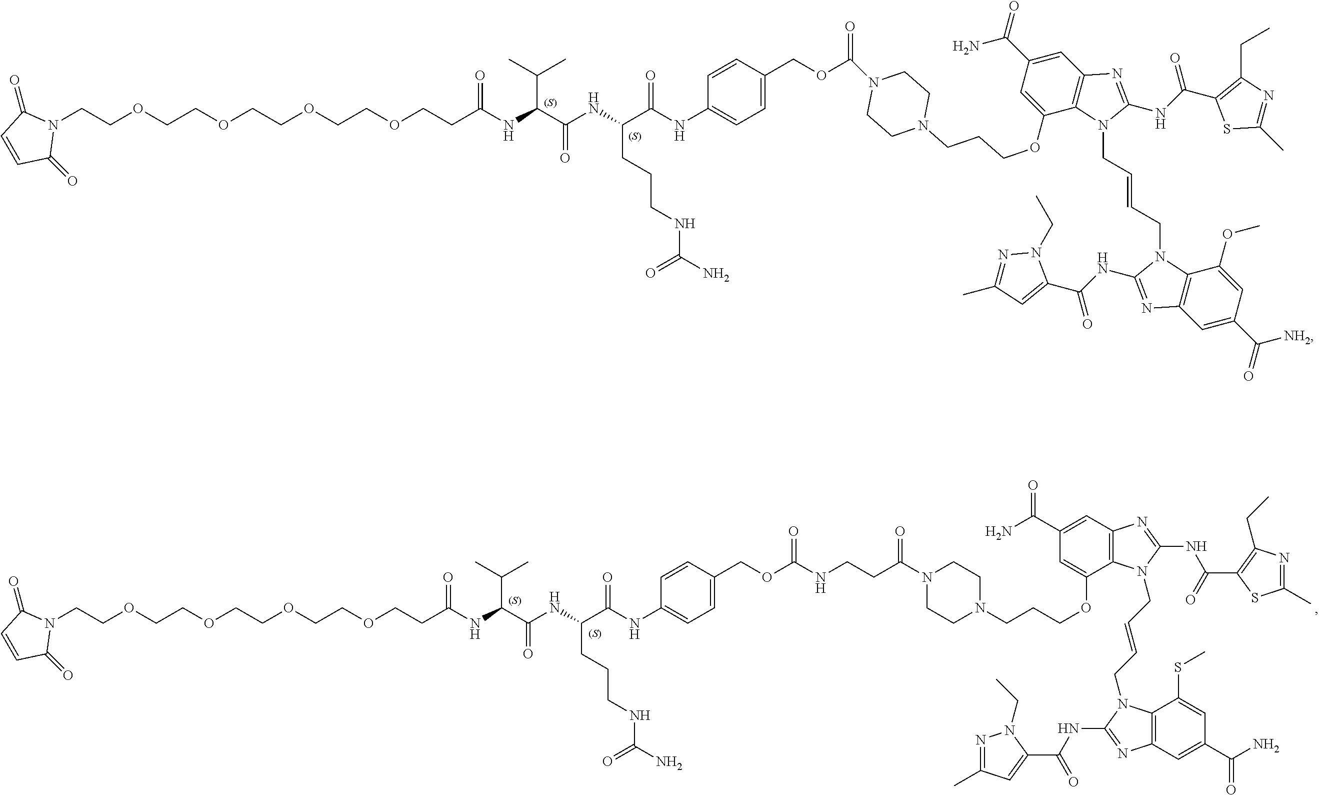

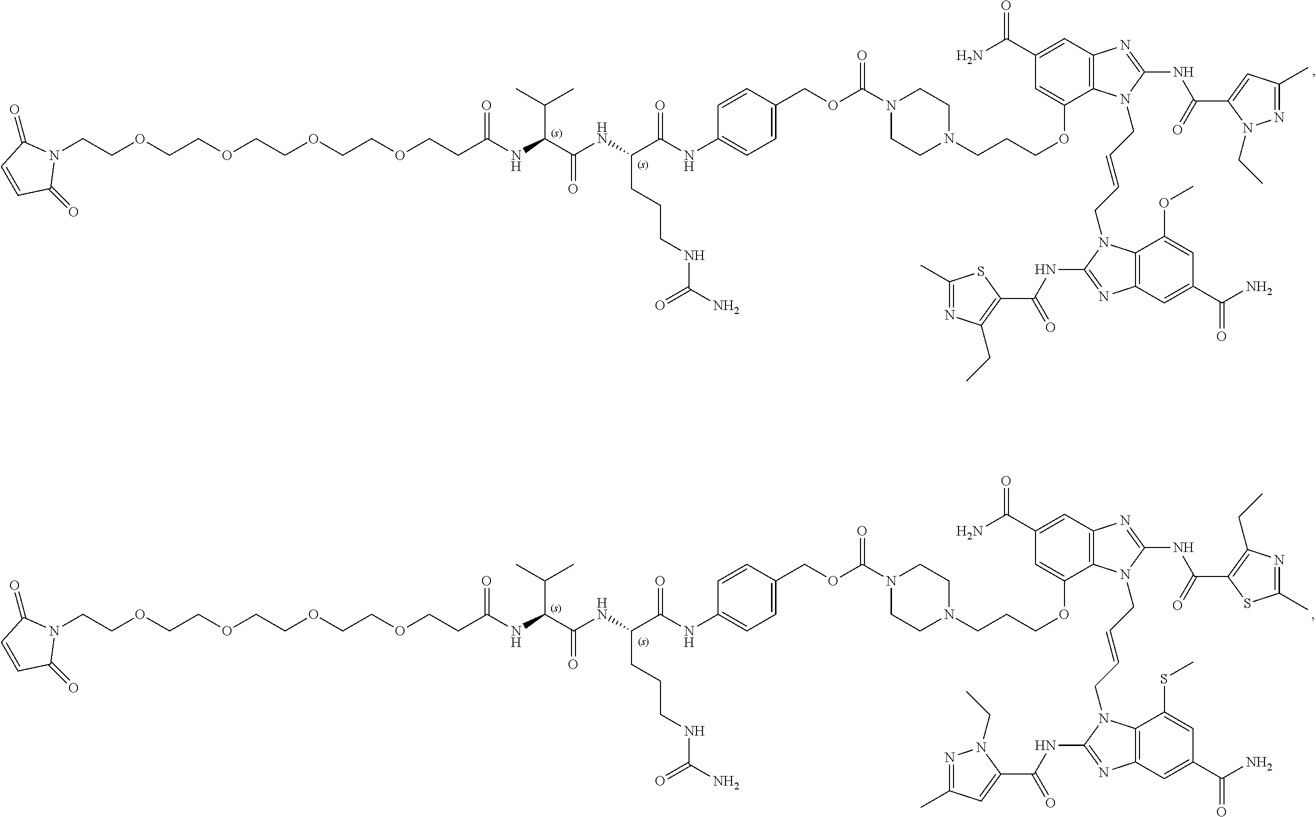 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c21 ; CCc1nc(C)sc1C(=O)Nc1nc2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(SC)c21]

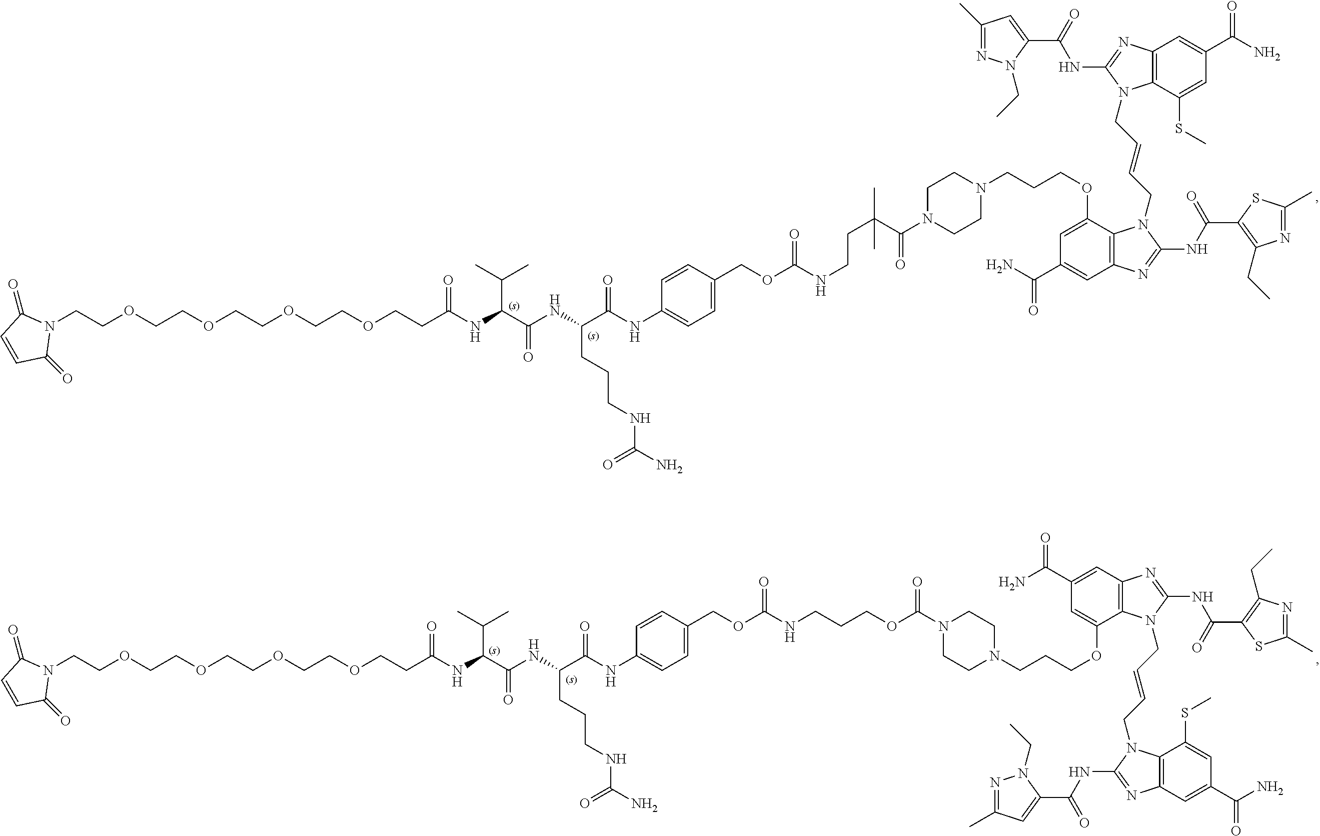 [SMILES: CCc1nc(C)sc1C(=O)Nc1nc2cc(C(N)=O)cc(OCCCN3CCN(C(=O)C(C)(C)CCNC(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)OCCCNC(=O)OCc4ccc(NC(=O)[C@H](CCCNC(N)=O)NC(=O)[C@@H](NC(=O)CCOCCOCCOCCOCCN5C(=O)C=CC5=O)C(C)C)cc4)CC3)c2n1C/C=C/Cn1c(NC(=O)c2cc(C)nn2CC)nc2cc(C(N)=O)cc(SC)c21]

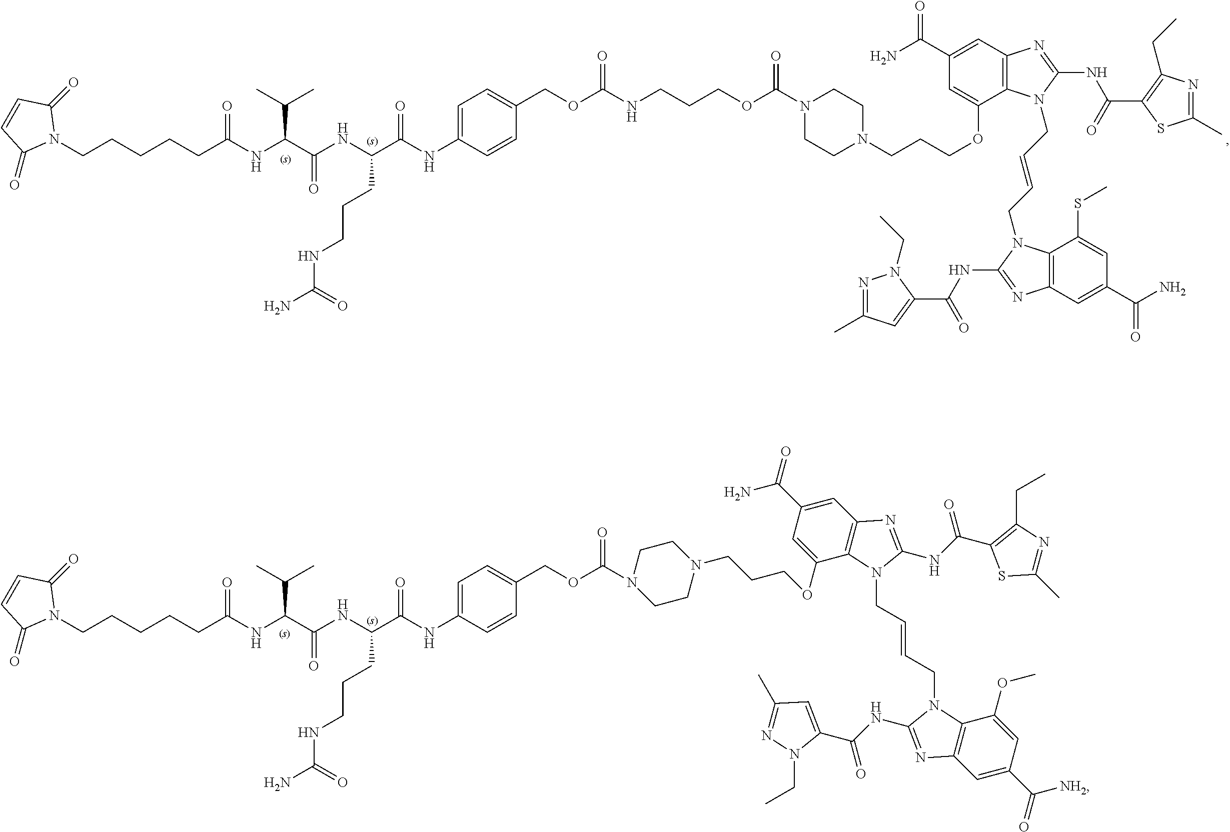 [SMILES: CCc1nc(C)sc1C(=O)Nc1nc2cc(C(N)=O)cc(OCCCN3CCN(C(=O)OCCCNC(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)OCc4ccc(NC(=O)[C@H](CCCNC(N)=O)NC(=O)[C@@H](NC(=O)CCCCCN5C(=O)C=CC5=O)C(C)C)cc4)CC3)c2n1C/C=C/Cn1c(NC(=O)c2cc(C)nn2CC)nc2cc(C(N)=O)cc(OC)c21]

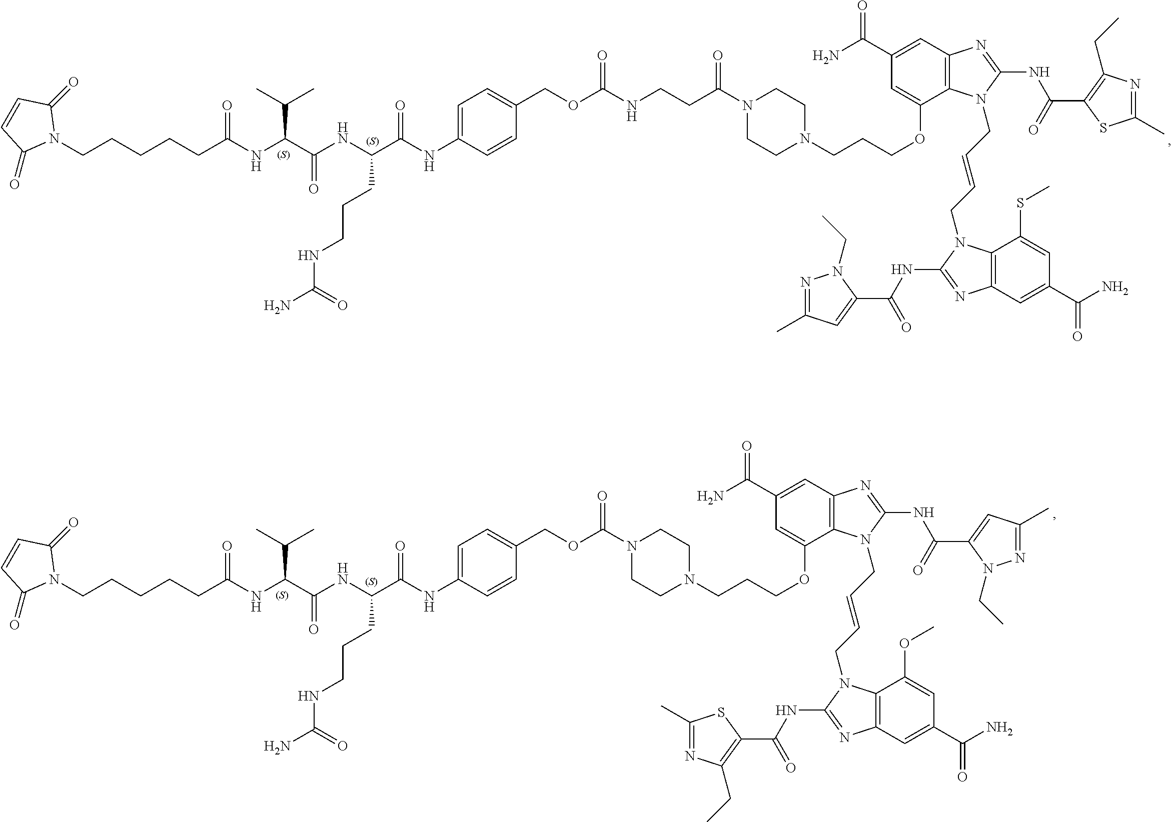

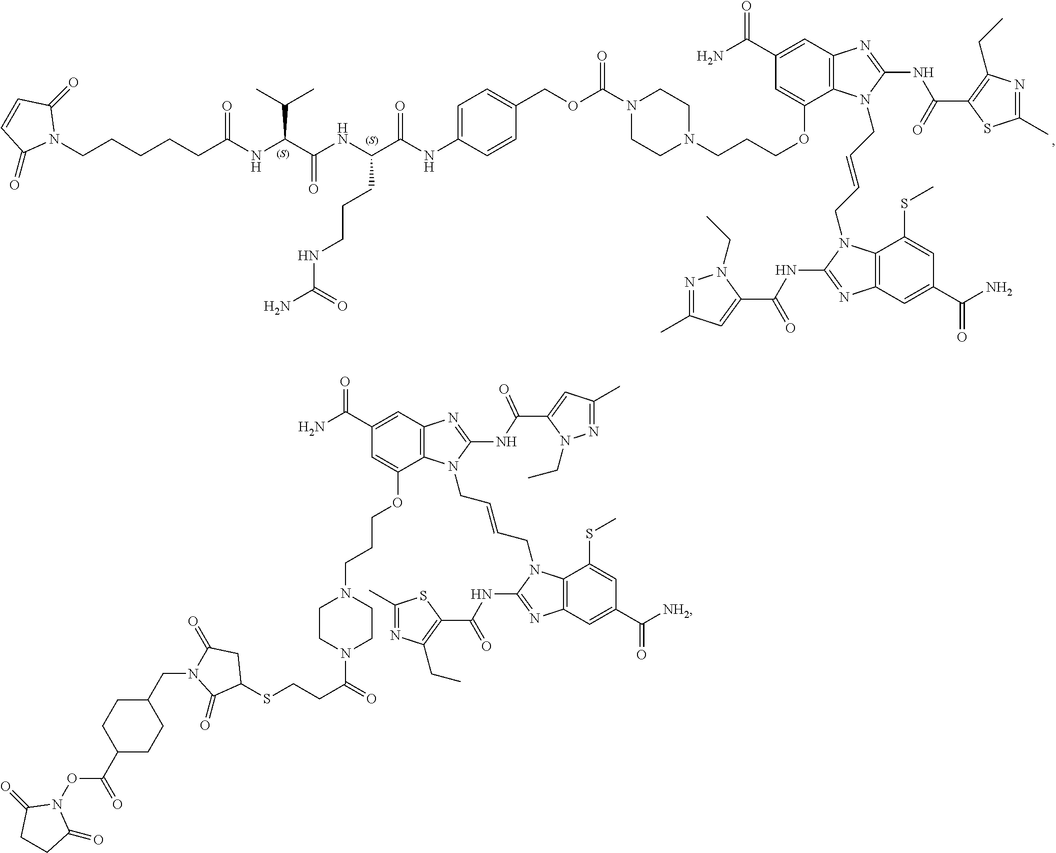

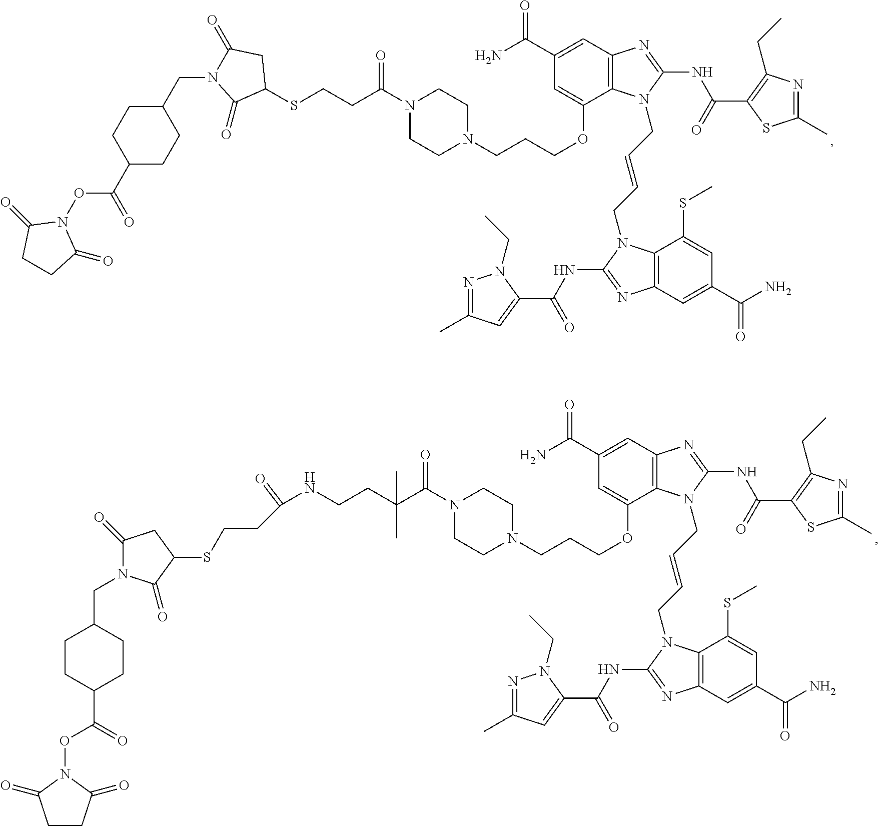 [SMILES: CCc1nc(C)sc1C(=O)Nc1nc2cc(C(N)=O)cc(OCCCN3CCN(C(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)C(C)(C)CCNC(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21]

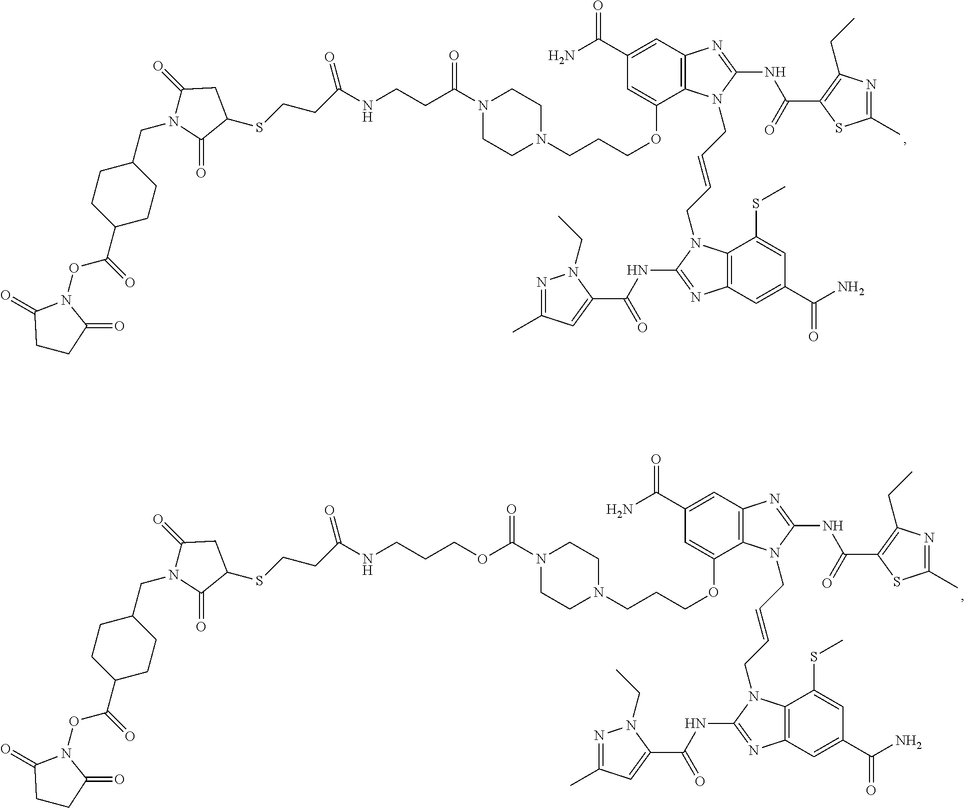 [SMILES: CCc1nc(C)sc1C(=O)Nc1nc2cc(C(N)=O)cc(OCCCN3CCN(C(=O)CCNC(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)OCCCNC(=O)CCSC4CC(=O)N(CC5CCC(C(=O)ON6C(=O)CCC6=O)CC5)C4=O)CC3)c2n1C/C=C/Cn1c(NC(=O)c2cc(C)nn2CC)nc2cc(C(N)=O)cc(SC)c21]

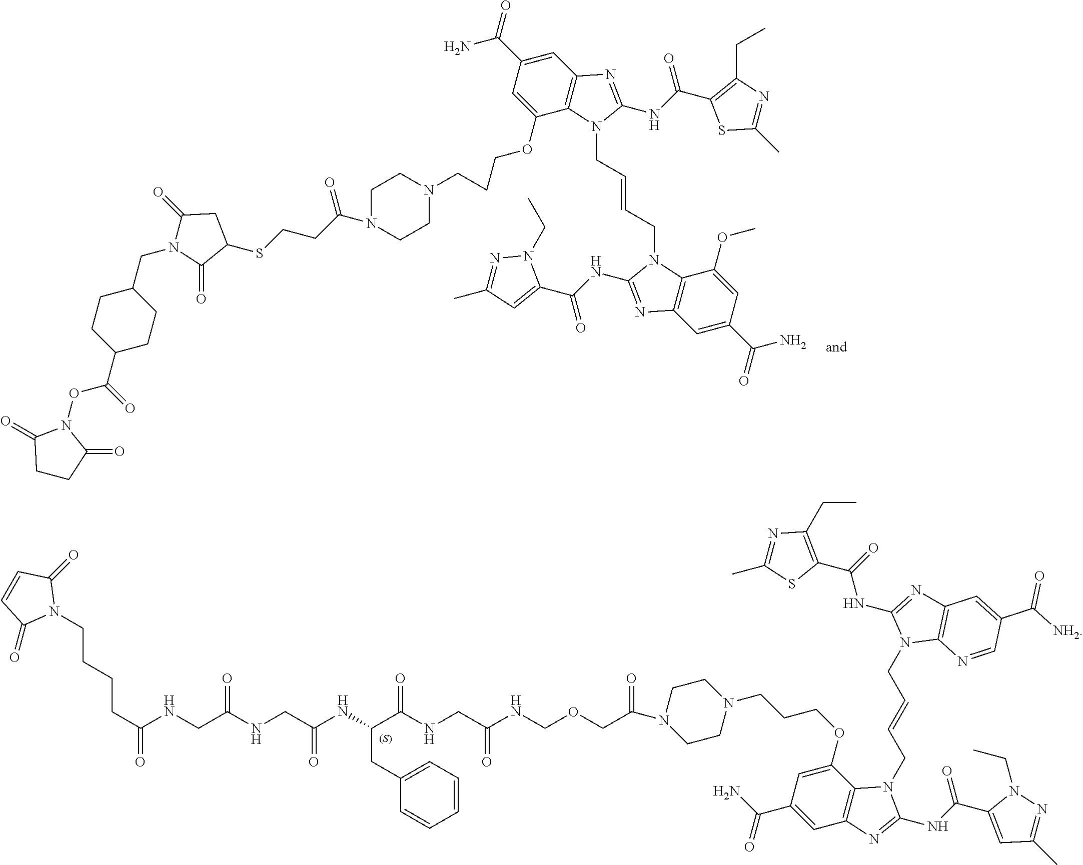

9. A compound of formula (II), or a deuterated compound, a stereoisomer, or a pharmaceutically acceptable salt thereof: 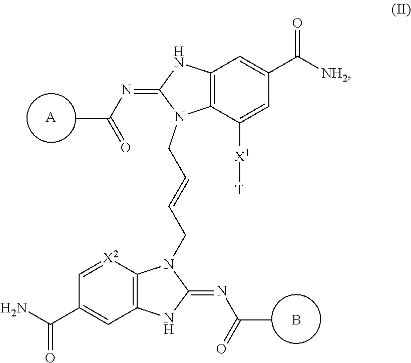
wherein ring A and ring B are independently selected from the group consisting of 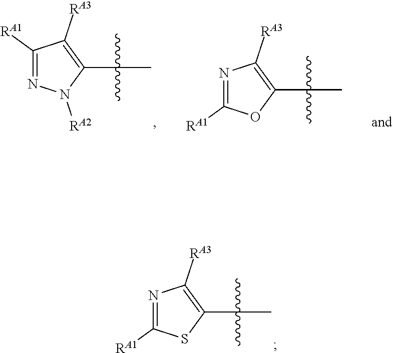
R^{A1} and R^{A3} are independently selected from the group consisting of hydrogen, halogen, —CN, unsubstituted —C₁₋₆ alkyl and halogenated —C₁₋₆ alkyl;
R^{A2} is selected from the group consisting of hydrogen, unsubstituted —C₁₋₆ alkyl and halogenated —C₁₋₆ alkyl;
X¹ is O or S;
X² is N or CR^{X};
R^{X} is selected from the group consisting of hydrogen, halogen, —CN, unsubstituted —C₁₋₆ alkyl, halogenated —C₁₋₆ alkyl, —R¹, —OR¹, —SR¹ and —NR¹R^{1′};
R¹ and R^{1′} are independently selected from the group consisting of hydrogen, —C₁₋₈ alkyl, —C₁₋₈ alkylene-NR²R^{2′}, —C₁₋₈ alkylene-OR², —C₀₋₈ alkylene-(3-10 membered cycloalkyl), —C₀₋₈ alkylene-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in an alkylene group are adapted to be replaced with an oxygen atom;
R² and R^{2′} are independently selected from the group consisting of hydrogen, —C₁₋₆ alkyl, —C₀₋₈ alkylene-(3-10 membered cycloalkyl), —C₀₋₈ alkyl-(3-10 membered heterocycloalkyl), —C₀₋₈ alkylene-(5-12 membered spirocycloalkyl), —C₀₋₈ alkylene-(5-12 membered spiroheterocycloalkyl), —C₀₋₈ alkylene-(5-12 membered bridged cycloalkyl) and —C₀₋₈ alkylene-(5-12 membered bridged heterocycloalkyl); wherein one or two carbon atoms in an alkylene group are adapted to be replaced with an oxygen atom;
T is -M-L^{T};
M is selected from the group consisting of —C₁₋₈ alkylene-, —C₀₋₈ alkylene-(3-10 membered cycloalkyl)-, —C₀₋₈ alkylene-(3-10 membered heterocycloalkyl)-, —C₀₋₈ alkylene-(5-12 membered spirocycloalkyl)-, —C₀₋₈ alkylene-(5-12 membered spiroheterocycloalkyl)-, —C₀₋₈ alkylene-(5-12 membered bridged cycloalkyl)- and —C₀₋₈ alkylene-(5-12 membered bridged heterocycloalkyl)-; wherein one or two carbon atoms in an alkylene group are adapted to be replaced with an oxygen atom;
L^{T} is selected from the group consisting of —C(O)—C₁₋₈ alkylene-NH₂, —C(O)—C₁₋₈ alkylene-NH(C₁₋₆ alkyl), —C(O)O—C₁₋₈ alkylene-NH₂, —C(O)O—C₁₋₈ alkylene-NH(C₁₋₆ alkyl), —C(O)NH—C₁₋₈ alkylene-NH₂, —C(O)NH—C₁₋₈ alkylene-NH(C₁₋₆ alkyl), —C₁₋₈ alkylene-NH₂, —C₁₋₈ alkylene-NH(C₁₋₆ alkyl), —C(O)—C₁₋₈ alkylene-(3-10 membered cycloalkyl), —C(O)O—C₁₋₈ alkylene-(3-10 membered cycloalkyl), —C(O)NH—C₁₋₈ alkylene-(3-10 membered cycloalkyl), —C₁₋₈ alkylene-(3-10 membered cycloalkyl), —C(O)—C₁₋₈ alkylene-(3-10 membered heterocycloalkyl), —C(O)O—C₁₋₈ alkylene-(3-10 membered heterocycloalkyl), —C(O)NH—C₁₋₈ alkylene-(3-10 membered heterocycloalkyl), —C₁₋₈ alkylene-(3-10 membered heterocycloalkyl), —C(O)—C₁₋₈ alkylene-(5-12 membered spirocycloalkyl), —C(O)O—C₁₋₈ alkylene-(5-12 membered spirocycloalkyl), —C(O)NH—C₁₋₈ alkylene-(5-12 membered spirocycloalkyl), —C₁₋₈ alkylene-(5-12 membered spirocycloalkyl), —C(O)—C₁₋₈ alkylene-(5-12 membered spiroheterocycloalkyl), —C(O)O—C₁₋₈ alkylene-(5-12 membered spiroheterocycloalkyl), —C(O)NH—C₁₋₈ alkylene-(5-12 membered spiroheterocycloalkyl), —C₁₋₈ alkylene-(5-12 membered spiroheterocycloalkyl), —C(O)—C₁₋₈ alkylene-(5-12 membered bridged cycloalkyl), —C(O)O—C₁₋₈ alkylene-(5-12 membered bridged cycloalkyl), —C(O)NH—C₁₋₈ alkylene-(5-12 membered bridged cycloalkyl), —C₁₋₈ alkylene-(5-12 membered bridged cycloalkyl), —C(O)—C₁₋₈ alkylene-(5-12 membered bridged heterocycloalkyl), —C(O)O—C₁₋₈ alkylene-(5-12 membered bridged heterocycloalkyl), —C(O)NH—C₁₋₈ alkylene-(5-12 membered bridged heterocycloalkyl), —C₁₋₈ alkylene-(5-12 membered bridged heterocycloalkyl), —C(O)—C₁₋₈ alkylene-OH, —C(O)O—C₁₋₈ alkylene-OH, —C(O)NH—C₁₋₈ alkylene-OH, —C₁₋₈ alkylene-OH, —S(O)₂—C₁₋₈ alkylene-NH₂, —S(O)₂—C₁₋₈ alkylene-NH(C₁₋₆ alkyl), —S(O)₂O—C₁₋₈ alkylene-NH₂, —S(O)₂O—C₁₋₈ alkylene-NH(C₁₋₆ alkyl), —S(O)₂NH—C₁₋₈ alkylene-NH₂, —S(O)₂NH—C₁₋₈ alkylene-NH(C₁₋₆ alkyl), —S(O)₂—C₁₋₈ alkylene-(3-10 membered cycloalkyl), —S(O)₂O—C₁₋₈ alkylene-(3-10 membered cycloalkyl), —S(O)₂NH—C₁₋₈ alkylene-(3-10 membered cycloalkyl), —S(O)₂—C₁₋₈ alkylene-(3-10 membered heterocycloalkyl), —S(O)₂O—C₁₋₈ alkylene-(3-10 membered heterocycloalkyl), —S(O)₂NH—C₁₋₈ alkylene-(3-10 membered heterocycloalkyl), —S(O)₂—C₁₋₈ alkylene-(5-12 membered spirocycloalkyl), —S(O)₂O—C₁₋₈ alkylene-(5-12 membered spirocycloalkyl), —S(O)₂NH—C₁₋₈ alkylene-(5-12 membered spirocycloalkyl), —S(O)₂—C₁₋₈ alkylene-(5-12 membered spiroheterocycloalkyl), —S(O)₂O—C₁₋₈ alkylene-(5-12 membered spiroheterocycloalkyl), —S(O)₂NH—C₁₋₈ alkylene-(5-12 membered spiroheterocycloalkyl), —S(O)₂—C₁₋₈ alkylene-(5-12 membered bridged cycloalkyl), —S(O)₂O—C₁₋₈ alkylene-(5-12 membered bridged cycloalkyl), —S(O)₂NH—C₁₋₈ alkylene-(5-12 membered bridged cycloalkyl), —S(O)₂—C₁₋₈ alkylene-(5-12 membered bridged heterocycloalkyl), —S(O)₂O—C₁₋₈ alkylene-(5-12 membered bridged heterocycloalkyl), —S(O)₂NH—C₁₋₈ alkylene-(5-12 membered bridged heterocycloalkyl), —S(O)₂—C₁₋₈ alkylene-OH, —S(O)₂O—C₁₋₈ alkylene-OH and —S(O)₂NH—C₁₋₈ alkylene-OH; wherein one or two carbon atoms in an alkylene group are adapted to be replaced with an oxygen atom.

10. The compound of claim 9, wherein M is selected from the group consisting of 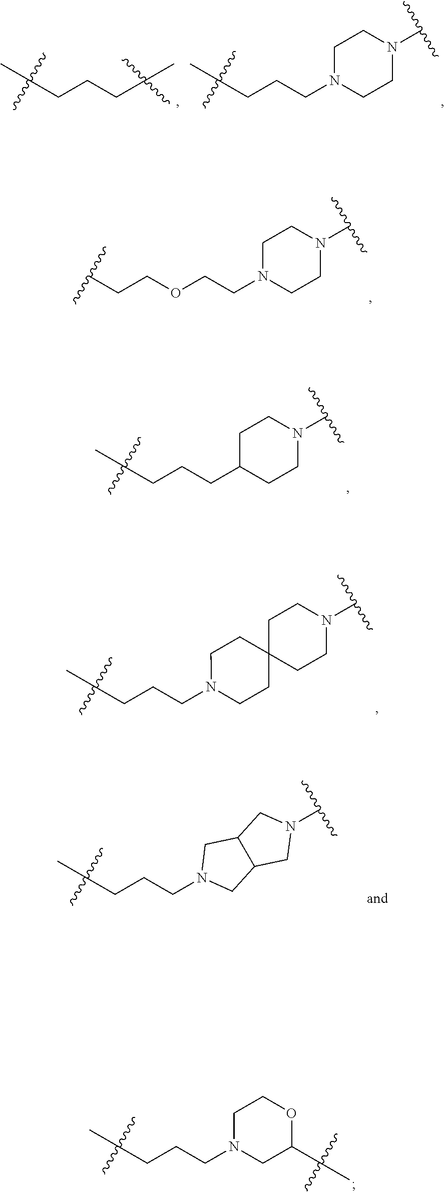
L^{T} is selected from the group consisting of 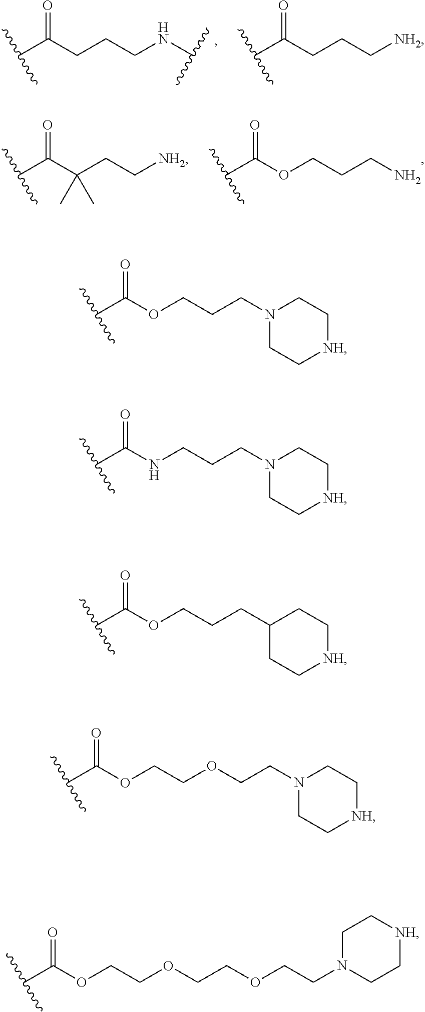 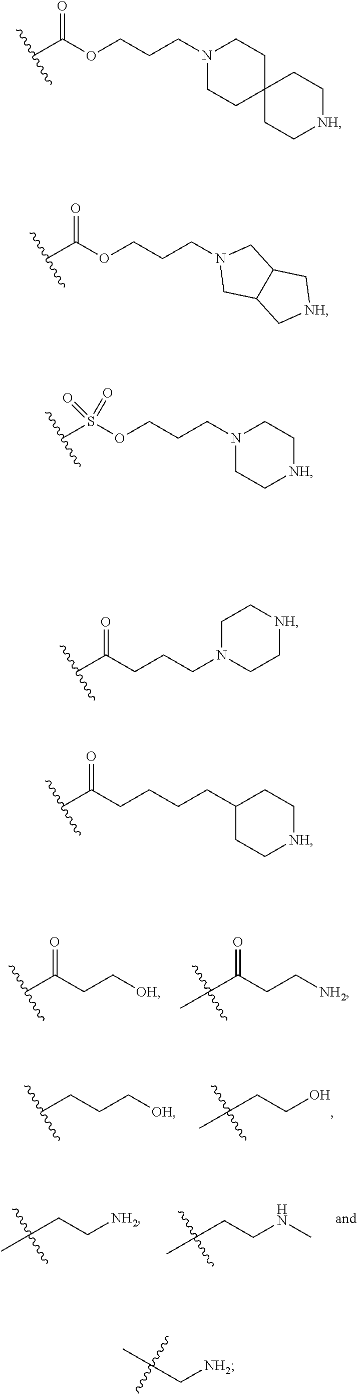
the ring A and the ring B are independently selected from the group consisting of 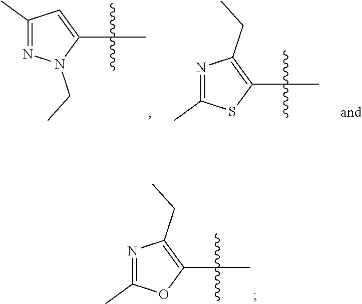
X² is N or CR^{X};
R^{X} is —OR¹ or —SR¹; and
R¹ is methyl or -propylidene-OH.

11. A compound of formula (III), or a deuterated compound, a stereoisomer, or a pharmaceutically acceptable salt thereof: 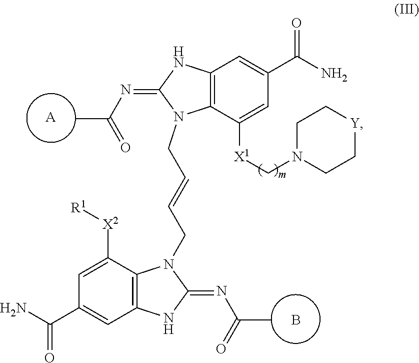
wherein ring A and ring B are independently 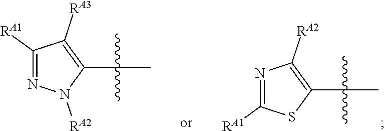
and at least one of the ring A and the ring B is 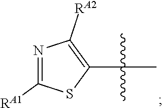
R^{A1} and R^{A2} are independently hydrogen, or —C₁₋₆ alkyl;
R^{A3} is selected from the group consisting of hydrogen, halogen and —C₁₋₆ alkyl;
X¹ and X² are independently O or S;
m is 1, 2, 3, 4, 5 or 6;
Y is O or NR^{Y};
R^{Y} is selected from the group consisting of —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆ alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in an alkylene group are adapted to be replaced with an oxygen atom;
R¹ is —C₁₋₆ alkyl, —C₁₋₆ alkylene-NH₂ or —C₁₋₆ alkylene-NH—R²; and when Y is O, R¹ is not —C₁₋₆ alkyl;
R² is selected from the group consisting of —C₁₋₆ alkyl, —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆ alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in an alkylene group are adapted to be replaced with an oxygen atom.

12. The compound of claim 11, wherein the compound is represented by 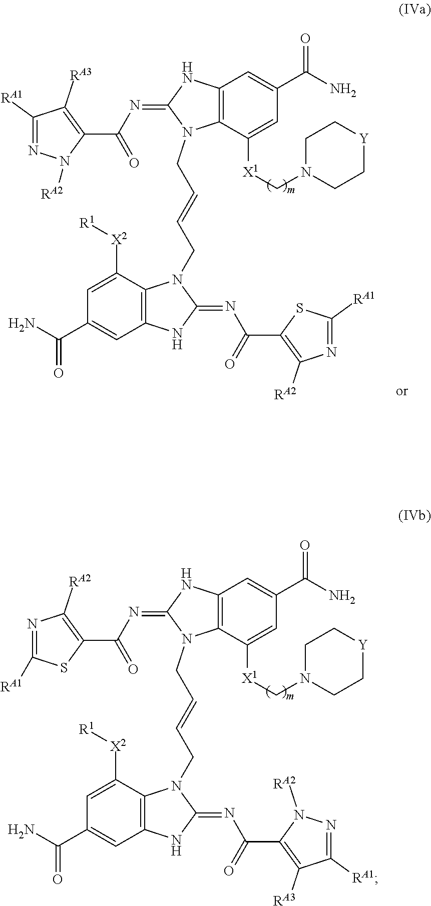
wherein R^{A1} is selected from the group consisting of methyl, ethyl, n-propyl and isopropyl; R^{A2} is selected from the group consisting of methyl, ethyl, n-propyl and isopropyl; and R^{A3} is selected from the group consisting of hydrogen, fluorine, chlorine, methyl and ethyl;
X¹ is O or S; X² is O or S;
m is 1, 2, 3, 4, 5 or 6;
R¹ is selected from the group consisting of methyl, ethyl, n-propyl and isopropyl;
Y is NR^{Y}; and
R^{Y} is selected from the group consisting of —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆ alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in an alkylene group are adapted to be replaced with an oxygen atom.

13. The compound of claim 12, wherein R^{Y} is selected from the group consisting of

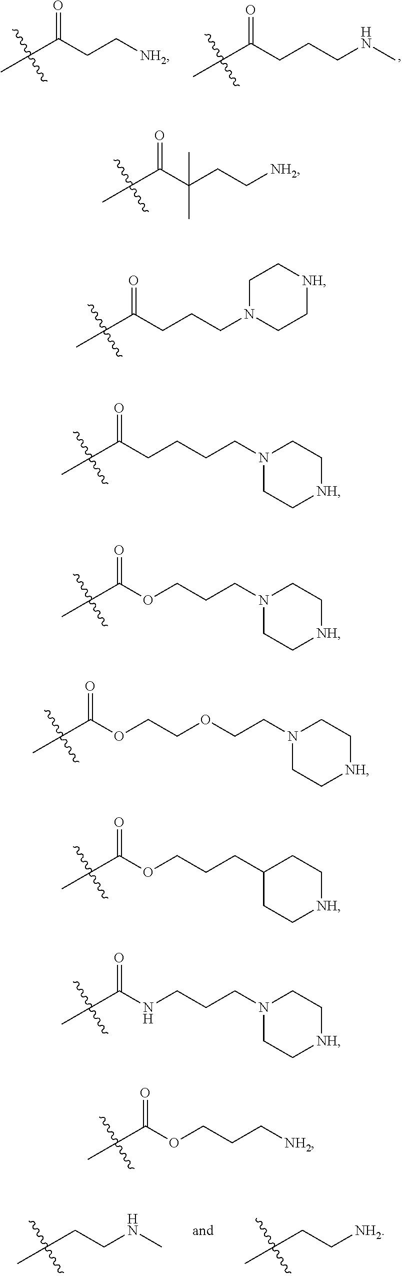

14. The compound of claim 11, wherein the compound is represented by 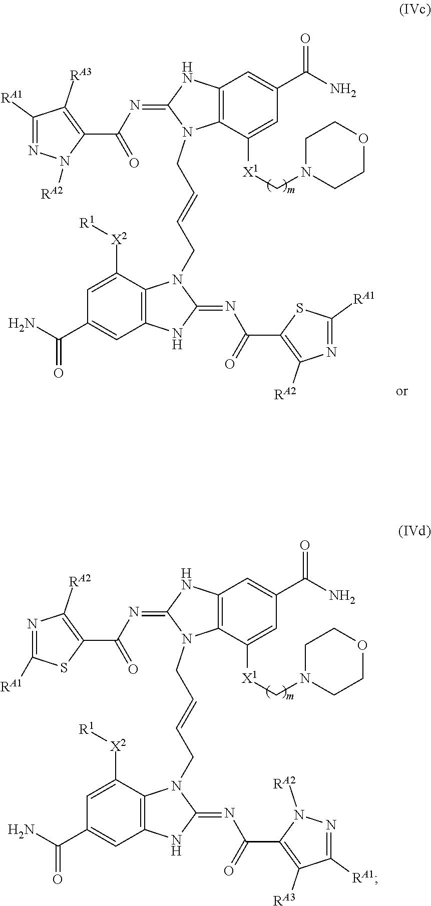
wherein R^{A1} is selected from the group consisting of methyl, ethyl, n-propyl and isopropyl; R^{A2} is selected from the group consisting of methyl, ethyl, n-propyl and isopropyl; and R^{A3} is selected from the group consisting of hydrogen, fluorine, chlorine, methyl and ethyl;
X¹ is O or S; X² is O or S;
m is 1, 2, 3, 4, 5 or 6;
R¹ is 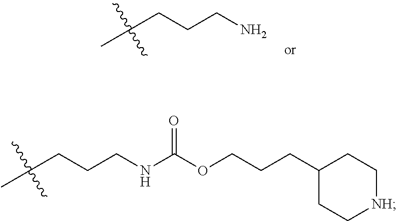 [SMILES: *CCCN ; *CCCNC(=O)OCCCC1CCNCC1]
R² is selected from the group consisting of —C₁₋₆ alkyl, —C(O)—C₁₋₆ alkylene-NH₂, —C(O)—C₁₋₆ alkylene-NH—C₁₋₆ alkyl, —C(O)O—C₁₋₆ alkylene-NH₂, —C(O)O—C₁₋₆ alkylene-NH—C₁₋₆ alkyl, —C(O)NH—C₁₋₆ alkylene-NH₂, —C(O)NH—C₁₋₆ alkylene-NH—C₁₋₆ alkyl, —C₁₋₆ alkylene-NH₂, —C₁₋₆ alkylene-NH—C₁₋₆ alkyl, —C(O)—C₁₋₆ alkylene-piperazinyl, —C(O)O—C₁₋₆ alkylene-piperazinyl, —C(O)NH—C₁₋₆ alkylene-piperazinyl, —C₁₋₆ alkylene-piperazinyl, —C(O)—C₁₋₆ alkylene-piperidyl, —C(O)O—C₁₋₆ alkylene-piperidyl, —C(O)NH—C₁₋₆ alkylene-piperidyl and —C₁₋₆ alkylene-piperidyl; wherein one or two carbon atoms in an alkylene group are adapted to be replaced with an oxygen atom.

15. The compound of claim 9, wherein the compound is selected from the group consisting of:

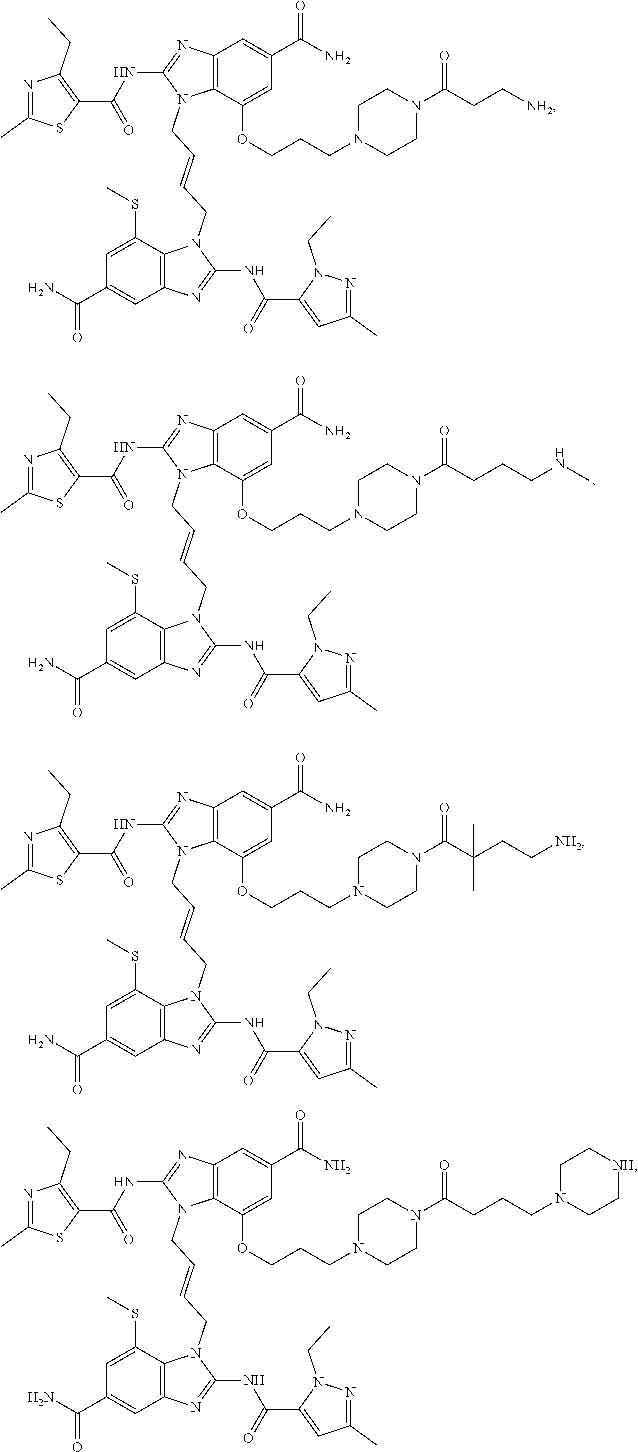

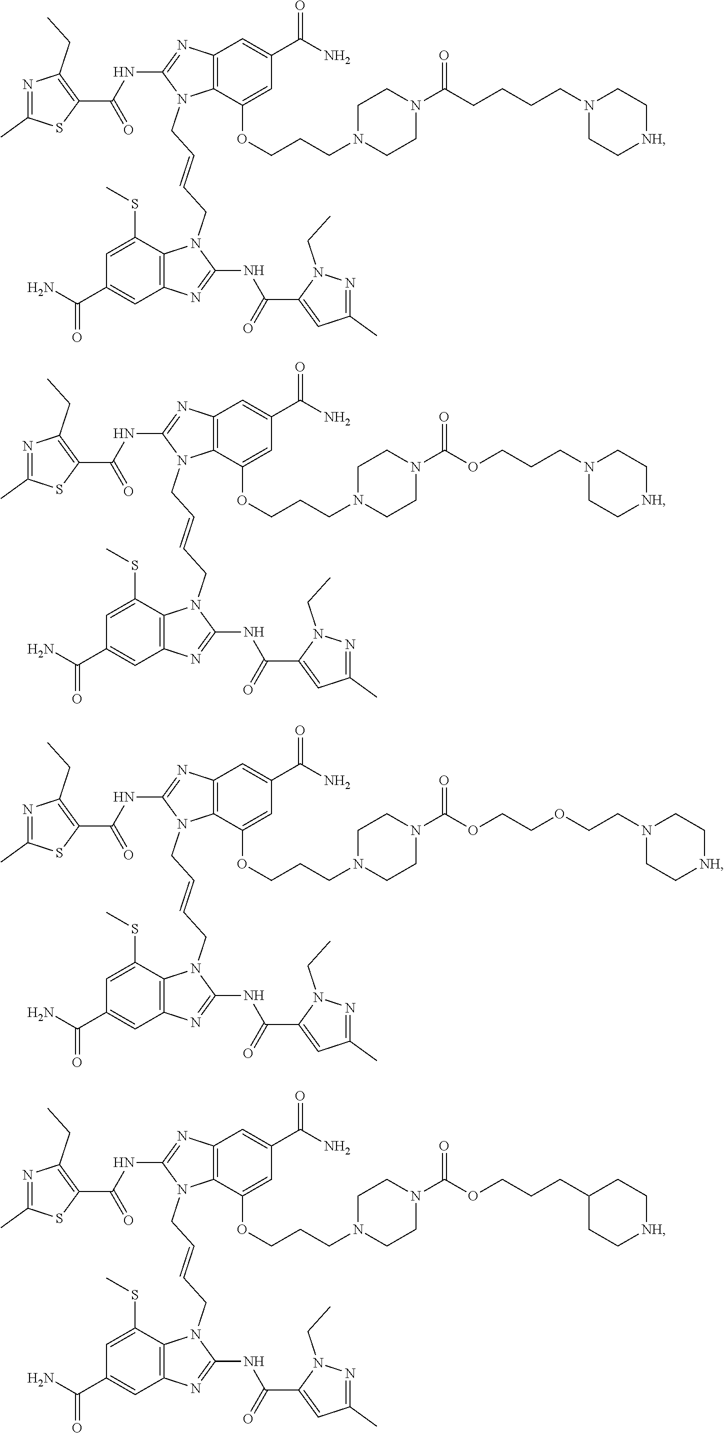

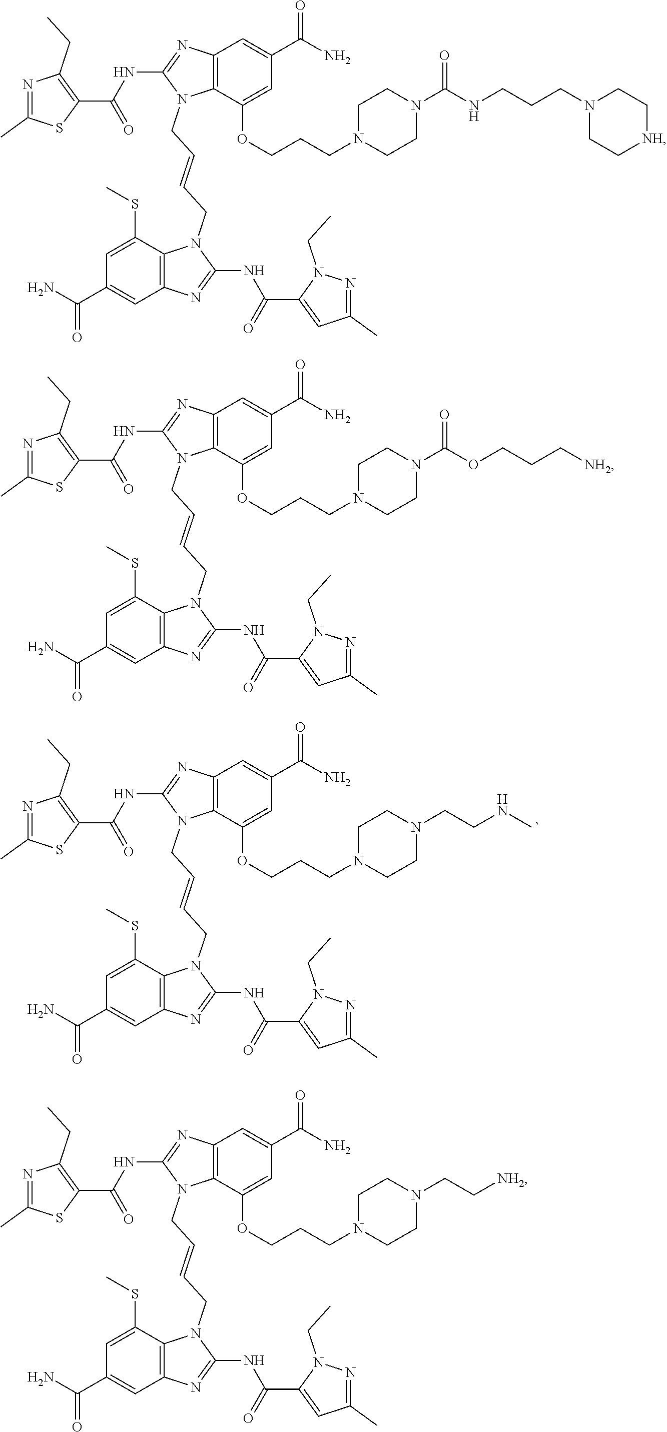

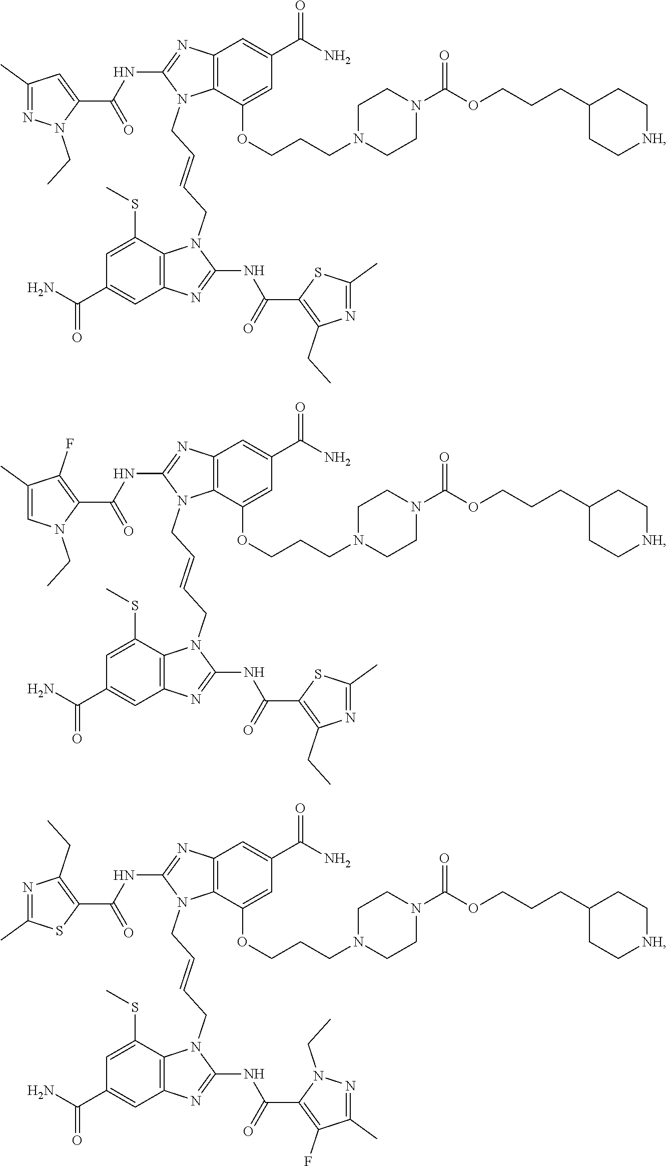

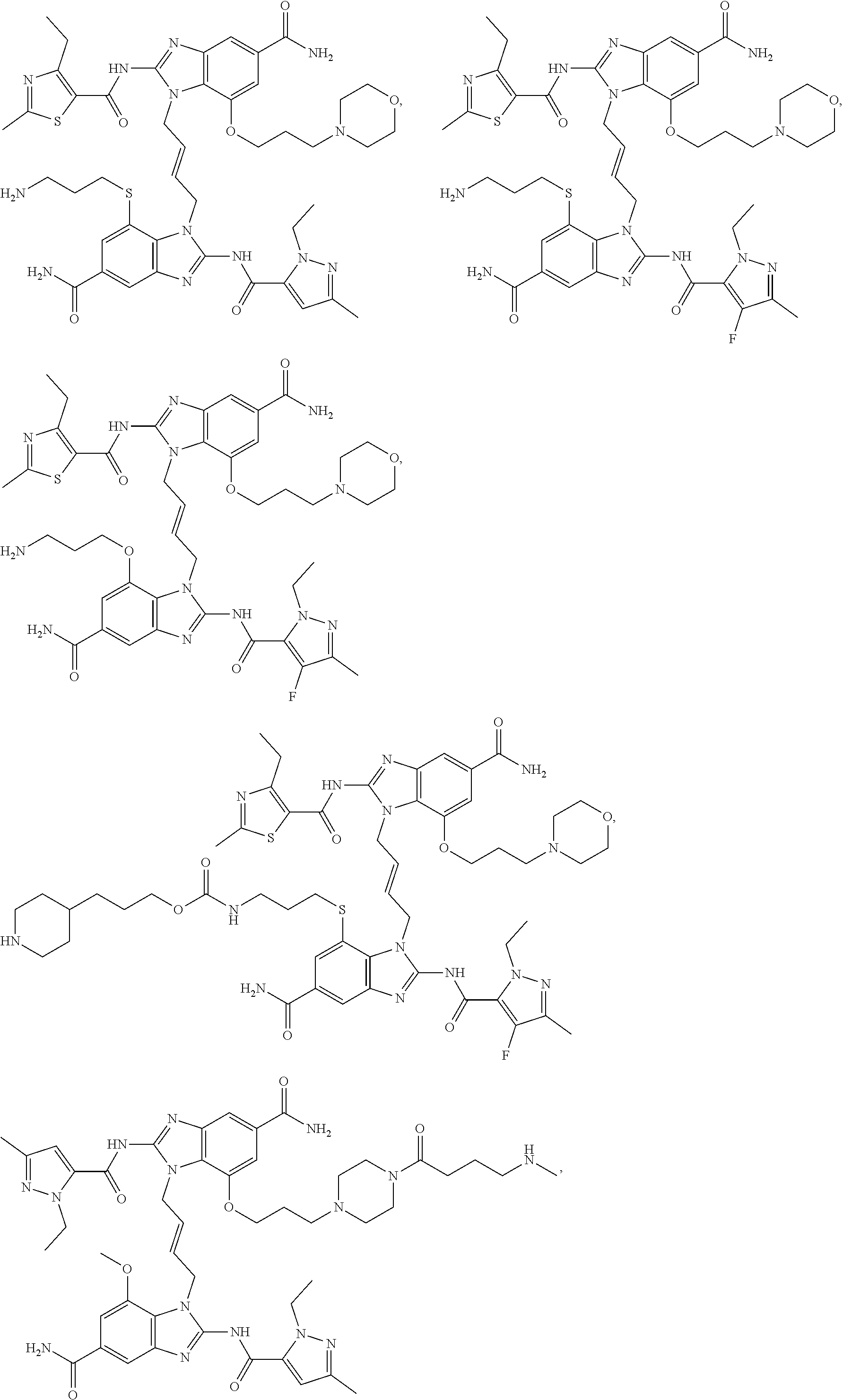

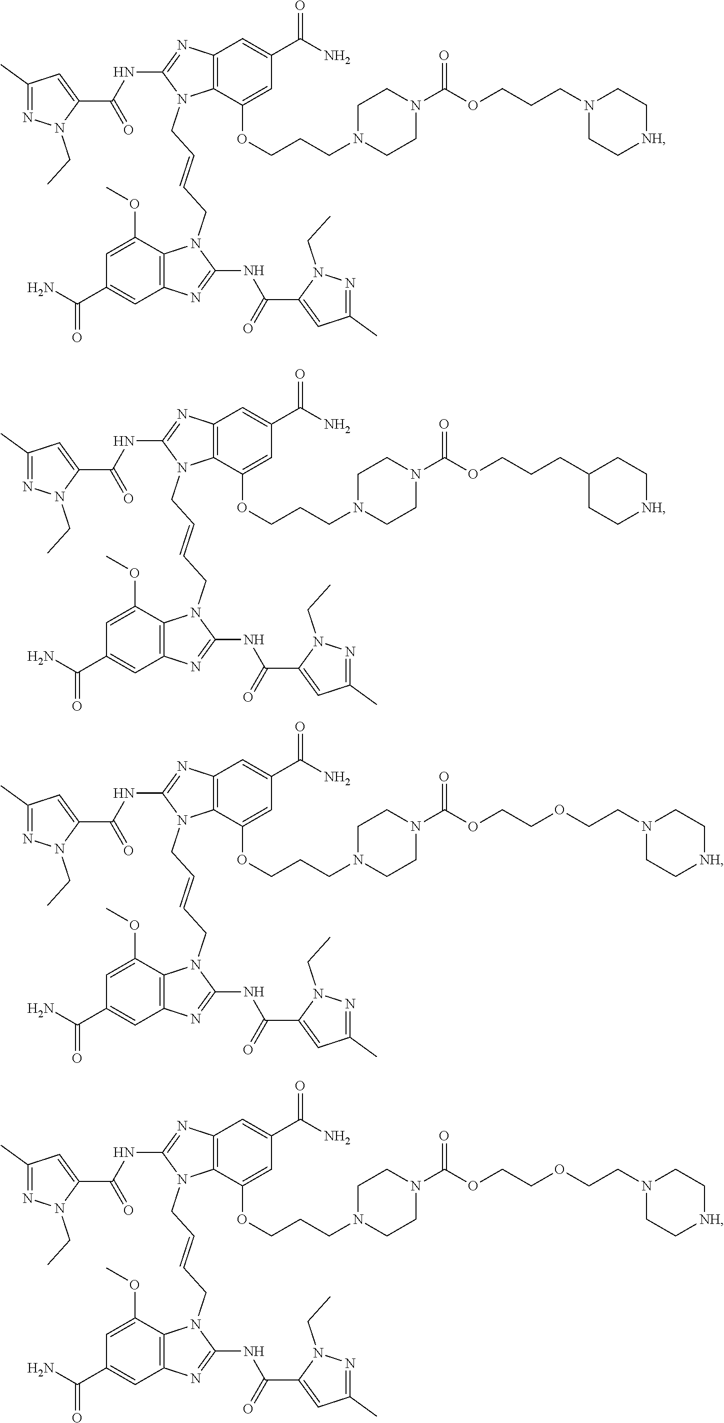

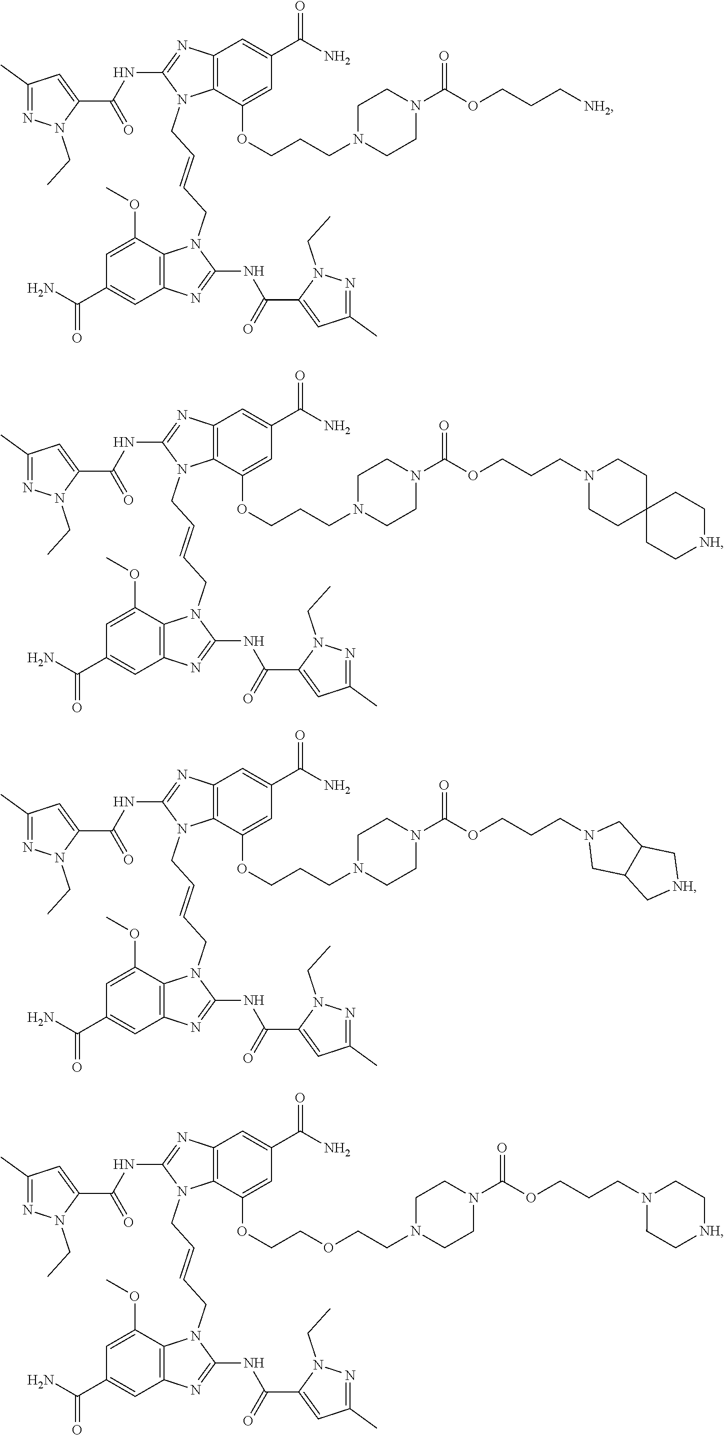

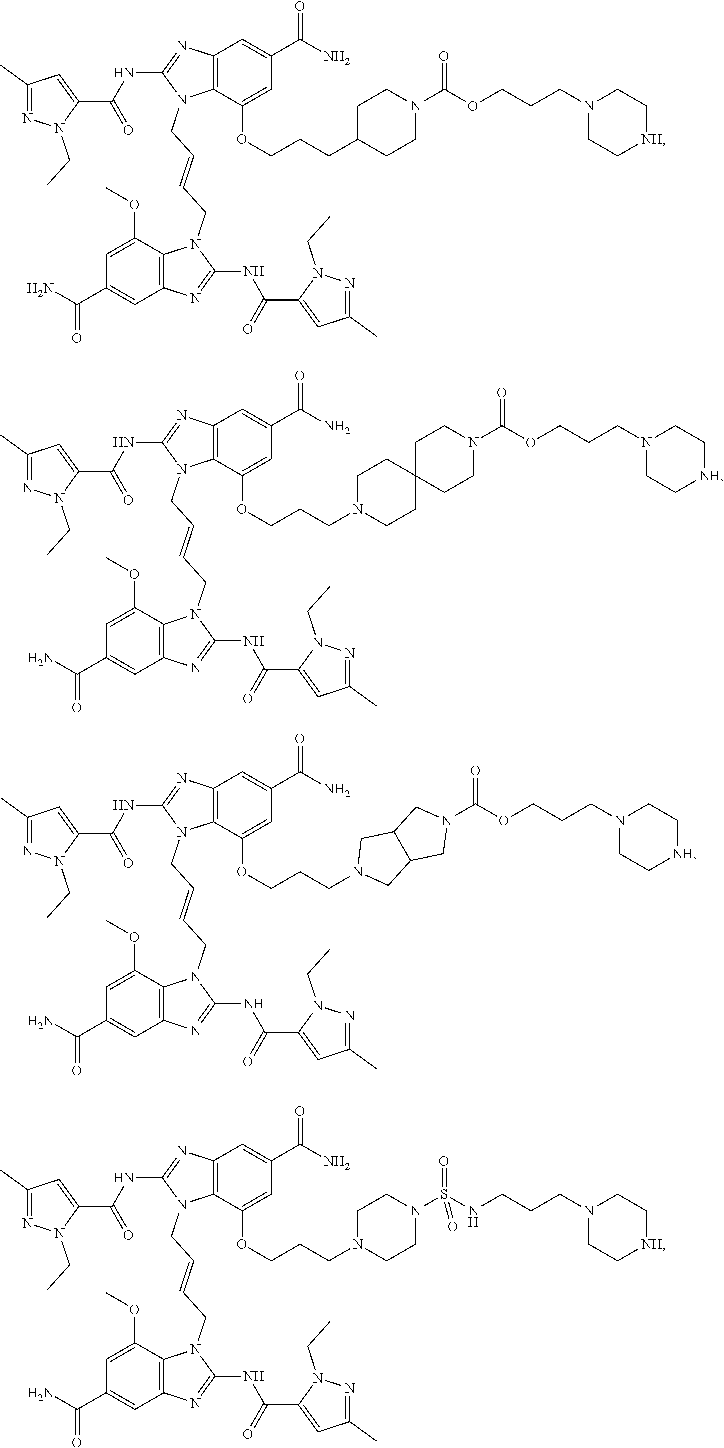

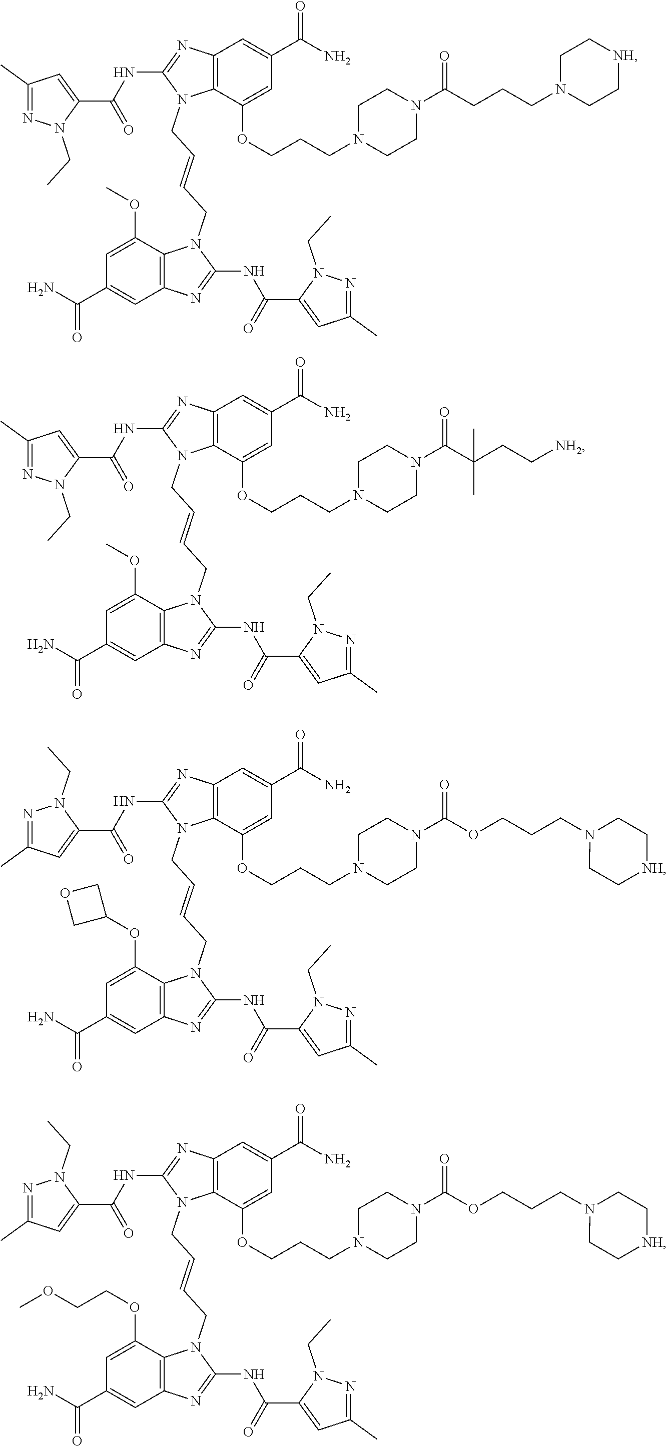

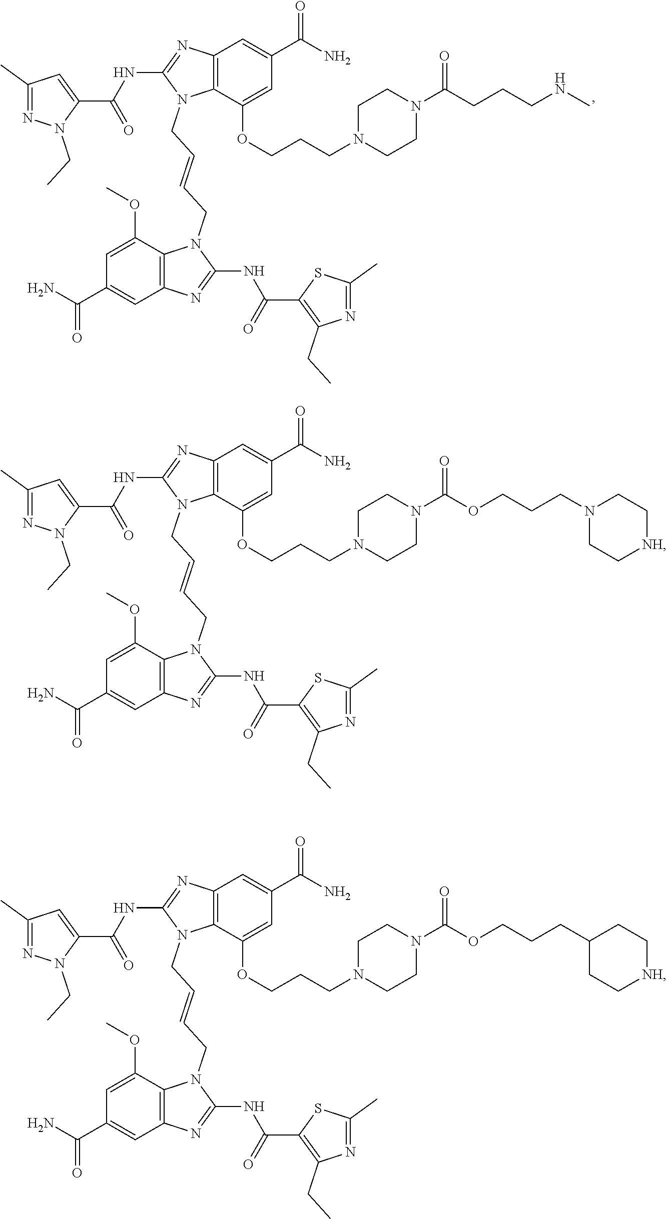

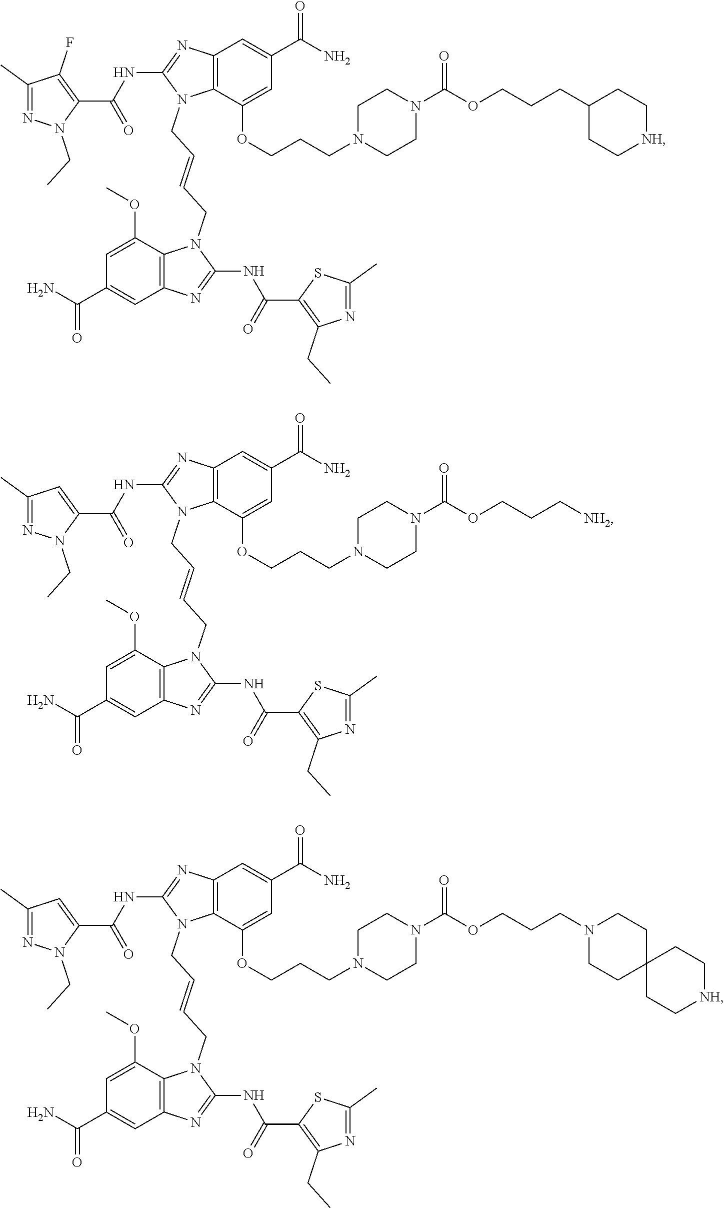

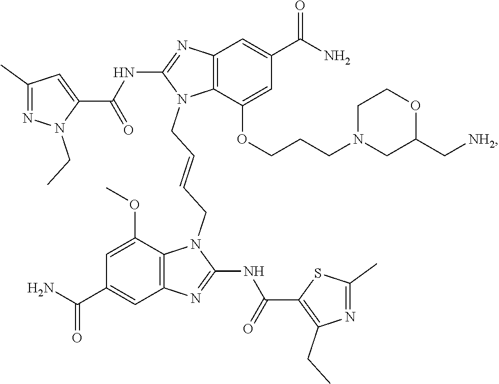 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCOC(CN)C3)c21]

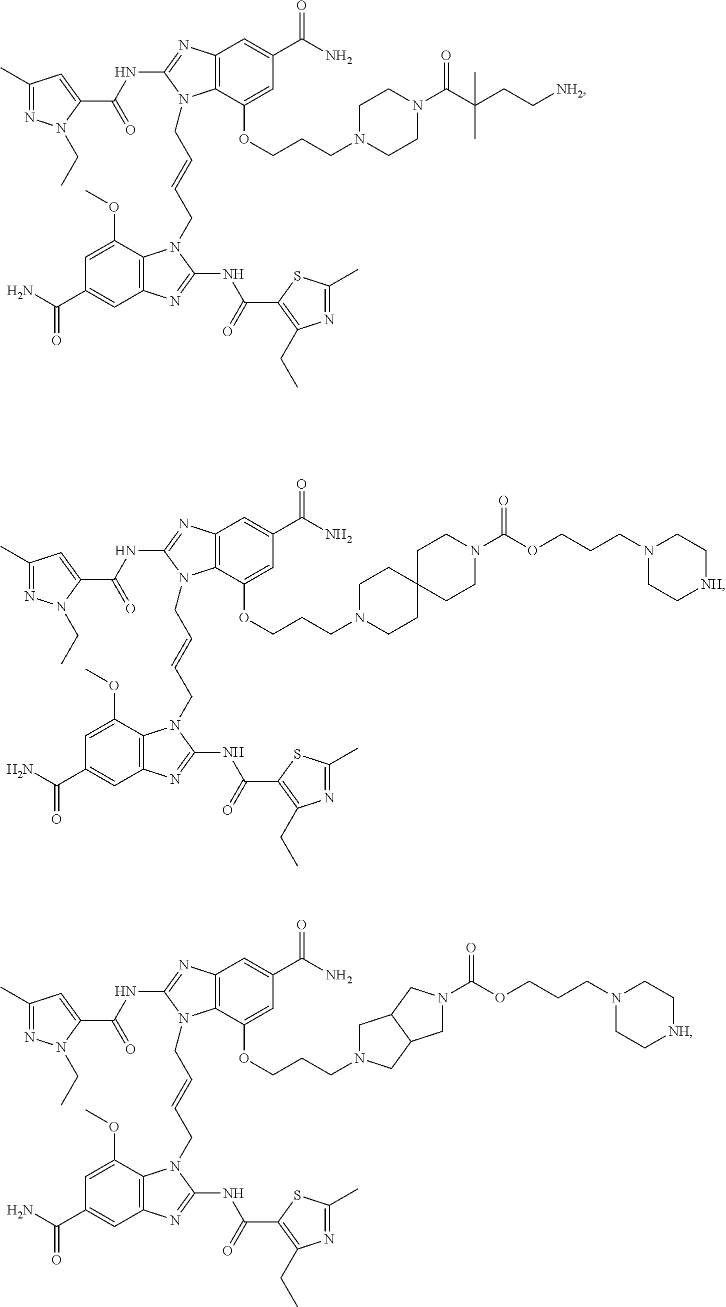 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)C(C)(C)CCN)CC3)c21 ; CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC4(CC3)CCN(C(=O)OCCCN3CCNCC3)CC4)c21 ; CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4CN(C(=O)OCCCN5CCNCC5)CC4C3)c21]

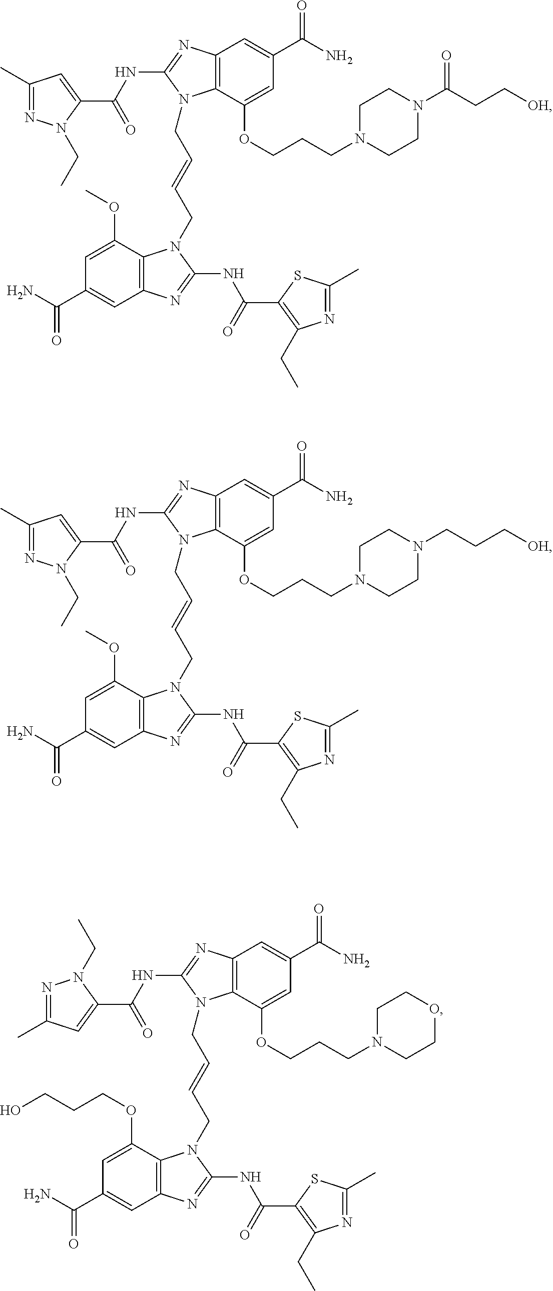 [SMILES: CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCO)CC3)c21 ; CCc1nc(C)sc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCCO)CC3)c21 ; CCc1nc(C)sc1C(=O)Nc1nc2cc(C(N)=O)cc(OCCCO)c2n1C/C=C/Cn1c(NC(=O)c2cc(C)nn2CC)nc2cc(C(N)=O)cc(OCCCN3CCOCC3)c21]

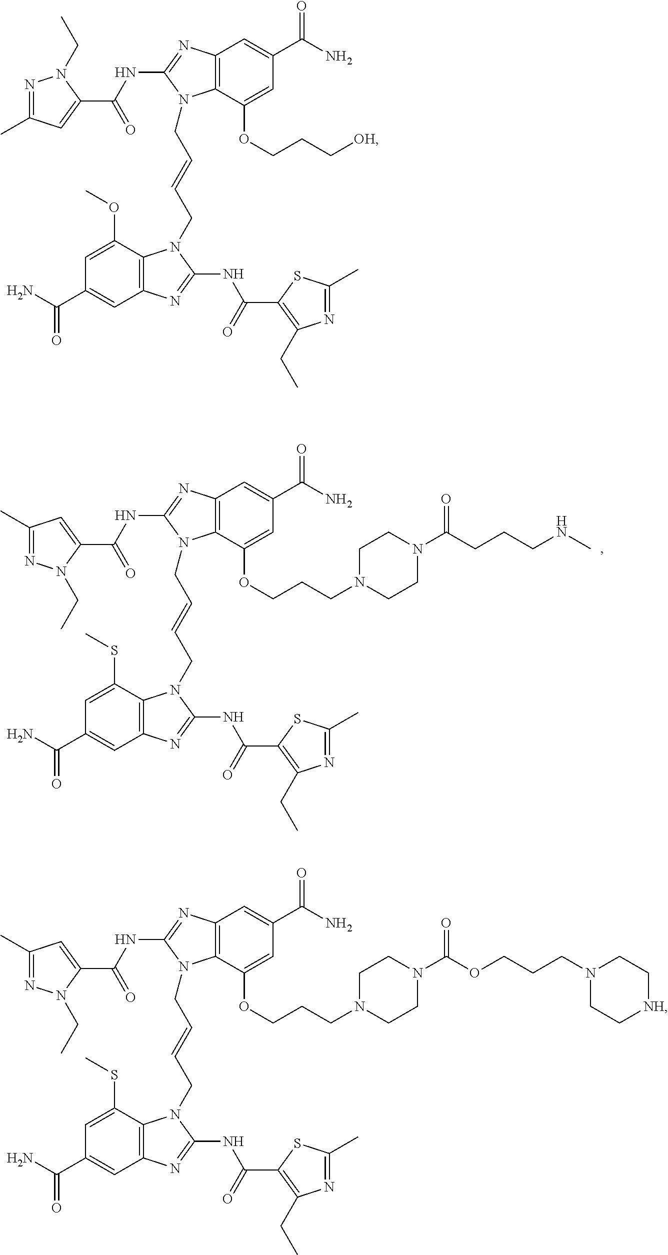

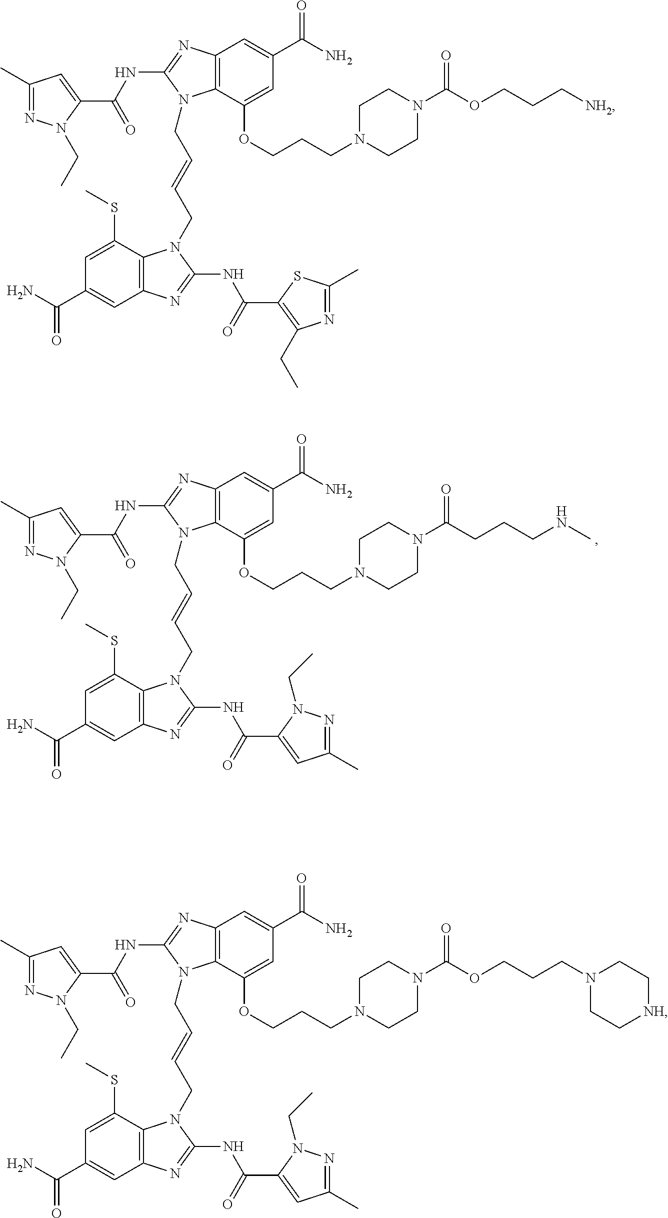

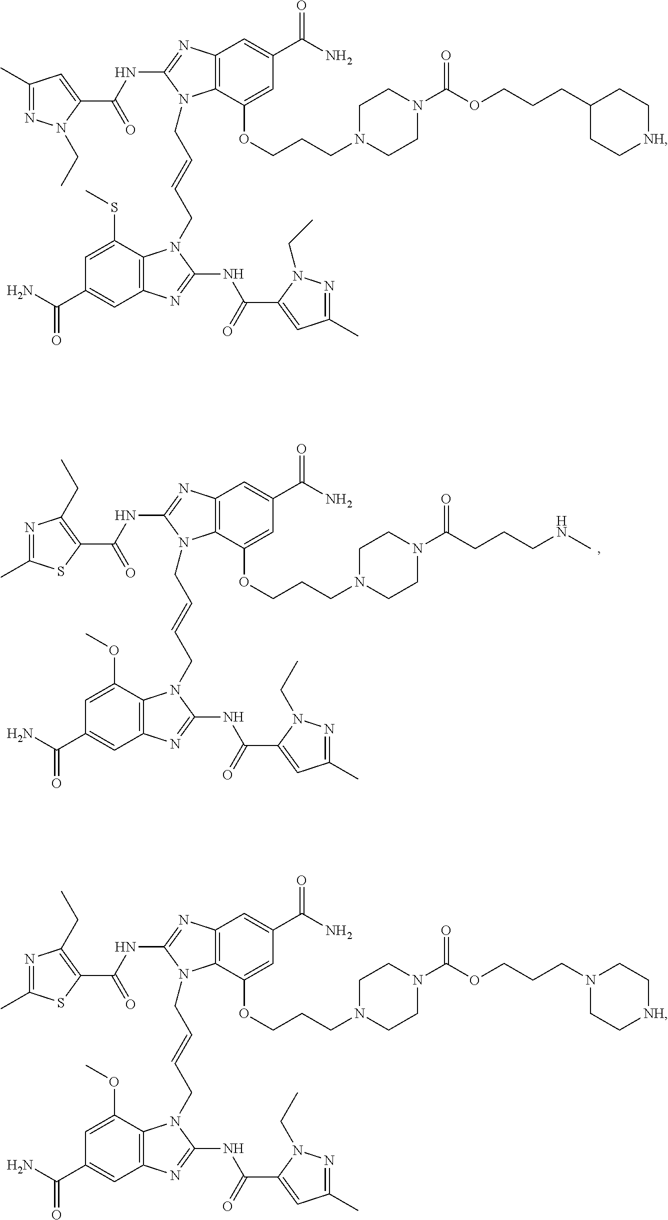

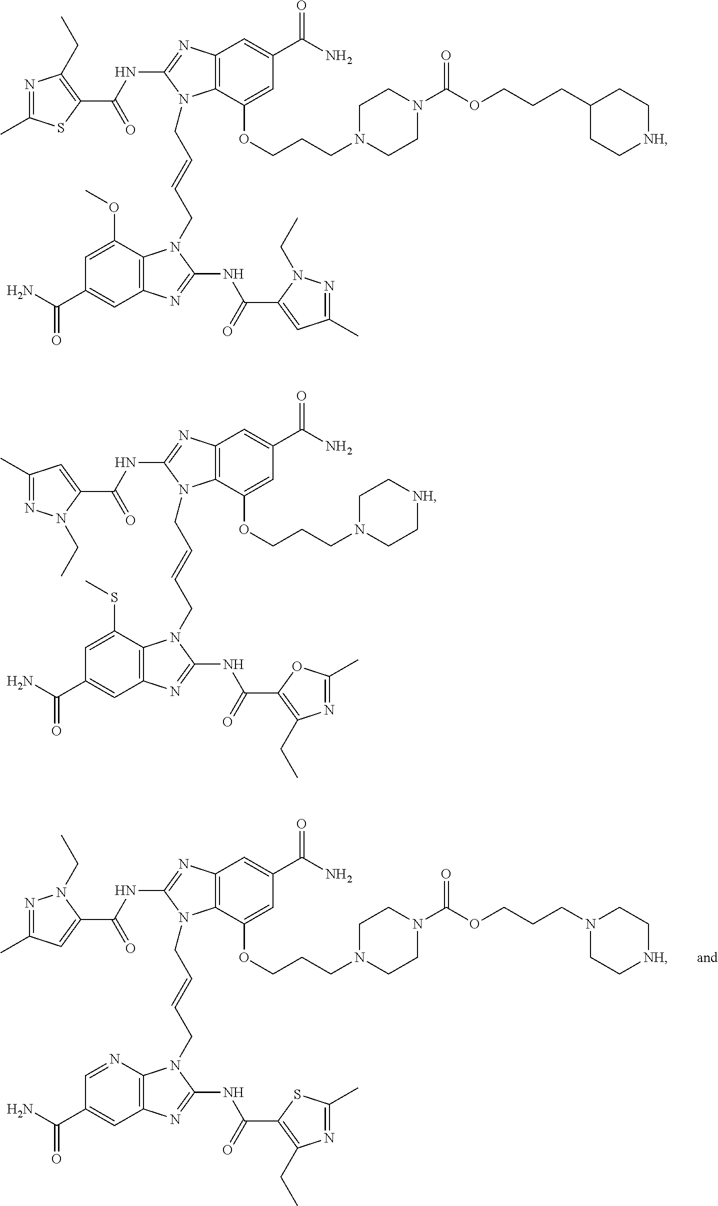

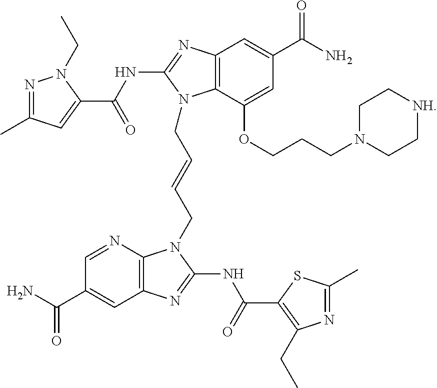 [SMILES: CCc1nc(C)sc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

16. The compound of claim 11, wherein the compound is selected from the group consisting of:

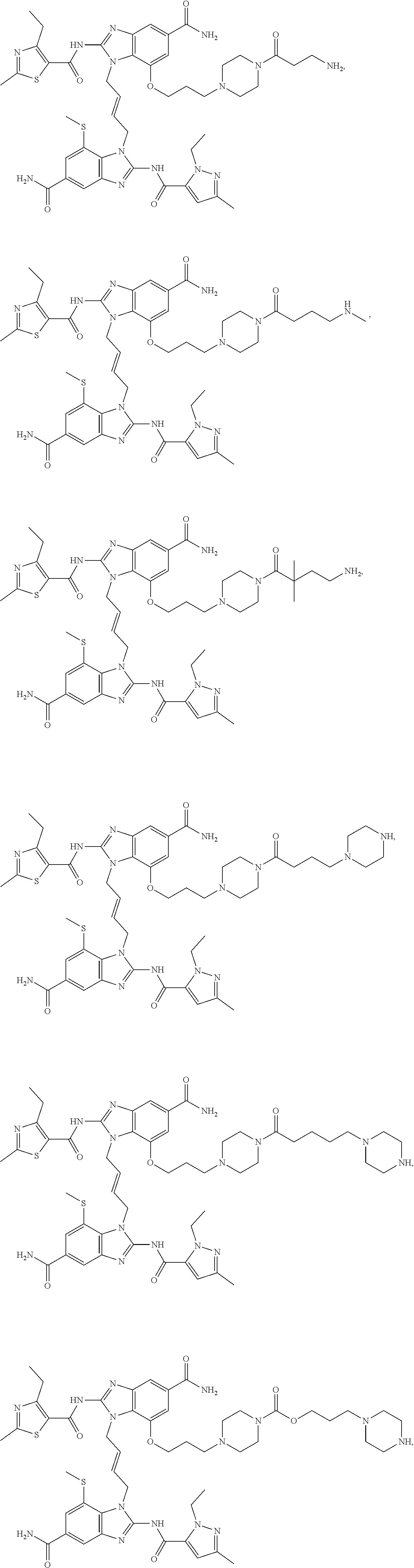

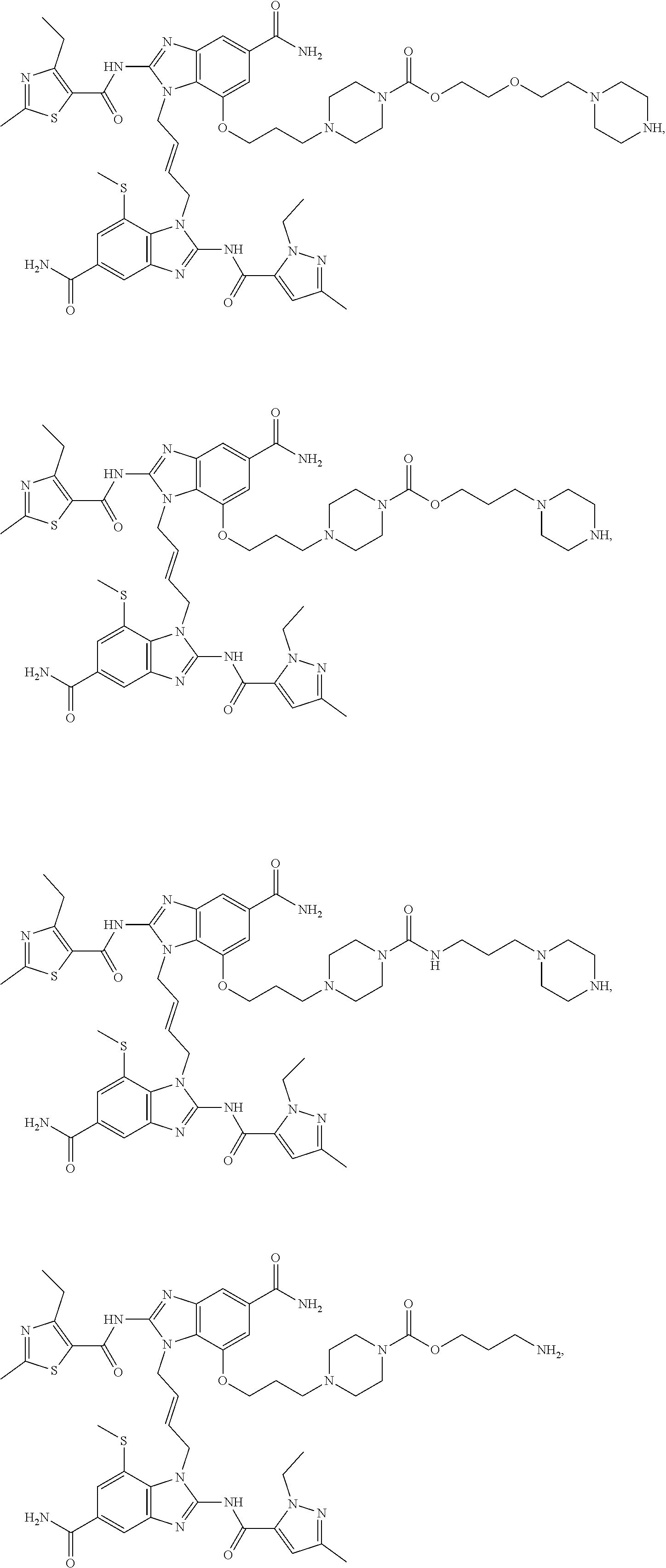 [SMILES: CCc1nc(C)sc1C(=O)Nc1nc2cc(C(N)=O)cc(OCCCN3CCN(C(=O)OCCOCCN4CCNCC4)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)OCCCN4CCNCC4)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)NCCCN4CCNCC4)CC3)c2n1C/C=C/Cn1c(NC(=O)c2cc(C)nn2CC)nc2cc(C(N)=O)cc(SC)c21 ; CCc1nc(C)sc1C(=O)Nc1nc2cc(C(N)=O)cc(OCCCN3CCN(C(=O)OCCCN)CC3)c2n1C/C=C/Cn1c(NC(=O)c2cc(C)nn2CC)nc2cc(C(N)=O)cc(SC)c21]

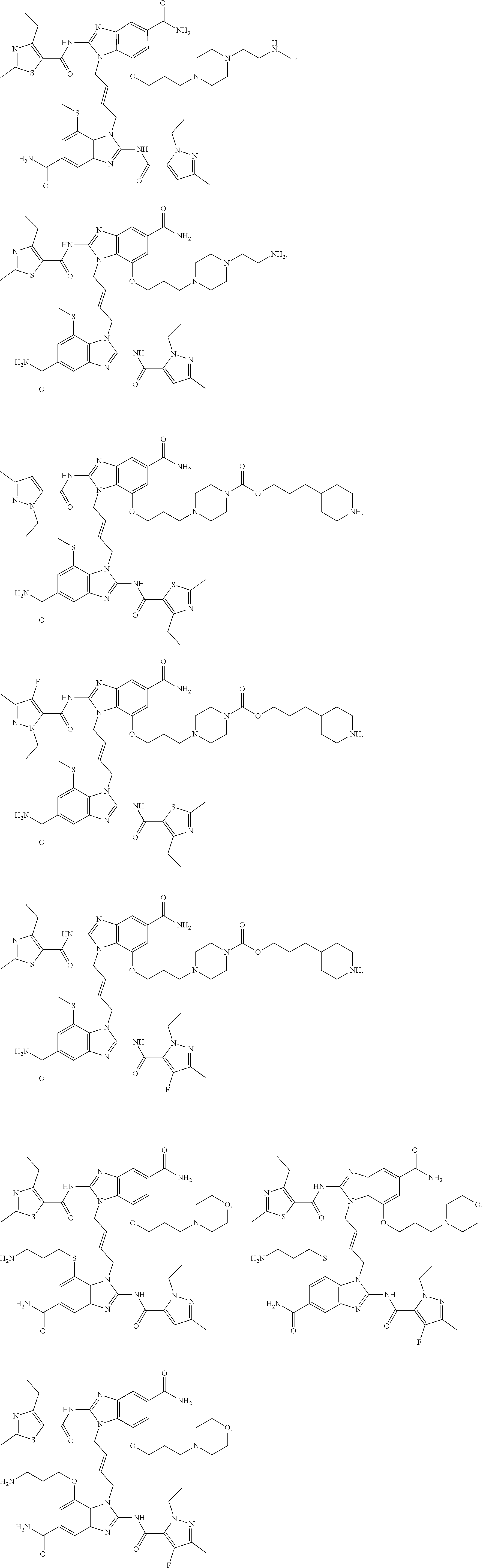

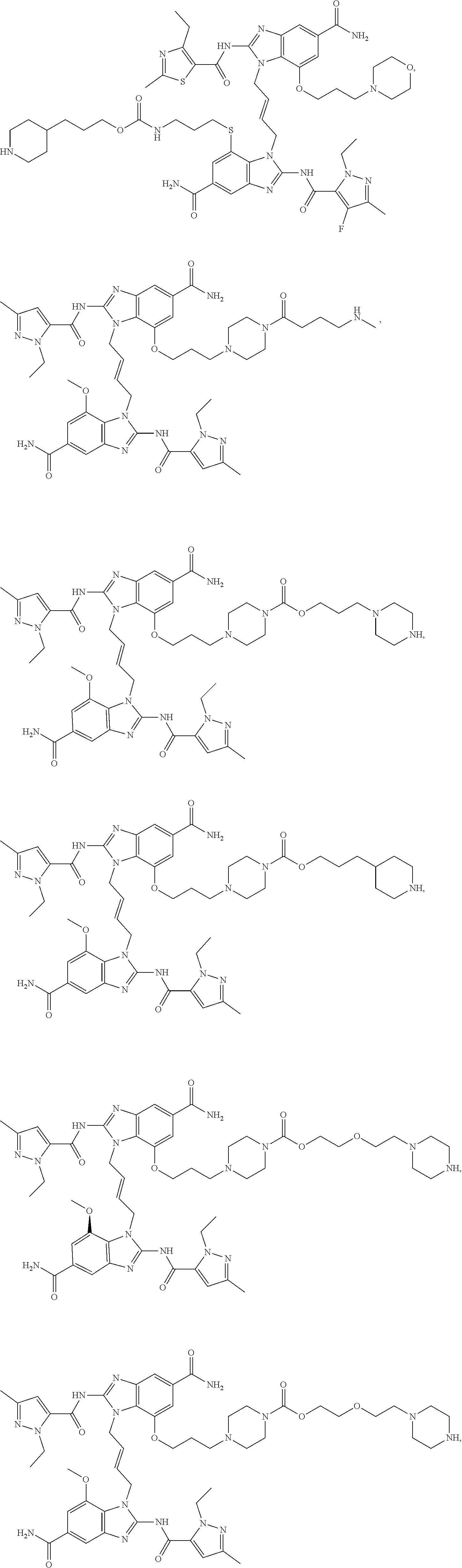

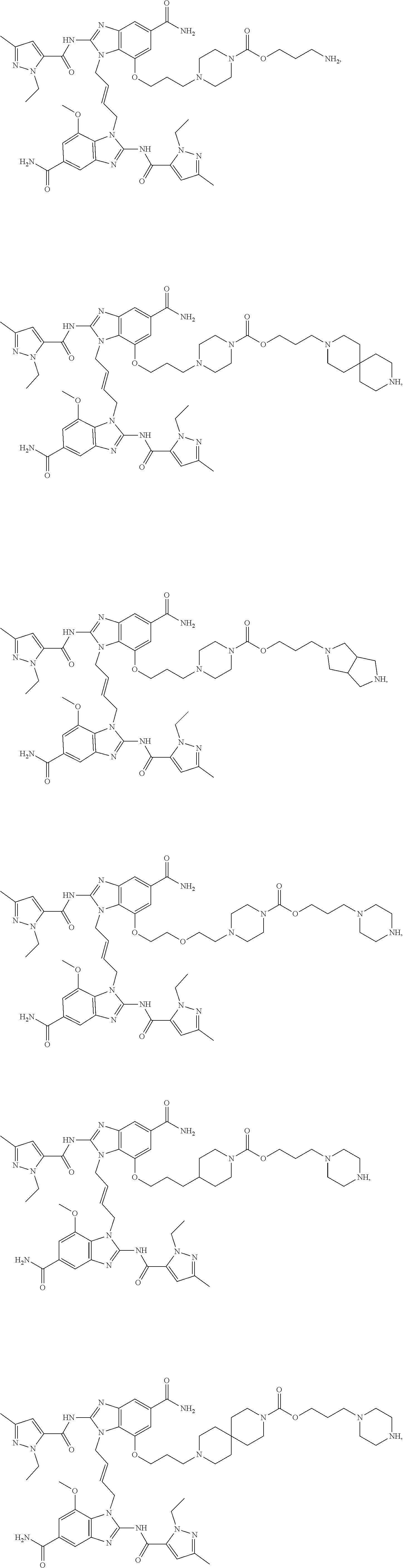

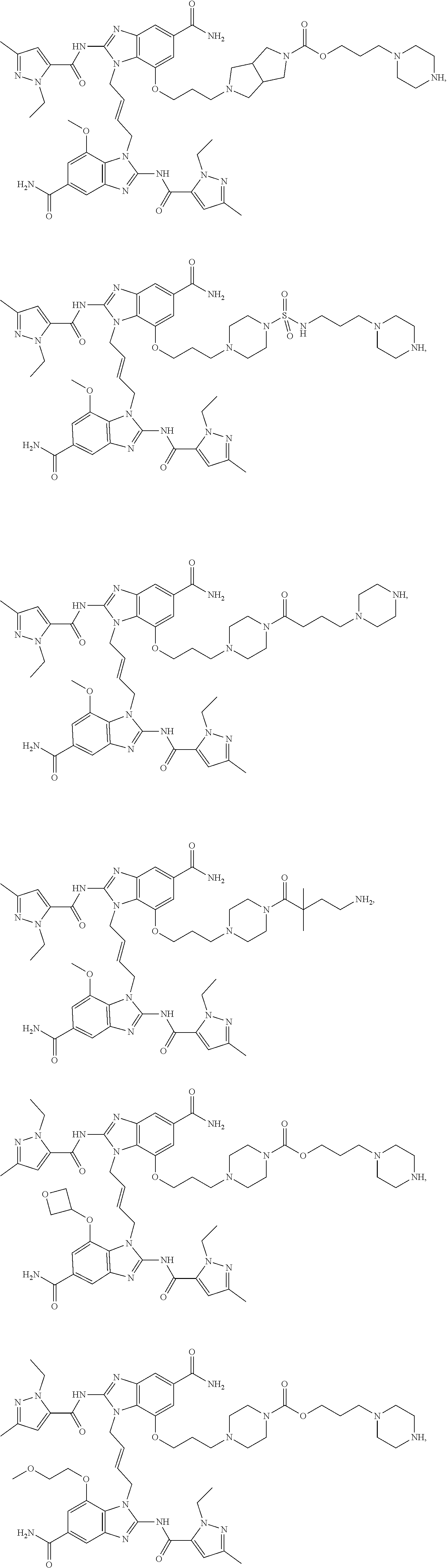

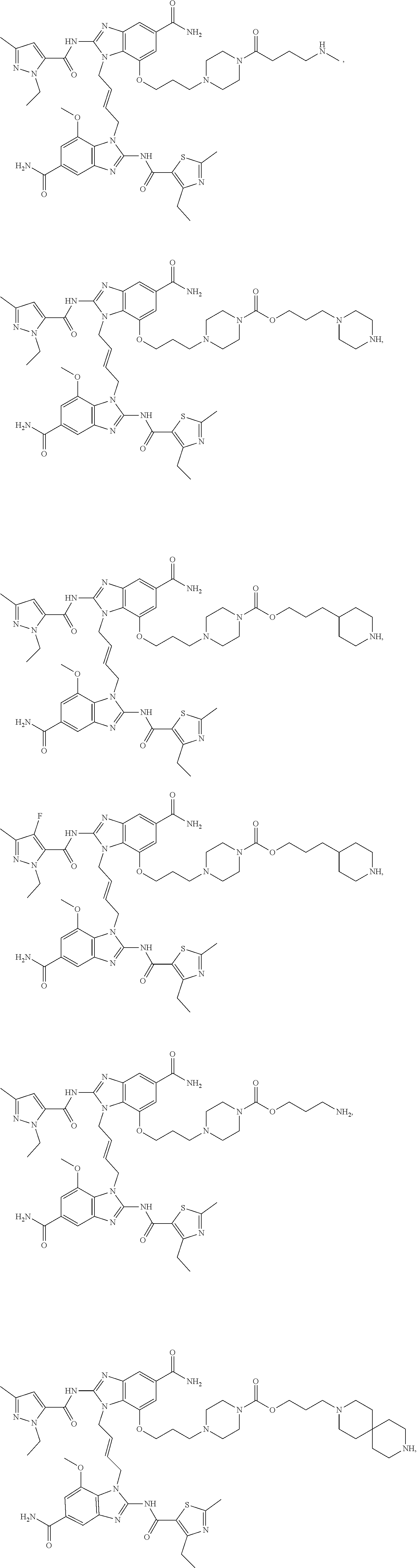

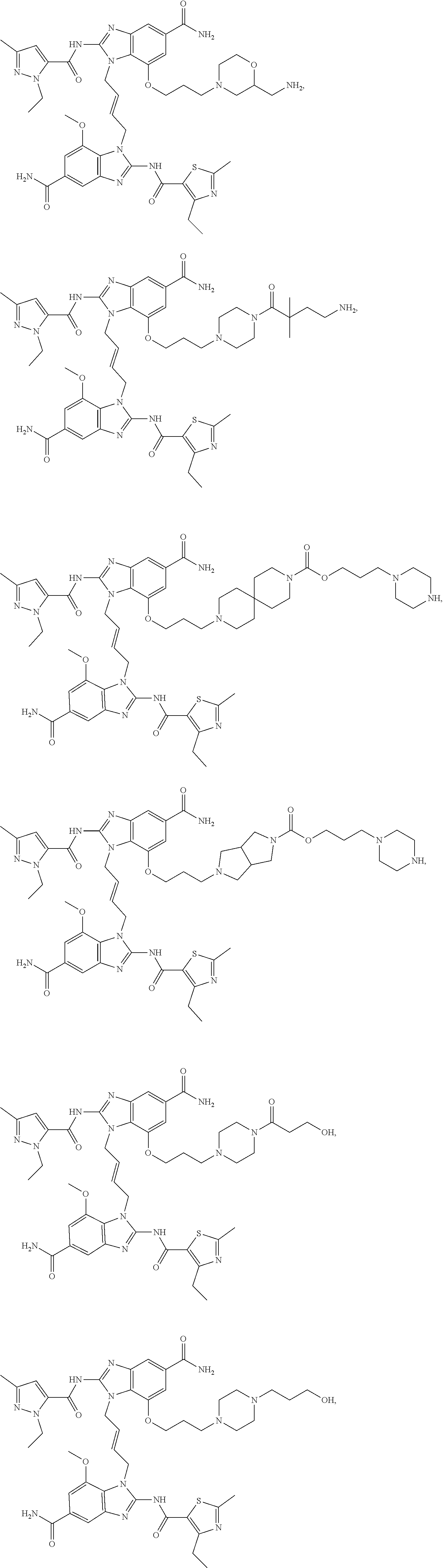

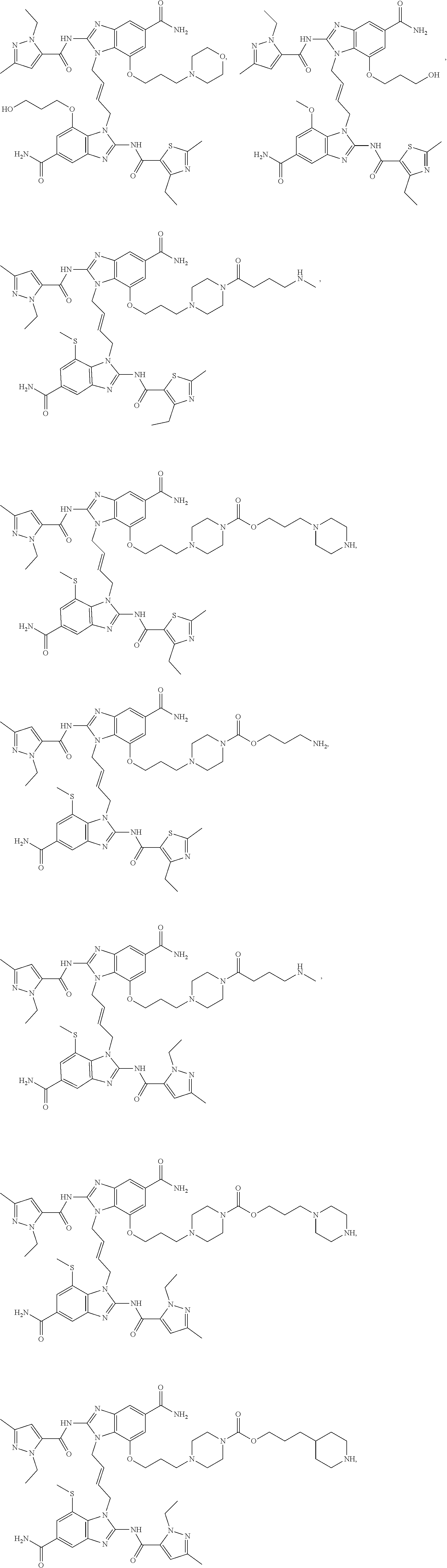

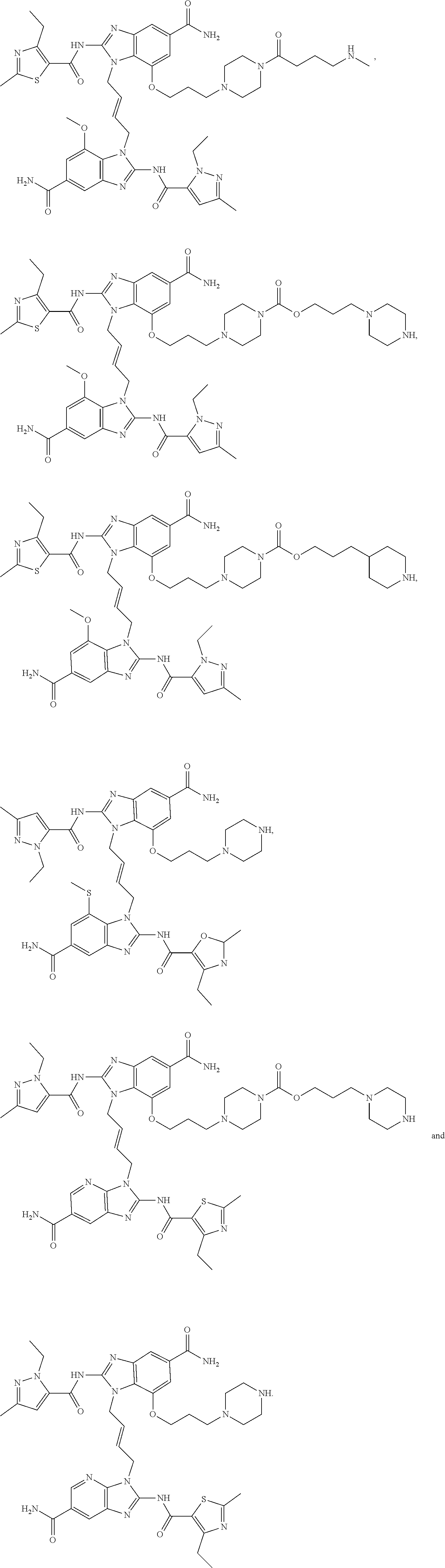

17. A method for preparing an antibody drug conjugate, comprising:
preparing the antibody drug conjugate from the compound-linker conjugate of claim 1;
wherein the compound-linker conjugate an intermediate of the antibody drug conjugate.

18. A method for preparing an antibody drug conjugate, comprising:
preparing the antibody drug conjugate from the compound of claim 9, or a deuterated compound, a stereoisomer or a pharmaceutically acceptable salt thereof,
wherein the compound, or a deuterated compound, a stereoisomer or a pharmaceutically acceptable salt thereof is an intermediate of the antibody drug conjugate.

19. An antibody drug conjugate, comprising:
the compound of claim 9, or a deuterated compound, a stereoisomer or a pharmaceutically acceptable salt thereof as a payload.

20. An antibody drug conjugate, comprising:
the compound of claim 11, or a deuterated compound, a stereoisomer or a pharmaceutically acceptable salt thereof as a payload.
